(19) United States
(12) Patent Application Publication
(10) Pub. No.: US 2004/0087500 A1
(43) Pub. Date: May 6, 2004

(54) Method of constructing three-dimensional structure of transmembrane protein

(76) Inventors: Hideaki Umeyama, Chiba (JP); Mitsuo Iwadate, Saitama (JP)

Correspondence Address: FOLEY AND LARDNER, SUITE 500, 3000 K STREET NW, WASHINGTON, DC, 20007, US

(21) Appl. No.: 10/380,196

(22) PCT Filed: Sep. 12, 2001
(86) PCT No.: PCT/JP01/07918

(30) Foreign Application Priority Data
Sep. 14, 2000 (JP) 2000-279155
Nov. 17, 2000 (JP) 2000-350378

(51) Int. Cl. A61K 38/00; G06F 19/00; G01N 33/48; G01N 33/50

(52) U.S. Cl. 514/12; 530/350; 702/19

(57) ABSTRACT

An object of the present invention is to provide a method for constructing the steric structure of an arbitrary 7-transmembrane G-protein-coupled receptor with good precision.

The essential feature of the present invention is a method for constructing the steric structure of a transmembrane protein, which comprises selecting the steric structure to be referred to from the known steric structures of 7-transmembrane G-protein-coupled receptors using a database of amino acid sequences of the receptors (GCRDb) as a profile, performing alignment of a desired protein having an unknown steric structure, and producing the steric structure of the desired protein having an unknown steric structure as a G-protein-coupled receptor to construct the steric structure of a transmembrane protein.

### TECHNICAL FIELD

The present invention relates to a method for preparing the steric structure of a protein. More particularly, this invention relates to a method for preparing the steric structure of a 7-transmembrane G-protein-coupled receptor with good precision, a database and the like containing atomic coordinates defining the steric structure of a protein obtained by the method.

It is said that a 7-transmembrane G-protein-coupled receptors account for more than half of receptors which have been cloned up to now, and the method and the database and the like of the present invention are extremely useful in the field of molecular design of medicines and agricultural chemicals.

### BACKGROUND ART

An approach has made it possible to construct the steric structure of a desired protein having an unknown steric structure based on information of the alignment with the desired protein wherein the alignment is obtained by utilizing information about proteins having a known steric structure. This approach is usually called homology modeling. Although the precision of the steric structure constructed by homology modeling has been remarkably improved recently, there are still many problems to be solved.

On the other hand, since a membrane protein is difficult to crystallize experimentally, it has been difficult to apply X-ray crystal diffraction which is a main stream for determining a structure in structural biology. As an attempt has been tried to determine a structure of a membrane protein with an electron microscope from a two-dimensional crystal, a structure with some extent of precision is obtained to an extent of a precision and, therefrom, it has become possible to construct a model. However, its precision has not yet reached that of single crystal diffraction by X-ray, and structural information of a loop part which is not a transmembrane region and of a side chain part of an amino acid has been poor.

Among membrane proteins, a 7-transmembrane G-protein-coupled receptor (guanine nucleotide-binding protein-coupled receptor; hereinafter this may be abbreviated as “GCPRs” or “GPCR”) is a protein which has the common nature of activating a G-protein when receives various extracellular stimulations such as calcium ion, amines, various hormones, peptides and proteins, and transmitting information into cells, which and has an important function in a living body. The common characteristics of structures of GPCRs are 7-transmembrane α helix structures and loop structures of a variety of lengths connecting them, and it is considered that these common characteristics determine the natures of GPCRs. As seen in trypsins of a serine protease having the characteristic relationship between its structure and function, also in GPCRs, it is presumed that the above-mentioned structural characteristics serve as an information transmitting mechanism.

Since GPCRs are particularly difficult to crystallize, information about their steric structures is poor. Even under this situation, regarding the model construction, for example, there are reports such as modeling combined with a site-specific mutation experiment of a CB (2) receptor (Eur J Pharmacol 2000 Jul. 28; 401 (1): 17-25), modeling combined with a site-specific mutation experiment of a CCK (1) receptor (Eur J Pharmacol 2000 Jul. 21; 400 (2-3): 185-194), a theoretical model referring to the function (Protein Eng 2000 July; 13 (7): 477-90), construction of a NK2 receptor combined with a mutation experiment (Biochem J 1999 Apr. 1; 339 (Pt 1): 55-61), modeling regarding frog rhodopsin (J Mol Biol 1998, Aug 28; 281 (4): 741-54), modeling of a transmembrane receptor combined with a site-specific mutation (Biophys J 1998 March; 74 (3): 1203-14) and the like. However, the satisfactory results have not been necessarily obtained in these modelings.

The cause why a decisive steric structure of a membrane protein has not been proposed up to now is that a membrane protein hardly becomes a single crystal due to the nature of the membrane protein orienting in a membrane of a lipid bilayer and thus it is difficult to apply X-ray diffraction. Since a membrane protein is fundamentally of α helix and there is no diversity in its steric structure, there is the background that it is difficult to determine the structure by normal approaches of NMR and a distance geometry method. For that reason, the structural information of a membrane protein is generally analyzed by electron beam diffraction of a two-dimensional crystal orienting in a membrane, but its resolution is 6 to 7 angstrom, not reaching the precision of crystal diffraction at all.

In addition, a modeling method combined with a site-specific mutation is a method of performing substitution of an amino acid at a site which is considered to be important for the activity, and examining the influence on the activity of an agonist or an antagonist. Although this method has such an advantage that detailed discussion can be made as to the surroundings of the active site, a model can not be proposed until a mutated strain of a bacterium producing a number of substituted bodies is provided, until a reaction mechanism of stimulation to GPCRs is well known and, additionally, until geometric information is obtained, and operations are troublesome and, thus, it can hardly be said that this method will give the results with good precision.

Like this, as for a membrane protein, there has been only a scarcely minute structure obtained by electron beam diffraction, or a structure from bacteriorhodopsin or the like which is not functionally equal to GPCRs, and the situation has been such that there were conventionally no choices for a reference protein. In addition, since details of a side chain of a reference protein are not determined, the situation has not been such that the performance of a software is fully displayed despite that it is possible to determine even the detailed structure of a side chain by the software. In addition, conventionally, a protein with low homology including GPCRs could not be modeled and, even when there is one experimentally determined structure, it is judged that construction of a model is impossible at an alignment stage before construction of a model in many cases.

On the other hand, it is considered that rhodopsin, one of a GPCRs family accounting for a majority of membrane proteins, is activated by light stimulation and transmits information. Recently, it has been reported that the steric structure of this protein (PDB ID:1F88) including a side chain is determined in detail by X-ray crystal analysis (Science, vol. 289, 4 August 2000, 739-745).

### DISCLOSURE OF THE INVENTION

In view of the above-mentioned situation, the present invention has been achieved in order to provide a method for constructing the steric structure of arbitrary GPCRs with good precision, a database and the like of atomic coordinates defining the steric structure of GPCRs which can be utilized for molecular design of medicines and agricultural chemicals.

The present inventors have intensively studied in order to attain the above-mentioned object and, as a result, found that the precision of the steric structure model of arbitrary GPCRs can be remarkably improved by using a database of amino acid sequences of GPCRs as a profile and referring to the steric structure of rhodopsin (PDB ID:1F88), compared to the time when only a conventional structure having a different helix arrangement and not being classified into GCRDb of bacteriorhodopsin has been obtained. That is, we found that the steric structure of arbitrary GPCRs can be constructed with good precision by using the database of amino acid sequences of PCRs “GCRDb” as a motif profile of PSI-BLAST, and performing alignment referring to the steric structure of rhodopsin (PDB ID:1F88). The present invention is completed based on these findings.

That is, according to the method of the present invention, there is provided (1) a method for constructing the steric structure of a transmembrane protein, which comprises selecting a protein having the steric structure to be referred to from the known steric structures of 7-transmembrane G-protein-coupled receptors using a database of amino acid sequences of the receptors as a profile, performing alignment of a desired protein having an unknown steric structure, and producing the steric structure of the desired protein having an unknown steric structure as a G-protein-coupled receptor.

According to a preferable aspect of the present invention, there are provided (2) the method described in the above (1), wherein alignment is performed by selecting a protein having the steric structure to be referred to from a database of known steric structures of G-protein-coupled receptors, and by juxtaposing an amino acid sequence of the selected reference protein and an amino acid sequence of the desired protein; (3) the method described in (2), wherein a structure to be referred to is the steric structure of rhodopsin (PDB ID:1F88); (4) the method described in any one of the above (1) to (4), wherein the database of amino acid sequences used as a profile is selected from the group consisting of GCRDb, GPCRDB, ExPASy and ORDB; and (5) the above-mentioned method, wherein the database of amino acid sequences is used as a motif profile of PSI-BLAST.

According to a preferable aspect of the present invention, there are provided (6) the method described in above (5), wherein in the alignment performed by PSI-BLAST, the identity between a final site-specific score matrix and the reference protein is calculated as an E value and, when the E value has a value of 0.1 or smaller, the steric structure is produced, (7) the method described in any one of the above (1) to (6), wherein regarding given alignment information, re-alignment is performed in view of a combination of disulfide bonds, and the steric structure is produced based on this re-alignment information, and (8) the method described in any one of the above (1) to (7), wherein production of the steric structure comprises obtaining a coordinate from the steric structure which is referred to with respect to Cα atoms in an amino acid based on the resulting alignment information, optimizing an atomic coordinate of Cα so as to minimize an objective function, adding other atoms of a main chain to an optimized atomic coordinate of Cα to optimize an atomic coordinate of a main chain so as to minimize an objective function, and adding other atoms of a side chain to an optimized atomic coordinate of a main chain to optimize so as to minimize an objective function.

According to another aspect of the present invention, there are provided (9) an atomic coordinate defining the steric structure of a 7-transmembrane G-protein-coupled receptor obtained by the method described in any one of the above (1) to (8), (10) a computer readable recording medium in which the atomic coordinate described in the above (9) is recorded, and (11) a database, which comprises the atomic coordinate described in the above (9).

According to still another aspect of the present invention, there is provided (12) a method of presuming the function of a protein, which method comprises selecting a protein having the steric structure to be referred to from the known steric structures of 7-transmembrane G-protein-coupled receptors using a database of amino acid sequences of the receptors as a profile, performing alignment of a desired protein having the unknown structure and, when the steric structure of the desired protein can be produced, judging that the desired protein is a 7-transmembrane G-protein-coupled receptor.

According to a preferable aspect of the present invention, there is provided (13) the method described in the above (12), wherein the alignment is performed by selecting a protein having the steric structure to be referred to from a database of the known steric structures of G-protein-coupled receptors, and juxtaposing an amino acid sequence of the selected reference protein and an amino acid sequence of the desired protein, the database of amino acid sequences used as a profile is selected from the group consisting of GCRDb, CPCRDB, ExPASy and ORDB and, when alignment has a reliability of 98% or more, it is judged that the steric structure of the desired protein can be produced.

According to still another aspect of the present invention, there are provided (14) a method of designing a drug molecule, which comprises identifying, retrieving, assessing or designing a desired drug molecule based on the interaction between the steric structure of a drug candidate molecule and the steric structure of a 7-transmembrane G-protein-coupled receptor produced by using the atomic coordinate as defined in the above (9), or the atomic coordinate of the recording medium as defined in the above (10) or of the database as defined in the above (11), (15) a method for screening a medicine or an agricultural chemical, which comprises examining the activity and the safety of the drug molecule obtained by the method as defined in the above (14) as a medicine or an agricultural chemical by a desired pharmacological or physiological test, and selecting a drug molecule having the desired nature, (16) a method for manufacturing a medicine or an agricultural chemical, which comprises formulating by blending a drug molecule obtained by the method as defined in the above (15) and a pharmaceutically or agriculturally or horticulturally acceptable carrier, and (17) a drug molecule obtained by the method as defined in the above (14).

### BRIEF DESCRIPTION OF THE DRAWINGS

FIG. 1 is a flowchart that shows one example of a method for constructing the steric structure of GPCRs of the present invention.

FIG. 2 is a view that shows a method for constructing a Cα atomic coordinate at the step 30. An identified part of alignment is obtained from a reference protein and, as to an absene part, minimum rmsd of overlapping of two residues having overlapped both N and C terminals is obtained from a database.

FIG. 3 is a view that shows local space homology (LSH). In calculating regarding T residue in the figure, netted (gray) residues are taken into consideration. A part surrounded by a square in an alignment at a low part of the figure is a residue pair to be considered, and a ratio at * mark is LSH. In this case, LSH is 56.2%.

FIG. 4 is a view that shows the relationship between LSH and a ratio at structurally conserved regions (SCRs). LSH is calculated from the overlapping of Cα atoms between the desired protein and the reference protein, and a ratio at SCRs is the number of residues in SCRs relative to the total number of residues of the desired protein.

FIG. 5 is a view that shows alignment of QRHUB2 (β adrenaline receptor) obtained by using 1F88 (rhodopsin) as a reference protein. In the figure, numerals on the right of QRHUB2 and 1F88 are the number of amino acids subjected to alignment in an amino acid sequence of each protein. In addition, the upper sequence indicates QRHUB2 (β adrenaline receptor) and the lower sequence indicates 1F88 (rhodopsin). An amino acid sequence of each protein is shown by a single letter symbol.

FIG. 6 is a printout of a display that shows bonding between the steric structure model constructed by the present method and an agonist (isoproterenol). The netted part in the figure indicates the agonist. A circumstance can be seen in which this agonist in R conformation is hydrogen-bonded to a receptor.

FIG. 7 is a printout of a display that shows the relative position of amino acid residues which are hydrogen-bonded to an agonist when the agonist in R form is introduced in seven α helices of a β2 adrenaline receptor in FIG. 6.

FIG. 8 is a printout of a display that shows bonding between the steric structure model constructed by the present method and an agonist (isoproterenol). The netted part in the figure indicates the agonist. A circumstance can be seen in which this agonist in S conformation is hydrogen-bonded to a receptor.

FIG. 9 is a printout of a display that shows the relative position of amino acid residues which are hydrogen-bonded to this agonist when an agonist in S form is introduced in seven α helices of a β2 adrenaline receptor in FIG. 8. A circumstance can be seen in which the hydrogen bond distance between this agonist and a receptor is instabilized to some extent due to its S comformation.

FIG. 10 is a printout of a display that shows bonding between the steric structure model constructed by the present method and an antagonist in R form (propranolol). In the figure, a netted part is an antagonist. A circumstance can be seen in which an antagonist in R form is introduced in seven α helices of a β2 adrenaline receptor.

FIG. 11 is a printout of a display that shows the relative position of amino acid residues which are hydrogen-bonded to this antagonist when an antagonist in R form is introduced in seven α helices of a β2 adrenaline receptor in FIG. 10. A circumstance can be seen in which a hydrogen bond distance between this antagonist and a receptor is extended due to its R comformation.

FIG. 12 is a printout of a display that shows bonding between the steric structure model constructed by the present method and an antagonist in S form (propranolol). In the figure, the netted part is the antagonist. A circumstance can be seen in which an antagonist in S form is introduced in seven α helices of a β2 adrenaline receptor.

FIG. 13 is a printout of a display that shows the relative position of amino acid residues which are and hydrogen-bonded to this antagonist with some inatalization when the antagonist in S form is introduced in seven α helices of a β2 adrenaline receptor in FIG. 12.

FIG. 14 is a printout of a display that shows the relative position of an antagonist (left) and an agonist (right) in a β2 adrenaline receptor in FIGS. 6 to 13. From the figure, it can be seen that an agonist and an antagonist are introduced into pockets at different positions, respectively, as expected from an experiment of site-specific mutation.

### BEST MODE FOR CARRYING OUT THE INVENTION

The present invention will be explained in more detail below.

In the present invention, a “desired protein” means a protein whose complete steric structure has not been determined by X-ray crystal analysis, NMR analysis or the like and which is a subject for steric structure construction and function presumption in the present invention. This protein includes proteins whose complete steric structure has not been obtained while whose partial structure has been analyzed, proteins whose functions have been already specified as GPCRs, proteins presumed to be GPCRs, and proteins whose functions are not clear at all while their amino acid sequences are determined. “A reference protein” is a protein whose minute steric structure has been already determined by X-ray crystal analysis or NMR analysis, and which is to be referred to for alignment and optimization of an atomic coordinate. “Alignment” means obtaining of the corresponding relationship between amino acid sequences of two or more kinds of proteins, and a method therefor will be described in detail below at explanation of each step.

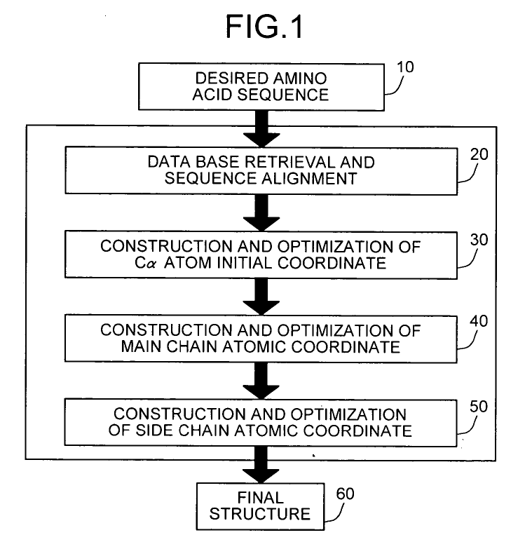

Then, the present invention will be explained by referring to the drawings. FIG. 1 is a flowchart that shows one example of a method for constructing the steric structure of GPCRS of the present invention.

First, at the step 10, an amino acid sequence of a desired protein (sequence) is inputted. At the step 20, a structure to be referred to is selected from database of steric structures, and alignment (juxtaposition) with an amino acid sequence of the selected reference protein is performed. At the step 30, based on information of the alignment, a coordinate with regard to a Cα atom which is one of the constituent atoms in the amino acid is obtained from the structure of the reference protein prescribed at the step 20, and an atomic coordinate of Cα is optimized so as to minimize an objective function by a simulated annealing method. At the step 40, other atoms of a main chain are added to an atomic coordinate of Cα at the step 30, followed by the optimization to minimize an objective function by a simulated annealing method. At the step 50, atoms of a side chain are added to an atomic coordinate of a main chain at the step 40, which is optimized so as to minimize an objective function by a simulated annealing method. Thus, the steric structure of GPCRs can be constructed with good precision.

Each step will be described in detail below.

Step 10: An Amino Acid Sequence of a Desired Protein

First, an amino acid sequence of a desired protein having an unknown steric structure is inputted. As the amino acid sequence used, it is particularly preferable to use sequences registered in a database as GPCRs. These sequences can be obtained from GCRDb (The G-protein-coupled Receptor Database), the details of which are described in “An Internet review: the complete neuroscientist scours the World Wide Web” Bloom FE, Science 1996; 274 (5290): 1104-9: http://www.gcrdb.uthscsa.edu/, GPCRDB: http://www.qpcr.org/7tm/, ExPASy: http://www.expasv.ch/cgi-bin/sm-gpcr.pl, ORDB: http://ycmi.med.vale.edu/senselab/ordb/ and the like.

Not only sequences registered in databases as GPCRs, but also any amino acid sequences of such as human (H. sapiens), drosophila (D. melanogaster), nematode (C. elagans), yeast (S. cerevisiae), and thalecress (A. thaliana) registered in databases such as GeneBank: ftp://ncbi.nlm.nih.gov/genbank/qenomes/, PIR: http://www-nbrf.georget own.edu/pir/(National Biomedical Research Foundation (NBRF)), Swiss Plot: http://www.expasy.ch/sprot/sprot-top.html (Swiss Institute of Bioinformatics (SIB), European Bioinfomatics Institute (EBI)), TrEMBL (URL and the administrator are both the same as those of Swiss Plot), TrEMBLNEW (URL and the administrator are both the same as those of Swiss Plot), NAD: ftp://ftp.ddbj.nig.ac.jp (Japanese DNA databank) and the like can be used. These databases are merely an example, and any databases can be used as far as they are databases in which amino acid sequences of proteins are registered.

Step 20: Database Retrieval and Sequence Alignment

After selecting a protein having a structure to be referred to from the steric structure database (Protein Data Bank) using a database of amino acid sequences of GPCRs as a profile, alignment juxtaposition) is performed between an amino acid sequence of the selected reference protein and an amino acid sequence of the desired protein. As a structure to be referred to, the steric structure of rhodopsin (PDB ID:1F88) is preferable. Hereinafter, alignment using a database of amino acid sequences as a profile may be simply referred to as “profile alignment”.

Herein, a “profile” adds information characterizing the position of a letter in a sequence to the letter. In the present invention, this means a matrix. This matrix is a round-robin combination of amino acids, and has 20*20 elements. This matrix also has meanings of information of the degree of amino acid conservation, the degree of susceptibility, and the similarity between amino acids.

As a software for performing profile alignment regarding an amino acid inputted at the step 10, it is preferable to use, for example, PSI-BLAST (Position-specific Iterated BLAST). PSI-BLAST is programmed so as to perform profile alignment. Details of PSI-BLAST are described in “Matching a protein sequence against a collection of PSI-BLAST-constructed position-specific score matrices.” Schaffer A A, Wolf Y I, Ponting C P, Koonin E V, Aravind L and Altschul S F, Bioinformatics 1999, 12, 1000-11.

Examples of an amino acid of GPCRs used as a profile include the aforementioned GCRDb, GPCRDB, ExPASy and ORDB which are public databases. These databases are used alone or as a combination thereof. Amino acid sequences of a database used as a profile is not particularly limited, but it is more preferable to use all sequences of a database used.

PSI-BLAST for performing profile alignment can be said to be a tool having the highest performance at present, for detecting a similarity of sequences. This program extracts information only from the alignment relation with significance in a database of a profile (herein, in GCRDb), and produces a site-specific score matrix of an amino acid. Then, in the interior of a program, a sequence having a high identity with a site-specific score matrix produced in place of the sequence of the desired protein is retrieved from a database, and the site-specific matrix is sequentially renewed every time until a more significant alignment is not detected. And, the identity between a final site-specific score matrix and a reference protein is calculated as the E value.

The E value quantitatively describes a random background noise present during matching between sequences. This also shows how much degree two sequences match, has the nature to decrease exponentially relative to a score, and is useful for a method of setting a significant threshold for the result. In the present invention, when the alignment has a reliability of 98% or more in general, preferably of 99% or more, it is suitable to judge that the desired protein is GPCRs and its steric structure should be so produced. In PSI-BLAST, this corresponds to when the E value statistically has a value of 0.1 or smaller in general, preferably of 0.01 or smaller.

Considering that GPCRs share the common nature of producing a disulfide bond at one position, it is preferred to perform re-alignment of GPCRs with covering all possible combinations of the disulfide bond at the position and with placing stress on the disulfide bond (SS bond) by managing parameters using a suitable alignment software, for example, the clustalW program.

As used herein, clustalW is an alignment program which enables to change a weighing function matrix and a gap penalty depending on a position on an amino acid sequence. In the present invention, since the disulfide bond is important for the steric structure as to GPCRs whose steric structures are the subject to be constructed, it is preferred to perform re-alignment after setting the weighing function such that the cysteine residue forming the disulfide bond in the alignment calculated by PSI-BLAST would match when the cysteine residue wouldn't match.

Details of ClustalW are described in Thompson, J. D., Higgins, D. G. and Gibson, T. J. (1994) CLUSTAL W: Improving the sensitivity of progressive multiple sequence alignment through sequence weighting, positions-specific gap penalties and weight matrix choice. Nucleic Acids Research, 22: 4673-4680.

That is, in re-alignment, (i) a three-dimensional structure of a protein to be referred to is preferably, for example, the aforementioned 1F88 of PDB (Protein Data Bank) (PDB ID:1F88), provided that 1F88 has an A chain and a B chain, and their coordinates have a great deficiency and are not complete, respectively. Then, it is preferable to perform modeling of the 1F88 structure using a modeling software, for example, FAMS (Journal of Molecular Graphics and Modeling 18, 258-272, 2000) and refer to the structure. (ii) A site of interest is a region of the desired protein corresponding to two parts, first site: YFVFGPTGC (SEQ ID NO.1) and second site: GWSRYIPEGMQCSCGIDYYTPHEETINNE (SEQ ID NO.2) which contains a cysteine residue. Outside of this is a secondary structure of an α helix and a β sheet in the 1F88 protein, and should not be moved respecting the alignment of PSI-BLAST. In addition, (iii) an object is to effect the matched alignments where the desired protein corresponds to C (cysteine) of the first site and C of the second site, respectively. In the first site, a secondary structure follows immediately after C, and there is little degree of freedom for changing the alignment of PSI-BLAST.

Specific preferable procedures for re-alignment are as the following (1) to (6):

(1) Definition File of Secondary Structure Part

First, a definition file for a secondary structure part of a reference protein, for example, the second site of 1F88:GWSRYIPEGMQCSCGIDYYTPHEETNNE (SEQ ID NO.2) is produced. This is for use in re-alignment for preventing a gap from entering into a secondary structure part as much as possible. Specifically, the definition file is the following form with the SwissProt file format as follows:

| ID | IF88A STANDARD; | | PRT; | 28AA. | |
|---|---|---|---|---|---|
| FT | STARND | 13 17 | | | |
| SQ | SEQUENCE | 28 | AA; | 000000 | MW; |

XXXXXXXXXXXXXXXX CRC64;

| | |
|---|---|
| GWSRYIPEGM | QXSCGIDYYTPHEETNNE |

(2) Excision of a Part of Interest from an Original Alignment File

Then, the original alignment file is read, and the sequence of the desired protein corresponding to the second site of the reference protein 1F88: GWSRYIPEGMQCSCGIDYYTPHEETNNE (SEQ ID NO.2) is written out (excised). As described in the above (iii), since a secondary structure follows immediately after C (cysteine) in the first site, and h there is little degree of freedom for changing the alignment of PSI-BLAST, only a sequence regarding a part corresponding to the second site is excised. Upon this, a file is produced in which a position of an excised part is recorded.

(3) Variation of a Part of Interest Corresponding to a Plurality of Cysteines (Cys)

When a part of interest of the desired protein contains a plurality of Cys, for attempting as a case study, the alignment in which Cys of the reference protein are definitely corresponded to respective Cys, only one Cys of the desired protein is left as it is, and the remaining Cys are masked with X to generate sequences of a part of interest by the number of corresponding Cys.

(4) Re-Alignment Only of a Part of Interest by an Alignment Program

Regarding short parts produced in the above (1) and (2), alignment for avoiding a gap in a secondary structure is performed paying serious attention to a SS bond. Specifically, for example, the following parameters are imparted to clustalW.

| | |
|---|---|
| General gap opening penalty | 2.0 |
| Helix part gap opening penalty | 9.0 |
| Strand part gap opening penalty | 9.0 |
| General gap extension penalty | 2.0 |

Elements of a mutation matrix are as follows, for example, paying serious attention to a SS bond.

| | |
|---|---|
| Consistency of Cys-Cys | 99 |
| Consistency of Trp-Trp | 16 |
| Consistency in general residues | 4 |
| Between Glu-Gln | 3 |
| Between Asp-Aspn | 3 |
| Between Arg-Lys | 3 |
| Other relationship | 0 |

(5) Incorporation of a Re-Aligned Part of Interest into a Whole Alignment

Incorporation of a re-aligned part of interest into a whole alignment is performed by putting the re-alignment of a part of interest obtained in (4) into the original broad alignment of PSI-BLAST, by referring to an excised place recorded in (2) and (3). Upon this, the parts masked with X (C of GMQCS of a second site of a reference protein and, when a part of interest of the desired protein contains a plurality of Cys, Cys situated at a place other than the relevant Cys) are returned to the original sequence.

(6) Checking of a Produced Alignment

(6-1) Checking of a SS Bond

In a process of the above (4), a sequence is given paying serious attention to a SS bond, but this is relative. Here, only the alignment having effected the of consistency of Cys-Cys is extracted.

(6-2) Checking of Uniqueness

An alignment file starting from the above (2) and (3) may be consistent with the original alignment of (2) in some cases. In addition, the file may be consistent with the already modeled alignment file in some cases. Modeling by the same alignment file leaves only a unique file in order to save an amount of calculation since a great number of modeling is required at present.

(6-3) Checking of Preservation

For caution's sake, it is checked that a sequence of the desired protein of the original alignment file and that of a reference protein are consistent except for a gap. This is for preventing incorrect data due to an operation mistake from being supplied to modeling.

By the aforementioned procedures, a re-alignment is produced in view of the common characteristic of GPCRs of making a disulfide bond at one place.

Based on the above-mentioned alignment information, construction of an atomic coordinate defining the steric structure, that is, production of the steric structure is performed. A method of producing the steric structure is not particularly limited, but for example, it is preferable to construct an atomic coordinate by a method of the following steps 30 to 50. In addition, calculation times, a constant, a cutoff value and the like described in explanation of the following steps 30 to 50 show one example of parameters which are thought to be most preferable by the present inventors, and this does not limit the scope of the present invention at all.

Step 30: Construction and Optimization of an Initial Coordinate of a Cα Atom

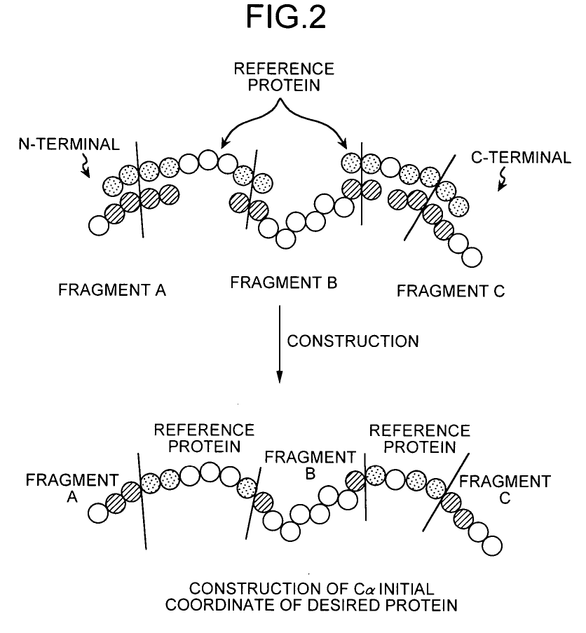

Receiving the results of the alignment from the step 20, information about the inserted and defective amino acid residues is obtained from a reference protein. A region having no gap in which consecutive three or more residues of amino acids are corresponding in the alignment is selected and, in this region, the same Cα atom as that of the reference protein is put into the desired protein in these residue pairs. When the Cα atom has not been obtained, a coordinate is adapted from a database of a pre-made fragment (see FIG. 2). As used herein, a Cα atom means a carbon atom which is a center of a skeleton of each amino acid. A Cβatom means a carbon atom binding to a side chain side of a Cα atom. In addition, a C atom means a carbon atom of a carbonyl group.

Step 31: Construction of a Cα Atom by a Simulated Annealing Method

The Cα atom produced in the above-mentioned step 30 is optimized using a function constructed of coordinates of a reference protein using a process of simulated annealing. This objective function is as described in the following equation (1):

U_{Cα}=Uₗₑₙ+U_{ang}+Uₚₒₛ+U_{vdw} (1).

Herein, Ulen relates to a distance between Cα atoms of a pair of adjacent residue and Cys residue on a sequence, and is set as in the following equation (2):

$${U}_{len} = {K}_{l} {\sum }_{i} {( {D}_{i , i + 1} - 3.8 )}^{2} + {K}_{ss} {\sum }_{i} {( {D}_{i}^{ss} - 5.4 )}^{2} . \qquad ( 2 )$$

Herein, Di, i+1 is a distance between Cαs of a residue i and a residue i+1. DISS is a distance between pairs of Cys residues forming a disulfide bond. K1 and KSS are constants, being set at 2 and 5, respectively.

Uang is a function as a binding angle of a Cα atom, and is as in the following equation (3):

$${U}_{ang} = {K}_{a} {\sum }_{i} {( {θ}_{i} - {θ}_{0} )}^{2} \qquad ( 3 )$$

Herein, θᵢ (rad) is an angle of an i^{th}, i+1^{th} or i+2^{th} residue Cα atom. θ₀ is set at (100/180)·π(rad) from the X-ray structure. Ka is a constant and is set at 1.

Upos is a function regarding a position of a Cα atom, and is as in the following equation (4):

$${U}_{pos} = {K}_{pos} {\sum }_{i} \frac{1}{{M}_{i}} {‖ {x}_{i} - ⟨ {w}_{i} {x}_{i} ⟩ ‖}^{2} . \qquad ( 4 )$$

Herein, ∥·∥ means norm, and Mi is an average distance between Cα atoms situated at structurally equivalent positions on an alignment based on a structure. When a value of Mi can not be obtained for a residue i, a value of Mi is set at 10. Herein, it is an average coordinate of a Cα atom, and is as in the following equation (5):

$$⟨ {w}_{i} {x}_{i} ⟩ = \frac{1}{W} {\sum }_{j} {w}_{i}^{j} {x}_{i}^{j} . \qquad ( 5 )$$

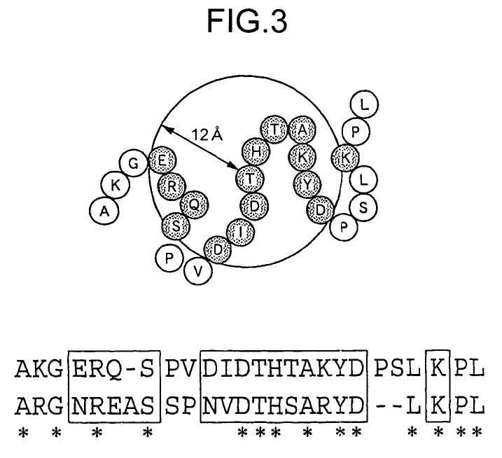

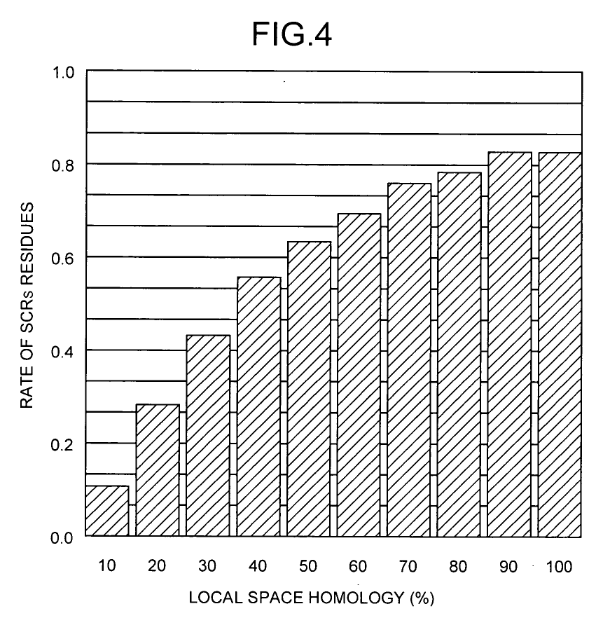

Herein, Xji is an atomic coordinate of Cα of an i^{th} residue of a j^{th} reference protein. Herein, wji is a weight of an i^{th} residue of a j^{th} reference protein. This weight is an important parameter for determining an approximate shape of the desired protein, and this is determined by a local value in the spatial vicinity within 12 Å of an attentioned part called Local Space Homology (LSH) (see FIG. 3). A correlation between LSH and a ratio of residue pairs present in a part in which a structure is well conserved (SCRs: Structural Conserved Regions) is very high as shown in FIG. 4. This means that, when the LSH value is high, a position of a Cα atom is statistically within 1.0 Å as compared with a reference protein structure.

Uvdw is as in the following equation (6):

$${U}_{vdw} = {K}_{vdw} {\sum }_{i , j ( > i + 2 )} \{ {( \frac{3.8}{{D}_{i , j}} )}^{12} - {( \frac{3.8}{{D}_{i , j}} )}^{6} \} . \qquad ( 6 )$$

Herein, Kvdw is set at 0.01 (Di,j<3.2 Å) and 0.001(Di,j>3.2 Å), and has a cutoff value of 6 Å.

A Cα atom is optimized using a simulated annealing method according to the equation (1). At this optimization stage, perturbation of a Cα atom is set to be within 1.0 Å. In addition, this annealing stage is calculated every 100 times regarding all Cα atoms. And, a parameter corresponding to a temperature is reduced from 25 by 0.01 every 0.5 time, and the parameter is constant thereafter.

These great two stages, obtaining of structural information and construction of a Cα atom are repeated 10 times, and a coordinate of a Cα atom having the minimum objective function value is calculated as an optimum solution.

Step 40: Construction and Optimization of a Main Chain Atomic Coordinate

Other atoms of a main chain are added to an atomic coordinate of Cα at the step 30, and an objective function is minimized by a simulated annealing method. First, steric overlapping of Cα atoms is performed, and residues having a distance between Cα atoms of 2.5 Å or smaller are taken. Atomic coordinates of a main chain except for Cα are obtained from a coordinate of a reference protein so that a distance between Cα atoms becomes minimum and, thus, a model structure is obtained.

When there is no corresponding residue in a reference protein, atomic coordinates of a main chain are produced from a corresponding protein fragment of four residues in a database. During this process, main chain atoms of a residue i are selected from a residue having the minimum rmsd value between i−1^{th} to i+2^{th} Cα atoms. Upon this, at a N-terminal residue, a range of overlapping of Cα atom coordinates is from i^{th} to i+3^{th} and, at a C-terminal residue and a residue one before, the ranges are from i−3^{th} to i^{th} and from i−2^{th} to i+1^{th}, similarly.

Based on an objective function of main chain atoms, optimization is performed by a simulated annealing method.

An objective function is as in the following equation (7):

Uₘₐᵢₙ=U_{bond}+U_{ang}+U_{non-bond}+Uₛₛ+Uₚₒₛ+Uₜₒᵣ+U_{chi}+U_{hydr} (7).

Ubond is as in the following equation (8):

$${U}_{bond} = {K}_{b} {\sum }_{i} {( {b}_{i} - {b}_{i}^{0} )}^{2} . \qquad ( 8 )$$

Herein, bi0 is a standard bond length, and is different depending on a kind of each chemical bond. Kb is a constant, and is set at 225.

Uang is a function of a bonding angle, and is as in the following equation (9):

$${U}_{ang} = {K}_{a} {\sum }_{i} {( {θ}_{i} - {θ}_{i}^{0} )}^{2} . \qquad ( 9 )$$

Herein, θi is an i^{th} bonding angle, and is different depending on a kind of a chemical bond. Ka is a constant, and is set at 45.

Unon-bond is a function of the non-bond interaction, and is as in the following equation (10):

$${U}_{non - bond} = {K}_{non} {\sum }_{i , j} {ϵ}_{i , j} \{ {( \frac{{r}_{i , j}^{*}}{{r}_{i , j}} )}^{12} - 2 {( \frac{{r}_{i , j}^{*}}{{r}_{i , j}} )}^{6} \} . \qquad ( 10 )$$

Herein, εi,j and ri,j * are constants, and are different depending on a kind of an atom.

Knon is a constant and is set at 0.25, and has cutoff of 8 Å.

USS is a function of a disulfide bond produced by the Cys residue, and is as in the following equation (11):

$${U}_{ss} = {\sum }_{i} \{ {{K}_{C α}^{ss} ( {D}_{i}^{C α} - 5.4 )}^{2} + {{K}_{C β}^{ss} ( {D}_{i}^{C β} - 3.8 )}^{2} \} . \qquad ( 11 )$$

Herein, KSSCα and KSSCβ are constants, and are set at 7.5.

Upos is a function regarding a position of an atom, and is as in the following equation (12):

$${U}_{pos} = {K}_{pos} {\sum }_{i} \frac{1}{{M}_{i}} {‖ {x}_{i} - ⟨ {w}_{i} {x}_{i} ⟩ ‖}^{2} . \qquad ( 12 )$$

Herein, <WiXi> is given as in the following equation (13):

$$⟨ {w}_{i} {x}_{i} ⟩ = \frac{1}{W} {\sum }_{j} w {}_{i}^{j}{x}{}_{ i}^{ j} . \qquad ( 13 )$$

<WiXi> in the equation (12) is obtained from overlapping of structures between the desired protein and the reference protein.

Kpos is a constant, and is set at 0.3.

Utor is related to a helix angle of a main chain, and is as in the following equation (14):

$${U}_{tor} = {K}_{t} {\sum }_{i} \sqrt{{( {ϕ}_{i} - {ϕ}_{i}^{0} )}^{2} + {( {ψ}_{i} - {ψ}_{i}^{0} )}^{2}} + {K}_{ω} {\sum }_{i} {( {ω}_{i} - {ω}_{i}^{0} )}^{2} . \qquad ( 14 )$$

Herein, φi0 and φi0 are let to be nearest helix angles θi and (φi on the Ramachandran map. In addition, ωi0 is let to be 0 and, only in the case of cis-Pro residue, is let to be π(radian). Kt and Kω are constants, and are let to be 10 and 50, respectively.

Uchi is related to chirality of Cα, and is as in the following equation (15):

$${U}_{chi} = {K}_{chi} {\sum }_{i} {( {τ}_{i} + \frac{2}{3} π )}^{2} . \qquad ( 15 )$$

Herein, πi is a helix angle defined by N—Cα-C β-C and Kchi is let to be 50.

Uhydr is related to a hydrogen bond of a main chain conserved in homologous proteins, and is determined as in the following equation (16):

$${U}_{hydr} = {K}_{hydr} {\sum }_{i , j} {( {D}_{i , j}^{N \text{-} O} - 2.9 )}^{2} . \qquad ( 16 )$$

A hydrogen bond is set when a distance between a N atom and a O atom is 2.9±0.5 Å.

Upon determining whether a hydrogen bond is present in a reference protein, when it is recognized that 75% or more of reference proteins are present, it is determined that a hydrogen bond is present. Khydr is a constant, and is let to be 0.6.

Optimization of main chain atoms including Cβ is performed by simulated annealing. During this annealing process, perturbation of atoms of a main chain and Cβ is let to be within 1.0 Å relative to initial positions. This annealing stage is performed 200 times on atoms of a main chain and Cβ. A parameter corresponding to a temperature starts from 50 or 25, is let to be 0.5-fold every time, and this is continued until 0.01 and, thereafter, is let to be a constant value.

In order to widely perform sampling of a steric arrangement of a main chain, according to the method of the present invention, the above-mentioned method is performed 6 times, and atomic coordinates of a main chain having the minimum objective function value are adopted as an optimum solution. And, a parameter corresponding to a temperature starts from 50 at first two times, and starts from 25 from third time.

Step 50: Construction and Optimization of Side Chain Atomic Coordinates

Construction of a side chain is roughly classified into two stages: “construction of a side chain at a structure conserved part” (step 51) and “construction of a total side chain” (step 52).

Step 51: Construction of a Side Chain at a Structure Conserved Part

Regarding calculated main chain atoms, a helix angle of a side chain is obtained from homologous proteins using the method in the previous study. Details of this method are described in “The role of played by environmental residues in side-chain torsional angles within homologous families of proteins: A new method of side chain modeling. “Ogata K and Umeyama H, Prot. Struct. Funct. Genet. 1998, 31, 255-369.

A ratio of side chains conserved in homologous proteins is calculated in this method, and, based on this information, modeling of a side chain is performed. Atomic coordinates of a side chain at a conserved site of a side chain are placed on main chain atoms. For example, when a χ¹ angle of an arginine residue is conserved in homologous proteins, a coordinate of a Cγ atom can be placed and, when χ1 and χ2 angles are conserved in a Phe residue, all side chain atoms can be placed. A process of optimization of simulated annealing using the equation (7) is such that only atoms of a main chain and Cβ are performed and perturbation of atoms is within 1.0 Å. This stage of annealing atoms of a main chain and Cβ are performed 200 times. And, a parameter corresponding to a temperature starts from 25, is let to be 0.5-fold every time, and is reduced until 0.01. Unon-bond in the equation (7) is performed on main chain atoms and partially produced side chain atoms. Upon this, coordinates of side chain atoms are rendered conserved during an optimization process.

Mi and a pair of N—O in a hydrogen bond which are information of a structure are used in an optimization process. In order to obtain arrangement of main chain atoms, the above-mentioned process is repeated three times, and coordinates of minimum main chain atoms of an objective function are adopted as a calculated structure.

Step 52: Construction of a Total Side Chain

Construction of a side chain is performed under fixed main chain and Cβ atoms. This is performed by the study results disclosed in the above-mentioned Ogata K and Umeyama H, Prot. Struct. Funct. Genet. 1998, 31, 255-369 and, by using this, a precise model can be given in a short time. Then, a main chain structure is optimized by a Monte Carlo method at a low temperature, a temperature is set at 0.001 and, by using an objective function Unon-bond of the equation (7), calculation is performed at all main chain and side chain atoms. And, during optimization of N, Cα, C and Cβ atoms, coordinates of a side chain are re-arranged so as to retain a helix angle of a side chain at the optimized state. Perturbation of atoms is let to be within 0.5 Å. Then, a side chain is deleted, and the above-mentioned construction of a side chain is repeated. This process is repeated until collision of 2.4 Å atoms is diminished, and a helix angle of N—Cα-Cβ—C becomes within a range of −120±15°.

Step 60: Construction of a Final Structure

Thus, by performing steps 10 to 50, atomic coordinates defining the steric structure of GPCRs can be obtained. The resulting atomic coordinates can be appropriately used for designing drug molecules such as medicines and agricultural chemicals described later, such as by producing the steric structure model of GPCR by a suitable program which can display the steric structure, for example, BIOCES (manufactured by NEC Corporation) and the like, and by assessing this and the bonding state with various ligands (drug molecules).

Each step regarding the aforementioned construction of the steric structure can be performed by using modeling software “FAMS” developed by the present inventors. Thereby, the steric structure can be constructed with excellent precision. Herein, details of FAMS are described in Koji Ogata and Hideaki Umeyama, Journal of Molecular Graphics and Modeling 18, 258-272, 2000.

Thus, atomic coordinates defining the steric structure of an arbitrary 7-transmembrane G-protein-coupled receptor can be obtained. Herein, an atomic coordinate describes the steric structure on a three-dimensional space. That is a relative distance in mutually perpendicular three directions letting a point on a space to be an origin, and is a vector consisting of three numerals per one atom except for a hydrogen atom present in a protein.

By containment of the resulting atomic coordinates in a suitable recording medium in a prescribed computer utilizable format, a database of the steric structure of a 7-transmembrane G-protein-coupled receptor can be constructed. The database of the present invention preferably contains, for example, information of alignment between a reference protein and a desired protein together with the above-mentioned atomic coordinates. In addition, in the present invention, a database means a computer system that writes the above-mentioned atomic coordinates in a suitable recording medium, and performs retrieval according to a prescribed program. Herein, examples of a suitable recording medium include magnetic media such as floppy disc, hard disk, magnetic tape and the like; optical disc, semiconductor memory and the like such as CD-ROM, MO, CD-R, CD-RW and the like.

According to another aspect of the present invention, there is provided a method for presuming the function of a protein, which comprises selecting the steric structure to be referred to from the known steric structure of a 7-transmembrane G-protein-coupled receptor using an amino acid sequence database of the receptor as a profile, performing alignment of a desired protein having the unknown function and, when the steric structure of the desired protein can be produced, judging that the desired protein is a 7-transmembrane G-protein-coupled receptor.

In the present invention, the steric structure of the desired protein having the unknown function is constructed by the above-mentioned method and, when construction is possible, it is judged that the desired protein is GPCRs. In the present invention, preferably, alignment is performed by selecting the steric structure to be referred to from a database of the known steric structure of a G-protein-coupled receptor, and aligning an amino acid sequence of the selected reference protein and an amino acid sequence of the desired protein, an amino acid sequence database used as a profile is selected from the group consisting of GCRDb, GPCRDB, ExPASy or ORDP and, when alignment has reliability of 98% or more, it is determined that the steric structure of the desired protein can be produced. The thus obtained atomic coordinates defining the steric structure of GPCRs are included in the database of the present invention.

Then, a method for designing a drug molecule using an atomic coordinate obtained by the present method will be described.

By using all or a part of atomic coordinates defining the steric structure (hereinafter, this is abbreviated as “steric structure coordinate”) of GPCR which is a target of a drug molecule obtained by the above-mentioned method (hereinafter, this is referred to as “target GPCR”), or a database in which they are recorded, or atomic coordinates contained in a recording medium, it is possible to perform identification, retrieval, assessment or design of a drug molecule which interacts with the target GPCR (antagonist or agonist).

Identification, retrieval, assessment or design of a drug molecule is performed based on the presence or the absence and a degree of interaction between the steric structure coordinate of GPCR obtained by the present method and the steric structure coordinate of a drug molecule, in a computer in which a suitable program that can design a drug molecule operates. In the present specification, identification, retrieval, assessment, design and the like of a drug molecule is simply referred to as drug molecule design.

A computer used upon molecule design based on interaction between the steric structure coordinate of GPCR and the steric structure coordinate of a drug candidate molecule is not particularly limited as far as it is a computer that is adjusted so as to operate a suitable program. In addition, a recording medium of a computer is not particularly limited. Examples of a program used in molecular design include a computer program Insight II and the like manufactured by Molecular Simulation. In particular, by using programs such as Ludi and DOCK which are especially produced for this purpose alone or in a combination thereof, a drug molecule can be more easily identified, retrieved, assessed, or designed. In addition, assessment of docking between the steric structure coordinate of GPCR and a drug molecule can be performed, for example, by using BIOCES (version 3.10) manufactured by NEC Corporation.

Herein, whether a drug molecule is a known drug molecule or a newly synthesized drug molecule having a novel chemical structure, any drug molecules can be used in the present method as far as the steric structure thereof can be obtained. The steric structure coordinate of a drug molecule may be a coordinate obtained by any method such as X-ray crystal analysis, modeling and the like. Determined steric structure coordinates can be obtained from a suitable database, for example, CCDC (Cambridge Crystallographic Data Centre: http://www.ccdc.cam.ac.uk/) and PDB (Protein Data Bank: http://www.rcsb.org/pdb/).

Further, a drug molecule can be designed also by the method described in Japanese Patent Application Laid-Open 2000-178209. Likewise, by using the steric structure coordinate of GPCR obtained by the present method, it becomes possible to design a drug molecule by a computer, provided that the molecular designing method of the present invention is not limited to methods using these programs and procedures.

There are usually conceptionally two stages in design of a drug molecule. The first stage is to find out a lead compound, and the next stage is to optimize the lead compound. Any of the stages can be performed by using the steric structure coordinate of target GPCR by a known per se method. Thereby, an optimum medicine and agricultural chemical candidate molecule can be obtained. The thus obtained drug molecule is included in the scope of the present invention.

Medicine and agricultural chemical candidate molecules identified, retrieved, assessed or designed by the above-mentioned method can be obtained, for example, by a known per se chemical synthesizing method depending on the natures of the molecules. However, a drug molecule may be a natural compound or a synthetic compound, or may be a high-molecular compound or a low-molecular compound. The obtained medicine and agricultural chemical candidate molecules are further examined for their activity and safety by a known per se method, or by an in vitro or in vivo pharmacological or physiological test, and medicine and agricultural chemical candidate molecules having the desired nature, that is, the activity and the safety necessary as a medicine or an agricultural chemical are selected (screening), whereby, molecules which can be actually applied as a medicine or an agricultural chemical can be obtained.

Medicines or agricultural chemicals, for example, medicine molecules selected by the above-mentioned screening method can be administered alone as they are to a patient with disease which is a subject of treatment, but can be also administered by mixing 2 or more active ingredients. In addition, it is preferable to prepare the substance into a pharmaceutical composition using a pharmacologically acceptable additive for pharmacy and administer the composition. For example, the substance may be used orally as sugar-coated tablets, capsules, granules, fine granules, powders, pills, microcapsules, liposome preparations, troches, sublingual agents, solutions, elixirs, emulsions, suspensions or the like, or parenterally as injectables prepared as sterile aqueous solutions or oily solutions, suppositories, ointments, patches or the like, as necessary. These may be prepared, for example, by kneading the substance together with physiologically acceptable carrier, flavor, excipient, vehicle, preservative, stabilizer and binder in a unit dosage form required for implementing generally recognized preparations, by using the well-known methods in the art such as filling and compression. An amount of an active ingredient in these pharmaceutical compositions allows a suitable dose in an indicated range to be obtained.

When agricultural chemical molecules are actually used as an agricultural chemical, they are used by mixing with agriculturally or horticulturally acceptable carrier or diluent, additive and supplementing agent by the known method, and preparing into preparation forms (composition) which are usually used as an agricultural chemical, for example, powders, granules, hydrated agents, emulsions, water-soluble agents, flowables and the like.

### EXAMPLES

The present invention will be further specifically explained by way of Examples below, but the following Examples should be regarded as helping obtain specific recognition of the present invention, and do not limit the scope of the present invention whatsoever.

### Example 1

### Construction of the Steric Structure of a β2 Adrenaline Receptor

According to the method described in detail in the aforementioned embodiments of the present invention, the steric structure model of a human-derived β2 adrenaline receptor was constructed, and a position into which an agonist (isoproterenol) is placed and a position into which an antagonist (propranolol) is placed were compared as follows:

Construction of the steric structure model was performed using workstation manufactured by NEC Corporation (Model: Express5800/120Rc-2, CPU:PentiumIII 933 MHz×2, OS: Red Hat Linux 6.2J, memory: 1024 Mbytes). An amino acid sequence of the desired β2 adrenalin receptor was obtained from PIR; http://www-nbrf.georgetown.edu/pir/ ID:QRHUB2. The amino acid sequence is shown in SEQ ID NO:3.

Letting an amino acid sequence of this β2 adrenaline receptor to be a sequence of the desired protein, alignment by PSI-BLAST was performed. Upon this, the full sequence 892 of GCRDb; http://www.gcrdb.uthscsa.edu/ was used as a motif profile. And, by using a structure of a B chain of PDB (http://www.rcsb.org/pdb/) ID:IF88 (rhodopsin) as the steric structure of a reference protein, alignment to this B chain was obtained. There was a dimer having the approximately same steric structure composed of an A chain and a B chain in a crystal lattice of 1F88 (rhodopsin), and the B chain was used as a structure to be referred to. In addition, there was a great defect in coordinates of the A chain and the B chain, respectively, being not complete. Modeling of the 1F88 structure was performed using modeling software “FAMS”, and that structure was referred to. An amino acid sequence of this 1F88 (rhodopsin) B chain is shown in SEQ ID NO.4.

As a result of the above-mentioned alignment, the E value calculated by PSI-BLAST was 1e-144 (minus 144 power of 10).

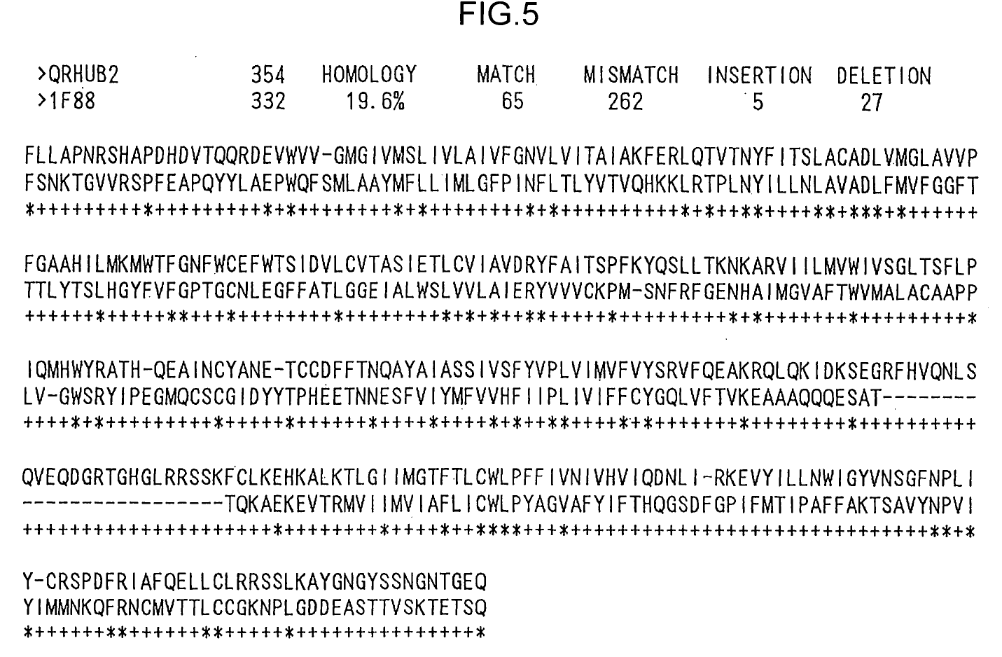

As described above, re-alignment was performed using a program of clutalW, paying serious attention to a disulfide bond. The resulting alignment information is shown in FIG. 5. In FIG. 5, an upper sequence denotes QRHUB2 (β adrenaline receptor), and a lower sequence denotes 1F88 (rhodopsin). As apparent from FIG. 5, homology between QRHUB2 and 1F88 was 19.6%.

Based on the thus obtained alignment information, using the modeling software “FAMS” developed by the present inventors, construction and optimization of an initial coordinate of a Cα atom, construction and optimization of a main chain atom coordinate, and construction of a total side chain were performed as in the above-mentioned steps 30 to 50, to obtain atomic coordinates defining the steric structure of QRHUB2.

The resulting atomic coordinates are shown in Table 1. Table 1 describes atomic coordinates according to the generally used format of Protein Data Bank (PDB) which is a method of expressing the steric structure (three-dimensional structure) coordinate of a protein. Table 1 describes a three-dimensional coordinate of each atom at 30^{th} line and thereafter except the last line. ATOM on a first column indicates that this row is a row of an atomic coordinate, a second column denotes an order of the atom, a third column denotes discrimination of atoms in an amino acid residue, a fourth column denotes an amino acid residue, a fifth column denotes an amino acid number corresponding to SEQ ID No.3 (corresponding to amino acid numbers 10 to 238, 264 to 363 in SEQ ID No.3), sixth, seventh and eighth columns denote a coordinate of the atom (in an order of a-axis, b-axis and c-axis directions, in Å unit), a ninth column denotes a rate of occupation of the atom, and a tenth column denotes a temperature factor of the atom. The last line denotes the last line in this table. A part of amino acid numbers 239 to 263 in SEQ ID No.3 is an insertion sequence part for which a structure to be referred to can not be obtained. In addition, since a rate of occupation and a temperature factor of an atom can not be calculated, “0.00” are placed into ninth and tenth columns.

TABLE 1
Atomic coordinate of β2 adrenaline receptor
HEADER mode 05 SEP. 2000 mode AUTHOR FAMS

| | | | |
|---|---|---|---|
| SEQRES | 1 | 329 | PHE LEU LEU ALA PRO ASN ARG SER HIS ALA PRO ASP HIS |
| SEQRES | 2 | 329 | ASP VAL THR GLN GLN ARG ASP GLU VAL TRP VAL VAL GLY |
| SEQRES | 3 | 329 | MET GLY ILE VAL MET SER LEU ILE VAL LEU ALA ILE VAL |
| SEQRES | 4 | 329 | PHE GLY ASN VAL LEU VAL ILE THR ALA ILE ALA LYS PHE |
| SEQRES | 5 | 329 | GLU ARG LEU GLN THR VAL THR ASN TYR PHE ILE THR SER |
| SEQRES | 6 | 329 | LEU ALA CYS ALA ASP LEU VAL MET GLY LED ALA VAL VAL |
| SEQRES | 7 | 329 | PRO PHE GLY ALA ALA HIS ILE LEU MET LYS MET TRP THR |
| SEQRES | 8 | 329 | PHE GLY ASN PHE TRP CYS GLU PHE TRP THR SER ILE ASP |
| SEQRES | 9 | 329 | VAL LEU CYS VAL THR ALA SER ILE GLU THR LEU CYS VAL |
| SEQRES | 10 | 329 | ILE ALA VAL ASP ARG TYR PHE ALA ILE THR SER PRO PHE |
| SEQRES | 11 | 329 | LYS TYR GLN SER LEU LEU THR LYS ASN LYS ALA ARG VAL |
| SEQRES | 12 | 329 | ILE ILE LEU MET VAL TRP ILE VAL SER GLY LEU THR SER |
| SEQRES | 13 | 329 | PHE LEU PRO ILE GLN MET HIS TRP TYR ARG ALA THR HIS |
| SEQRES | 14 | 329 | GLN GLU ALA ILE ASN CYS TYR ALA ASN GLU THR CYS CYS |
| SEQRES | 15 | 329 | ASP PHE PHE THR ASN GLN ALA TYR ALA ILE ALA SER SER |
| SEQRES | 16 | 329 | ILE VAL SER PHE TYR VAL PRO LEU VAL ILE MET VAL PHE |
| SEQRES | 17 | 329 | VAL TYR SER ARG VAL PHE GLN GLU ALA LYS ARG GLN LEU |
| SEQRES | 18 | 329 | GLN LYS ILE ASP LYS SER GLU GLY PHE CYS LEU LYS GLU |
| SEQRES | 19 | 329 | HIS LYS ALA LEU LYS THR LEU GLY ILE ILE MET GLY THR |
| SEQRES | 20 | 329 | PHE THR LEU CYS TRP LEU PRO PHE PHE ILE VAL ASN ILE |
| SEQRES | 21 | 329 | VAL HIS VAL ILE GLN ASP ASN LEU ILE ARG LYS GLU VAL |
| SEQRES | 22 | 329 | TYR ILE LEU LEU ASN TRP ILE GLY TYR VAL ASN SER GLY |
| SEQRES | 23 | 329 | PHE ASN PRO LEU ILE TYR CYS ARG SER PRO ASP PHE ARG |
| SEQRES | 24 | 329 | ILE ALA PHE GLN GLU LEU LEU CYS LEU ARG ARG SER SER |
| SEQRES | 25 | 329 | LEU LYS ALA TYR GLY ASN GLY TYR SER SER ASN GLY ASN |
| SEQRES | 26 | 329 | THR GLY GLU GLN |

SSBOND 1 CYS 97 CYS 175

| | | | | | | | | | |
|---|---|---|---|---|---|---|---|---|---|
| ATOM | 1 | N | PHE | 1 | 43.073 | 0.386 | −20.078 | 0.00 | 0.00 |
| ATOM | 2 | CA | PHE | 1 | 41.753 | −0.201 | −20.163 | 0.00 | 0.00 |
| ATOM | 3 | C | PHE | 1 | 41.414 | −0.419 | −21.636 | 0.00 | 0.00 |
| ATOM | 4 | O | PHE | 1 | 41.225 | 0.577 | −22.359 | 0.00 | 0.00 |
| ATOM | 5 | CB | PHE | 1 | 40.766 | 0.713 | −19.432 | 0.00 | 0.00 |
| ATOM | 6 | CG | PHE | 1 | 39.626 | −0.008 | −18.812 | 0.00 | 0.00 |
| ATOM | 7 | CD1 | PHE | 1 | 38.307 | 0.247 | −19.185 | 0.00 | 0.00 |
| ATOM | 8 | CD2 | PHE | 1 | 39.869 | −0.865 | −17.741 | 0.00 | 0.00 |
| ATOM | 9 | CE1 | PHE | 1 | 37.253 | −0.332 | −18.502 | 0.00 | 0.00 |
| ATOM | 10 | CE2 | PHE | 1 | 38.817 | −1.448 | −17.050 | 0.00 | 0.00 |
| ATOM | 11 | CZ | PHE | 1 | 37.506 | −1.180 | −17.431 | 0.00 | 0.00 |
| ATOM | 12 | N | LEU | 2 | 40.955 | −1.616 | −21.879 | 0.00 | 0.00 |
| ATOM | 13 | CA | LEU | 2 | 40.600 | −2.066 | −23.178 | 0.00 | 0.00 |
| ATOM | 14 | C | LEU | 2 | 39.522 | −1.130 | −23.782 | 0.00 | 0.00 |
| ATOM | 15 | O | LEU | 2 | 38.456 | −0.903 | −23.189 | 0.00 | 0.00 |
| ATOM | 16 | CB | LEU | 2 | 40.177 | −3.558 | −22.990 | 0.00 | 0.00 |
| ATOM | 17 | CG | LEU | 2 | 40.585 | −4.520 | −24.108 | 0.00 | 0.00 |
| ATOM | 18 | CD1 | LEU | 2 | 39.574 | −5.662 | −24.157 | 0.00 | 0.00 |
| ATOM | 19 | CD2 | LEU | 2 | 40.661 | −3.884 | −25.485 | 0.00 | 0.00 |
| ATOM | 20 | N | LEU | 3 | 39.652 | −1.018 | −25.078 | 0.00 | 0.00 |
| ATOM | 21 | CA | LEU | 3 | 38.777 | −0.250 | −25.976 | 0.00 | 0.00 |
| ATOM | 22 | C | LEU | 3 | 37.698 | −1.167 | −26.669 | 0.00 | 0.00 |
| ATOM | 23 | O | LEU | 3 | 37.353 | −0.941 | −27.843 | 0.00 | 0.00 |
| ATOM | 24 | CB | LEU | 3 | 39.720 | 0.447 | −26.998 | 0.00 | 0.00 |
| ATOM | 25 | CG | LEU | 3 | 39.142 | 1.650 | −27.822 | 0.00 | 0.00 |
| ATOM | 26 | CD1 | LEU | 3 | 39.404 | 1.543 | −29.337 | 0.00 | 0.00 |
| ATOM | 27 | CD2 | LEU | 3 | 37.641 | 1.886 | −27.688 | 0.00 | 0.00 |
| ATOM | 28 | N | ALA | 4 | 37.400 | −2.343 | −26.054 | 0.00 | 0.00 |
| ATOM | 29 | CA | ALA | 4 | 36.477 | −3.272 | −26.597 | 0.00 | 0.00 |
| ATOM | 30 | C | ALA | 4 | 35.117 | −2.525 | −26.707 | 0.00 | 0.00 |
| ATOM | 31 | O | ALA | 4 | 34.699 | −2.282 | −27.891 | 0.00 | 0.00 |
| ATOM | 32 | CB | ALA | 4 | 36.410 | −4.512 | −25.679 | 0.00 | 0.00 |
| ATOM | 33 | N | PRO | 5 | 34.358 | −2.111 | −25.644 | 0.00 | 0.00 |
| ATOM | 34 | CA | PRO | 5 | 33.084 | −1.488 | −25.861 | 0.00 | 0.00 |
| ATOM | 35 | C | PRO | 5 | 33.311 | −0.006 | −26.324 | 0.00 | 0.00 |
| ATOM | 36 | O | PRO | 5 | 32.302 | 0.657 | −26.536 | 0.00 | 0.00 |
| ATOM | 37 | CB | PRO | 5 | 32.239 | −1.431 | −24.582 | 0.00 | 0.00 |
| ATOM | 38 | CG | PRO | 5 | 33.171 | −2.182 | −23.598 | 0.00 | 0.00 |
| ATOM | 39 | CD | PRO | 5 | 34.594 | −2.101 | −24.154 | 0.00 | 0.00 |
| ATOM | 40 | N | ASN | 6 | 34.520 | 0.564 | −26.479 | 0.00 | 0.00 |
| ATOM | 41 | CA | ASN | 6 | 34.653 | 1.953 | −27.011 | 0.00 | 0.00 |
| ATOM | 42 | C | ASN | 6 | 34.068 | 3.074 | −26.086 | 0.00 | 0.00 |
| ATOM | 43 | O | ASN | 6 | 33.839 | 4.201 | −26.540 | 0.00 | 0.00 |
| ATOM | 44 | CB | ASN | 6 | 34.000 | 2.124 | −28.366 | 0.00 | 0.00 |
| ATOM | 45 | CG | ASN | 6 | 33.779 | 1.138 | −29.437 | 0.00 | 0.00 |
| ATOM | 46 | OD1 | ASN | 6 | 32.786 | 1.165 | −30.245 | 0.00 | 0.00 |
| ATOM | 47 | ND2 | ASN | 6 | 34.585 | 0.044 | −29.583 | 0.00 | 0.00 |
| ATOM | 48 | N | ARG | 7 | 34.407 | 2.920 | −24.839 | 0.00 | 0.00 |
| ATOM | 49 | CA | ARG | 7 | 34.120 | 3.835 | −23.733 | 0.00 | 0.00 |
| ATOM | 50 | C | ARG | 7 | 35.410 | 4.653 | −23.343 | 0.00 | 0.00 |
| ATOM | 51 | O | ARG | 7 | 35.233 | 5.799 | −22.940 | 0.00 | 0.00 |
| ATOM | 52 | CB | ARG | 7 | 33.580 | 3.010 | −22.551 | 0.00 | 0.00 |
| ATOM | 53 | CG | ARG | 7 | 33.328 | 3.924 | −21.350 | 0.00 | 0.00 |
| ATOM | 54 | CD | ARG | 7 | 32.202 | 4.900 | −21.560 | 0.00 | 0.00 |
| ATOM | 55 | NE | ARG | 7 | 30.908 | 4.251 | −21.675 | 0.00 | 0.00 |
| ATOM | 56 | CZ | ARG | 7 | 29.740 | 4.902 | −21.547 | 0.00 | 0.00 |
| ATOM | 57 | NH1 | ARG | 7 | 29.675 | 6.216 | −21.315 | 0.00 | 0.00 |
| ATOM | 58 | NH2 | ARG | 7 | 28.609 | 4.208 | −21.638 | 0.00 | 0.00 |
| ATOM | 59 | N | SER | 8 | 36.654 | 4.190 | −23.656 | 0.00 | 0.00 |
| ATOM | 60 | CA | SER | 8 | 37.904 | 4.876 | −23.437 | 0.00 | 0.00 |
| ATOM | 61 | C | SER | 8 | 37.932 | 6.234 | −24.183 | 0.00 | 0.00 |
| ATOM | 62 | O | SER | 8 | 37.958 | 6.283 | −25.421 | 0.00 | 0.00 |
| ATOM | 63 | CB | SER | 8 | 39.089 | 3.986 | −23.904 | 0.00 | 0.00 |
| ATOM | 64 | OG | SER | 8 | 40.409 | 4.526 | −23.618 | 0.00 | 0.00 |
| ATOM | 65 | N | HIS | 9 | 38.165 | 7.300 | −23.402 | 0.00 | 0.00 |
| ATOM | 66 | CA | HIS | 9 | 38.329 | 8.653 | −23.973 | 0.00 | 0.00 |
| ATOM | 67 | C | HIS | 9 | 39.745 | 9.163 | −23.460 | 0.00 | 0.00 |
| ATOM | 68 | O | HIS | 9 | 39.917 | 9.246 | −22.240 | 0.00 | 0.00 |
| ATOM | 69 | CB | HIS | 9 | 37.146 | 9.564 | −23.685 | 0.00 | 0.00 |
| ATOM | 70 | CG | HIS | 9 | 37.047 | 10.796 | −24.493 | 0.00 | 0.00 |
| ATOM | 71 | ND1 | HIS | 9 | 36.064 | 11.704 | −24.557 | 0.00 | 0.00 |
| ATOM | 72 | CD2 | HIS | 9 | 37.990 | 11.194 | −25.416 | 0.00 | 0.00 |
| ATOM | 73 | CE1 | HIS | 9 | 36.376 | 12.602 | −25.463 | 0.00 | 0.00 |
| ATOM | 74 | NE2 | HIS | 9 | 37.544 | 12.281 | −25.972 | 0.00 | 0.00 |
| ATOM | 75 | N | ALA | 10 | 40.613 | 9.714 | −24.336 | 0.00 | 0.00 |
| ATOM | 76 | CA | ALA | 10 | 41.978 | 10.099 | −23.914 | 0.00 | 0.00 |
| ATOM | 77 | C | ALA | 10 | 41.939 | 10.930 | −22.615 | 0.00 | 0.00 |
| ATOM | 78 | O | ALA | 10 | 41.034 | 11.817 | −22.560 | 0.00 | 0.00 |
| ATOM | 79 | CB | ALA | 10 | 42.624 | 10.929 | −25.068 | 0.00 | 0.00 |
| ATOM | 80 | N | PRO | 11 | 42.636 | 10.598 | −21.488 | 0.00 | 0.00 |
| ATOM | 81 | CA | PRO | 11 | 42.443 | 11.430 | −20.339 | 0.00 | 0.00 |
| ATOM | 82 | C | PRO | 11 | 42.462 | 12.969 | −20.636 | 0.00 | 0.00 |
| ATOM | 83 | O | PRO | 11 | 41.726 | 13.657 | −19.918 | 0.00 | 0.00 |
| ATOM | 84 | CB | PRO | 11 | 43.351 | 11.045 | −19.192 | 0.00 | 0.00 |
| ATOM | 85 | CG | PRO | 11 | 44.598 | 10.601 | −20.045 | 0.00 | 0.00 |
| ATOM | 86 | CD | PRO | 11 | 44.017 | 9.941 | −21.287 | 0.00 | 0.00 |
| ATOM | 87 | N | ASP | 12 | 43.185 | 13.472 | −21.606 | 0.00 | 0.00 |
| ATOM | 88 | CA | ASP | 12 | 43.119 | 14.896 | −21.854 | 0.00 | 0.00 |
| ATOM | 89 | C | ASP | 12 | 41.647 | 15.423 | −22.107 | 0.00 | 0.00 |
| ATOM | 90 | O | ASP | 12 | 41.238 | 16.320 | −21.367 | 0.00 | 0.00 |
| ATOM | 91 | CB | ASP | 12 | 44.036 | 15.208 | −23.051 | 0.00 | 0.00 |
| ATOM | 92 | CG | ASP | 12 | 45.467 | 14.821 | −22.771 | 0.00 | 0.00 |
| ATOM | 93 | OD1 | ASP | 12 | 46.035 | 14.195 | −23.582 | 0.00 | 0.00 |
| ATOM | 94 | OD2 | ASP | 12 | 45.951 | 15.218 | −21.705 | 0.00 | 0.00 |
| ATOM | 95 | N | HIS | 13 | 40.922 | 14.971 | −23.139 | 0.00 | 0.00 |
| ATOM | 96 | CA | HIS | 13 | 39.565 | 15.432 | −23.459 | 0.00 | 0.00 |
| ATOM | 97 | C | HIS | 13 | 38.527 | 15.140 | −22.337 | 0.00 | 0.00 |
| ATOM | 98 | O | HIS | 13 | 37.979 | 16.107 | −21.805 | 0.00 | 0.00 |
| ATOM | 99 | CB | HIS | 13 | 39.144 | 14.813 | −24.774 | 0.00 | 0.00 |
| ATOM | 100 | CG | HIS | 13 | 39.899 | 15.091 | −25.990 | 0.00 | 0.00 |
| ATOM | 101 | ND1 | HIS | 13 | 39.346 | 15.454 | −27.190 | 0.00 | 0.00 |
| ATOM | 102 | CD2 | HIS | 13 | 41.238 | 15.064 | −26.209 | 0.00 | 0.00 |
| ATOM | 103 | CE1 | HIS | 13 | 40.314 | 15.638 | −28.078 | 0.00 | 0.00 |
| ATOM | 104 | NE2 | HIS | 13 | 41.467 | 15.402 | −27.511 | 0.00 | 0.00 |
| ATOM | 105 | N | ASP | 14 | 38.258 | 13.862 | −21.957 | 0.00 | 0.00 |
| ATOM | 106 | CA | ASP | 14 | 37.253 | 13.533 | −20.930 | 0.00 | 0.00 |
| ATOM | 107 | C | ASP | 14 | 37.791 | 12.698 | −19.741 | 0.00 | 0.00 |
| ATOM | 108 | O | ASP | 14 | 38.666 | 11.838 | −19.929 | 0.00 | 0.00 |
| ATOM | 109 | CB | ASP | 14 | 35.903 | 13.055 | −21.485 | 0.00 | 0.00 |
| ATOM | 110 | CG | ASP | 14 | 35.199 | 13.882 | −22.507 | 0.00 | 0.00 |
| ATOM | 111 | OD1 | ASP | 14 | 34.360 | 13.247 | −23.276 | 0.00 | 0.00 |
| ATOM | 112 | OD2 | ASP | 14 | 35.353 | 15.131 | −22.637 | 0.00 | 0.00 |
| ATOM | 113 | N | VAL | 15 | 36.898 | 12.656 | −18.710 | 0.00 | 0.00 |
| ATOM | 114 | CA | VAL | 15 | 37.136 | 12.036 | −17.475 | 0.00 | 0.00 |
| ATOM | 115 | C | VAL | 15 | 37.399 | 10.517 | −17.579 | 0.00 | 0.00 |
| ATOM | 116 | O | VAL | 15 | 36.947 | 9.837 | −18.475 | 0.00 | 0.00 |
| ATOM | 117 | CB | VAL | 15 | 36.014 | 12.360 | −16.536 | 0.00 | 0.00 |
| ATOM | 118 | CG1 | VAL | 15 | 35.140 | 13.518 | −16.971 | 0.00 | 0.00 |
| ATOM | 119 | CG2 | VAL | 15 | 35.167 | 11.104 | −16.240 | 0.00 | 0.00 |
| ATOM | 120 | N | THR | 16 | 38.144 | 10.027 | −16.591 | 0.00 | 0.00 |
| ATOM | 121 | CA | THR | 16 | 38.439 | 8.589 | −16.520 | 0.00 | 0.00 |
| ATOM | 122 | C | THR | 16 | 37.305 | 7.828 | −15.707 | 0.00 | 0.00 |
| ATOM | 123 | O | THR | 16 | 37.664 | 6.837 | −15.059 | 0.00 | 0.00 |
| ATOM | 124 | CB | THR | 16 | 39.934 | 8.199 | −16.206 | 0.00 | 0.00 |
| ATOM | 125 | OG1 | THR | 16 | 40.373 | 7.068 | −17.053 | 0.00 | 0.00 |
| ATOM | 126 | CG2 | THR | 16 | 40.471 | 7.943 | −14.793 | 0.00 | 0.00 |
| ATOM | 127 | N | GLN | 17 | 36.092 | 8.409 | −15.467 | 0.00 | 0.00 |
| ATOM | 128 | CA | GLN | 17 | 35.022 | 7.662 | −14.758 | 0.00 | 0.00 |
| ATOM | 129 | C | GLN | 17 | 34.965 | 6.183 | −15.162 | 0.00 | 0.00 |
| ATOM | 130 | O | GLN | 17 | 34.657 | 5.399 | −14.270 | 0.00 | 0.00 |
| ATOM | 131 | CB | GLN | 17 | 33.586 | 8.287 | −14.893 | 0.00 | 0.00 |
| ATOM | 132 | CG | GLN | 17 | 33.654 | 9.770 | −14.559 | 0.00 | 0.00 |
| ATOM | 133 | CD | GLN | 17 | 34.068 | 9.953 | −13.109 | 0.00 | 0.00 |
| ATOM | 134 | OE1 | GLN | 17 | 33.406 | 9.484 | −12.174 | 0.00 | 0.00 |
| ATOM | 135 | NE2 | GLN | 17 | 35.158 | 10.661 | −12.865 | 0.00 | 0.00 |
| ATOM | 136 | N | GLN | 18 | 34.862 | 5.891 | −16.491 | 0.00 | 0.00 |
| ATOM | 137 | CA | GLN | 18 | 34.747 | 4.512 | −16.869 | 0.00 | 0.00 |
| ATOM | 138 | C | GLN | 18 | 35.809 | 3.595 | −16.138 | 0.00 | 0.00 |
| ATOM | 139 | O | GLN | 18 | 35.450 | 2.468 | −15.813 | 0.00 | 0.00 |
| ATOM | 140 | CB | GLN | 18 | 34.920 | 4.249 | −18.388 | 0.00 | 0.00 |
| ATOM | 141 | CG | GLN | 18 | 36.218 | 4.759 | −19.045 | 0.00 | 0.00 |
| ATOM | 142 | CD | GLN | 18 | 37.257 | 3.731 | −19.366 | 0.00 | 0.00 |
| ATOM | 143 | OE1 | GLN | 18 | 37.392 | 2.667 | −18.765 | 0.00 | 0.00 |
| ATOM | 144 | NE2 | GLN | 18 | 38.080 | 4.012 | −20.401 | 0.00 | 0.00 |
| ATOM | 145 | N | ARG | 19 | 37.083 | 4.023 | −15.906 | 0.00 | 0.00 |
| ATOM | 146 | CA | ARG | 19 | 38.097 | 3.241 | −15.140 | 0.00 | 0.00 |
| ATOM | 147 | C | ARG | 19 | 37.579 | 3.043 | −13.679 | 0.00 | 0.00 |
| ATOM | 148 | O | ARG | 19 | 37.479 | 1.863 | −13.285 | 0.00 | 0.00 |
| ATOM | 149 | CB | ARG | 19 | 39.523 | 3.759 | −15.273 | 0.00 | 0.00 |
| ATOM | 150 | CG | ARG | 19 | 40.143 | 3.416 | −16.595 | 0.00 | 0.00 |
| ATOM | 151 | CD | ARG | 19 | 41.240 | 4.369 | −17.113 | 0.00 | 0.00 |
| ATOM | 152 | NE | ARG | 19 | 41.647 | 3.953 | −18.459 | 0.00 | 0.00 |
| ATOM | 153 | CZ | ARG | 19 | 41.337 | 4.641 | −19.576 | 0.00 | 0.00 |
| ATOM | 154 | NH1 | ARG | 19 | 40.636 | 5.769 | −19.498 | 0.00 | 0.00 |
| ATOM | 155 | NH2 | ARG | 19 | 41.662 | 4.113 | −20.774 | 0.00 | 0.00 |
| ATOM | 156 | N | ASP | 20 | 37.268 | 4.090 | −12.935 | 0.00 | 0.00 |
| ATOM | 157 | CA | ASP | 20 | 36.690 | 3.915 | −11.600 | 0.00 | 0.00 |
| ATOM | 158 | C | ASP | 20 | 35.630 | 5.023 | −11.287 | 0.00 | 0.00 |
| ATOM | 159 | O | ASP | 20 | 35.723 | 6.176 | −11.743 | 0.00 | 0.00 |
| ATOM | 160 | CB | ASP | 20 | 37.822 | 3.783 | −10.606 | 0.00 | 0.00 |
| ATOM | 161 | CG | ASP | 20 | 38.595 | 5.053 | −10.375 | 0.00 | 0.00 |
| ATOM | 162 | OD1 | ASP | 20 | 38.044 | 6.107 | −10.114 | 0.00 | 0.00 |
| ATOM | 163 | OD2 | ASP | 20 | 39.837 | 5.007 | −10.540 | 0.00 | 0.00 |
| ATOM | 164 | N | GLU | 21 | 34.700 | 4.679 | −10.415 | 0.00 | 0.00 |
| ATOM | 165 | CA | GLU | 21 | 33.606 | 5.526 | −10.071 | 0.00 | 0.00 |
| ATOM | 166 | C | GLU | 21 | 34.041 | 6.910 | −9.445 | 0.00 | 0.00 |
| ATOM | 167 | O | GLU | 21 | 35.248 | 7.163 | −9.339 | 0.00 | 0.00 |
| ATOM | 168 | CB | GLU | 21 | 32.738 | 4.723 | −9.043 | 0.00 | 0.00 |
| ATOM | 169 | CG | GLU | 21 | 33.123 | 3.256 | −8.825 | 0.00 | 0.00 |
| ATOM | 170 | CD | GLU | 21 | 32.520 | 2.472 | −7.697 | 0.00 | 0.00 |
| ATOM | 171 | OE1 | GLU | 21 | 33.064 | 1.646 | −6.924 | 0.00 | 0.00 |
| ATOM | 172 | OE2 | GLU | 21 | 31.270 | 2.655 | −7.518 | 0.00 | 0.00 |
| ATOM | 173 | N | VAL | 22 | 33.169 | 7.904 | −9.663 | 0.00 | 0.00 |
| ATOM | 174 | CA | VAL | 22 | 33.328 | 9.295 | −9.162 | 0.00 | 0.00 |
| ATOM | 175 | C | VAL | 22 | 33.760 | 9.406 | −7.664 | 0.00 | 0.00 |
| ATOM | 176 | O | VAL | 22 | 34.388 | 10.426 | −7.367 | 0.00 | 0.00 |
| ATOM | 177 | CB | VAL | 22 | 31.998 | 10.004 | −9.510 | 0.00 | 0.00 |
| ATOM | 178 | CG1 | VAL | 22 | 30.860 | 9.381 | −8.711 | 0.00 | 0.00 |
| ATOM | 179 | CG2 | VAL | 22 | 32.112 | 11.487 | −9.103 | 0.00 | 0.00 |
| ATOM | 180 | N | TRP | 23 | 33.027 | 8.787 | −6.696 | 0.00 | 0.00 |
| ATOM | 181 | CA | TRP | 23 | 33.531 | 8.852 | −5.337 | 0.00 | 0.00 |
| ATOM | 182 | C | TRP | 23 | 35.021 | 8.365 | −5.294 | 0.00 | 0.00 |
| ATOM | 183 | O | TRP | 23 | 35.705 | 8.687 | −4.312 | 0.00 | 0.00 |
| ATOM | 184 | CB | TRP | 23 | 32.668 | 8.048 | −4.390 | 0.00 | 0.00 |
| ATOM | 185 | CG | TRP | 23 | 32.763 | 6.567 | −4.496 | 0.00 | 0.00 |
| ATOM | 186 | CD1 | TRP | 23 | 31.994 | 5.798 | −5.290 | 0.00 | 0.00 |
| ATOM | 187 | CD2 | TRP | 23 | 33.717 | 5.714 | −3.855 | 0.00 | 0.00 |
| ATOM | 188 | NE1 | TRP | 23 | 32.382 | 4.481 | −5.168 | 0.00 | 0.00 |
| ATOM | 189 | CE2 | TRP | 23 | 33.417 | 4.433 | −4.338 | 0.00 | 0.00 |
| ATOM | 190 | CE3 | TRP | 23 | 34.777 | 5.905 | −2.952 | 0.00 | 0.00 |
| ATOM | 191 | CZ2 | TRP | 23 | 34.172 | 3.333 | −3.908 | 0.00 | 0.00 |
| ATOM | 192 | CZ3 | TRP | 23 | 35.557 | 4.817 | −2.517 | 0.00 | 0.00 |
| ATOM | 193 | CH2 | TRP | 23 | 35.228 | 3.556 | −3.021 | 0.00 | 0.00 |
| ATOM | 194 | N | VAL | 24 | 35.504 | 7.655 | −6.351 | 0.00 | 0.00 |
| ATOM | 195 | CA | VAL | 24 | 36.836 | 7.229 | −6.418 | 0.00 | 0.00 |
| ATOM | 196 | C | VAL | 24 | 37.702 | 8.493 | −6.828 | 0.00 | 0.00 |
| ATOM | 197 | O | VAL | 24 | 38.714 | 8.260 | −7.443 | 0.00 | 0.00 |
| ATOM | 198 | CB | VAL | 24 | 37.029 | 6.004 | −7.320 | 0.00 | 0.00 |
| ATOM | 199 | CG1 | VAL | 24 | 38.548 | 5.664 | −7.451 | 0.00 | 0.00 |
| ATOM | 200 | CG2 | VAL | 24 | 36.202 | 4.829 | −6.966 | 0.00 | 0.00 |
| ATOM | 201 | N | VAL | 25 | 37.129 | 9.702 | −6.987 | 0.00 | 0.00 |
| ATOM | 202 | CA | VAL | 25 | 38.023 | 10.840 | −7.175 | 0.00 | 0.00 |
| ATOM | 203 | C | VAL | 25 | 38.870 | 10.750 | −5.866 | 0.00 | 0.00 |
| ATOM | 204 | O | VAL | 25 | 39.748 | 9.821 | −5.841 | 0.00 | 0.00 |
| ATOM | 205 | CB | VAL | 25 | 37.298 | 12.177 | −7.417 | 0.00 | 0.00 |
| ATOM | 206 | CG1 | VAL | 25 | 36.635 | 12.311 | −8.764 | 0.00 | 0.00 |
| ATOM | 207 | CG2 | VAL | 25 | 36.505 | 12.774 | −6.310 | 0.00 | 0.00 |
| ATOM | 208 | N | GLY | 26 | 38.250 | 11.098 | −4.694 | 0.00 | 0.00 |
| ATOM | 209 | CA | GLY | 26 | 38.795 | 10.938 | −3.307 | 0.00 | 0.00 |
| ATOM | 210 | C | GLY | 26 | 40.361 | 11.172 | −3.168 | 0.00 | 0.00 |
| ATOM | 211 | O | GLY | 26 | 40.894 | 10.843 | −2.115 | 0.00 | 0.00 |
| ATOM | 212 | N | MET | 27 | 41.021 | 11.449 | −4.261 | 0.00 | 0.00 |
| ATOM | 213 | CA | MET | 27 | 42.388 | 11.791 | −4.408 | 0.00 | 0.00 |
| ATOM | 214 | C | MET | 27 | 42.477 | 13.331 | −4.440 | 0.00 | 0.00 |
| ATOM | 215 | O | MET | 27 | 43.599 | 13.810 | −4.420 | 0.00 | 0.00 |
| ATOM | 216 | CB | MET | 27 | 42.822 | 11.231 | −5.750 | 0.00 | 0.00 |
| ATOM | 217 | CG | MET | 27 | 42.927 | 9.726 | −5.779 | 0.00 | 0.00 |
| ATOM | 218 | SD | MET | 27 | 44.503 | 9.174 | −5.069 | 0.00 | 0.00 |
| ATOM | 219 | CE | MET | 27 | 45.560 | 9.399 | −6.527 | 0.00 | 0.00 |
| ATOM | 220 | N | GLY | 28 | 41.416 | 14.039 | −4.820 | 0.00 | 0.00 |
| ATOM | 221 | CA | GLY | 28 | 41.311 | 15.472 | −4.810 | 0.00 | 0.00 |
| ATOM | 222 | C | GLY | 28 | 41.292 | 15.888 | −3.308 | 0.00 | 0.00 |
| ATOM | 223 | O | GLY | 28 | 41.587 | 17.061 | −3.075 | 0.00 | 0.00 |
| ATOM | 224 | N | ILE | 29 | 40.434 | 15.237 | −2.504 | 0.00 | 0.00 |
| ATOM | 225 | CA | ILE | 29 | 40.315 | 15.392 | −1.034 | 0.00 | 0.00 |
| ATOM | 226 | C | ILE | 29 | 41.750 | 15.108 | −0.437 | 0.00 | 0.00 |
| ATOM | 227 | O | ILE | 29 | 42.191 | 15.893 | 0.402 | 0.00 | 0.00 |
| ATOM | 228 | CB | ILE | 29 | 39.150 | 14.517 | −0.452 | 0.00 | 0.00 |
| ATOM | 229 | CG1 | ILE | 29 | 39.210 | 14.583 | 1.105 | 0.00 | 0.00 |
| ATOM | 230 | CG2 | ILE | 29 | 39.307 | 12.999 | −0.848 | 0.00 | 0.00 |
| ATOM | 231 | CD1 | ILE | 29 | 38.727 | 15.958 | 1.665 | 0.00 | 0.00 |
| ATOM | 232 | N | VAL | 30 | 42.298 | 13.939 | −0.711 | 0.00 | 0.00 |
| ATOM | 233 | CA | VAL | 30 | 43.643 | 13.534 | −0.379 | 0.00 | 0.00 |
| ATOM | 234 | C | VAL | 30 | 44.676 | 14.603 | −0.879 | 0.00 | 0.00 |
| ATOM | 235 | O | VAL | 30 | 45.661 | 14.801 | −0.166 | 0.00 | 0.00 |
| ATOM | 236 | CB | VAL | 30 | 43.919 | 12.071 | −0.804 | 0.00 | 0.00 |
| ATOM | 237 | CG1 | VAL | 30 | 42.932 | 11.054 | −0.159 | 0.00 | 0.00 |
| ATOM | 238 | CG2 | VAL | 30 | 44.133 | 11.933 | −2.305 | 0.00 | 0.00 |
| ATOM | 239 | N | MET | 31 | 44.600 | 15.005 | −2.173 | 0.00 | 0.00 |
| ATOM | 240 | CA | MET | 31 | 45.440 | 16.108 | −2.693 | 0.00 | 0.00 |
| ATOM | 241 | C | MET | 31 | 45.337 | 17.387 | −1.831 | 0.00 | 0.00 |
| ATOM | 242 | O | MET | 31 | 46.326 | 18.112 | −1.784 | 0.00 | 0.00 |
| ATOM | 243 | CB | MET | 31 | 45.211 | 16.441 | −4.208 | 0.00 | 0.00 |
| ATOM | 244 | CG | MET | 31 | 45.684 | 15.368 | −5.126 | 0.00 | 0.00 |
| ATOM | 245 | SD | MET | 31 | 47.375 | 14.817 | −4.763 | 0.00 | 0.00 |
| ATOM | 246 | CE | MET | 31 | 48.290 | 16.334 | −4.787 | 0.00 | 0.00 |
| ATOM | 247 | N | SER | 32 | 44.088 | 17.882 | −1.686 | 0.00 | 0.00 |
| ATOM | 248 | CA | SER | 32 | 43.770 | 19.048 | −0.888 | 0.00 | 0.00 |
| ATOM | 249 | C | SER | 32 | 44.547 | 18.975 | 0.463 | 0.00 | 0.00 |
| ATOM | 250 | O | SER | 32 | 45.061 | 20.025 | 0.848 | 0.00 | 0.00 |
| ATOM | 251 | CB | SER | 32 | 42.242 | 19.126 | −0.706 | 0.00 | 0.00 |
| ATOM | 252 | OG | SER | 32 | 41.481 | 19.637 | −1.788 | 0.00 | 0.00 |
| ATOM | 253 | N | LEU | 33 | 44.411 | 17.917 | 1.294 | 0.00 | 0.00 |
| ATOM | 254 | CA | LEU | 33 | 45.140 | 17.701 | 2.506 | 0.00 | 0.00 |
| ATOM | 255 | C | LEU | 33 | 46.690 | 17.860 | 2.286 | 0.00 | 0.00 |
| ATOM | 256 | O | LEU | 33 | 47.300 | 18.439 | 3.186 | 0.00 | 0.00 |
| ATOM | 257 | CB | LEU | 33 | 44.779 | 16.312 | 3.036 | 0.00 | 0.00 |
| ATOM | 258 | CG | LEU | 33 | 43.521 | 16.067 | 3.847 | 0.00 | 0.00 |
| ATOM | 259 | CD1 | LEU | 33 | 42.327 | 16.943 | 3.499 | 0.00 | 0.00 |
| ATOM | 260 | CD2 | LEU | 33 | 43.158 | 14.652 | 3.511 | 0.00 | 0.00 |
| ATOM | 261 | N | ILE | 34 | 47.348 | 17.141 | 1.332 | 0.00 | 0.00 |
| ATOM | 262 | CA | ILE | 34 | 48.774 | 17.315 | 1.001 | 0.00 | 0.00 |
| ATOM | 263 | C | ILE | 34 | 49.064 | 18.836 | 0.762 | 0.00 | 0.00 |
| ATOM | 264 | O | ILE | 34 | 49.780 | 19.368 | 1.583 | 0.00 | 0.00 |
| ATOM | 265 | CB | ILE | 34 | 49.155 | 16.445 | −0.241 | 0.00 | 0.00 |
| ATOM | 266 | CG1 | ILE | 34 | 49.439 | 14.983 | 0.179 | 0.00 | 0.00 |
| ATOM | 267 | CG2 | ILE | 34 | 50.414 | 17.047 | −0.970 | 0.00 | 0.00 |
| ATOM | 268 | CD1 | ILE | 34 | 49.621 | 13.952 | −0.963 | 0.00 | 0.00 |
| ATOM | 269 | N | VAL | 35 | 48.286 | 19.586 | −0.065 | 0.00 | 0.00 |
| ATOM | 270 | CA | VAL | 35 | 48.409 | 21.059 | −0.311 | 0.00 | 0.00 |
| ATOM | 271 | C | VAL | 35 | 48.439 | 21.835 | 1.055 | 0.00 | 0.00 |
| ATOM | 272 | O | VAL | 35 | 49.440 | 22.516 | 1.327 | 0.00 | 0.00 |
| ATOM | 273 | CB | VAL | 35 | 47.406 | 21.632 | −1.382 | 0.00 | 0.00 |
| ATOM | 274 | CG1 | VAL | 35 | 47.302 | 23.184 | −1.439 | 0.00 | 0.00 |
| ATOM | 275 | CG2 | VAL | 35 | 47.778 | 20.968 | −2.758 | 0.00 | 0.00 |
| ATOM | 276 | N | LEU | 36 | 47.425 | 21.716 | 1.896 | 0.00 | 0.00 |
| ATOM | 277 | CA | LEU | 36 | 47.336 | 22.397 | 3.181 | 0.00 | 0.00 |
| ATOM | 278 | C | LEU | 36 | 48.558 | 22.038 | 4.110 | 0.00 | 0.00 |
| ATOM | 279 | O | LEU | 36 | 49.215 | 22.986 | 4.544 | 0.00 | 0.00 |
| ATOM | 280 | CB | LEU | 36 | 45.971 | 22.117 | 3.834 | 0.00 | 0.00 |
| ATOM | 281 | CG | LEU | 36 | 45.593 | 22.954 | 5.060 | 0.00 | 0.00 |
| ATOM | 282 | CD1 | LEU | 36 | 45.595 | 24.465 | 4.776 | 0.00 | 0.00 |
| ATOM | 283 | CD2 | LEU | 36 | 44.252 | 22.445 | 5.501 | 0.00 | 0.00 |
| ATOM | 284 | N | ALA | 37 | 48.858 | 20.753 | 4.422 | 0.00 | 0.00 |
| ATOM | 285 | CA | ALA | 37 | 50.031 | 20.359 | 5.217 | 0.00 | 0.00 |
| ATOM | 286 | C | ALA | 37 | 51.432 | 20.733 | 4.571 | 0.00 | 0.00 |
| ATOM | 287 | O | ALA | 37 | 52.292 | 21.121 | 5.351 | 0.00 | 0.00 |
| ATOM | 288 | CB | ALA | 37 | 49.903 | 18.854 | 5.489 | 0.00 | 0.00 |
| ATOM | 289 | N | ILE | 38 | 51.750 | 20.251 | 3.368 | 0.00 | 0.00 |
| ATOM | 290 | CA | ILE | 38 | 52.980 | 20.529 | 2.602 | 0.00 | 0.00 |
| ATOM | 291 | C | ILE | 38 | 53.289 | 22.083 | 2.533 | 0.00 | 0.00 |
| ATOM | 292 | O | ILE | 38 | 54.465 | 22.424 | 2.683 | 0.00 | 0.00 |
| ATOM | 293 | CB | ILE | 38 | 52.911 | 19.954 | 1.168 | 0.00 | 0.00 |
| ATOM | 294 | CG1 | ILE | 38 | 52.062 | 20.725 | 0.185 | 0.00 | 0.00 |
| ATOM | 295 | CG2 | ILE | 38 | 52.649 | 18.434 | 1.186 | 0.00 | 0.00 |
| ATOM | 296 | CD1 | ILE | 38 | 52.006 | 20.193 | −1.262 | 0.00 | 0.00 |
| ATOM | 297 | N | VAL | 39 | 52.315 | 22.992 | 2.240 | 0.00 | 0.00 |
| ATOM | 298 | CA | VAL | 39 | 52.524 | 24.429 | 2.267 | 0.00 | 0.00 |
| ATOM | 299 | C | VAL | 39 | 52.976 | 24.906 | 3.691 | 0.00 | 0.00 |
| ATOM | 300 | O | VAL | 39 | 54.143 | 25.255 | 3.802 | 0.00 | 0.00 |
| ATOM | 301 | CB | VAL | 39 | 51.259 | 25.174 | 1.793 | 0.00 | 0.00 |
| ATOM | 302 | CG1 | VAL | 39 | 51.415 | 26.689 | 2.035 | 0.00 | 0.00 |
| ATOM | 303 | CG2 | VAL | 39 | 50.982 | 24.940 | 0.328 | 0.00 | 0.00 |
| ATOM | 304 | N | PHE | 40 | 52.202 | 24.694 | 4.792 | 0.00 | 0.00 |
| ATOM | 305 | CA | PHE | 40 | 52.564 | 25.112 | 6.152 | 0.00 | 0.00 |
| ATOM | 306 | C | PHE | 40 | 53.850 | 24.412 | 6.718 | 0.00 | 0.00 |
| ATOM | 307 | O | PHE | 40 | 54.665 | 25.121 | 7.324 | 0.00 | 0.00 |
| ATOM | 308 | CB | PHE | 40 | 51.338 | 24.878 | 7.040 | 0.00 | 0.00 |
| ATOM | 309 | CG | PHE | 40 | 50.398 | 26.065 | 7.048 | 0.00 | 0.00 |
| ATOM | 310 | CD1 | PHE | 40 | 50.668 | 27.222 | 6.305 | 0.00 | 0.00 |
| ATOM | 311 | CD2 | PHE | 40 | 49.264 | 26.003 | 7.856 | 0.00 | 0.00 |
| ATOM | 312 | CE1 | PHE | 40 | 49.815 | 28.317 | 6.371 | 0.00 | 0.00 |
| ATOM | 313 | CE2 | PHE | 40 | 48.412 | 27.107 | 7.918 | 0.00 | 0.00 |
| ATOM | 314 | CZ | PHE | 40 | 48.686 | 28.262 | 7.179 | 0.00 | 0.00 |
| ATOM | 315 | N | GLY | 41 | 53.993 | 23.063 | 6.631 | 0.00 | 0.00 |
| ATOM | 316 | CA | GLY | 41 | 55.180 | 22.285 | 7.024 | 0.00 | 0.00 |
| ATOM | 317 | C | GLY | 41 | 56.512 | 22.798 | 6.425 | 0.00 | 0.00 |
| ATOM | 318 | O | GLY | 41 | 57.129 | 23.578 | 7.116 | 0.00 | 0.00 |
| ATOM | 319 | N | ASN | 42 | 56.594 | 22.877 | 5.093 | 0.00 | 0.00 |
| ATOM | 320 | CA | ASN | 42 | 57.792 | 23.265 | 4.293 | 0.00 | 0.00 |
| ATOM | 321 | C | ASN | 42 | 57.958 | 24.798 | 4.372 | 0.00 | 0.00 |
| ATOM | 322 | O | ASN | 42 | 59.012 | 25.199 | 4.835 | 0.00 | 0.00 |
| ATOM | 323 | CB | ASN | 42 | 57.500 | 22.770 | 2.902 | 0.00 | 0.00 |
| ATOM | 324 | CG | ASN | 42 | 57.785 | 21.337 | 2.647 | 0.00 | 0.00 |
| ATOM | 325 | OD1 | ASN | 42 | 58.920 | 20.891 | 2.857 | 0.00 | 0.00 |
| ATOM | 326 | ND2 | ASN | 42 | 56.784 | 20.533 | 2.285 | 0.00 | 0.00 |
| ATOM | 327 | N | VAL | 43 | 56.952 | 25.639 | 4.033 | 0.00 | 0.00 |
| ATOM | 328 | CA | VAL | 43 | 57.030 | 27.100 | 4.193 | 0.00 | 0.00 |
| ATOM | 329 | C | VAL | 43 | 57.552 | 27.464 | 5.619 | 0.00 | 0.00 |
| ATOM | 330 | O | VAL | 43 | 58.544 | 28.204 | 5.657 | 0.00 | 0.00 |
| ATOM | 331 | CB | VAL | 43 | 55.682 | 27.782 | 3.894 | 0.00 | 0.00 |
| ATOM | 332 | CG1 | VAL | 43 | 55.568 | 29.204 | 4.437 | 0.00 | 0.00 |
| ATOM | 333 | CG2 | VAL | 43 | 55.388 | 27.745 | 2.400 | 0.00 | 0.00 |
| ATOM | 334 | N | LEU | 44 | 56.889 | 27.059 | 6.728 | 0.00 | 0.00 |
| ATOM | 335 | CA | LEU | 44 | 57.389 | 27.306 | 8.090 | 0.00 | 0.00 |
| ATOM | 336 | C | LEU | 44 | 58.876 | 26.847 | 8.198 | 0.00 | 0.00 |
| ATOM | 337 | O | LEU | 44 | 59.634 | 27.613 | 8.785 | 0.00 | 0.00 |
| ATOM | 338 | CB | LEU | 44 | 56.481 | 26.610 | 9.133 | 0.00 | 0.00 |
| ATOM | 339 | CG | LEU | 44 | 57.118 | 26.694 | 10.532 | 0.00 | 0.00 |
| ATOM | 340 | CD1 | LEU | 44 | 57.194 | 28.153 | 10.959 | 0.00 | 0.00 |
| ATOM | 341 | CD2 | LEU | 44 | 56.281 | 25.913 | 11.508 | 0.00 | 0.00 |
| ATOM | 342 | N | VAL | 45 | 59.229 | 25.601 | 7.866 | 0.00 | 0.00 |
| ATOM | 343 | CA | VAL | 45 | 60.593 | 25.117 | 7.837 | 0.00 | 0.00 |
| ATOM | 344 | C | VAL | 45 | 61.557 | 26.056 | 7.003 | 0.00 | 0.00 |
| ATOM | 345 | O | VAL | 45 | 62.654 | 26.307 | 7.502 | 0.00 | 0.00 |
| ATOM | 346 | CB | VAL | 45 | 60.640 | 23.661 | 7.422 | 0.00 | 0.00 |
| ATOM | 347 | CG1 | VAL | 45 | 62.036 | 23.062 | 7.418 | 0.00 | 0.00 |
| ATOM | 348 | CG2 | VAL | 45 | 59.620 | 22.786 | 8.143 | 0.00 | 0.00 |
| ATOM | 349 | N | ILE | 46 | 61.217 | 26.503 | 5.771 | 0.00 | 0.00 |
| ATOM | 350 | CA | ILE | 46 | 62.034 | 27.441 | 5.020 | 0.00 | 0.00 |
| ATOM | 351 | C | ILE | 46 | 62.139 | 28.791 | 5.786 | 0.00 | 0.00 |
| ATOM | 352 | O | ILE | 46 | 63.279 | 29.249 | 5.902 | 0.00 | 0.00 |
| ATOM | 353 | CB | ILE | 46 | 61.552 | 27.527 | 3.540 | 0.00 | 0.00 |
| ATOM | 354 | CG1 | ILE | 46 | 61.918 | 28.714 | 2.740 | 0.00 | 0.00 |
| ATOM | 355 | CG2 | ILE | 46 | 60.083 | 27.254 | 3.380 | 0.00 | 0.00 |
| ATOM | 356 | CD1 | ILE | 46 | 61.887 | 28.442 | 1.233 | 0.00 | 0.00 |
| ATOM | 357 | N | THR | 47 | 61.034 | 29.522 | 6.094 | 0.00 | 0.00 |
| ATOM | 358 | CA | THR | 47 | 61.131 | 30.729 | 6.902 | 0.00 | 0.00 |
| ATOM | 359 | C | THR | 47 | 62.028 | 30.554 | 8.196 | 0.00 | 0.00 |
| ATOM | 360 | O | THR | 47 | 62.634 | 31.550 | 8.590 | 0.00 | 0.00 |
| ATOM | 361 | CB | THR | 47 | 59.727 | 31.284 | 7.299 | 0.00 | 0.00 |
| ATOM | 362 | OG1 | THR | 47 | 58.778 | 31.267 | 6.200 | 0.00 | 0.00 |
| ATOM | 363 | CG2 | THR | 47 | 59.736 | 32.715 | 7.921 | 0.00 | 0.00 |
| ATOM | 364 | N | ALA | 48 | 61.918 | 29.467 | 8.987 | 0.00 | 0.00 |
| ATOM | 365 | CA | ALA | 48 | 62.757 | 29.154 | 10.148 | 0.00 | 0.00 |
| ATOM | 366 | C | ALA | 48 | 64.295 | 29.072 | 9.829 | 0.00 | 0.00 |
| ATOM | 367 | O | ALA | 48 | 65.030 | 29.710 | 10.590 | 0.00 | 0.00 |
| ATOM | 368 | CB | ALA | 48 | 62.185 | 27.881 | 10.783 | 0.00 | 0.00 |
| ATOM | 369 | N | ILE | 49 | 64.779 | 28.257 | 8.845 | 0.00 | 0.00 |
| ATOM | 370 | CA | ILE | 49 | 66.248 | 28.277 | 8.554 | 0.00 | 0.00 |
| ATOM | 371 | C | ILE | 49 | 66.712 | 29.683 | 8.023 | 0.00 | 0.00 |
| ATOM | 372 | O | ILE | 49 | 67.720 | 30.165 | 8.559 | 0.00 | 0.00 |
| ATOM | 373 | CB | ILE | 49 | 66.736 | 27.113 | 7.671 | 0.00 | 0.00 |
| ATOM | 374 | CG1 | ILE | 49 | 66.148 | 25.754 | 8.022 | 0.00 | 0.00 |
| ATOM | 375 | CG2 | ILE | 49 | 68.304 | 27.045 | 7.582 | 0.00 | 0.00 |
| ATOM | 376 | CD1 | ILE | 49 | 66.548 | 24.575 | 7.114 | 0.00 | 0.00 |
| ATOM | 377 | N | ALA | 50 | 66.029 | 30.333 | 7.071 | 0.00 | 0.00 |
| ATOM | 378 | CA | ALA | 50 | 66.343 | 31.691 | 6.590 | 0.00 | 0.00 |
| ATOM | 379 | C | ALA | 50 | 66.416 | 32.737 | 7.756 | 0.00 | 0.00 |
| ATOM | 380 | O | ALA | 50 | 67.465 | 33.403 | 7.836 | 0.00 | 0.00 |
| ATOM | 381 | CB | ALA | 50 | 65.279 | 32.050 | 5.523 | 0.00 | 0.00 |
| ATOM | 382 | N | LYS | 51 | 65.372 | 32.949 | 8.560 | 0.00 | 0.00 |
| ATOM | 383 | CA | LYS | 51 | 65.443 | 33.901 | 9.677 | 0.00 | 0.00 |
| ATOM | 384 | C | LYS | 51 | 66.449 | 33.509 | 10.824 | 0.00 | 0.00 |
| ATOM | 385 | O | LYS | 51 | 67.030 | 34.455 | 11.366 | 0.00 | 0.00 |
| ATOM | 386 | CB | LYS | 51 | 64.027 | 34.061 | 10.235 | 0.00 | 0.00 |
| ATOM | 387 | CG | LYS | 51 | 63.747 | 35.082 | 11.344 | 0.00 | 0.00 |
| ATOM | 388 | CD | LYS | 51 | 62.155 | 35.241 | 11.442 | 0.00 | 0.00 |
| ATOM | 389 | CE | LYS | 51 | 61.659 | 35.425 | 10.051 | 0.00 | 0.00 |
| ATOM | 390 | NZ | LYS | 51 | 61.721 | 36.763 | 9.595 | 0.00 | 0.00 |
| ATOM | 391 | N | PHE | 52 | 66.819 | 32.218 | 11.035 | 0.00 | 0.00 |
| ATOM | 392 | CA | PHE | 52 | 67.673 | 31.889 | 12.184 | 0.00 | 0.00 |
| ATOM | 393 | C | PHE | 52 | 69.182 | 32.007 | 11.745 | 0.00 | 0.00 |
| ATOM | 394 | O | PHE | 52 | 69.649 | 31.199 | 10.924 | 0.00 | 0.00 |
| ATOM | 395 | CB | PHE | 52 | 67.169 | 30.460 | 12.505 | 0.00 | 0.00 |
| ATOM | 396 | CG | PHE | 52 | 66.297 | 30.478 | 13.789 | 0.00 | 0.00 |
| ATOM | 397 | CD1 | PHE | 52 | 65.357 | 31.493 | 13.904 | 0.00 | 0.00 |
| ATOM | 398 | CD2 | PHE | 52 | 66.379 | 29.479 | 14.760 | 0.00 | 0.00 |
| ATOM | 399 | CE1 | PHE | 52 | 64.499 | 31.551 | 14.998 | 0.00 | 0.00 |
| ATOM | 400 | CE2 | PHE | 52 | 65.499 | 29.512 | 15.863 | 0.00 | 0.00 |
| ATOM | 401 | CZ | PHE | 52 | 64.559 | 30.535 | 15.950 | 0.00 | 0.00 |
| ATOM | 402 | N | GLU | 53 | 69.838 | 33.121 | 12.141 | 0.00 | 0.00 |
| ATOM | 403 | CA | GLU | 53 | 71.224 | 33.483 | 11.772 | 0.00 | 0.00 |
| ATOM | 404 | C | GLU | 53 | 72.253 | 32.300 | 11.846 | 0.00 | 0.00 |
| ATOM | 405 | O | GLU | 53 | 72.905 | 32.097 | 10.813 | 0.00 | 0.00 |
| ATOM | 406 | CB | GLU | 53 | 71.714 | 34.639 | 12.639 | 0.00 | 0.00 |
| ATOM | 407 | CG | GLU | 53 | 71.258 | 35.992 | 12.140 | 0.00 | 0.00 |
| ATOM | 408 | CD | GLU | 53 | 71.530 | 37.134 | 13.113 | 0.00 | 0.00 |
| ATOM | 409 | OE1 | GLU | 53 | 72.631 | 37.187 | 13.681 | 0.00 | 0.00 |
| ATOM | 410 | OE2 | GLU | 53 | 70.624 | 37.956 | 13.302 | 0.00 | 0.00 |
| ATOM | 411 | N | ARG | 54 | 72.279 | 31.449 | 12.896 | 0.00 | 0.00 |
| ATOM | 412 | CA | ARG | 54 | 73.320 | 30.406 | 12.978 | 0.00 | 0.00 |
| ATOM | 413 | C | ARG | 54 | 72.856 | 28.970 | 12.536 | 0.00 | 0.00 |
| ATOM | 414 | O | ARG | 54 | 73.621 | 28.032 | 12.746 | 0.00 | 0.00 |
| ATOM | 415 | CB | ARG | 54 | 73.838 | 30.307 | 14.395 | 0.00 | 0.00 |
| ATOM | 416 | CG | ARG | 54 | 74.320 | 31.547 | 15.050 | 0.00 | 0.00 |
| ATOM | 417 | CD | ARG | 54 | 75.345 | 32.183 | 14.170 | 0.00 | 0.00 |
| ATOM | 418 | NE | ARG | 54 | 76.577 | 31.436 | 13.999 | 0.00 | 0.00 |
| ATOM | 419 | CZ | ARG | 54 | 77.478 | 31.159 | 14.947 | 0.00 | 0.00 |
| ATOM | 420 | NH1 | ARG | 54 | 77.308 | 31.512 | 16.210 | 0.00 | 0.00 |
| ATOM | 421 | NH2 | ARG | 54 | 78.551 | 30.420 | 14.622 | 0.00 | 0.00 |
| ATOM | 422 | N | LEU | 55 | 71.690 | 28.775 | 11.887 | 0.00 | 0.00 |
| ATOM | 423 | CA | LEU | 55 | 71.224 | 27.467 | 11.342 | 0.00 | 0.00 |
| ATOM | 424 | C | LEU | 55 | 71.903 | 27.331 | 9.952 | 0.00 | 0.00 |
| ATOM | 425 | O | LEU | 55 | 71.219 | 27.473 | 8.931 | 0.00 | 0.00 |
| ATOM | 426 | CB | LEU | 55 | 69.687 | 27.446 | 11.266 | 0.00 | 0.00 |
| ATOM | 427 | CG | LEU | 55 | 69.112 | 26.484 | 12.343 | 0.00 | 0.00 |
| ATOM | 428 | CD1 | LEU | 55 | 69.750 | 26.636 | 13.709 | 0.00 | 0.00 |
| ATOM | 429 | CD2 | LEU | 55 | 67.618 | 26.568 | 12.475 | 0.00 | 0.00 |
| ATOM | 430 | N | GLN | 56 | 73.210 | 27.002 | 9.883 | 0.00 | 0.00 |
| ATOM | 431 | CA | GLN | 56 | 74.010 | 26.942 | 8.626 | 0.00 | 0.00 |
| ATOM | 432 | C | GLN | 56 | 74.605 | 25.556 | 8.299 | 0.00 | 0.00 |
| ATOM | 433 | O | GLN | 56 | 75.197 | 25.468 | 7.220 | 0.00 | 0.00 |
| ATOM | 434 | CB | GLN | 56 | 75.130 | 27.966 | 8.827 | 0.00 | 0.00 |
| ATOM | 435 | CG | GLN | 56 | 74.724 | 29.415 | 8.955 | 0.00 | 0.00 |
| ATOM | 436 | CD | GLN | 56 | 73.896 | 29.994 | 7.840 | 0.00 | 0.00 |
| ATOM | 437 | OE1 | GLN | 56 | 74.087 | 29.682 | 6.659 | 0.00 | 0.00 |
| ATOM | 438 | NE2 | GLN | 56 | 72.948 | 30.872 | 8.162 | 0.00 | 0.00 |
| ATOM | 439 | N | THR | 57 | 74.518 | 24.524 | 9.178 | 0.00 | 0.00 |
| ATOM | 440 | CA | THR | 57 | 75.128 | 23.264 | 8.924 | 0.00 | 0.00 |
| ATOM | 441 | C | THR | 57 | 74.657 | 22.707 | 7.557 | 0.00 | 0.00 |
| ATOM | 442 | O | THR | 57 | 73.564 | 23.065 | 7.093 | 0.00 | 0.00 |
| ATOM | 443 | CB | THR | 57 | 74.815 | 22.261 | 10.075 | 0.00 | 0.00 |
| ATOM | 444 | OG1 | THR | 57 | 73.485 | 21.652 | 9.991 | 0.00 | 0.00 |
| ATOM | 445 | CG2 | THR | 57 | 74.956 | 22.914 | 11.495 | 0.00 | 0.00 |
| ATOM | 446 | N | VAL | 58 | 75.524 | 22.016 | 6.819 | 0.00 | 0.00 |
| ATOM | 447 | CA | VAL | 58 | 75.086 | 21.382 | 5.576 | 0.00 | 0.00 |
| ATOM | 448 | C | VAL | 58 | 73.712 | 20.657 | 5.759 | 0.00 | 0.00 |
| ATOM | 449 | O | VAL | 58 | 72.947 | 20.662 | 4.811 | 0.00 | 0.00 |
| ATOM | 450 | CB | VAL | 58 | 76.245 | 20.473 | 5.141 | 0.00 | 0.00 |
| ATOM | 451 | CG1 | VAL | 58 | 75.900 | 19.381 | 4.113 | 0.00 | 0.00 |
| ATOM | 452 | CG2 | VAL | 58 | 77.380 | 21.381 | 4.563 | 0.00 | 0.00 |
| ATOM | 453 | N | THR | 59 | 73.454 | 19.942 | 6.890 | 0.00 | 0.00 |
| ATOM | 454 | CA | THR | 59 | 72.179 | 19.288 | 7.241 | 0.00 | 0.00 |
| ATOM | 455 | C | THR | 59 | 71.018 | 20.323 | 7.216 | 0.00 | 0.00 |
| ATOM | 456 | O | THR | 59 | 69.941 | 19.931 | 6.724 | 0.00 | 0.00 |
| ATOM | 457 | CB | THR | 59 | 72.290 | 18.586 | 8.638 | 0.00 | 0.00 |
| ATOM | 458 | OG1 | THR | 59 | 73.500 | 17.829 | 8.813 | 0.00 | 0.00 |
| ATOM | 459 | CG2 | THR | 59 | 71.066 | 17.647 | 8.949 | 0.00 | 0.00 |
| ATOM | 460 | N | ASN | 60 | 71.046 | 21.394 | 8.015 | 0.00 | 0.00 |
| ATOM | 461 | CA | ASN | 60 | 70.020 | 22.454 | 7.954 | 0.00 | 0.00 |
| ATOM | 462 | C | ASN | 60 | 69.914 | 22.946 | 6.495 | 0.00 | 0.00 |
| ATOM | 463 | O | ASN | 60 | 68.772 | 23.194 | 6.091 | 0.00 | 0.00 |
| ATOM | 464 | CB | ASN | 60 | 70.343 | 23.575 | 8.951 | 0.00 | 0.00 |
| ATOM | 465 | CG | ASN | 60 | 70.133 | 23.181 | 10.410 | 0.00 | 0.00 |
| ATOM | 466 | OD1 | ASN | 60 | 70.419 | 23.854 | 11.414 | 0.00 | 0.00 |
| ATOM | 467 | ND2 | ASN | 60 | 69.588 | 21.981 | 10.594 | 0.00 | 0.00 |
| ATOM | 468 | N | TYR | 61 | 71.008 | 23.287 | 5.784 | 0.00 | 0.00 |
| ATOM | 469 | CA | TYR | 61 | 70.860 | 23.638 | 4.386 | 0.00 | 0.00 |
| ATOM | 470 | C | TYR | 61 | 70.143 | 22.593 | 3.532 | 0.00 | 0.00 |
| ATOM | 471 | O | TYR | 61 | 69.377 | 23.026 | 2.671 | 0.00 | 0.00 |
| ATOM | 472 | CB | TYR | 61 | 72.201 | 23.958 | 3.716 | 0.00 | 0.00 |
| ATOM | 473 | CG | TYR | 61 | 72.672 | 25.362 | 3.823 | 0.00 | 0.00 |
| ATOM | 474 | CD1 | TYR | 61 | 71.959 | 26.362 | 3.188 | 0.00 | 0.00 |
| ATOM | 475 | CD2 | TYR | 61 | 73.823 | 25.649 | 4.536 | 0.00 | 0.00 |
| ATOM | 476 | CE1 | TYR | 61 | 72.399 | 27.664 | 3.268 | 0.00 | 0.00 |
| ATOM | 477 | CE2 | TYR | 61 | 74.264 | 26.946 | 4.619 | 0.00 | 0.00 |
| ATOM | 478 | CZ | TYR | 61 | 73.548 | 27.931 | 3.981 | 0.00 | 0.00 |
| ATOM | 479 | OH | TYR | 61 | 73.994 | 29.232 | 4.034 | 0.00 | 0.00 |
| ATOM | 480 | N | PHE | 62 | 70.659 | 21.371 | 3.469 | 0.00 | 0.00 |
| ATOM | 481 | CA | PHE | 62 | 70.031 | 20.299 | 2.783 | 0.00 | 0.00 |
| ATOM | 482 | C | PHE | 62 | 68.487 | 20.433 | 2.997 | 0.00 | 0.00 |
| ATOM | 483 | O | PHE | 62 | 67.706 | 20.195 | 2.077 | 0.00 | 0.00 |
| ATOM | 484 | CB | PHE | 62 | 70.484 | 19.071 | 3.571 | 0.00 | 0.00 |
| ATOM | 485 | CG | PHE | 62 | 71.608 | 18.266 | 3.087 | 0.00 | 0.00 |
| ATOM | 486 | CD1 | PHE | 62 | 71.725 | 17.825 | 1.770 | 0.00 | 0.00 |
| ATOM | 487 | CD2 | PHE | 62 | 72.631 | 18.033 | 3.983 | 0.00 | 0.00 |
| ATOM | 488 | CE1 | PHE | 62 | 72.882 | 17.156 | 1.343 | 0.00 | 0.00 |
| ATOM | 489 | CE2 | PHE | 62 | 73.793 | 17.367 | 3.599 | 0.00 | 0.00 |
| ATOM | 490 | CZ | PHE | 62 | 73.899 | 16.939 | 2.265 | 0.00 | 0.00 |
| ATOM | 491 | N | ILE | 63 | 68.162 | 20.467 | 4.318 | 0.00 | 0.00 |
| ATOM | 492 | CA | ILE | 63 | 66.816 | 20.627 | 4.871 | 0.00 | 0.00 |
| ATOM | 493 | C | ILE | 63 | 66.065 | 21.792 | 4.176 | 0.00 | 0.00 |
| ATOM | 494 | O | ILE | 63 | 64.907 | 21.553 | 3.835 | 0.00 | 0.00 |
| ATOM | 495 | CB | ILE | 63 | 66.760 | 20.684 | 6.437 | 0.00 | 0.00 |
| ATOM | 496 | CG1 | ILE | 63 | 66.410 | 19.331 | 7.006 | 0.00 | 0.00 |
| ATOM | 497 | CG2 | ILE | 63 | 65.718 | 21.758 | 6.976 | 0.00 | 0.00 |
| ATOM | 498 | CD1 | ILE | 63 | 67.215 | 18.884 | 8.242 | 0.00 | 0.00 |
| ATOM | 499 | N | THR | 64 | 66.622 | 23.038 | 4.071 | 0.00 | 0.00 |
| ATOM | 500 | CA | THR | 64 | 65.923 | 24.159 | 3.360 | 0.00 | 0.00 |
| ATOM | 501 | C | THR | 64 | 65.602 | 23.756 | 1.888 | 0.00 | 0.00 |
| ATOM | 502 | O | THR | 64 | 64.622 | 24.298 | 1.389 | 0.00 | 0.00 |
| ATOM | 503 | CB | THR | 64 | 66.630 | 25.566 | 3.308 | 0.00 | 0.00 |
| ATOM | 504 | OG1 | THR | 64 | 67.414 | 25.876 | 4.440 | 0.00 | 0.00 |
| ATOM | 505 | CG2 | THR | 64 | 65.519 | 26.672 | 3.144 | 0.00 | 0.00 |
| ATOM | 506 | N | SER | 65 | 66.620 | 23.365 | 1.105 | 0.00 | 0.00 |
| ATOM | 507 | CA | SER | 65 | 66.515 | 22.882 | −0.285 | 0.00 | 0.00 |
| ATOM | 508 | C | SER | 65 | 65.246 | 21.985 | −0.468 | 0.00 | 0.00 |
| ATOM | 509 | O | SER | 65 | 64.694 | 22.045 | −1.574 | 0.00 | 0.00 |
| ATOM | 510 | CB | SER | 65 | 67.814 | 22.182 | −0.704 | 0.00 | 0.00 |
| ATOM | 511 | OG | SER | 65 | 67.936 | 21.886 | −2.083 | 0.00 | 0.00 |
| ATOM | 512 | N | LEU | 66 | 65.101 | 20.903 | 0.304 | 0.00 | 0.00 |
| ATOM | 513 | CA | LEU | 66 | 63.927 | 20.083 | 0.279 | 0.00 | 0.00 |
| ATOM | 514 | C | LEU | 66 | 62.685 | 20.936 | 0.643 | 0.00 | 0.00 |
| ATOM | 515 | O | LEU | 66 | 61.646 | 20.570 | 0.167 | 0.00 | 0.00 |
| ATOM | 516 | CB | LEU | 66 | 64.146 | 18.983 | 1.319 | 0.00 | 0.00 |
| ATOM | 517 | CG | LEU | 66 | 65.340 | 18.064 | 1.164 | 0.00 | 0.00 |
| ATOM | 518 | CD1 | LEU | 66 | 65.317 | 17.154 | 2.376 | 0.00 | 0.00 |
| ATOM | 519 | CD2 | LEU | 66 | 65.376 | 17.397 | −0.202 | 0.00 | 0.00 |
| ATOM | 520 | N | ALA | 67 | 62.755 | 21.902 | 1.593 | 0.00 | 0.00 |
| ATOM | 521 | CA | ALA | 67 | 61.619 | 22.777 | 1.871 | 0.00 | 0.00 |
| ATOM | 522 | C | ALA | 67 | 61.197 | 23.477 | 0.532 | 0.00 | 0.00 |
| ATOM | 523 | O | ALA | 67 | 59.998 | 23.677 | 0.367 | 0.00 | 0.00 |
| ATOM | 524 | CB | ALA | 67 | 61.984 | 23.728 | 3.022 | 0.00 | 0.00 |
| ATOM | 525 | N | CYS | 68 | 62.125 | 24.149 | −0.184 | 0.00 | 0.00 |
| ATOM | 526 | CA | CYS | 68 | 61.902 | 24.758 | −1.483 | 0.00 | 0.00 |
| ATOM | 527 | C | CYS | 68 | 61.383 | 23.682 | −2.493 | 0.00 | 0.00 |
| ATOM | 528 | O | CYS | 68 | 60.549 | 24.078 | −3.305 | 0.00 | 0.00 |
| ATOM | 529 | CB | CYS | 68 | 63.246 | 25.371 | −1.948 | 0.00 | 0.00 |
| ATOM | 530 | SG | CYS | 68 | 63.781 | 26.812 | −0.973 | 0.00 | 0.00 |
| ATOM | 531 | N | ALA | 69 | 62.118 | 22.565 | −2.742 | 0.00 | 0.00 |
| ATOM | 532 | CA | ALA | 69 | 61.679 | 21.451 | −3.598 | 0.00 | 0.00 |
| ATOM | 533 | C | ALA | 69 | 60.227 | 21.037 | −3.248 | 0.00 | 0.00 |
| ATOM | 534 | O | ALA | 69 | 59.416 | 21.135 | −4.143 | 0.00 | 0.00 |
| ATOM | 535 | CB | ALA | 69 | 62.683 | 20.283 | −3.494 | 0.00 | 0.00 |
| ATOM | 536 | N | ASP | 70 | 59.915 | 20.905 | −1.984 | 0.00 | 0.00 |
| ATOM | 537 | CA | ASP | 70 | 58.576 | 20.568 | −1.425 | 0.00 | 0.00 |
| ATOM | 538 | C | ASP | 70 | 57.528 | 21.674 | −1.822 | 0.00 | 0.00 |
| ATOM | 539 | O | ASP | 70 | 56.351 | 21.324 | −1.907 | 0.00 | 0.00 |
| ATOM | 540 | CB | ASP | 70 | 58.804 | 20.403 | 0.045 | 0.00 | 0.00 |
| ATOM | 541 | CG | ASP | 70 | 59.473 | 19.109 | 0.404 | 0.00 | 0.00 |
| ATOM | 542 | OD1 | ASP | 70 | 59.912 | 18.352 | −0.517 | 0.00 | 0.00 |
| ATOM | 543 | OD2 | ASP | 70 | 59.766 | 18.842 | 1.626 | 0.00 | 0.00 |
| ATOM | 544 | N | LEU | 71 | 57.841 | 22.978 | −1.696 | 0.00 | 0.00 |
| ATOM | 545 | CA | LEU | 71 | 56.974 | 24.056 | −2.136 | 0.00 | 0.00 |
| ATOM | 546 | C | LEU | 71 | 56.684 | 23.864 | −3.659 | 0.00 | 0.00 |
| ATOM | 547 | O | LEU | 71 | 55.557 | 24.164 | −4.027 | 0.00 | 0.00 |
| ATOM | 548 | CB | LEU | 71 | 57.564 | 25.426 | −1.840 | 0.00 | 0.00 |
| ATOM | 549 | CG | LEU | 71 | 57.746 | 25.748 | −0.374 | 0.00 | 0.00 |
| ATOM | 550 | CD1 | LEU | 71 | 58.330 | 27.165 | −0.266 | 0.00 | 0.00 |
| ATOM | 551 | CD2 | LEU | 71 | 56.360 | 25.708 | 0.283 | 0.00 | 0.00 |
| ATOM | 552 | N | VAL | 72 | 57.730 | 23.837 | −4.517 | 0.00 | 0.00 |
| ATOM | 553 | CA | VAL | 72 | 57.679 | 23.569 | −5.962 | 0.00 | 0.00 |
| ATOM | 554 | C | VAL | 72 | 56.673 | 22.374 | −6.180 | 0.00 | 0.00 |
| ATOM | 555 | O | VAL | 72 | 55.878 | 22.483 | −7.085 | 0.00 | 0.00 |
| ATOM | 556 | CB | VAL | 72 | 59.134 | 23.230 | −6.448 | 0.00 | 0.00 |
| ATOM | 557 | CG1 | VAL | 72 | 59.102 | 22.586 | −7.880 | 0.00 | 0.00 |
| ATOM | 558 | CG2 | VAL | 72 | 59.986 | 24.450 | −6.432 | 0.00 | 0.00 |
| ATOM | 559 | N | MET | 73 | 56.793 | 21.254 | −5.430 | 0.00 | 0.00 |
| ATOM | 560 | CA | MET | 73 | 55.899 | 20.123 | −5.437 | 0.00 | 0.00 |
| ATOM | 561 | C | MET | 73 | 54.406 | 20.583 | −5.354 | 0.00 | 0.00 |
| ATOM | 562 | O | MET | 73 | 53.852 | 20.797 | −6.427 | 0.00 | 0.00 |
| ATOM | 563 | CB | MET | 73 | 56.274 | 19.313 | −4.151 | 0.00 | 0.00 |
| ATOM | 564 | CG | MET | 73 | 57.737 | 18.821 | −4.289 | 0.00 | 0.00 |
| ATOM | 565 | SD | MET | 73 | 58.046 | 17.715 | −2.884 | 0.00 | 0.00 |
| ATOM | 566 | CE | MET | 73 | 57.288 | 16.214 | −3.402 | 0.00 | 0.00 |
| ATOM | 567 | N | GLY | 74 | 54.096 | 21.264 | −4.258 | 0.00 | 0.00 |
| ATOM | 568 | CA | GLY | 74 | 52.791 | 21.725 | −3.868 | 0.00 | 0.00 |
| ATOM | 569 | C | GLY | 74 | 52.077 | 22.650 | −4.840 | 0.00 | 0.00 |
| ATOM | 570 | O | GLY | 74 | 50.873 | 22.741 | −4.686 | 0.00 | 0.00 |
| ATOM | 571 | N | LEU | 75 | 52.775 | 23.789 | −5.096 | 0.00 | 0.00 |
| ATOM | 572 | CA | LEU | 75 | 52.356 | 24.815 | −6.051 | 0.00 | 0.00 |
| ATOM | 573 | C | LEU | 75 | 52.496 | 24.411 | −7.563 | 0.00 | 0.00 |
| ATOM | 574 | O | LEU | 75 | 51.766 | 25.018 | −8.370 | 0.00 | 0.00 |
| ATOM | 575 | CB | LEU | 75 | 53.205 | 26.082 | −5.784 | 0.00 | 0.00 |
| ATOM | 576 | CG | LEU | 75 | 52.992 | 26.791 | −4.437 | 0.00 | 0.00 |
| ATOM | 577 | CD1 | LEU | 75 | 53.866 | 28.026 | −4.320 | 0.00 | 0.00 |
| ATOM | 578 | CD2 | LEU | 75 | 51.540 | 27.182 | −4.219 | 0.00 | 0.00 |
| ATOM | 579 | N | ALA | 76 | 53.634 | 23.835 | −8.017 | 0.00 | 0.00 |
| ATOM | 580 | CA | ALA | 76 | 53.818 | 23.540 | −9.422 | 0.00 | 0.00 |
| ATOM | 581 | C | ALA | 76 | 53.054 | 22.245 | −9.915 | 0.00 | 0.00 |
| ATOM | 582 | O | ALA | 76 | 52.456 | 22.310 | −10.991 | 0.00 | 0.00 |
| ATOM | 583 | CB | ALA | 76 | 55.296 | 23.583 | −9.759 | 0.00 | 0.00 |
| ATOM | 584 | N | VAL | 77 | 53.247 | 21.068 | −9.337 | 0.00 | 0.00 |
| ATOM | 585 | CA | VAL | 77 | 52.512 | 19.849 | −9.655 | 0.00 | 0.00 |
| ATOM | 586 | C | VAL | 77 | 51.286 | 19.674 | −8.775 | 0.00 | 0.00 |
| ATOM | 587 | O | VAL | 77 | 50.241 | 19.408 | −9.348 | 0.00 | 0.00 |
| ATOM | 588 | CB | VAL | 77 | 53.415 | 18.625 | −9.625 | 0.00 | 0.00 |
| ATOM | 589 | CG1 | VAL | 77 | 54.599 | 18.651 | −10.571 | 0.00 | 0.00 |
| ATOM | 590 | CG2 | VAL | 77 | 53.996 | 18.357 | −8.233 | 0.00 | 0.00 |
| ATOM | 591 | N | VAL | 78 | 51.388 | 20.001 | −7.529 | 0.00 | 0.00 |
| ATOM | 592 | CA | VAL | 78 | 50.328 | 19.783 | −6.625 | 0.00 | 0.00 |
| ATOM | 593 | C | VAL | 78 | 49.017 | 20.507 | −7.078 | 0.00 | 0.00 |
| ATOM | 594 | O | VAL | 78 | 48.057 | 19.725 | −7.315 | 0.00 | 0.00 |
| ATOM | 595 | CB | VAL | 78 | 50.717 | 19.856 | −5.170 | 0.00 | 0.00 |
| ATOM | 596 | CG1 | VAL | 78 | 49.527 | 19.543 | −4.202 | 0.00 | 0.00 |
| ATOM | 597 | CG2 | VAL | 78 | 51.960 | 19.158 | −4.787 | 0.00 | 0.00 |
| ATOM | 598 | N | PRO | 79 | 48.813 | 21.853 | −7.222 | 0.00 | 0.00 |
| ATOM | 599 | CA | PRO | 79 | 47.516 | 22.255 | −7.587 | 0.00 | 0.00 |
| ATOM | 600 | C | PRO | 79 | 47.038 | 21.553 | −8.890 | 0.00 | 0.00 |
| ATOM | 601 | O | PRO | 79 | 45.834 | 21.535 | −9.108 | 0.00 | 0.00 |
| ATOM | 602 | CB | PRO | 79 | 47.378 | 23.815 | −7.791 | 0.00 | 0.00 |
| ATOM | 603 | CG | PRO | 79 | 48.830 | 24.251 | −7.527 | 0.00 | 0.00 |
| ATOM | 604 | CD | PRO | 79 | 49.721 | 23.085 | −7.224 | 0.00 | 0.00 |
| ATOM | 605 | N | PHE | 80 | 47.929 | 21.268 | −9.810 | 0.00 | 0.00 |
| ATOM | 606 | CA | PHE | 80 | 47.668 | 20.504 | −11.022 | 0.00 | 0.00 |
| ATOM | 607 | C | PHE | 80 | 46.922 | 19.188 | −10.797 | 0.00 | 0.00 |
| ATOM | 608 | O | PHE | 80 | 45.723 | 19.187 | −11.069 | 0.00 | 0.00 |
| ATOM | 609 | CB | PHE | 80 | 48.990 | 20.376 | −11.801 | 0.00 | 0.00 |
| ATOM | 610 | CG | PHE | 80 | 49.405 | 21.419 | −12.763 | 0.00 | 0.00 |
| ATOM | 611 | CD1 | PHE | 80 | 50.305 | 21.070 | −13.793 | 0.00 | 0.00 |
| ATOM | 612 | CD2 | PHE | 80 | 48.907 | 22.720 | −12.714 | 0.00 | 0.00 |
| ATOM | 613 | CE1 | PHE | 80 | 50.746 | 22.039 | −14.671 | 0.00 | 0.00 |
| ATOM | 614 | CE2 | PHE | 80 | 49.323 | 23.694 | −13.621 | 0.00 | 0.00 |
| ATOM | 615 | CZ | PHE | 80 | 50.228 | 23.329 | −14.629 | 0.00 | 0.00 |
| ATOM | 616 | N | GLY | 81 | 47.479 | 18.285 | −10.045 | 0.00 | 0.00 |
| ATOM | 617 | CA | GLY | 81 | 46.890 | 17.015 | −9.659 | 0.00 | 0.00 |
| ATOM | 618 | C | GLY | 81 | 45.477 | 17.246 | −8.974 | 0.00 | 0.00 |
| ATOM | 619 | O | GLY | 81 | 44.649 | 16.387 | −9.158 | 0.00 | 0.00 |
| ATOM | 620 | N | ALA | 82 | 45.337 | 18.201 | −8.017 | 0.00 | 0.00 |
| ATOM | 621 | CA | ALA | 82 | 44.100 | 18.608 | −7.364 | 0.00 | 0.00 |
| ATOM | 622 | C | ALA | 82 | 42.976 | 18.981 | −8.373 | 0.00 | 0.00 |
| ATOM | 623 | O | ALA | 82 | 41.993 | 18.260 | −8.360 | 0.00 | 0.00 |
| ATOM | 624 | CB | ALA | 82 | 44.435 | 19.755 | −6.399 | 0.00 | 0.00 |
| ATOM | 625 | N | ALA | 83 | 43.136 | 19.965 | −9.268 | 0.00 | 0.00 |
| ATOM | 626 | CA | ALA | 83 | 42.141 | 20.304 | −10.312 | 0.00 | 0.00 |
| ATOM | 627 | C | ALA | 83 | 41.828 | 19.077 | −11.268 | 0.00 | 0.00 |
| ATOM | 628 | O | ALA | 83 | 40.811 | 19.103 | −11.971 | 0.00 | 0.00 |
| ATOM | 629 | CB | ALA | 83 | 42.650 | 21.550 | −11.054 | 0.00 | 0.00 |
| ATOM | 630 | N | HIS | 84 | 42.891 | 18.376 | −11.576 | 0.00 | 0.00 |
| ATOM | 631 | CA | HIS | 84 | 42.958 | 17.165 | −12.398 | 0.00 | 0.00 |
| ATOM | 632 | C | HIS | 84 | 42.028 | 16.030 | −11.809 | 0.00 | 0.00 |
| ATOM | 633 | O | HIS | 84 | 41.235 | 15.500 | −12.565 | 0.00 | 0.00 |
| ATOM | 634 | CB | HIS | 84 | 44.382 | 16.640 | −12.463 | 0.00 | 0.00 |
| ATOM | 635 | CG | HIS | 84 | 44.760 | 15.548 | −13.463 | 0.00 | 0.00 |
| ATOM | 636 | ND1 | HIS | 84 | 44.935 | 15.646 | −14.803 | 0.00 | 0.00 |
| ATOM | 637 | CD2 | HIS | 84 | 44.994 | 14.210 | −13.119 | 0.00 | 0.00 |
| ATOM | 638 | CE1 | HIS | 84 | 45.264 | 14.440 | −15.259 | 0.00 | 0.00 |
| ATOM | 639 | NE2 | HIS | 84 | 45.296 | 13.589 | −14.240 | 0.00 | 0.00 |
| ATOM | 640 | N | ILE | 85 | 42.346 | 15.531 | −10.644 | 0.00 | 0.00 |
| ATOM | 641 | CA | ILE | 85 | 41.509 | 14.569 | −9.975 | 0.00 | 0.00 |
| ATOM | 642 | C | ILE | 85 | 40.053 | 15.091 | −9.816 | 0.00 | 0.00 |
| ATOM | 643 | O | ILE | 85 | 39.108 | 14.292 | −9.698 | 0.00 | 0.00 |
| ATOM | 644 | CB | ILE | 85 | 42.022 | 14.229 | −8.542 | 0.00 | 0.00 |
| ATOM | 645 | CG1 | ILE | 85 | 43.554 | 13.922 | −8.531 | 0.00 | 0.00 |
| ATOM | 646 | CG2 | ILE | 85 | 41.268 | 12.935 | −7.991 | 0.00 | 0.00 |
| ATOM | 647 | CD1 | ILE | 85 | 44.279 | 13.738 | −7.202 | 0.00 | 0.00 |
| ATOM | 648 | N | LEU | 86 | 39.926 | 16.424 | −9.611 | 0.00 | 0.00 |
| ATOM | 649 | CA | LEU | 86 | 38.609 | 17.112 | −9.540 | 0.00 | 0.00 |
| ATOM | 650 | C | LEU | 86 | 37.823 | 16.861 | −10.867 | 0.00 | 0.00 |
| ATOM | 651 | O | LEU | 86 | 36.620 | 16.648 | −10.742 | 0.00 | 0.00 |
| ATOM | 652 | CB | LEU | 86 | 38.735 | 18.596 | −9.181 | 0.00 | 0.00 |
| ATOM | 653 | CG | LEU | 86 | 39.164 | 18.771 | −7.733 | 0.00 | 0.00 |
| ATOM | 654 | CD1 | LEU | 86 | 39.621 | 20.240 | −7.600 | 0.00 | 0.00 |
| ATOM | 655 | CD2 | LEU | 86 | 37.928 | 18.518 | −6.870 | 0.00 | 0.00 |
| ATOM | 656 | N | MET | 87 | 38.436 | 16.928 | −12.084 | 0.00 | 0.00 |
| ATOM | 657 | CA | MET | 87 | 37.655 | 16.568 | −13.299 | 0.00 | 0.00 |
| ATOM | 658 | C | MET | 87 | 37.911 | 15.103 | −13.854 | 0.00 | 0.00 |
| ATOM | 659 | O | MET | 87 | 37.087 | 14.667 | −14.659 | 0.00 | 0.00 |
| ATOM | 660 | CB | MET | 87 | 37.960 | 17.651 | −14.309 | 0.00 | 0.00 |
| ATOM | 661 | CG | MET | 87 | 37.282 | 18.980 | −13.996 | 0.00 | 0.00 |
| ATOM | 662 | SD | MET | 87 | 35.620 | 18.906 | −13.432 | 0.00 | 0.00 |
| ATOM | 663 | CE | MET | 87 | 34.771 | 18.066 | −14.859 | 0.00 | 0.00 |
| ATOM | 664 | N | LYS | 88 | 38.614 | 14.263 | −13.045 | 0.00 | 0.00 |
| ATOM | 665 | CA | LYS | 88 | 39.049 | 12.872 | −13.345 | 0.00 | 0.00 |
| ATOM | 666 | C | LYS | 88 | 39.857 | 12.716 | −14.618 | 0.00 | 0.00 |
| ATOM | 667 | O | LYS | 88 | 40.062 | 11.530 | −15.008 | 0.00 | 0.00 |
| ATOM | 668 | CB | LYS | 88 | 37.959 | 11.882 | −13.158 | 0.00 | 0.00 |
| ATOM | 669 | CG | LYS | 88 | 38.262 | 10.416 | −13.094 | 0.00 | 0.00 |
| ATOM | 670 | CD | LYS | 88 | 38.756 | 10.031 | −11.699 | 0.00 | 0.00 |
| ATOM | 671 | CE | LYS | 88 | 37.640 | 10.036 | −10.635 | 0.00 | 0.00 |
| ATOM | 672 | NZ | LYS | 88 | 36.697 | 8.934 | −10.803 | 0.00 | 0.00 |
| ATOM | 673 | N | MET | 89 | 40.835 | 13.607 | −14.726 | 0.00 | 0.00 |
| ATOM | 674 | CA | MET | 89 | 41.588 | 13.659 | −15.977 | 0.00 | 0.00 |
| ATOM | 675 | C | MET | 89 | 42.569 | 14.781 | −16.113 | 0.00 | 0.00 |
| ATOM | 676 | O | MET | 89 | 42.388 | 15.689 | −15.315 | 0.00 | 0.00 |
| ATOM | 677 | CB | MET | 89 | 40.620 | 14.185 | −16.906 | 0.00 | 0.00 |
| ATOM | 678 | CG | MET | 89 | 39.791 | 15.253 | −17.061 | 0.00 | 0.00 |
| ATOM | 679 | SD | MET | 89 | 38.239 | 15.215 | −18.089 | 0.00 | 0.00 |
| ATOM | 680 | CE | MET | 89 | 37.447 | 16.632 | −17.336 | 0.00 | 0.00 |
| ATOM | 681 | N | TRP | 90 | 43.046 | 15.075 | −17.322 | 0.00 | 0.00 |
| ATOM | 682 | CA | TRP | 90 | 43.870 | 16.271 | −17.376 | 0.00 | 0.00 |
| ATOM | 683 | C | TRP | 90 | 43.157 | 17.473 | −18.127 | 0.00 | 0.00 |
| ATOM | 684 | O | TRP | 90 | 43.256 | 17.525 | −19.355 | 0.00 | 0.00 |
| ATOM | 685 | CB | TRP | 90 | 45.035 | 15.810 | −18.252 | 0.00 | 0.00 |
| ATOM | 686 | CG | TRP | 90 | 46.280 | 15.330 | −17.500 | 0.00 | 0.00 |
| ATOM | 687 | CD1 | TRP | 90 | 46.622 | 14.011 | −17.620 | 0.00 | 0.00 |
| ATOM | 688 | CD2 | TRP | 90 | 47.086 | 15.997 | −16.588 | 0.00 | 0.00 |
| ATOM | 689 | NE1 | TRP | 90 | 47.629 | 13.833 | −16.787 | 0.00 | 0.00 |
| ATOM | 690 | CE2 | TRP | 90 | 47.934 | 14.985 | −16.163 | 0.00 | 0.00 |
| ATOM | 691 | CE3 | TRP | 90 | 47.243 | 17.267 | −16.067 | 0.00 | 0.00 |
| ATOM | 692 | CZ2 | TRP | 90 | 48.911 | 15.211 | −15.222 | 0.00 | 0.00 |
| ATOM | 693 | CZ3 | TRP | 90 | 48.227 | 17.498 | −15.122 | 0.00 | 0.00 |
| ATOM | 694 | CH2 | TRP | 90 | 49.061 | 16.482 | −14.708 | 0.00 | 0.00 |
| ATOM | 695 | N | THR | 91 | 42.186 | 18.199 | −17.532 | 0.00 | 0.00 |
| ATOM | 696 | CA | THR | 91 | 41.525 | 19.409 | −18.104 | 0.00 | 0.00 |
| ATOM | 697 | C | THR | 91 | 42.543 | 20.439 | −18.731 | 0.00 | 0.00 |
| ATOM | 698 | O | THR | 91 | 42.223 | 20.924 | −19.822 | 0.00 | 0.00 |
| ATOM | 699 | CB | THR | 91 | 40.683 | 20.109 | −16.980 | 0.00 | 0.00 |
| ATOM | 700 | OG1 | THR | 91 | 39.722 | 19.201 | −16.374 | 0.00 | 0.00 |
| ATOM | 701 | CG2 | THR | 91 | 40.033 | 21.465 | −17.357 | 0.00 | 0.00 |
| ATOM | 702 | N | PHE | 92 | 43.801 | 20.601 | −18.218 | 0.00 | 0.00 |
| ATOM | 703 | CA | PHE | 92 | 44.763 | 21.604 | −18.739 | 0.00 | 0.00 |
| ATOM | 704 | C | PHE | 92 | 45.739 | 21.075 | −19.868 | 0.00 | 0.00 |
| ATOM | 705 | O | PHE | 92 | 46.684 | 21.818 | −20.202 | 0.00 | 0.00 |
| ATOM | 706 | CB | PHE | 92 | 45.489 | 22.310 | −17.588 | 0.00 | 0.00 |
| ATOM | 707 | CG | PHE | 92 | 44.619 | 22.895 | −16.507 | 0.00 | 0.00 |
| ATOM | 708 | CD1 | PHE | 92 | 43.531 | 23.683 | −16.857 | 0.00 | 0.00 |
| ATOM | 709 | CD2 | PHE | 92 | 44.911 | 22.684 | −15.182 | 0.00 | 0.00 |
| ATOM | 710 | CE1 | PHE | 92 | 42.767 | 24.242 | −15.861 | 0.00 | 0.00 |
| ATOM | 711 | CE2 | PHE | 92 | 44.140 | 23.247 | −14.196 | 0.00 | 0.00 |
| ATOM | 712 | CZ | PHE | 92 | 43.063 | 24.031 | −14.529 | 0.00 | 0.00 |
| ATOM | 713 | N | GLY | 93 | 45.474 | 19.943 | −20.558 | 0.00 | 0.00 |
| ATOM | 714 | CA | GLY | 93 | 46.323 | 19.501 | −21.684 | 0.00 | 0.00 |
| ATOM | 715 | C | GLY | 93 | 47.591 | 18.760 | −21.176 | 0.00 | 0.00 |
| ATOM | 716 | O | GLY | 93 | 47.600 | 18.306 | −20.027 | 0.00 | 0.00 |
| ATOM | 717 | N | ASN | 94 | 48.385 | 18.193 | −22.085 | 0.00 | 0.00 |
| ATOM | 718 | CA | ASN | 94 | 49.652 | 17.570 | −21.646 | 0.00 | 0.00 |
| ATOM | 719 | C | ASN | 94 | 50.538 | 18.566 | −20.791 | 0.00 | 0.00 |
| ATOM | 720 | O | ASN | 94 | 51.581 | 18.103 | −20.350 | 0.00 | 0.00 |
| ATOM | 721 | CB | ASN | 94 | 50.435 | 17.120 | −22.880 | 0.00 | 0.00 |
| ATOM | 722 | CG | ASN | 94 | 49.988 | 15.808 | −23.443 | 0.00 | 0.00 |
| ATOM | 723 | OD1 | ASN | 94 | 50.879 | 15.087 | −23.971 | 0.00 | 0.00 |
| ATOM | 724 | ND2 | ASN | 94 | 48.838 | 15.363 | −23.064 | 0.00 | 0.00 |
| ATOM | 725 | N | PHE | 95 | 50.495 | 19.898 | −21.086 | 0.00 | 0.00 |
| ATOM | 726 | CA | PHE | 95 | 51.204 | 20.883 | −20.275 | 0.00 | 0.00 |
| ATOM | 727 | C | PHE | 95 | 50.808 | 20.740 | −18.792 | 0.00 | 0.00 |
| ATOM | 728 | O | PHE | 95 | 51.649 | 21.040 | −17.959 | 0.00 | 0.00 |
| ATOM | 729 | CB | PHE | 95 | 50.854 | 22.312 | −20.793 | 0.00 | 0.00 |
| ATOM | 730 | CG | PHE | 95 | 51.129 | 22.563 | −22.264 | 0.00 | 0.00 |
| ATOM | 731 | CD1 | PHE | 95 | 52.366 | 22.194 | −22.828 | 0.00 | 0.00 |
| ATOM | 732 | CD2 | PHE | 95 | 50.213 | 23.229 | −23.065 | 0.00 | 0.00 |
| ATOM | 733 | CE1 | PHE | 95 | 52.598 | 22.522 | −24.146 | 0.00 | 0.00 |
| ATOM | 734 | CE2 | PHE | 95 | 50.470 | 23.565 | −24.370 | 0.00 | 0.00 |
| ATOM | 735 | CZ | PHE | 95 | 51.678 | 23.174 | −24.937 | 0.00 | 0.00 |
| ATOM | 736 | N | TRP | 96 | 49.605 | 20.216 | −18.469 | 0.00 | 0.00 |
| ATOM | 737 | CA | TRP | 96 | 49.185 | 20.011 | −17.147 | 0.00 | 0.00 |
| ATOM | 738 | C | TRP | 96 | 49.967 | 18.697 | −16.740 | 0.00 | 0.00 |
| ATOM | 739 | O | TRP | 96 | 50.722 | 18.721 | −15.744 | 0.00 | 0.00 |
| ATOM | 740 | CB | TRP | 96 | 47.644 | 20.024 | −17.218 | 0.00 | 0.00 |
| ATOM | 741 | CG | TRP | 96 | 47.053 | 20.008 | −15.764 | 0.00 | 0.00 |
| ATOM | 742 | CD1 | TRP | 96 | 47.715 | 20.551 | −14.701 | 0.00 | 0.00 |
| ATOM | 743 | CD2 | TRP | 96 | 45.770 | 19.628 | −15.445 | 0.00 | 0.00 |
| ATOM | 744 | NE1 | TRP | 96 | 46.862 | 20.533 | −13.727 | 0.00 | 0.00 |
| ATOM | 745 | CE2 | TRP | 96 | 45.692 | 19.997 | −14.112 | 0.00 | 0.00 |
| ATOM | 746 | CE3 | TRP | 96 | 44.686 | 19.042 | −16.056 | 0.00 | 0.00 |
| ATOM | 747 | CZ2 | TRP | 96 | 44.544 | 19.797 | −13.368 | 0.00 | 0.00 |
| ATOM | 748 | CZ3 | TRP | 96 | 43.534 | 18.843 | −15.308 | 0.00 | 0.00 |
| ATOM | 749 | CH2 | TRP | 96 | 43.461 | 19.220 | −13.971 | 0.00 | 0.00 |
| ATOM | 750 | N | CYS | 97 | 49.987 | 17.655 | −17.604 | 0.00 | 0.00 |
| ATOM | 751 | CA | CYS | 97 | 50.725 | 16.429 | −17.401 | 0.00 | 0.00 |
| ATOM | 752 | C | CYS | 97 | 52.245 | 16.736 | −17.168 | 0.00 | 0.00 |
| ATOM | 753 | O | CYS | 97 | 52.633 | 16.626 | −16.014 | 0.00 | 0.00 |
| ATOM | 754 | CB | CYS | 97 | 50.428 | 15.492 | −18.525 | 0.00 | 0.00 |
| ATOM | 755 | SG | CYS | 97 | 51.381 | 13.975 | −18.578 | 0.00 | 0.00 |
| ATOM | 756 | N | GLU | 98 | 52.879 | 17.602 | −17.987 | 0.00 | 0.00 |
| ATOM | 757 | CA | GLU | 98 | 54.245 | 18.070 | −17.865 | 0.00 | 0.00 |
| ATOM | 758 | C | GLU | 98 | 54.545 | 18.732 | −16.505 | 0.00 | 0.00 |
| ATOM | 759 | O | GLU | 98 | 55.623 | 18.429 | −15.996 | 0.00 | 0.00 |
| ATOM | 760 | CB | GLU | 98 | 54.557 | 19.032 | −19.023 | 0.00 | 0.00 |
| ATOM | 761 | CG | GLU | 98 | 54.864 | 18.271 | −20.301 | 0.00 | 0.00 |
| ATOM | 762 | CD | GLU | 98 | 54.979 | 19.029 | −21.579 | 0.00 | 0.00 |
| ATOM | 763 | OE1 | GLU | 98 | 55.744 | 18.638 | −22.463 | 0.00 | 0.00 |
| ATOM | 764 | OE2 | GLU | 98 | 54.173 | 19.993 | −21.663 | 0.00 | 0.00 |
| ATOM | 765 | N | PHE | 99 | 53.811 | 19.759 | −16.056 | 0.00 | 0.00 |
| ATOM | 766 | CA | PHE | 99 | 54.094 | 20.249 | −14.742 | 0.00 | 0.00 |
| ATOM | 767 | C | PHE | 99 | 53.884 | 19.218 | −13.604 | 0.00 | 0.00 |
| ATOM | 768 | O | PHE | 99 | 54.907 | 18.768 | −13.170 | 0.00 | 0.00 |
| ATOM | 769 | CB | PHE | 99 | 53.285 | 21.488 | −14.493 | 0.00 | 0.00 |
| ATOM | 770 | CG | PHE | 99 | 53.606 | 22.791 | −15.057 | 0.00 | 0.00 |
| ATOM | 771 | CD1 | PHE | 99 | 52.961 | 23.287 | −16.189 | 0.00 | 0.00 |
| ATOM | 772 | CD2 | PHE | 99 | 54.631 | 23.594 | −14.505 | 0.00 | 0.00 |
| ATOM | 773 | CE1 | PHE | 99 | 53.274 | 24.513 | −16.781 | 0.00 | 0.00 |
| ATOM | 774 | CE2 | PHE | 99 | 54.965 | 24.803 | −15.088 | 0.00 | 0.00 |
| ATOM | 775 | CZ | PHE | 99 | 54.280 | 25.277 | −16.208 | 0.00 | 0.00 |
| ATOM | 776 | N | TRP | 100 | 52.784 | 18.419 | −13.607 | 0.00 | 0.00 |
| ATOM | 777 | CA | TRP | 100 | 52.538 | 17.527 | −12.479 | 0.00 | 0.00 |
| ATOM | 778 | C | TRP | 100 | 53.638 | 16.415 | −12.443 | 0.00 | 0.00 |
| ATOM | 779 | O | TRP | 100 | 53.840 | 15.885 | −11.364 | 0.00 | 0.00 |
| ATOM | 780 | CB | TRP | 100 | 51.061 | 17.129 | −12.291 | 0.00 | 0.00 |
| ATOM | 781 | CG | TRP | 100 | 50.775 | 15.985 | −11.326 | 0.00 | 0.00 |
| ATOM | 782 | CD1 | TRP | 100 | 50.257 | 14.801 | −11.764 | 0.00 | 0.00 |
| ATOM | 783 | CD2 | TRP | 100 | 50.829 | 16.054 | −9.952 | 0.00 | 0.00 |
| ATOM | 784 | NE1 | TRP | 100 | 49.964 | 14.125 | −10.688 | 0.00 | 0.00 |
| ATOM | 785 | CE2 | TRP | 100 | 50.280 | 14.818 | −9.587 | 0.00 | 0.00 |
| ATOM | 786 | CE3 | TRP | 100 | 51.240 | 16.960 | −8.964 | 0.00 | 0.00 |
| ATOM | 787 | CZ2 | TRP | 100 | 50.135 | 14.476 | −8.242 | 0.00 | 0.00 |
| ATOM | 788 | CZ3 | TRP | 100 | 51.096 | 16.612 | −7.617 | 0.00 | 0.00 |
| ATOM | 789 | CH2 | TRP | 100 | 50.547 | 15.379 | −17.259 | 0.00 | 0.00 |
| ATOM | 790 | N | THR | 101 | 54.096 | 15.831 | −13.586 | 0.00 | 0.00 |
| ATOM | 791 | CA | THR | 101 | 55.199 | 14.829 | −13.645 | 0.00 | 0.00 |
| ATOM | 792 | C | THR | 101 | 56.541 | 15.514 | −13.136 | 0.00 | 0.00 |
| ATOM | 793 | O | THR | 101 | 57.270 | 14.858 | −12.411 | 0.00 | 0.00 |
| ATOM | 794 | CB | THR | 101 | 55.487 | 14.330 | −15.114 | 0.00 | 0.00 |
| ATOM | 795 | OG1 | THR | 101 | 56.617 | 14.807 | −15.774 | 0.00 | 0.00 |
| ATOM | 796 | CG2 | THR | 101 | 54.249 | 14.399 | −16.034 | 0.00 | 0.00 |
| ATOM | 797 | N | SER | 102 | 56.977 | 16.562 | −13.891 | 0.00 | 0.00 |
| ATOM | 798 | CA | SER | 102 | 58.230 | 17.288 | −13.695 | 0.00 | 0.00 |
| ATOM | 799 | C | SER | 102 | 58.493 | 17.726 | −12.252 | 0.00 | 0.00 |
| ATOM | 800 | O | SER | 102 | 59.643 | 18.049 | −12.004 | 0.00 | 0.00 |
| ATOM | 801 | CB | SER | 102 | 58.290 | 18.541 | −14.597 | 0.00 | 0.00 |
| ATOM | 802 | OG | SER | 102 | 57.326 | 19.604 | −14.314 | 0.00 | 0.00 |
| ATOM | 803 | N | ILE | 103 | 57.498 | 18.194 | −11.504 | 0.00 | 0.00 |
| ATOM | 804 | CA | ILE | 103 | 57.805 | 18.592 | −10.129 | 0.00 | 0.00 |
| ATOM | 805 | C | ILE | 103 | 57.642 | 17.333 | −9.192 | 0.00 | 0.00 |
| ATOM | 806 | O | ILE | 103 | 58.307 | 17.340 | −8.200 | 0.00 | 0.00 |
| ATOM | 807 | CB | ILE | 103 | 56.927 | 19.793 | −9.660 | 0.00 | 0.00 |
| ATOM | 808 | CG1 | ILE | 103 | 56.867 | 21.005 | −10.559 | 0.00 | 0.00 |
| ATOM | 809 | CG2 | ILE | 103 | 57.038 | 20.140 | −8.128 | 0.00 | 0.00 |
| ATOM | 810 | CD1 | ILE | 103 | 58.118 | 21.867 | −10.467 | 0.00 | 0.00 |
| ATOM | 811 | N | ASP | 104 | 56.814 | 16.307 | −9.549 | 0.00 | 0.00 |
| ATOM | 812 | CA | ASP | 104 | 56.778 | 15.101 | −8.699 | 0.00 | 0.00 |
| ATOM | 813 | C | ASP | 104 | 58.226 | 14.467 | −8.795 | 0.00 | 0.00 |
| ATOM | 814 | O | ASP | 104 | 58.541 | 13.694 | −7.856 | 0.00 | 0.00 |
| ATOM | 815 | CB | ASP | 104 | 55.674 | 14.064 | −9.083 | 0.00 | 0.00 |
| ATOM | 816 | CG | ASP | 104 | 56.166 | 12.599 | −9.100 | 0.00 | 0.00 |
| ATOM | 817 | OD1 | ASP | 104 | 57.343 | 12.261 | −8.980 | 0.00 | 0.00 |
| ATOM | 818 | OD2 | ASP | 104 | 56.699 | 12.249 | −10.146 | 0.00 | 0.00 |
| ATOM | 819 | N | VAL | 105 | 58.786 | 14.256 | −9.999 | 0.00 | 0.00 |
| ATOM | 820 | CA | VAL | 105 | 60.153 | 13.771 | −10.254 | 0.00 | 0.00 |
| ATOM | 821 | C | VAL | 105 | 61.193 | 14.809 | −9.731 | 0.00 | 0.00 |
| ATOM | 822 | O | VAL | 105 | 62.197 | 14.345 | −9.219 | 0.00 | 0.00 |
| ATOM | 823 | CB | VAL | 105 | 60.394 | 13.461 | −11.753 | 0.00 | 0.00 |
| ATOM | 824 | CG1 | VAL | 105 | 61.719 | 12.731 | −12.116 | 0.00 | 0.00 |
| ATOM | 825 | CG2 | VAL | 105 | 59.213 | 12.710 | −12.348 | 0.00 | 0.00 |
| ATOM | 826 | N | LEU | 106 | 61.182 | 16.101 | −10.177 | 0.00 | 0.00 |
| ATOM | 827 | CA | LEU | 106 | 62.138 | 17.132 | −9.695 | 0.00 | 0.00 |
| ATOM | 828 | C | LEU | 106 | 62.276 | 17.070 | −8.133 | 0.00 | 0.00 |
| ATOM | 829 | O | LEU | 106 | 63.369 | 17.421 | −7.669 | 0.00 | 0.00 |
| ATOM | 830 | CB | LEU | 106 | 61.697 | 18.542 | −10.136 | 0.00 | 0.00 |
| ATOM | 831 | CG | LEU | 106 | 62.609 | 19.695 | −9.718 | 0.00 | 0.00 |
| ATOM | 832 | CD1 | LEU | 106 | 63.952 | 19.564 | −10.420 | 0.00 | 0.00 |
| ATOM | 833 | CD2 | LEU | 106 | 61.976 | 21.041 | −10.005 | 0.00 | 0.00 |
| ATOM | 834 | N | CYS | 107 | 61.177 | 17.064 | −7.378 | 0.00 | 0.00 |
| ATOM | 835 | CA | CYS | 107 | 61.166 | 16.951 | −5.934 | 0.00 | 0.00 |
| ATOM | 836 | C | CYS | 107 | 61.858 | 15.619 | −5.442 | 0.00 | 0.00 |
| ATOM | 837 | O | CYS | 107 | 62.426 | 15.683 | −4.358 | 0.00 | 0.00 |
| ATOM | 838 | CB | CYS | 107 | 59.715 | 17.074 | −5.552 | 0.00 | 0.00 |
| ATOM | 839 | SG | CYS | 107 | 58.552 | 15.718 | −5.932 | 0.00 | 0.00 |
| ATOM | 840 | N | VAL | 108 | 61.441 | 14.446 | −5.908 | 0.00 | 0.00 |
| ATOM | 841 | CA | VAL | 108 | 61.990 | 13.169 | −5.539 | 0.00 | 0.00 |
| ATOM | 842 | C | VAL | 108 | 63.511 | 13.138 | −5.808 | 0.00 | 0.00 |
| ATOM | 843 | O | VAL | 108 | 64.173 | 12.308 | −5.153 | 0.00 | 0.00 |
| ATOM | 844 | CB | VAL | 108 | 61.216 | 12.105 | −6.342 | 0.00 | 0.00 |
| ATOM | 845 | CG1 | VAL | 108 | 61.931 | 10.740 | −6.173 | 0.00 | 0.00 |
| ATOM | 846 | CG2 | VAL | 108 | 59.787 | 11.961 | −5.804 | 0.00 | 0.00 |
| ATOM | 847 | N | THR | 109 | 63.825 | 13.360 | −7.095 | 0.00 | 0.00 |
| ATOM | 848 | CA | THR | 109 | 65.172 | 13.475 | −7.545 | 0.00 | 0.00 |
| ATOM | 849 | C | THR | 109 | 65.960 | 14.396 | −6.661 | 0.00 | 0.00 |
| ATOM | 850 | O | THR | 109 | 67.131 | 14.080 | −6.469 | 0.00 | 0.00 |
| ATOM | 851 | CB | THR | 109 | 65.313 | 13.934 | −9.044 | 0.00 | 0.00 |
| ATOM | 852 | OG1 | THR | 109 | 64.849 | 15.322 | −9.254 | 0.00 | 0.00 |
| ATOM | 853 | CG2 | THR | 109 | 64.839 | 12.860 | −10.004 | 0.00 | 0.00 |
| ATOM | 854 | N | ALA | 110 | 65.516 | 15.665 | −6.497 | 0.00 | 0.00 |
| ATOM | 855 | CA | ALA | 110 | 66.155 | 16.591 | −5.576 | 0.00 | 0.00 |
| ATOM | 856 | C | ALA | 110 | 66.255 | 15.873 | −4.194 | 0.00 | 0.00 |
| ATOM | 857 | O | ALA | 110 | 67.276 | 16.076 | −3.556 | 0.00 | 0.00 |
| ATOM | 858 | CB | ALA | 110 | 65.408 | 17.935 | −5.515 | 0.00 | 0.00 |
| ATOM | 859 | N | SER | 111 | 65.236 | 15.077 | −3.746 | 0.00 | 0.00 |
| ATOM | 860 | CA | SER | 111 | 65.227 | 14.269 | −2.529 | 0.00 | 0.00 |
| ATOM | 861 | C | SER | 111 | 66.295 | 13.114 | −2.570 | 0.00 | 0.00 |
| ATOM | 862 | O | SER | 111 | 66.920 | 12.929 | −1.525 | 0.00 | 0.00 |
| ATOM | 863 | CB | SER | 111 | 63.818 | 13.726 | −2.274 | 0.00 | 0.00 |
| ATOM | 864 | OG | SER | 111 | 62.863 | 14.656 | −1.786 | 0.00 | 0.00 |
| ATOM | 865 | N | ILE | 112 | 66.285 | 12.165 | −3.551 | 0.00 | 0.00 |
| ATOM | 866 | CA | ILE | 112 | 67.334 | 11.114 | −3.636 | 0.00 | 0.00 |
| ATOM | 867 | C | ILE | 112 | 68.779 | 11.694 | −3.829 | 0.00 | 0.00 |
| ATOM | 868 | O | ILE | 112 | 69.693 | 11.056 | −3.308 | 0.00 | 0.00 |
| ATOM | 869 | CB | ILE | 112 | 66.898 | 10.050 | −4.695 | 0.00 | 0.00 |
| ATOM | 870 | CG1 | ILE | 112 | 67.645 | 8.707 | −4.422 | 0.00 | 0.00 |
| ATOM | 871 | CG2 | ILE | 112 | 67.059 | 10.589 | −6.130 | 0.00 | 0.00 |
| ATOM | 872 | CD1 | ILE | 112 | 67.444 | 7.643 | −5.511 | 0.00 | 0.00 |
| ATOM | 873 | N | GLU | 113 | 69.000 | 12.773 | −4.601 | 0.00 | 0.00 |
| ATOM | 874 | CA | GLU | 113 | 70.311 | 13.372 | −4.746 | 0.00 | 0.00 |
| ATOM | 875 | C | GLU | 113 | 70.751 | 14.076 | −3.406 | 0.00 | 0.00 |
| ATOM | 876 | O | GLU | 113 | 71.967 | 14.174 | −3.217 | 0.00 | 0.00 |
| ATOM | 877 | CB | GLU | 113 | 70.229 | 14.416 | −5.878 | 0.00 | 0.00 |
| ATOM | 878 | CG | GLU | 113 | 70.477 | 13.968 | −7.305 | 0.00 | 0.00 |
| ATOM | 879 | CD | GLU | 113 | 71.163 | 12.635 | −7.313 | 0.00 | 0.00 |
| ATOM | 880 | OE1 | GLU | 113 | 70.737 | 11.633 | −7.846 | 0.00 | 0.00 |
| ATOM | 881 | OE2 | GLU | 113 | 72.203 | 12.723 | −6.630 | 0.00 | 0.00 |
| ATOM | 882 | N | THR | 114 | 69.871 | 14.816 | −2.700 | 0.00 | 0.00 |
| ATOM | 883 | CA | THR | 114 | 70.150 | 15.420 | −1.399 | 0.00 | 0.00 |
| ATOM | 884 | C | THR | 114 | 70.643 | 14.297 | −0.442 | 0.00 | 0.00 |
| ATOM | 885 | O | THR | 114 | 71.465 | 14.642 | 0.413 | 0.00 | 0.00 |
| ATOM | 886 | CB | THR | 114 | 68.951 | 16.274 | −0.884 | 0.00 | 0.00 |
| ATOM | 887 | OG1 | THR | 114 | 69.270 | 17.185 | 0.202 | 0.00 | 0.00 |
| ATOM | 888 | CG2 | THR | 114 | 67.600 | 15.671 | −0.696 | 0.00 | 0.00 |
| ATOM | 889 | N | LEU | 115 | 69.983 | 13.126 | −0.388 | 0.00 | 0.00 |
| ATOM | 890 | CA | LEU | 115 | 70.445 | 11.994 | 0.402 | 0.00 | 0.00 |
| ATOM | 891 | C | LEU | 115 | 71.899 | 11.624 | −0.004 | 0.00 | 0.00 |
| ATOM | 892 | O | LEU | 115 | 72.579 | 11.041 | 0.858 | 0.00 | 0.00 |
| ATOM | 893 | CB | LEU | 115 | 69.493 | 10.830 | 0.166 | 0.00 | 0.00 |
| ATOM | 894 | CG | LEU | 115 | 68.152 | 10.983 | 0.836 | 0.00 | 0.00 |
| ATOM | 895 | CD1 | LEU | 115 | 67.237 | 9.771 | 0.651 | 0.00 | 0.00 |
| ATOM | 896 | CD2 | LEU | 115 | 68.250 | 11.198 | 2.346 | 0.00 | 0.00 |
| ATOM | 897 | N | CYS | 116 | 72.286 | 11.660 | −1.293 | 0.00 | 0.00 |
| ATOM | 898 | CA | CYS | 116 | 73.668 | 11.408 | −1.755 | 0.00 | 0.00 |
| ATOM | 899 | C | CYS | 116 | 74.596 | 12.440 | −1.068 | 0.00 | 0.00 |
| ATOM | 900 | O | CYS | 116 | 75.490 | 11.995 | −0.365 | 0.00 | 0.00 |
| ATOM | 901 | CB | CYS | 116 | 73.797 | 11.419 | −3.275 | 0.00 | 0.00 |
| ATOM | 902 | SG | CYS | 116 | 75.530 | 11.348 | −3.777 | 0.00 | 0.00 |
| ATOM | 903 | N | VAL | 117 | 74.299 | 13.684 | −1.141 | 0.00 | 0.00 |
| ATOM | 904 | CA | VAL | 117 | 75.167 | 14.619 | −0.418 | 0.00 | 0.00 |
| ATOM | 905 | C | VAL | 117 | 75.291 | 14.292 | 1.060 | 0.00 | 0.00 |
| ATOM | 906 | O | VAL | 117 | 76.414 | 14.462 | 1.571 | 0.00 | 0.00 |
| ATOM | 907 | CB | VAL | 117 | 74.769 | 16.096 | −0.607 | 0.00 | 0.00 |
| ATOM | 908 | CG1 | VAL | 117 | 75.598 | 17.039 | 0.227 | 0.00 | 0.00 |
| ATOM | 909 | CG2 | VAL | 117 | 74.706 | 16.504 | −2.058 | 0.00 | 0.00 |
| ATOM | 910 | N | ILE | 118 | 74.148 | 14.231 | 1.752 | 0.00 | 0.00 |
| ATOM | 911 | CA | ILE | 118 | 74.120 | 13.901 | 3.157 | 0.00 | 0.00 |
| ATOM | 912 | C | ILE | 118 | 74.972 | 12.690 | 3.547 | 0.00 | 0.00 |
| ATOM | 913 | O | ILE | 118 | 75.595 | 12.812 | 4.610 | 0.00 | 0.00 |
| ATOM | 914 | CB | ILE | 118 | 72.633 | 13.622 | 3.493 | 0.00 | 0.00 |
| ATOM | 915 | CG1 | ILE | 118 | 71.823 | 14.882 | 3.577 | 0.00 | 0.00 |
| ATOM | 916 | CG2 | ILE | 118 | 72.533 | 12.995 | 4.913 | 0.00 | 0.00 |
| ATOM | 917 | CD1 | ILE | 118 | 70.275 | 14.737 | 3.570 | 0.00 | 0.00 |
| ATOM | 918 | N | ALA | 119 | 74.753 | 11.501 | 2.984 | 0.00 | 0.00 |
| ATOM | 919 | CA | ALA | 119 | 75.521 | 10.310 | 3.193 | 0.00 | 0.00 |
| ATOM | 920 | C | ALA | 119 | 77.043 | 10.591 | 3.206 | 0.00 | 0.00 |
| ATOM | 921 | O | ALA | 119 | 77.679 | 10.162 | 4.163 | 0.00 | 0.00 |
| ATOM | 922 | CB | ALA | 119 | 75.108 | 9.254 | 2.142 | 0.00 | 0.00 |
| ATOM | 923 | N | VAL | 120 | 77.635 | 11.167 | 2.145 | 0.00 | 0.00 |
| ATOM | 924 | CA | VAL | 120 | 79.070 | 11.568 | 2.079 | 0.00 | 0.00 |
| ATOM | 925 | C | VAL | 120 | 79.413 | 12.579 | 3.207 | 0.00 | 0.00 |
| ATOM | 926 | O | VAL | 120 | 80.571 | 12.565 | 3.614 | 0.00 | 0.00 |
| ATOM | 927 | CB | VAL | 120 | 79.427 | 12.188 | 0.717 | 0.00 | 0.00 |
| ATOM | 928 | CG1 | VAL | 120 | 80.872 | 12.662 | 0.544 | 0.00 | 0.00 |
| ATOM | 929 | CG2 | VAL | 120 | 79.086 | 11.320 | −0.468 | 0.00 | 0.00 |
| ATOM | 930 | N | ASP | 121 | 78.549 | 13.579 | 3.461 | 0.00 | 0.00 |
| ATOM | 931 | CA | ASP | 121 | 78.766 | 14.500 | 4.557 | 0.00 | 0.00 |
| ATOM | 932 | C | ASP | 121 | 78.988 | 13.688 | 5.883 | 0.00 | 0.00 |
| ATOM | 933 | O | ASP | 121 | 79.942 | 14.049 | 6.576 | 0.00 | 0.00 |
| ATOM | 934 | CB | ASP | 121 | 77.635 | 15.519 | 4.591 | 0.00 | 0.00 |
| ATOM | 935 | CG | ASP | 121 | 77.951 | 16.731 | 5.370 | 0.00 | 0.00 |
| ATOM | 936 | OD1 | ASP | 121 | 79.163 | 16.803 | 5.650 | 0.00 | 0.00 |
| ATOM | 937 | OD2 | ASP | 121 | 77.082 | 17.535 | 5.712 | 0.00 | 0.00 |
| ATOM | 938 | N | ARG | 122 | 78.040 | 12.839 | 6.355 | 0.00 | 0.00 |
| ATOM | 939 | CA | ARG | 122 | 78.207 | 11.978 | 7.529 | 0.00 | 0.00 |
| ATOM | 940 | C | ARG | 122 | 79.440 | 11.028 | 7.420 | 0.00 | 0.00 |
| ATOM | 941 | O | ARG | 122 | 79.798 | 10.489 | 8.459 | 0.00 | 0.00 |
| ATOM | 942 | CB | ARG | 122 | 76.924 | 11.348 | 8.039 | 0.00 | 0.00 |
| ATOM | 943 | CG | ARG | 122 | 75.687 | 12.159 | 8.261 | 0.00 | 0.00 |
| ATOM | 944 | CD | ARG | 122 | 75.843 | 13.430 | 9.133 | 0.00 | 0.00 |
| ATOM | 945 | NE | ARG | 122 | 76.220 | 14.653 | 8.409 | 0.00 | 0.00 |
| ATOM | 946 | CZ | ARG | 122 | 75.826 | 15.885 | 8.799 | 0.00 | 0.00 |
| ATOM | 947 | NH1 | ARG | 122 | 75.074 | 16.078 | 9.884 | 0.00 | 0.00 |
| ATOM | 948 | NH2 | ARG | 122 | 76.189 | 16.966 | 8.101 | 0.00 | 0.00 |
| ATOM | 949 | N | TYR | 123 | 79.859 | 10.558 | 6.204 | 0.00 | 0.00 |
| ATOM | 950 | CA | TYR | 123 | 81.049 | 9.753 | 5.968 | 0.00 | 0.00 |
| ATOM | 951 | C | TYR | 123 | 82.288 | 10.614 | 6.317 | 0.00 | 0.00 |
| ATOM | 952 | O | TYR | 123 | 82.879 | 10.316 | 7.360 | 0.00 | 0.00 |
| ATOM | 953 | CB | TYR | 123 | 81.120 | 9.261 | 4.533 | 0.00 | 0.00 |
| ATOM | 954 | CG | TYR | 123 | 82.321 | 8.407 | 4.234 | 0.00 | 0.00 |
| ATOM | 955 | CD1 | TYR | 123 | 82.330 | 7.060 | 4.602 | 0.00 | 0.00 |
| ATOM | 956 | CD2 | TYR | 123 | 83.456 | 8.936 | 3.622 | 0.00 | 0.00 |
| ATOM | 957 | CE1 | TYR | 123 | 83.431 | 6.260 | 4.379 | 0.00 | 0.00 |
| ATOM | 958 | CE2 | TYR | 123 | 84.564 | 8.142 | 3.390 | 0.00 | 0.00 |
| ATOM | 959 | CZ | TYR | 123 | 84.543 | 6.802 | 3.773 | 0.00 | 0.00 |
| ATOM | 960 | OH | TYR | 123 | 85.632 | 5.992 | 3.549 | 0.00 | 0.00 |
| ATOM | 961 | N | PHE | 124 | 82.526 | 11.773 | 5.665 | 0.00 | 0.00 |
| ATOM | 962 | CA | PHE | 124 | 83.617 | 12.690 | 5.992 | 0.00 | 0.00 |
| ATOM | 963 | C | PHE | 124 | 83.653 | 13.056 | 7.515 | 0.00 | 0.00 |
| ATOM | 964 | O | PHE | 124 | 84.752 | 12.987 | 8.077 | 0.00 | 0.00 |
| ATOM | 965 | CB | PHE | 124 | 83.501 | 13.938 | 5.138 | 0.00 | 0.00 |
| ATOM | 966 | CG | PHE | 124 | 83.784 | 13.818 | 3.671 | 0.00 | 0.00 |
| ATOM | 967 | CD1 | PHE | 124 | 84.853 | 13.053 | 3.210 | 0.00 | 0.00 |
| ATOM | 968 | CD2 | PHE | 124 | 83.013 | 14.534 | 2.770 | 0.00 | 0.00 |
| ATOM | 969 | CE1 | PHE | 124 | 85.154 | 12.994 | 1.841 | 0.00 | 0.00 |
| ATOM | 970 | CE2 | PHE | 124 | 83.269 | 14.475 | 1.386 | 0.00 | 0.00 |
| ATOM | 971 | CZ | PHE | 124 | 84.334 | 13.673 | 0.940 | 0.00 | 0.00 |
| ATOM | 972 | N | ALA | 125 | 82.551 | 13.485 | 8.129 | 0.00 | 0.00 |
| ATOM | 973 | CA | ALA | 125 | 82.450 | 13.854 | 9.541 | 0.00 | 0.00 |
| ATOM | 974 | C | ALA | 125 | 82.577 | 12.638 | 10.541 | 0.00 | 0.00 |
| ATOM | 975 | O | ALA | 125 | 83.512 | 12.706 | 11.351 | 0.00 | 0.00 |
| ATOM | 976 | CB | ALA | 125 | 81.124 | 14.613 | 9.708 | 0.00 | 0.00 |
| ATOM | 977 | N | ILE | 126 | 81.724 | 11.612 | 10.503 | 0.00 | 0.00 |
| ATOM | 978 | CA | ILE | 126 | 81.675 | 10.489 | 11.428 | 0.00 | 0.00 |
| ATOM | 979 | C | ILE | 126 | 82.649 | 9.309 | 11.082 | 0.00 | 0.00 |
| ATOM | 980 | O | ILE | 126 | 83.173 | 8.760 | 12.042 | 0.00 | 0.00 |
| ATOM | 981 | CB | ILE | 126 | 80.185 | 10.023 | 11.506 | 0.00 | 0.00 |
| ATOM | 982 | CG1 | ILE | 126 | 79.238 | 11.203 | 11.707 | 0.00 | 0.00 |
| ATOM | 983 | CG2 | ILE | 126 | 79.967 | 8.876 | 12.534 | 0.00 | 0.00 |
| ATOM | 984 | CD1 | ILE | 126 | 77.798 | 10.883 | 11.185 | 0.00 | 0.00 |
| ATOM | 985 | N | THR | 127 | 82.737 | 8.759 | 9.833 | 0.00 | 0.00 |
| ATOM | 986 | CA | THR | 127 | 83.734 | 7.704 | 9.468 | 0.00 | 0.00 |
| ATOM | 987 | C | THR | 127 | 85.221 | 8.238 | 9.633 | 0.00 | 0.00 |
| ATOM | 988 | O | THR | 127 | 86.114 | 7.399 | 9.803 | 0.00 | 0.00 |
| ATOM | 989 | CB | THR | 127 | 83.404 | 7.037 | 8.085 | 0.00 | 0.00 |
| ATOM | 990 | OG1 | THR | 127 | 82.073 | 6.422 | 8.089 | 0.00 | 0.00 |
| ATOM | 991 | CG2 | THR | 127 | 84.462 | 5.941 | 7.748 | 0.00 | 0.00 |
| ATOM | 992 | N | SER | 128 | 85.346 | 9.526 | 10.025 | 0.00 | 0.00 |
| ATOM | 993 | CA | SER | 128 | 86.559 | 10.230 | 10.268 | 0.00 | 0.00 |
| ATOM | 994 | C | SER | 128 | 87.536 | 10.270 | 9.077 | 0.00 | 0.00 |
| ATOM | 995 | O | SER | 128 | 88.728 | 10.428 | 9.357 | 0.00 | 0.00 |
| ATOM | 996 | CB | SER | 128 | 87.230 | 9.596 | 11.492 | 0.00 | 0.00 |
| ATOM | 997 | OG | SER | 128 | 86.518 | 9.491 | 12.695 | 0.00 | 0.00 |
| ATOM | 998 | N | PRO | 129 | 87.124 | 10.335 | 7.774 | 0.00 | 0.00 |
| ATOM | 999 | CA | PRO | 129 | 88.092 | 10.475 | 6.707 | 0.00 | 0.00 |
| ATOM | 1000 | C | PRO | 129 | 88.773 | 11.905 | 6.768 | 0.00 | 0.00 |
| ATOM | 1001 | O | PRO | 129 | 88.502 | 12.723 | 7.646 | 0.00 | 0.00 |
| ATOM | 1002 | CB | PRO | 129 | 87.433 | 10.181 | 5.307 | 0.00 | 0.00 |
| ATOM | 1003 | CG | PRO | 129 | 85.933 | 10.303 | 5.665 | 0.00 | 0.00 |
| ATOM | 1004 | CD | PRO | 129 | 85.739 | 10.164 | 7.155 | 0.00 | 0.00 |
| ATOM | 1005 | N | PHE | 130 | 89.412 | 12.196 | 5.637 | 0.00 | 0.00 |
| ATOM | 1006 | CA | PHE | 130 | 90.233 | 13.323 | 5.403 | 0.00 | 0.00 |
| ATOM | 1007 | C | PHE | 130 | 91.324 | 13.284 | 6.513 | 0.00 | 0.00 |
| ATOM | 1008 | O | PHE | 130 | 91.857 | 12.160 | 6.716 | 0.00 | 0.00 |
| ATOM | 1009 | CB | PHE | 130 | 89.441 | 14.572 | 5.319 | 0.00 | 0.00 |
| ATOM | 1010 | CG | PHE | 130 | 89.016 | 15.047 | 4.005 | 0.00 | 0.00 |
| ATOM | 1011 | CD1 | PHE | 130 | 88.232 | 14.217 | 3.216 | 0.00 | 0.00 |
| ATOM | 1012 | CD2 | PHE | 130 | 89.337 | 16.313 | 3.521 | 0.00 | 0.00 |
| ATOM | 1013 | CE1 | PHE | 130 | 87.765 | 14.601 | 1.970 | 0.00 | 0.00 |
| ATOM | 1014 | CE2 | PHE | 130 | 88.886 | 16.741 | 2.273 | 0.00 | 0.00 |
| ATOM | 1015 | CZ | PHE | 130 | 88.103 | 15.876 | 1.515 | 0.00 | 0.00 |
| ATOM | 1016 | N | LYS | 131 | 91.942 | 14.333 | 6.873 | 0.00 | 0.00 |
| ATOM | 1017 | CA | LYS | 131 | 92.906 | 14.276 | 7.987 | 0.00 | 0.00 |
| ATOM | 1018 | C | LYS | 131 | 93.203 | 15.687 | 8.526 | 0.00 | 0.00 |
| ATOM | 1019 | O | LYS | 131 | 93.553 | 15.750 | 9.718 | 0.00 | 0.00 |
| ATOM | 1020 | CB | LYS | 131 | 94.165 | 13.458 | 7.631 | 0.00 | 0.00 |
| ATOM | 1021 | CG | LYS | 131 | 95.046 | 13.905 | 6.493 | 0.00 | 0.00 |
| ATOM | 1022 | CD | LYS | 131 | 96.315 | 13.128 | 6.784 | 0.00 | 0.00 |
| ATOM | 1023 | CE | LYS | 131 | 97.259 | 13.103 | 5.596 | 0.00 | 0.00 |
| ATOM | 1024 | NZ | LYS | 131 | 98.543 | 12.573 | 6.028 | 0.00 | 0.00 |
| ATOM | 1025 | N | TYR | 132 | 93.659 | 16.525 | 7.543 | 0.00 | 0.00 |
| ATOM | 1026 | CA | TYR | 132 | 94.073 | 17.831 | 7.924 | 0.00 | 0.00 |
| ATOM | 1027 | C | TYR | 132 | 92.842 | 18.705 | 8.212 | 0.00 | 0.00 |
| ATOM | 1028 | O | TYR | 132 | 92.453 | 19.571 | 7.419 | 0.00 | 0.00 |
| ATOM | 1029 | CB | TYR | 132 | 94.985 | 18.438 | 6.842 | 0.00 | 0.00 |
| ATOM | 1030 | CG | TYR | 132 | 95.949 | 19.482 | 7.410 | 0.00 | 0.00 |
| ATOM | 1031 | CD1 | TYR | 132 | 95.872 | 20.813 | 6.970 | 0.00 | 0.00 |
| ATOM | 1032 | CD2 | TYR | 132 | 96.943 | 19.210 | 8.365 | 0.00 | 0.00 |
| ATOM | 1033 | CE1 | TYR | 132 | 96.770 | 21.807 | 7.381 | 0.00 | 0.00 |
| ATOM | 1034 | CE2 | TYR | 132 | 97.853 | 20.179 | 8.801 | 0.00 | 0.00 |
| ATOM | 1035 | CZ | TYR | 132 | 97.779 | 21.491 | 8.302 | 0.00 | 0.00 |
| ATOM | 1036 | OH | TYR | 132 | 98.676 | 22.453 | 8.660 | 0.00 | 0.00 |
| ATOM | 1037 | N | GLN | 133 | 92.138 | 18.248 | 9.240 | 0.00 | 0.00 |
| ATOM | 1038 | CA | GLN | 133 | 91.018 | 18.854 | 9.828 | 0.00 | 0.00 |
| ATOM | 1039 | C | GLN | 133 | 89.976 | 19.469 | 8.797 | 0.00 | 0.00 |
| ATOM | 1040 | O | GLN | 133 | 89.541 | 20.605 | 9.064 | 0.00 | 0.00 |
| ATOM | 1041 | CB | GLN | 133 | 91.574 | 19.853 | 10.849 | 0.00 | 0.00 |
| ATOM | 1042 | CG | GLN | 133 | 92.221 | 21.074 | 10.257 | 0.00 | 0.00 |
| ATOM | 1043 | CD | GLN | 133 | 92.996 | 21.792 | 11.345 | 0.00 | 0.00 |
| ATOM | 1044 | OE1 | GLN | 133 | 93.344 | 22.968 | 11.218 | 0.00 | 0.00 |
| ATOM | 1045 | NE2 | GLN | 133 | 93.336 | 21.157 | 12.454 | 0.00 | 0.00 |
| ATOM | 1046 | N | SER | 134 | 89.390 | 18.713 | 7.853 | 0.00 | 0.00 |
| ATOM | 1047 | CA | SER | 134 | 88.367 | 19.342 | 6.961 | 0.00 | 0.00 |
| ATOM | 1048 | C | SER | 134 | 86.948 | 19.406 | 7.645 | 0.00 | 0.00 |
| ATOM | 1049 | O | SER | 134 | 86.437 | 18.338 | 8.020 | 0.00 | 0.00 |
| ATOM | 1050 | CB | SER | 134 | 88.185 | 18.534 | 5.679 | 0.00 | 0.00 |
| ATOM | 1051 | OG | SER | 134 | 87.903 | 17.158 | 5.817 | 0.00 | 0.00 |
| ATOM | 1052 | N | LEU | 135 | 86.343 | 20.593 | 7.908 | 0.00 | 0.00 |
| ATOM | 1053 | CA | LEU | 135 | 84.986 | 20.686 | 8.449 | 0.00 | 0.00 |
| ATOM | 1054 | C | LEU | 135 | 83.978 | 21.632 | 7.663 | 0.00 | 0.00 |
| ATOM | 1055 | O | LEU | 135 | 84.002 | 22.856 | 7.858 | 0.00 | 0.00 |
| ATOM | 1056 | CB | LEU | 135 | 85.059 | 21.148 | 9.908 | 0.00 | 0.00 |
| ATOM | 1057 | CG | LEU | 135 | 83.661 | 21.190 | 10.628 | 0.00 | 0.00 |
| ATOM | 1058 | CD1 | LEU | 135 | 83.021 | 19.795 | 10.637 | 0.00 | 0.00 |
| ATOM | 1059 | CD2 | LEU | 135 | 83.842 | 21.695 | 12.048 | 0.00 | 0.00 |
| ATOM | 1060 | N | LEU | 136 | 83.400 | 21.126 | 6.543 | 0.00 | 0.00 |
| ATOM | 1061 | CA | LEU | 136 | 82.356 | 21.714 | 5.654 | 0.00 | 0.00 |
| ATOM | 1062 | C | LEU | 136 | 82.240 | 23.262 | 5.735 | 0.00 | 0.00 |
| ATOM | 1063 | O | LEU | 136 | 83.253 | 23.882 | 5.435 | 0.00 | 0.00 |
| ATOM | 1064 | CB | LEU | 136 | 81.075 | 21.036 | 5.993 | 0.00 | 0.00 |
| ATOM | 1065 | CG | LEU | 136 | 80.619 | 19.641 | 5.823 | 0.00 | 0.00 |
| ATOM | 1066 | CD1 | LEU | 136 | 79.383 | 19.419 | 6.657 | 0.00 | 0.00 |
| ATOM | 1067 | CD2 | LEU | 136 | 80.358 | 19.272 | 4.384 | 0.00 | 0.00 |
| ATOM | 1068 | N | THR | 137 | 81.030 | 23.867 | 5.532 | 0.00 | 0.00 |
| ATOM | 1069 | CA | THR | 137 | 80.694 | 25.318 | 5.666 | 0.00 | 0.00 |
| ATOM | 1070 | C | THR | 137 | 79.420 | 25.517 | 4.839 | 0.00 | 0.00 |
| ATOM | 1071 | O | THR | 137 | 78.557 | 24.637 | 4.730 | 0.00 | 0.00 |
| ATOM | 1072 | CB | THR | 137 | 81.795 | 26.387 | 5.306 | 0.00 | 0.00 |
| ATOM | 1073 | OG1 | THR | 137 | 82.122 | 26.317 | 3.857 | 0.00 | 0.00 |
| ATOM | 1074 | CG2 | THR | 137 | 82.912 | 26.327 | 6.306 | 0.00 | 0.00 |
| ATOM | 1075 | N | LYS | 138 | 79.086 | 26.774 | 4.709 | 0.00 | 0.00 |
| ATOM | 1076 | CA | LYS | 138 | 77.993 | 27.296 | 3.904 | 0.00 | 0.00 |
| ATOM | 1077 | C | LYS | 138 | 78.325 | 26.967 | 2.391 | 0.00 | 0.00 |
| ATOM | 1078 | O | LYS | 138 | 77.375 | 26.719 | 1.664 | 0.00 | 0.00 |
| ATOM | 1079 | CB | LYS | 138 | 78.023 | 28.822 | 4.135 | 0.00 | 0.00 |
| ATOM | 1080 | CG | LYS | 138 | 77.173 | 29.184 | 5.353 | 0.00 | 0.00 |
| ATOM | 1081 | CD | LYS | 138 | 76.874 | 30.674 | 5.437 | 0.00 | 0.00 |
| ATOM | 1082 | CE | LYS | 138 | 76.812 | 31.202 | 6.901 | 0.00 | 0.00 |
| ATOM | 1083 | NZ | LYS | 138 | 78.150 | 31.653 | 7.456 | 0.00 | 0.00 |
| ATOM | 1084 | N | ASN | 139 | 79.511 | 27.430 | 1.899 | 0.00 | 0.00 |
| ATOM | 1085 | CA | ASN | 139 | 79.969 | 27.231 | 0.550 | 0.00 | 0.00 |
| ATOM | 1086 | C | ASN | 139 | 79.905 | 25.729 | 0.037 | 0.00 | 0.00 |
| ATOM | 1087 | O | ASN | 139 | 79.597 | 25.547 | −1.139 | 0.00 | 0.00 |
| ATOM | 1088 | CB | ASN | 139 | 81.236 | 28.015 | 0.183 | 0.00 | 0.00 |
| ATOM | 1089 | CG | ASN | 139 | 82.349 | 27.691 | 1.208 | 0.00 | 0.00 |
| ATOM | 1090 | OD1 | ASN | 139 | 82.352 | 28.376 | 2.259 | 0.00 | 0.00 |
| ATOM | 1091 | ND2 | ASN | 139 | 83.199 | 26.710 | 0.999 | 0.00 | 0.00 |
| ATOM | 1092 | N | LYS | 140 | 80.445 | 24.709 | 0.754 | 0.00 | 0.00 |
| ATOM | 1093 | CA | LYS | 140 | 80.326 | 23.249 | 0.435 | 0.00 | 0.00 |
| ATOM | 1094 | C | LYS | 140 | 78.793 | 22.868 | 0.384 | 0.00 | 0.00 |
| ATOM | 1095 | O | LYS | 140 | 78.457 | 22.067 | −0.465 | 0.00 | 0.00 |
| ATOM | 1096 | CB | LYS | 140 | 81.089 | 22.373 | 1.426 | 0.00 | 0.00 |
| ATOM | 1097 | CG | LYS | 140 | 82.495 | 22.623 | 1.885 | 0.00 | 0.00 |
| ATOM | 1098 | CD | LYS | 140 | 83.473 | 21.961 | 0.898 | 0.00 | 0.00 |
| ATOM | 1099 | CE | LYS | 140 | 84.897 | 22.455 | 1.072 | 0.00 | 0.00 |
| ATOM | 1100 | NZ | LYS | 140 | 85.704 | 21.656 | 0.070 | 0.00 | 0.00 |
| ATOM | 1101 | N | ALA | 141 | 77.998 | 23.281 | 1.381 | 0.00 | 0.00 |
| ATOM | 1102 | CA | ALA | 141 | 76.555 | 23.120 | 1.464 | 0.00 | 0.00 |
| ATOM | 1103 | C | ALA | 141 | 75.877 | 23.676 | 0.146 | 0.00 | 0.00 |
| ATOM | 1104 | O | ALA | 141 | 74.754 | 23.247 | −0.092 | 0.00 | 0.00 |
| ATOM | 1105 | CB | ALA | 141 | 76.040 | 23.850 | 2.716 | 0.00 | 0.00 |
| ATOM | 1106 | N | ARG | 142 | 76.258 | 24.862 | −0.403 | 0.00 | 0.00 |
| ATOM | 1107 | CA | ARG | 142 | 75.758 | 25.401 | −1.671 | 0.00 | 0.00 |
| ATOM | 1108 | C | ARG | 142 | 76.288 | 24.480 | −2.833 | 0.00 | 0.00 |
| ATOM | 1109 | O | ARG | 142 | 75.710 | 24.586 | −3.910 | 0.00 | 0.00 |
| ATOM | 1110 | CB | ARG | 142 | 76.189 | 26.877 | −1.904 | 0.00 | 0.00 |
| ATOM | 1111 | CG | ARG | 142 | 75.685 | 27.464 | −3.272 | 0.00 | 0.00 |
| ATOM | 1112 | CD | ARG | 142 | 74.172 | 27.451 | −3.325 | 0.00 | 0.00 |
| ATOM | 1113 | NE | ARG | 142 | 73.773 | 28.183 | −4.523 | 0.00 | 0.00 |
| ATOM | 1114 | CZ | ARG | 142 | 73.578 | 29.502 | −4.551 | 0.00 | 0.00 |
| ATOM | 1115 | NH1 | ARG | 142 | 73.721 | 30.238 | −3.451 | 0.00 | 0.00 |
| ATOM | 1116 | NH2 | ARG | 142 | 73.242 | 30.048 | −5.718 | 0.00 | 0.00 |
| ATOM | 1117 | N | VAL | 143 | 77.539 | 23.956 | −2.810 | 0.00 | 0.00 |
| ATOM | 1118 | CA | VAL | 143 | 78.058 | 23.010 | −3.803 | 0.00 | 0.00 |
| ATOM | 1119 | C | VAL | 143 | 77.146 | 21.756 | −3.867 | 0.00 | 0.00 |
| ATOM | 1120 | O | VAL | 143 | 77.005 | 21.265 | −4.966 | 0.00 | 0.00 |
| ATOM | 1121 | CB | VAL | 143 | 79.529 | 22.650 | −3.522 | 0.00 | 0.00 |
| ATOM | 1122 | CG1 | VAL | 143 | 79.992 | 21.496 | −4.403 | 0.00 | 0.00 |
| ATOM | 1123 | CG2 | VAL | 143 | 80.444 | 23.863 | −3.618 | 0.00 | 0.00 |
| ATOM | 1124 | N | ILE | 144 | 76.784 | 21.121 | −2.753 | 0.00 | 0.00 |
| ATOM | 1125 | CA | ILE | 144 | 75.871 | 20.018 | −2.668 | 0.00 | 0.00 |
| ATOM | 1126 | C | ILE | 144 | 74.396 | 20.377 | −3.069 | 0.00 | 0.00 |
| ATOM | 1127 | O | ILE | 144 | 73.710 | 19.462 | −3.507 | 0.00 | 0.00 |
| ATOM | 1128 | CB | ILE | 144 | 76.030 | 19.338 | −1.262 | 0.00 | 0.00 |
| ATOM | 1129 | CG1 | ILE | 144 | 75.955 | 20.321 | −0.084 | 0.00 | 0.00 |
| ATOM | 1130 | CG2 | ILE | 144 | 77.319 | 18.476 | −1.197 | 0.00 | 0.00 |
| ATOM | 1131 | CD1 | ILE | 144 | 75.655 | 19.620 | 1.286 | 0.00 | 0.00 |
| ATOM | 1132 | N | ILE | 145 | 73.790 | 21.504 | −2.598 | 0.00 | 0.00 |
| ATOM | 1133 | CA | ILE | 145 | 72.468 | 21.974 | −2.985 | 0.00 | 0.00 |
| ATOM | 1134 | C | ILE | 145 | 72.537 | 22.281 | −4.534 | 0.00 | 0.00 |
| ATOM | 1135 | O | ILE | 145 | 71.513 | 22.105 | −5.192 | 0.00 | 0.00 |
| ATOM | 1136 | CB | ILE | 145 | 71.867 | 23.141 | −2.122 | 0.00 | 0.00 |
| ATOM | 1137 | CG1 | ILE | 145 | 71.196 | 22.653 | −0.784 | 0.00 | 0.00 |
| ATOM | 1138 | CG2 | ILE | 145 | 70.718 | 23.933 | −2.859 | 0.00 | 0.00 |
| ATOM | 1139 | CD1 | ILE | 145 | 70.965 | 23.783 | 0.260 | 0.00 | 0.00 |
| ATOM | 1140 | N | LEU | 146 | 73.496 | 23.088 | −5.044 | 0.00 | 0.00 |
| ATOM | 1141 | CA | LEU | 146 | 73.700 | 23.348 | −6.479 | 0.00 | 0.00 |
| ATOM | 1142 | C | LEU | 146 | 73.816 | 21.999 | −7.245 | 0.00 | 0.00 |
| ATOM | 1143 | O | LEU | 146 | 73.073 | 21.828 | −8.213 | 0.00 | 0.00 |
| ATOM | 1144 | CB | LEU | 146 | 74.906 | 24.256 | −6.738 | 0.00 | 0.00 |
| ATOM | 1145 | CG | LEU | 146 | 75.209 | 24.584 | −8.196 | 0.00 | 0.00 |
| ATOM | 1146 | CD1 | LEU | 146 | 74.077 | 25.426 | −8.764 | 0.00 | 0.00 |
| ATOM | 1147 | CD2 | LEU | 146 | 76.540 | 25.290 | −8.348 | 0.00 | 0.00 |
| ATOM | 1148 | N | MET | 147 | 74.654 | 21.048 | −6.790 | 0.00 | 0.00 |
| ATOM | 1149 | CA | MET | 147 | 74.786 | 19.722 | −7.338 | 0.00 | 0.00 |
| ATOM | 1150 | C | MET | 147 | 73.445 | 18.924 | −7.294 | 0.00 | 0.00 |
| ATOM | 1151 | O | MET | 147 | 73.185 | 18.247 | −8.295 | 0.00 | 0.00 |
| ATOM | 1152 | CB | MET | 147 | 75.928 | 19.018 | −6.589 | 0.00 | 0.00 |
| ATOM | 1153 | CG | MET | 147 | 75.850 | 17.518 | −6.809 | 0.00 | 0.00 |
| ATOM | 1154 | SD | MET | 147 | 77.118 | 16.743 | −5.821 | 0.00 | 0.00 |
| ATOM | 1155 | CE | MET | 147 | 78.101 | 16.256 | −7.187 | 0.00 | 0.00 |
| ATOM | 1156 | N | VAL | 148 | 72.669 | 18.911 | −6.190 | 0.00 | 0.00 |
| ATOM | 1157 | CA | VAL | 148 | 71.389 | 18.229 | −6.189 | 0.00 | 0.00 |
| ATOM | 1158 | C | VAL | 148 | 70.479 | 18.726 | −7.366 | 0.00 | 0.00 |
| ATOM | 1159 | O | VAL | 148 | 69.728 | 17.883 | −7.844 | 0.00 | 0.00 |
| ATOM | 1160 | CB | VAL | 148 | 70.720 | 18.239 | −4.802 | 0.00 | 0.00 |
| ATOM | 1161 | CG1 | VAL | 148 | 71.533 | 17.505 | −3.740 | 0.00 | 0.00 |
| ATOM | 1162 | CG2 | VAL | 148 | 70.359 | 19.674 | −4.394 | 0.00 | 0.00 |
| ATOM | 1163 | N | TRP | 149 | 70.187 | 20.036 | −7.496 | 0.00 | 0.00 |
| ATOM | 1164 | CA | TRP | 149 | 69.448 | 20.603 | −8.599 | 0.00 | 0.00 |
| ATOM | 1165 | C | TRP | 149 | 69.992 | 20.141 | −9.985 | 0.00 | 0.00 |
| ATOM | 1166 | O | TRP | 149 | 69.160 | 20.098 | −10.899 | 0.00 | 0.00 |
| ATOM | 1167 | CB | TRP | 149 | 69.479 | 22.113 | −8.499 | 0.00 | 0.00 |
| ATOM | 1168 | CG | TRP | 149 | 68.602 | 22.778 | −7.514 | 0.00 | 0.00 |
| ATOM | 1169 | CD1 | TRP | 149 | 68.840 | 22.953 | −6.178 | 0.00 | 0.00 |
| ATOM | 1170 | CD2 | TRP | 149 | 67.319 | 23.365 | −7.777 | 0.00 | 0.00 |
| ATOM | 1171 | NE1 | TRP | 149 | 67.784 | 23.629 | −5.572 | 0.00 | 0.00 |
| ATOM | 1172 | CE2 | TRP | 149 | 66.848 | 23.888 | −6.545 | 0.00 | 0.00 |
| ATOM | 1173 | CE3 | TRP | 149 | 66.547 | 23.515 | −8.924 | 0.00 | 0.00 |
| ATOM | 1174 | CZ2 | TRP | 149 | 65.624 | 24.541 | −6.444 | 0.00 | 0.00 |
| ATOM | 1175 | CZ3 | TRP | 149 | 65.324 | 24.146 | −8.825 | 0.00 | 0.00 |
| ATOM | 1176 | CH2 | TRP | 149 | 64.878 | 24.659 | −7.615 | 0.00 | 0.00 |
| ATOM | 1177 | N | ILE | 150 | 71.311 | 20.225 | −10.275 | 0.00 | 0.00 |
| ATOM | 1178 | CA | ILE | 150 | 71.825 | 19.705 | −11.512 | 0.00 | 0.00 |
| ATOM | 1179 | C | ILE | 150 | 71.385 | 18.232 | −11.749 | 0.00 | 0.00 |
| ATOM | 1180 | O | ILE | 150 | 71.025 | 17.944 | −12.889 | 0.00 | 0.00 |
| ATOM | 1181 | CB | ILE | 150 | 73.385 | 19.823 | −11.568 | 0.00 | 0.00 |
| ATOM | 1182 | CG1 | ILE | 150 | 73.924 | 21.225 | −11.346 | 0.00 | 0.00 |
| ATOM | 1183 | CG2 | ILE | 150 | 73.951 | 19.323 | −12.935 | 0.00 | 0.00 |
| ATOM | 1184 | CD1 | ILE | 150 | 75.439 | 21.137 | −11.044 | 0.00 | 0.00 |
| ATOM | 1185 | N | VAL | 151 | 71.712 | 17.257 | −10.882 | 0.00 | 0.00 |
| ATOM | 1186 | CA | VAL | 151 | 71.310 | 15.871 | −11.121 | 0.00 | 0.00 |
| ATOM | 1187 | C | VAL | 151 | 69.751 | 15.692 | −11.153 | 0.00 | 0.00 |
| ATOM | 1188 | O | VAL | 151 | 69.326 | 14.763 | −11.854 | 0.00 | 0.00 |
| ATOM | 1189 | CB | VAL | 151 | 71.990 | 14.872 | −10.198 | 0.00 | 0.00 |
| ATOM | 1190 | CG1 | VAL | 151 | 71.490 | 13.431 | −10.275 | 0.00 | 0.00 |
| ATOM | 1191 | CG2 | VAL | 151 | 73.478 | 15.016 | −10.190 | 0.00 | 0.00 |
| ATOM | 1192 | N | SER | 152 | 68.958 | 16.244 | −10.213 | 0.00 | 0.00 |
| ATOM | 1193 | CA | SER | 152 | 67.514 | 16.173 | −10.293 | 0.00 | 0.00 |
| ATOM | 1194 | C | SER | 152 | 66.999 | 16.703 | −11.659 | 0.00 | 0.00 |
| ATOM | 1195 | O | SER | 152 | 65.934 | 16.247 | −12.084 | 0.00 | 0.00 |
| ATOM | 1196 | CB | SER | 152 | 67.021 | 17.108 | −9.211 | 0.00 | 0.00 |
| ATOM | 1197 | OG | SER | 152 | 67.112 | 18.506 | −9.380 | 0.00 | 0.00 |
| ATOM | 1198 | N | GLY | 153 | 67.488 | 17.903 | −12.070 | 0.00 | 0.00 |
| ATOM | 1199 | CA | GLY | 153 | 67.223 | 18.518 | −13.331 | 0.00 | 0.00 |
| ATOM | 1200 | C | GLY | 153 | 67.565 | 17.555 | −14.478 | 0.00 | 0.00 |
| ATOM | 1201 | O | GLY | 153 | 66.837 | 17.561 | −15.460 | 0.00 | 0.00 |
| ATOM | 1202 | N | LEU | 154 | 68.752 | 16.944 | −14.485 | 0.00 | 0.00 |
| ATOM | 1203 | CA | LEU | 154 | 69.164 | 15.935 | −15.451 | 0.00 | 0.00 |
| ATOM | 1204 | C | LEU | 154 | 68.039 | 14.851 | −15.634 | 0.00 | 0.00 |
| ATOM | 1205 | O | LEU | 154 | 67.363 | 14.957 | −16.641 | 0.00 | 0.00 |
| ATOM | 1206 | CB | LEU | 154 | 70.432 | 15.388 | −14.907 | 0.00 | 0.00 |
| ATOM | 1207 | CG | LEU | 154 | 71.298 | 14.293 | −15.421 | 0.00 | 0.00 |
| ATOM | 1208 | CD1 | LEU | 154 | 70.537 | 12.958 | −15.552 | 0.00 | 0.00 |
| ATOM | 1209 | CD2 | LEU | 154 | 71.873 | 14.658 | −16.799 | 0.00 | 0.00 |
| ATOM | 1210 | N | THR | 155 | 67.541 | 14.182 | −14.559 | 0.00 | 0.00 |
| ATOM | 1211 | CA | THR | 155 | 66.568 | 13.105 | −14.633 | 0.00 | 0.00 |
| ATOM | 1212 | C | THR | 155 | 65.176 | 13.583 | −15.212 | 0.00 | 0.00 |
| ATOM | 1213 | O | THR | 155 | 64.808 | 13.101 | −16.278 | 0.00 | 0.00 |
| ATOM | 1214 | CB | THR | 155 | 66.472 | 12.515 | −13.196 | 0.00 | 0.00 |
| ATOM | 1215 | OG1 | THR | 155 | 67.702 | 11.884 | −12.752 | 0.00 | 0.00 |
| ATOM | 1216 | CG2 | THR | 155 | 65.282 | 11.510 | −13.166 | 0.00 | 0.00 |
| ATOM | 1217 | N | SER | 156 | 64.526 | 14.641 | −14.694 | 0.00 | 0.00 |
| ATOM | 1218 | CA | SER | 156 | 63.173 | 15.097 | −15.101 | 0.00 | 0.00 |
| ATOM | 1219 | C | SER | 156 | 63.145 | 16.097 | −16.300 | 0.00 | 0.00 |
| ATOM | 1220 | O | SER | 156 | 62.032 | 16.247 | −16.850 | 0.00 | 0.00 |
| ATOM | 1221 | CB | SER | 156 | 62.543 | 15.725 | −13.848 | 0.00 | 0.00 |
| ATOM | 1222 | OG | SER | 156 | 63.112 | 16.913 | −13.318 | 0.00 | 0.00 |
| ATOM | 1223 | N | PHE | 157 | 64.010 | 17.121 | −16.337 | 0.00 | 0.00 |
| ATOM | 1224 | CA | PHE | 157 | 64.070 | 18.017 | −17.525 | 0.00 | 0.00 |
| ATOM | 1225 | C | PHE | 157 | 63.996 | 17.212 | −18.847 | 0.00 | 0.00 |
| ATOM | 1226 | O | PHE | 157 | 63.479 | 17.799 | −19.820 | 0.00 | 0.00 |
| ATOM | 1227 | CB | PHE | 157 | 65.284 | 18.948 | −17.417 | 0.00 | 0.00 |
| ATOM | 1228 | CG | PHE | 157 | 65.486 | 19.799 | −18.667 | 0.00 | 0.00 |
| ATOM | 1229 | CD1 | PHE | 157 | 64.673 | 20.896 | −18.875 | 0.00 | 0.00 |
| ATOM | 1230 | CD2 | PHE | 157 | 66.464 | 19.489 | −19.591 | 0.00 | 0.00 |
| ATOM | 1231 | CE1 | PHE | 157 | 64.842 | 21.664 | −20.000 | 0.00 | 0.00 |
| ATOM | 1232 | CE2 | PHE | 157 | 66.625 | 20.269 | −20.720 | 0.00 | 0.00 |
| ATOM | 1233 | CZ | PHE | 157 | 65.810 | 21.357 | −20.926 | 0.00 | 0.00 |
| ATOM | 1234 | N | LEU | 158 | 64.568 | 16.006 | −18.947 | 0.00 | 0.00 |
| ATOM | 1235 | CA | LEU | 158 | 64.627 | 15.178 | −20.116 | 0.00 | 0.00 |
| ATOM | 1236 | C | LEU | 158 | 63.267 | 14.919 | −20.769 | 0.00 | 0.00 |
| ATOM | 1237 | O | LEU | 158 | 63.162 | 15.293 | −21.931 | 0.00 | 0.00 |
| ATOM | 1238 | CB | LEU | 158 | 65.386 | 13.916 | −19.870 | 0.00 | 0.00 |
| ATOM | 1239 | CG | LEU | 158 | 66.636 | 13.880 | −19.076 | 0.00 | 0.00 |
| ATOM | 1240 | CD1 | LEU | 158 | 66.946 | 12.438 | −18.637 | 0.00 | 0.00 |
| ATOM | 1241 | CD2 | LEU | 158 | 67.724 | 14.481 | −19.912 | 0.00 | 0.00 |
| ATOM | 1242 | N | PRO | 159 | 62.236 | 14.264 | −20.152 | 0.00 | 0.00 |
| ATOM | 1243 | CA | PRO | 159 | 61.055 | 14.057 | −20.917 | 0.00 | 0.00 |
| ATOM | 1244 | C | PRO | 159 | 60.487 | 15.351 | −21.586 | 0.00 | 0.00 |
| ATOM | 1245 | O | PRO | 159 | 59.764 | 15.170 | −22.537 | 0.00 | 0.00 |
| ATOM | 1246 | CB | PRO | 159 | 60.030 | 13.299 | −20.015 | 0.00 | 0.00 |
| ATOM | 1247 | CG | PRO | 159 | 60.466 | 13.762 | −18.573 | 0.00 | 0.00 |
| ATOM | 1248 | CD | PRO | 159 | 61.922 | 14.162 | −18.675 | 0.00 | 0.00 |
| ATOM | 1249 | N | ILE | 160 | 60.340 | 16.503 | −20.887 | 0.00 | 0.00 |
| ATOM | 1250 | CA | ILE | 160 | 59.812 | 17.704 | −21.609 | 0.00 | 0.00 |
| ATOM | 1251 | C | ILE | 160 | 60.605 | 17.968 | −22.926 | 0.00 | 0.00 |
| ATOM | 1252 | O | ILE | 160 | 59.955 | 17.977 | −23.969 | 0.00 | 0.00 |
| ATOM | 1253 | CB | ILE | 160 | 59.797 | 19.021 | −20.762 | 0.00 | 0.00 |
| ATOM | 1254 | CG1 | ILE | 160 | 58.789 | 18.889 | −19.608 | 0.00 | 0.00 |
| ATOM | 1255 | CG2 | ILE | 160 | 59.482 | 20.267 | −21.672 | 0.00 | 0.00 |
| ATOM | 1256 | CD1 | ILE | 160 | 58.618 | 20.116 | −18.691 | 0.00 | 0.00 |
| ATOM | 1257 | N | GLN | 161 | 61.950 | 18.049 | −22.922 | 0.00 | 0.00 |
| ATOM | 1258 | CA | GLN | 161 | 62.810 | 18.298 | −24.090 | 0.00 | 0.00 |
| ATOM | 1259 | C | GLN | 161 | 63.031 | 17.005 | −24.918 | 0.00 | 0.00 |
| ATOM | 1260 | O | GLN | 161 | 63.983 | 16.256 | −24.683 | 0.00 | 0.00 |
| ATOM | 1261 | CB | GLN | 161 | 64.119 | 18.937 | −23.632 | 0.00 | 0.00 |
| ATOM | 1262 | CG | GLN | 161 | 64.938 | 19.448 | −24.835 | 0.00 | 0.00 |
| ATOM | 1263 | CD | GLN | 161 | 66.422 | 19.469 | −24.639 | 0.00 | 0.00 |
| ATOM | 1264 | OE1 | GLN | 161 | 66.997 | 19.469 | −23.554 | 0.00 | 0.00 |
| ATOM | 1265 | NE2 | GLN | 161 | 67.121 | 19.459 | −25.807 | 0.00 | 0.00 |
| ATOM | 1266 | N | MET | 162 | 62.348 | 17.028 | −26.053 | 0.00 | 0.00 |
| ATOM | 1267 | CA | MET | 162 | 62.245 | 15.948 | −27.040 | 0.00 | 0.00 |
| ATOM | 1268 | C | MET | 162 | 61.839 | 14.598 | −26.367 | 0.00 | 0.00 |
| ATOM | 1269 | O | MET | 162 | 61.861 | 13.576 | −27.070 | 0.00 | 0.00 |
| ATOM | 1270 | CB | MET | 162 | 63.552 | 15.847 | −27.838 | 0.00 | 0.00 |
| ATOM | 1271 | CG | MET | 162 | 63.986 | 17.239 | −28.345 | 0.00 | 0.00 |
| ATOM | 1272 | SD | MET | 162 | 63.029 | 17.571 | −29.840 | 0.00 | 0.00 |
| ATOM | 1273 | CE | MET | 162 | 63.690 | 19.114 | −30.489 | 0.00 | 0.00 |
| ATOM | 1274 | N | HIS | 163 | 61.170 | 14.678 | −25.195 | 0.00 | 0.00 |
| ATOM | 1275 | CA | HIS | 163 | 60.629 | 13.543 | −24.484 | 0.00 | 0.00 |
| ATOM | 1276 | C | HIS | 163 | 61.816 | 12.526 | −24.347 | 0.00 | 0.00 |
| ATOM | 1277 | O | HIS | 163 | 61.816 | 11.656 | −25.276 | 0.00 | 0.00 |
| ATOM | 1278 | CB | HIS | 163 | 59.452 | 13.084 | −25.324 | 0.00 | 0.00 |
| ATOM | 1279 | CG | HIS | 163 | 58.194 | 13.821 | −24.989 | 0.00 | 0.00 |
| ATOM | 1280 | ND1 | HIS | 163 | 57.029 | 13.256 | −24.501 | 0.00 | 0.00 |
| ATOM | 1281 | CD2 | HIS | 163 | 57.950 | 15.161 | −25.091 | 0.00 | 0.00 |
| ATOM | 1282 | CE1 | HIS | 163 | 56.120 | 14.244 | −24.400 | 0.00 | 0.00 |
| ATOM | 1283 | NE2 | HIS | 163 | 56.639 | 15.400 | −24.679 | 0.00 | 0.00 |
| ATOM | 1284 | N | TRP | 164 | 63.022 | 12.908 | −23.827 | 0.00 | 0.00 |
| ATOM | 1285 | CA | TRP | 164 | 63.883 | 11.795 | −23.609 | 0.00 | 0.00 |
| ATOM | 1286 | C | TRP | 164 | 62.859 | 10.988 | −22.729 | 0.00 | 0.00 |
| ATOM | 1287 | O | TRP | 164 | 62.382 | 11.526 | −21.720 | 0.00 | 0.00 |
| ATOM | 1288 | CB | TRP | 164 | 65.221 | 12.146 | −22.981 | 0.00 | 0.00 |
| ATOM | 1289 | CG | TRP | 164 | 66.215 | 11.049 | −23.091 | 0.00 | 0.00 |
| ATOM | 1290 | CD1 | TRP | 164 | 66.175 | 9.966 | −23.933 | 0.00 | 0.00 |
| ATOM | 1291 | CD2 | TRP | 164 | 67.394 | 10.895 | −22.291 | 0.00 | 0.00 |
| ATOM | 1292 | NE1 | TRP | 164 | 67.249 | 9.139 | −23.689 | 0.00 | 0.00 |
| ATOM | 1293 | CE2 | TRP | 164 | 68.013 | 9.684 | −22.688 | 0.00 | 0.00 |
| ATOM | 1294 | CE3 | TRP | 164 | 67.982 | 11.653 | −21.269 | 0.00 | 0.00 |
| ATOM | 1295 | CZ2 | TRP | 164 | 69.189 | 9.216 | −22.094 | 0.00 | 0.00 |
| ATOM | 1296 | CZ3 | TRP | 164 | 69.142 | 11.188 | −20.681 | 0.00 | 0.00 |
| ATOM | 1297 | CH2 | TRP | 164 | 69.737 | 9.978 | −21.094 | 0.00 | 0.00 |
| ATOM | 1298 | N | TYR | 165 | 62.251 | 9.990 | −23.297 | 0.00 | 0.00 |
| ATOM | 1299 | CA | TYR | 165 | 61.150 | 9.255 | −22.749 | 0.00 | 0.00 |
| ATOM | 1300 | C | TYR | 165 | 59.969 | 10.319 | −22.761 | 0.00 | 0.00 |
| ATOM | 1301 | O | TYR | 165 | 59.694 | 10.799 | −23.849 | 0.00 | 0.00 |
| ATOM | 1302 | CB | TYR | 165 | 61.378 | 8.508 | −21.415 | 0.00 | 0.00 |
| ATOM | 1303 | CG | TYR | 165 | 61.070 | 9.315 | −20.200 | 0.00 | 0.00 |
| ATOM | 1304 | CD1 | TYR | 165 | 59.816 | 9.147 | −19.601 | 0.00 | 0.00 |
| ATOM | 1305 | CD2 | TYR | 165 | 62.084 | 10.139 | −19.637 | 0.00 | 0.00 |
| ATOM | 1306 | CE1 | TYR | 165 | 59.522 | 9.856 | −18.427 | 0.00 | 0.00 |
| ATOM | 1307 | CE2 | TYR | 165 | 61.758 | 10.818 | −18.415 | 0.00 | 0.00 |
| ATOM | 1308 | CZ | TYR | 165 | 60.502 | 10.672 | −17.872 | 0.00 | 0.00 |
| ATOM | 1309 | OH | TYR | 165 | 60.189 | 11.329 | −16.710 | 0.00 | 0.00 |
| ATOM | 1310 | N | ARG | 166 | 59.031 | 10.400 | −21.769 | 0.00 | 0.00 |
| ATOM | 1311 | CA | ARG | 166 | 57.887 | 11.357 | −21.841 | 0.00 | 0.00 |
| ATOM | 1312 | C | ARG | 166 | 57.049 | 11.484 | −20.508 | 0.00 | 0.00 |
| ATOM | 1313 | O | ARG | 166 | 56.975 | 10.518 | −19.742 | 0.00 | 0.00 |
| ATOM | 1314 | CB | ARG | 166 | 56.910 | 10.805 | −22.914 | 0.00 | 0.00 |
| ATOM | 1315 | CG | ARG | 166 | 56.518 | 9.394 | −22.681 | 0.00 | 0.00 |
| ATOM | 1316 | CD | ARG | 166 | 55.152 | 8.979 | −23.067 | 0.00 | 0.00 |
| ATOM | 1317 | NE | ARG | 166 | 55.003 | 8.503 | −24.402 | 0.00 | 0.00 |
| ATOM | 1318 | CZ | ARG | 166 | 54.167 | 7.507 | −24.678 | 0.00 | 0.00 |
| ATOM | 1319 | NH1 | ARG | 166 | 53.443 | 6.865 | −23.764 | 0.00 | 0.00 |
| ATOM | 1320 | NH2 | ARG | 166 | 53.933 | 7.240 | −25.955 | 0.00 | 0.00 |
| ATOM | 1321 | N | ALA | 167 | 56.192 | 12.498 | −20.476 | 0.00 | 0.00 |
| ATOM | 1322 | CA | ALA | 167 | 55.243 | 12.749 | −19.394 | 0.00 | 0.00 |
| ATOM | 1323 | C | ALA | 167 | 53.833 | 12.230 | −19.848 | 0.00 | 0.00 |
| ATOM | 1324 | O | ALA | 167 | 53.296 | 12.701 | −20.854 | 0.00 | 0.00 |
| ATOM | 1325 | CB | ALA | 167 | 55.233 | 14.257 | −19.132 | 0.00 | 0.00 |
| ATOM | 1326 | N | THR | 168 | 53.444 | 11.045 | −19.340 | 0.00 | 0.00 |
| ATOM | 1327 | CA | THR | 168 | 52.130 | 10.421 | −19.544 | 0.00 | 0.00 |
| ATOM | 1328 | C | THR | 168 | 51.461 | 10.222 | −18.126 | 0.00 | 0.00 |
| ATOM | 1329 | O | THR | 168 | 52.117 | 9.675 | −17.232 | 0.00 | 0.00 |
| ATOM | 1330 | CB | THR | 168 | 52.268 | 9.125 | −20.420 | 0.00 | 0.00 |
| ATOM | 1331 | OG1 | THR | 168 | 51.005 | 8.631 | −20.938 | 0.00 | 0.00 |
| ATOM | 1332 | CG2 | THR | 168 | 53.002 | 7.973 | −19.687 | 0.00 | 0.00 |
| ATOM | 1333 | N | HIS | 169 | 50.156 | 10.039 | −18.162 | 0.00 | 0.00 |
| ATOM | 1334 | CA | HIS | 169 | 49.234 | 9.927 | −16.992 | 0.00 | 0.00 |
| ATOM | 1335 | C | HIS | 169 | 49.137 | 8.493 | −16.322 | 0.00 | 0.00 |
| ATOM | 1336 | O | HIS | 169 | 49.429 | 7.520 | −17.025 | 0.00 | 0.00 |
| ATOM | 1337 | CB | HIS | 169 | 47.835 | 10.257 | −17.561 | 0.00 | 0.00 |
| ATOM | 1338 | CG | HIS | 169 | 47.359 | 9.347 | −18.666 | 0.00 | 0.00 |
| ATOM | 1339 | ND1 | HIS | 169 | 47.572 | 9.655 | −20.008 | 0.00 | 0.00 |
| ATOM | 1340 | CD2 | HIS | 169 | 46.724 | 8.148 | −18.647 | 0.00 | 0.00 |
| ATOM | 1341 | CE1 | HIS | 169 | 47.077 | 8.671 | −20.738 | 0.00 | 0.00 |
| ATOM | 1342 | NE2 | HIS | 169 | 46.569 | 7.764 | −19.940 | 0.00 | 0.00 |
| ATOM | 1343 | N | GLN | 170 | 48.562 | 8.340 | −15.097 | 0.00 | 0.00 |
| ATOM | 1344 | CA | GLN | 170 | 48.363 | 6.995 | −14.497 | 0.00 | 0.00 |
| ATOM | 1345 | C | GLN | 170 | 46.822 | 6.648 | −14.459 | 0.00 | 0.00 |
| ATOM | 1346 | O | GLN | 170 | 46.415 | 5.923 | −15.362 | 0.00 | 0.00 |
| ATOM | 1347 | CB | GLN | 170 | 49.011 | 6.969 | −13.160 | 0.00 | 0.00 |
| ATOM | 1348 | CG | GLN | 170 | 48.706 | 8.057 | −12.229 | 0.00 | 0.00 |
| ATOM | 1349 | CD | GLN | 170 | 49.500 | 7.898 | −10.966 | 0.00 | 0.00 |
| ATOM | 1350 | OE1 | GLN | 170 | 50.636 | 8.378 | −10.871 | 0.00 | 0.00 |
| ATOM | 1351 | NE2 | GLN | 170 | 48.938 | 7.170 | −9.997 | 0.00 | 0.00 |
| ATOM | 1352 | N | GLU | 171 | 45.994 | 6.953 | −13.416 | 0.00 | 0.00 |
| ATOM | 1353 | CA | GLU | 171 | 44.504 | 6.819 | −13.444 | 0.00 | 0.00 |
| ATOM | 1354 | C | GLU | 171 | 43.985 | 8.168 | −14.008 | 0.00 | 0.00 |
| ATOM | 1355 | O | GLU | 171 | 42.909 | 8.639 | −13.617 | 0.00 | 0.00 |
| ATOM | 1356 | CB | GLU | 171 | 43.995 | 6.773 | −12.015 | 0.00 | 0.00 |
| ATOM | 1357 | CG | GLU | 171 | 44.527 | 7.725 | −10.986 | 0.00 | 0.00 |
| ATOM | 1358 | CD | GLU | 171 | 43.952 | 7.627 | −9.671 | 0.00 | 0.00 |
| ATOM | 1359 | OE1 | GLU | 171 | 44.714 | 8.195 | −8.888 | 0.00 | 0.00 |
| ATOM | 1360 | OE2 | GLU | 171 | 42.809 | 7.181 | −9.196 | 0.00 | 0.00 |
| ATOM | 1361 | N | ALA | 172 | 44.476 | 8.513 | −15.167 | 0.00 | 0.00 |
| ATOM | 1362 | CA | ALA | 172 | 44.180 | 9.811 | −15.750 | 0.00 | 0.00 |
| ATOM | 1363 | C | ALA | 172 | 44.234 | 10.923 | −14.615 | 0.00 | 0.00 |
| ATOM | 1364 | O | ALA | 172 | 43.353 | 11.810 | −14.677 | 0.00 | 0.00 |
| ATOM | 1365 | CB | ALA | 172 | 42.820 | 9.727 | −16.461 | 0.00 | 0.00 |
| ATOM | 1366 | N | ILE | 173 | 45.089 | 10.783 | −13.624 | 0.00 | 0.00 |
| ATOM | 1367 | CA | ILE | 173 | 45.277 | 11.664 | −12.455 | 0.00 | 0.00 |
| ATOM | 1368 | C | ILE | 173 | 46.743 | 12.104 | −12.232 | 0.00 | 0.00 |
| ATOM | 1369 | O | ILE | 173 | 46.971 | 13.200 | −11.640 | 0.00 | 0.00 |
| ATOM | 1370 | CB | ILE | 173 | 44.658 | 11.116 | −11.140 | 0.00 | 0.00 |
| ATOM | 1371 | CG1 | ILE | 173 | 43.103 | 11.105 | −11.296 | 0.00 | 0.00 |
| ATOM | 1372 | CG2 | ILE | 173 | 45.045 | 11.857 | −9.849 | 0.00 | 0.00 |
| ATOM | 1373 | CD1 | ILE | 173 | 42.433 | 10.015 | −10.434 | 0.00 | 0.00 |
| ATOM | 1374 | N | ASN | 174 | 47.753 | 11.415 | −12.843 | 0.00 | 0.00 |
| ATOM | 1375 | CA | ASN | 174 | 49.105 | 11.798 | −12.562 | 0.00 | 0.00 |
| ATOM | 1376 | C | ASN | 174 | 50.016 | 11.334 | −13.707 | 0.00 | 0.00 |
| ATOM | 1377 | O | ASN | 174 | 50.269 | 10.139 | −13.819 | 0.00 | 0.00 |
| ATOM | 1378 | CB | ASN | 174 | 49.544 | 11.002 | −11.302 | 0.00 | 0.00 |
| ATOM | 1379 | CG | ASN | 174 | 50.988 | 10.972 | −10.923 | 0.00 | 0.00 |
| ATOM | 1380 | OD1 | ASN | 174 | 51.828 | 10.527 | −11.731 | 0.00 | 0.00 |
| ATOM | 1381 | ND2 | ASN | 174 | 51.368 | 11.437 | −9.752 | 0.00 | 0.00 |
| ATOM | 1382 | N | CYS | 175 | 50.950 | 12.176 | −13.938 | 0.00 | 0.00 |
| ATOM | 1383 | CA | CYS | 175 | 52.003 | 11.899 | −14.937 | 0.00 | 0.00 |
| ATOM | 1384 | C | CYS | 175 | 53.397 | 11.625 | −14.346 | 0.00 | 0.00 |
| ATOM | 1385 | O | CYS | 175 | 53.787 | 12.104 | −13.275 | 0.00 | 0.00 |
| ATOM | 1386 | CB | CYS | 175 | 52.018 | 12.948 | −16.035 | 0.00 | 0.00 |
| ATOM | 1387 | SG | CYS | 175 | 50.563 | 13.014 | −17.024 | 0.00 | 0.00 |
| ATOM | 1388 | N | TYR | 176 | 54.137 | 10.840 | −15.158 | 0.00 | 0.00 |
| ATOM | 1389 | CA | TYR | 176 | 55.496 | 10.410 | −14.930 | 0.00 | 0.00 |
| ATOM | 1390 | C | TYR | 176 | 55.920 | 9.579 | −16.213 | 0.00 | 0.00 |
| ATOM | 1391 | O | TYR | 176 | 55.703 | 10.032 | −17.355 | 0.00 | 0.00 |
| ATOM | 1392 | CB | TYR | 176 | 55.67 | 19.762 | −13.508 | 0.00 | 0.00 |
| ATOM | 1393 | CG | TYR | 176 | 57.129 | 9.219 | −13.443 | 0.00 | 0.00 |
| ATOM | 1394 | CD1 | TYR | 176 | 58.136 | 10.146 | −13.197 | 0.00 | 0.00 |
| ATOM | 1395 | CD2 | TYR | 176 | 57.445 | 7.863 | −13.509 | 0.00 | 0.00 |
| ATOM | 1396 | CE1 | TYR | 176 | 59.455 | 9.730 | −13.015 | 0.00 | 0.00 |
| ATOM | 1397 | CE2 | TYR | 176 | 58.766 | 7.439 | −13.331 | 0.00 | 0.00 |
| ATOM | 1398 | CZ | TYR | 176 | 59.774 | 8.373 | −13.078 | 0.00 | 0.00 |
| ATOM | 1399 | OH | TYR | 176 | 61.088 | 7.975 | −12.850 | 0.00 | 0.00 |
| ATOM | 1400 | N | ALA | 177 | 56.821 | 8.590 | −16.032 | 0.00 | 0.00 |
| ATOM | 1401 | CA | ALA | 177 | 57.395 | 7.772 | −17.032 | 0.00 | 0.00 |
| ATOM | 1402 | C | ALA | 177 | 56.322 | 7.156 | −18.000 | 0.00 | 0.00 |
| ATOM | 1403 | O | ALA | 177 | 55.290 | 6.672 | −17.536 | 0.00 | 0.00 |
| ATOM | 1404 | CB | ALA | 177 | 58.252 | 6.729 | −16.295 | 0.00 | 0.00 |
| ATOM | 1405 | N | ASN | 178 | 56.870 | 6.642 | −19.090 | 0.00 | 0.00 |
| ATOM | 1406 | CA | ASN | 178 | 56.211 | 6.151 | −20.336 | 0.00 | 0.00 |
| ATOM | 1407 | C | ASN | 178 | 55.139 | 4.979 | −20.379 | 0.00 | 0.00 |
| ATOM | 1408 | O | ASN | 178 | 53.979 | 5.333 | −20.553 | 0.00 | 0.00 |
| ATOM | 1409 | CB | ASN | 178 | 57.336 | 5.907 | −21.279 | 0.00 | 0.00 |
| ATOM | 1410 | CG | ASN | 178 | 57.867 | 4.418 | −21.201 | 0.00 | 0.00 |
| ATOM | 1411 | OD1 | ASN | 178 | 57.617 | 3.541 | −22.027 | 0.00 | 0.00 |
| ATOM | 1412 | ND2 | ASN | 178 | 58.600 | 4.106 | −20.140 | 0.00 | 0.00 |
| ATOM | 1413 | N | GLU | 179 | 55.461 | 3.669 | −20.530 | 0.00 | 0.00 |
| ATOM | 1414 | CA | GLU | 179 | 54.462 | 2.575 | −20.747 | 0.00 | 0.00 |
| ATOM | 1415 | C | GLU | 179 | 54.183 | 2.215 | −22.255 | 0.00 | 0.00 |
| ATOM | 1416 | O | GLU | 179 | 53.412 | 1.262 | −22.476 | 0.00 | 0.00 |
| ATOM | 1417 | CB | GLU | 179 | 53.138 | 2.788 | −19.965 | 0.00 | 0.00 |
| ATOM | 1418 | CG | GLU | 179 | 53.288 | 2.758 | −18.428 | 0.00 | 0.00 |
| ATOM | 1419 | CD | GLU | 179 | 51.951 | 2.966 | −17.833 | 0.00 | 0.00 |
| ATOM | 1420 | OE1 | GLU | 179 | 50.905 | 3.254 | −18.403 | 0.00 | 0.00 |
| ATOM | 1421 | OE2 | GLU | 179 | 51.914 | 2.573 | −16.610 | 0.00 | 0.00 |
| ATOM | 1422 | N | THR | 180 | 54.961 | 2.704 | −23.239 | 0.00 | 0.00 |
| ATOM | 1423 | CA | THR | 180 | 54.877 | 2.372 | −24.659 | 0.00 | 0.00 |
| ATOM | 1424 | C | THR | 180 | 56.327 | 2.461 | −25.191 | 0.00 | 0.00 |
| ATOM | 1425 | O | THR | 180 | 57.014 | 3.468 | −24.913 | 0.00 | 0.00 |
| ATOM | 1426 | CB | THR | 180 | 53.925 | 3.295 | −25.476 | 0.00 | 0.00 |
| ATOM | 1427 | OG1 | THR | 180 | 54.490 | 4.644 | −25.636 | 0.00 | 0.00 |
| ATOM | 1428 | CG2 | THR | 180 | 52.513 | 3.389 | −24.894 | 0.00 | 0.00 |
| ATOM | 1429 | N | CYS | 181 | 56.792 | 1.423 | −25.905 | 0.00 | 0.00 |
| ATOM | 1430 | CA | CYS | 181 | 58.126 | 1.502 | −26.519 | 0.00 | 0.00 |
| ATOM | 1431 | C | CYS | 181 | 58.362 | 2.935 | −27.116 | 0.00 | 0.00 |
| ATOM | 1432 | O | CYS | 181 | 59.325 | 3.554 | −26.652 | 0.00 | 0.00 |
| ATOM | 1433 | CB | CYS | 181 | 58.313 | 0.370 | −27.547 | 0.00 | 0.00 |
| ATOM | 1434 | SG | CYS | 181 | 59.819 | 0.712 | −28.515 | 0.00 | 0.00 |
| ATOM | 1435 | N | CYS | 182 | 57.463 | 3.513 | −27.919 | 0.00 | 0.00 |
| ATOM | 1436 | CA | CYS | 182 | 57.591 | 4.869 | −28.411 | 0.00 | 0.00 |
| ATOM | 1437 | C | CYS | 182 | 58.968 | 5.101 | −29.103 | 0.00 | 0.00 |
| ATOM | 1438 | O | CYS | 182 | 59.784 | 5.844 | −28.541 | 0.00 | 0.00 |
| ATOM | 1439 | CB | CYS | 182 | 57.261 | 5.896 | −27.337 | 0.00 | 0.00 |
| ATOM | 1440 | SG | CYS | 182 | 56.750 | 7.500 | −28.058 | 0.00 | 0.00 |
| ATOM | 1441 | N | ASP | 183 | 59.361 | 4.224 | −30.073 | 0.00 | 0.00 |
| ATOM | 1442 | CA | ASP | 183 | 60.652 | 4.296 | −30.767 | 0.00 | 0.00 |
| ATOM | 1443 | C | ASP | 183 | 61.065 | 5.756 | −31.153 | 0.00 | 0.00 |
| ATOM | 1444 | O | ASP | 183 | 62.276 | 5.992 | −31.116 | 0.00 | 0.00 |
| ATOM | 1445 | CB | ASP | 183 | 60.520 | 3.343 | −31.918 | 0.00 | 0.00 |
| ATOM | 1446 | CG | ASP | 183 | 60.336 | 3.577 | −33.367 | 0.00 | 0.00 |
| ATOM | 1447 | OD1 | ASP | 183 | 60.764 | 2.631 | −34.200 | 0.00 | 0.00 |
| ATOM | 1448 | OD2 | ASP | 183 | 59.795 | 4.594 | −33.942 | 0.00 | 0.00 |
| ATOM | 1449 | N | PHE | 184 | 60.180 | 6.619 | −31.722 | 0.00 | 0.00 |
| ATOM | 1450 | CA | PHE | 184 | 60.452 | 8.026 | −32.021 | 0.00 | 0.00 |
| ATOM | 1451 | C | PHE | 184 | 61.160 | 8.739 | −30.823 | 0.00 | 0.00 |
| ATOM | 1452 | O | PHE | 184 | 62.276 | 9.227 | −31.038 | 0.00 | 0.00 |
| ATOM | 1453 | CB | PHE | 184 | 59.147 | 8.722 | −32.442 | 0.00 | 0.00 |
| ATOM | 1454 | CG | PHE | 184 | 59.347 | 10.072 | −33.102 | 0.00 | 0.00 |
| ATOM | 1455 | CD1 | PHE | 184 | 60.114 | 10.115 | −34.265 | 0.00 | 0.00 |
| ATOM | 1456 | CD2 | PHE | 184 | 58.773 | 11.234 | −32.488 | 0.00 | 0.00 |
| ATOM | 1457 | CE1 | PHE | 184 | 60.293 | 11.374 | −34.869 | 0.00 | 0.00 |
| ATOM | 1458 | CE2 | PHE | 184 | 59.053 | 12.437 | −33.105 | 0.00 | 0.00 |
| ATOM | 1459 | CZ | PHE | 184 | 59.741 | 12.580 | −34.265 | 0.00 | 0.00 |
| ATOM | 1460 | N | PHE | 185 | 60.626 | 8.715 | −29.587 | 0.00 | 0.00 |
| ATOM | 1461 | CA | PHE | 185 | 61.187 | 9.437 | −28.413 | 0.00 | 0.00 |
| ATOM | 1462 | C | PHE | 185 | 62.133 | 8.581 | −27.498 | 0.00 | 0.00 |
| ATOM | 1463 | O | PHE | 185 | 62.673 | 9.155 | −26.543 | 0.00 | 0.00 |
| ATOM | 1464 | CB | PHE | 185 | 60.015 | 9.997 | −27.612 | 0.00 | 0.00 |
| ATOM | 1465 | CG | PHE | 185 | 59.121 | 10.914 | −28.391 | 0.00 | 0.00 |
| ATOM | 1466 | CD1 | PHE | 185 | 59.597 | 12.241 | −28.559 | 0.00 | 0.00 |
| ATOM | 1467 | CD2 | PHE | 185 | 57.928 | 10.510 | −28.993 | 0.00 | 0.00 |
| ATOM | 1468 | CE1 | PHE | 185 | 58.793 | 13.119 | −29.350 | 0.00 | 0.00 |
| ATOM | 1469 | CE2 | PHE | 185 | 57.167 | 11.396 | −29.763 | 0.00 | 0.00 |
| ATOM | 1470 | CZ | PHE | 185 | 57.603 | 12.719 | −29.943 | 0.00 | 0.00 |
| ATOM | 1471 | N | THR | 186 | 62.465 | 7.332 | −27.833 | 0.00 | 0.00 |
| ATOM | 1472 | CA | THR | 186 | 63.421 | 6.452 | −27.121 | 0.00 | 0.00 |
| ATOM | 1473 | C | THR | 186 | 63.129 | 6.250 | −25.598 | 0.00 | 0.00 |
| ATOM | 1474 | O | THR | 186 | 63.852 | 6.830 | −24.779 | 0.00 | 0.00 |
| ATOM | 1475 | CB | THR | 186 | 64.930 | 6.695 | −27.510 | 0.00 | 0.00 |
| ATOM | 1476 | OG1 | THR | 186 | 65.970 | 6.484 | −26.535 | 0.00 | 0.00 |
| ATOM | 1477 | CG2 | THR | 186 | 65.151 | 7.860 | −28.424 | 0.00 | 0.00 |
| ATOM | 1478 | N | ASN | 187 | 61.915 | 5.863 | −25.257 | 0.00 | 0.00 |
| ATOM | 1479 | CA | ASN | 187 | 61.395 | 5.692 | −23.887 | 0.00 | 0.00 |
| ATOM | 1480 | C | ASN | 187 | 62.252 | 4.623 | −23.104 | 0.00 | 0.00 |
| ATOM | 1481 | O | ASN | 187 | 62.358 | 4.788 | −21.892 | 0.00 | 0.00 |
| ATOM | 1482 | CB | ASN | 187 | 59.949 | 5.375 | −24.110 | 0.00 | 0.00 |
| ATOM | 1483 | CG | ASN | 187 | 59.050 | 6.548 | −24.023 | 0.00 | 0.00 |
| ATOM | 1484 | OD1 | ASN | 187 | 58.435 | 6.871 | −25.018 | 0.00 | 0.00 |
| ATOM | 1485 | ND2 | ASN | 187 | 59.027 | 7.259 | −22.908 | 0.00 | 0.00 |
| ATOM | 1486 | N | GLN | 188 | 62.464 | 3.380 | −23.641 | 0.00 | 0.00 |
| ATOM | 1487 | CA | GLN | 188 | 63.324 | 2.342 | −23.058 | 0.00 | 0.00 |
| ATOM | 1488 | C | GLN | 188 | 64.761 | 2.883 | −22.757 | 0.00 | 0.00 |
| ATOM | 1489 | O | GLN | 188 | 65.273 | 2.536 | −21.698 | 0.00 | 0.00 |
| ATOM | 1490 | CB | GLN | 188 | 63.319 | 1.130 | −24.006 | 0.00 | 0.00 |
| ATOM | 1491 | CG | GLN | 188 | 64.136 | −0.035 | −23.399 | 0.00 | 0.00 |
| ATOM | 1492 | CD | GLN | 188 | 64.113 | −1.202 | −24.371 | 0.00 | 0.00 |
| ATOM | 1493 | OE1 | GLN | 188 | 64.145 | −0.998 | −25.583 | 0.00 | 0.00 |
| ATOM | 1494 | NE2 | GLN | 188 | 64.034 | −2.400 | −23.811 | 0.00 | 0.00 |
| ATOM | 1495 | N | ALA | 189 | 65.487 | 3.471 | −23.744 | 0.00 | 0.00 |
| ATOM | 1496 | CA | ALA | 189 | 66.797 | 4.079 | −23.483 | 0.00 | 0.00 |
| ATOM | 1497 | C | ALA | 189 | 66.644 | 5.078 | −22.301 | 0.00 | 0.00 |
| ATOM | 1498 | O | ALA | 189 | 67.593 | 5.130 | −21.527 | 0.00 | 0.00 |
| ATOM | 1499 | CB | ALA | 189 | 67.475 | 4.650 | −24.719 | 0.00 | 0.00 |
| ATOM | 1500 | N | TYR | 190 | 65.634 | 5.986 | −22.264 | 0.00 | 0.00 |
| ATOM | 1501 | CA | TYR | 190 | 65.543 | 6.800 | −21.038 | 0.00 | 0.00 |
| ATOM | 1502 | C | TYR | 190 | 65.364 | 5.930 | −19.762 | 0.00 | 0.00 |
| ATOM | 1503 | O | TYR | 190 | 65.923 | 6.341 | −18.766 | 0.00 | 0.00 |
| ATOM | 1504 | CB | TYR | 190 | 64.426 | 7.798 | −21.030 | 0.00 | 0.00 |
| ATOM | 1505 | CG | TYR | 190 | 64.314 | 8.646 | −19.817 | 0.00 | 0.00 |
| ATOM | 1506 | CD1 | TYR | 190 | 63.557 | 8.064 | −18.795 | 0.00 | 0.00 |
| ATOM | 1507 | CD2 | TYR | 190 | 64.992 | 9.866 | −19.627 | 0.00 | 0.00 |
| ATOM | 1508 | CE1 | TYR | 190 | 63.418 | 8.717 | −17.572 | 0.00 | 0.00 |
| ATOM | 1509 | CE2 | TYR | 190 | 64.872 | 10.496 | −18.397 | 0.00 | 0.00 |
| ATOM | 1510 | CZ | TYR | 190 | 64.073 | 9.938 | −17.403 | 0.00 | 0.00 |
| ATOM | 1511 | OH | TYR | 190 | 63.892 | 10.585 | −16.167 | 0.00 | 0.00 |
| ATOM | 1512 | N | ALA | 191 | 64.291 | 5.111 | −19.669 | 0.00 | 0.00 |
| ATOM | 1513 | CA | ALA | 191 | 63.971 | 4.236 | −18.553 | 0.00 | 0.00 |
| ATOM | 1514 | C | ALA | 191 | 65.233 | 3.535 | −17.982 | 0.00 | 0.00 |
| ATOM | 1515 | O | ALA | 191 | 65.266 | 3.395 | −16.761 | 0.00 | 0.00 |
| ATOM | 1516 | CB | ALA | 191 | 62.889 | 3.247 | −19.015 | 0.00 | 0.00 |
| ATOM | 1517 | N | ILE | 192 | 66.094 | 2.889 | −18.783 | 0.00 | 0.00 |
| ATOM | 1518 | CA | ILE | 192 | 67.349 | 2.290 | −18.353 | 0.00 | 0.00 |
| ATOM | 1519 | C | ILE | 192 | 68.275 | 3.404 | −17.802 | 0.00 | 0.00 |
| ATOM | 1520 | O | ILE | 192 | 68.830 | 3.175 | −16.713 | 0.00 | 0.00 |
| ATOM | 1521 | CB | ILE | 192 | 67.941 | 1.506 | −19.537 | 0.00 | 0.00 |
| ATOM | 1522 | CG1 | ILE | 192 | 67.026 | 0.387 | −20.006 | 0.00 | 0.00 |
| ATOM | 1523 | CG2 | ILE | 192 | 69.320 | 0.927 | −19.109 | 0.00 | 0.00 |
| ATOM | 1524 | CD1 | ILE | 192 | 66.973 | −0.924 | −19.203 | 0.00 | 0.00 |
| ATOM | 1525 | N | ALA | 193 | 68.621 | 4.442 | −18.589 | 0.00 | 0.00 |
| ATOM | 1526 | CA | ALA | 193 | 69.397 | 5.573 | −18.105 | 0.00 | 0.00 |
| ATOM | 1527 | C | ALA | 193 | 68.837 | 6.064 | −16.710 | 0.00 | 0.00 |
| ATOM | 1528 | O | ALA | 193 | 69.674 | 6.273 | −15.827 | 0.00 | 0.00 |
| ATOM | 1529 | CB | ALA | 193 | 69.401 | 6.670 | −19.178 | 0.00 | 0.00 |
| ATOM | 1530 | N | SER | 194 | 67.523 | 6.339 | −16.532 | 0.00 | 0.00 |
| ATOM | 1531 | CA | SER | 194 | 66.933 | 6.820 | −15.270 | 0.00 | 0.00 |
| ATOM | 1532 | C | SER | 194 | 67.095 | 5.801 | −14.097 | 0.00 | 0.00 |
| ATOM | 1533 | O | SER | 194 | 67.325 | 6.287 | −12.982 | 0.00 | 0.00 |
| ATOM | 1534 | CB | SER | 194 | 65.437 | 7.169 | −15.518 | 0.00 | 0.00 |
| ATOM | 1535 | OG | SER | 194 | 64.562 | 6.019 | −15.438 | 0.00 | 0.00 |
| ATOM | 1536 | N | SER | 195 | 66.618 | 4.542 | −14.211 | 0.00 | 0.00 |
| ATOM | 1537 | CA | SER | 195 | 66.850 | 3.552 | −13.175 | 0.00 | 0.00 |
| ATOM | 1538 | C | SER | 195 | 68.365 | 3.454 | −12.836 | 0.00 | 0.00 |
| ATOM | 1539 | O | SER | 195 | 68.726 | 4.016 | −11.820 | 0.00 | 0.00 |
| ATOM | 1540 | CB | SER | 195 | 66.251 | 2.189 | −13.572 | 0.00 | 0.00 |
| ATOM | 1541 | OG | SER | 195 | 64.837 | 2.058 | −13.615 | 0.00 | 0.00 |
| ATOM | 1542 | N | ILE | 196 | 69.251 | 3.077 | −13.806 | 0.00 | 0.00 |
| ATOM | 1543 | CA | ILE | 196 | 70.663 | 2.938 | −13.423 | 0.00 | 0.00 |
| ATOM | 1544 | C | ILE | 196 | 71.288 | 4.228 | −12.826 | 0.00 | 0.00 |
| ATOM | 1545 | O | ILE | 196 | 71.397 | 4.245 | −11.627 | 0.00 | 0.00 |
| ATOM | 1546 | CB | ILE | 196 | 71.490 | 2.383 | −14.598 | 0.00 | 0.00 |
| ATOM | 1547 | CG1 | ILE | 196 | 71.042 | 1.022 | −15.008 | 0.00 | 0.00 |
| ATOM | 1548 | CG2 | ILE | 196 | 72.966 | 2.291 | −14.248 | 0.00 | 0.00 |
| ATOM | 1549 | CD1 | ILE | 196 | 71.199 | 0.687 | −16.493 | 0.00 | 0.00 |
| ATOM | 1550 | N | VAL | 197 | 71.349 | 5.358 | −13.574 | 0.00 | 0.00 |
| ATOM | 1551 | CA | VAL | 197 | 71.986 | 6.535 | −12.982 | 0.00 | 0.00 |
| ATOM | 1552 | C | VAL | 197 | 71.254 | 7.108 | −11.698 | 0.00 | 0.00 |
| ATOM | 1553 | O | VAL | 197 | 71.990 | 7.589 | −10.831 | 0.00 | 0.00 |
| ATOM | 1554 | CB | VAL | 197 | 72.100 | 7.610 | −14.077 | 0.00 | 0.00 |
| ATOM | 1555 | CG1 | VAL | 197 | 72.870 | 8.834 | −13.555 | 0.00 | 0.00 |
| ATOM | 1556 | CG2 | VAL | 197 | 72.739 | 7.091 | −15.352 | 0.00 | 0.00 |
| ATOM | 1557 | N | SER | 198 | 69.952 | 7.510 | −11.746 | 0.00 | 0.00 |
| ATOM | 1558 | CA | SER | 198 | 69.241 | 8.064 | −10.608 | 0.00 | 0.00 |
| ATOM | 1559 | C | SER | 198 | 68.981 | 7.012 | −9.470 | 0.00 | 0.00 |
| ATOM | 1560 | O | SER | 198 | 69.046 | 7.442 | −8.321 | 0.00 | 0.00 |
| ATOM | 1561 | CB | SER | 198 | 67.951 | 8.737 | −11.117 | 0.00 | 0.00 |
| ATOM | 1562 | OG | SER | 198 | 67.105 | 9.256 | −10.086 | 0.00 | 0.00 |
| ATOM | 1563 | N | PHE | 199 | 68.175 | 5.976 | −9.759 | 0.00 | 0.00 |
| ATOM | 1564 | CA | PHE | 199 | 67.726 | 4.953 | −8.827 | 0.00 | 0.00 |
| ATOM | 1565 | C | PHE | 199 | 68.837 | 4.031 | −8.287 | 0.00 | 0.00 |
| ATOM | 1566 | O | PHE | 199 | 69.305 | 4.341 | −7.198 | 0.00 | 0.00 |
| ATOM | 1567 | CB | PHE | 199 | 66.576 | 4.224 | −9.526 | 0.00 | 0.00 |
| ATOM | 1568 | CG | PHE | 199 | 65.813 | 3.155 | −8.857 | 0.00 | 0.00 |
| ATOM | 1569 | CD1 | PHE | 199 | 65.366 | 3.283 | −7.537 | 0.00 | 0.00 |
| ATOM | 1570 | CD2 | PHE | 199 | 65.478 | 1.977 | −9.545 | 0.00 | 0.00 |
| ATOM | 1571 | CE1 | PHE | 199 | 64.590 | 2.292 | −6.929 | 0.00 | 0.00 |
| ATOM | 1572 | CE2 | PHE | 199 | 64.734 | 0.993 | −8.956 | 0.00 | 0.00 |
| ATOM | 1573 | CZ | PHE | 199 | 64.291 | 1.129 | −7.635 | 0.00 | 0.00 |
| ATOM | 1574 | N | TYR | 200 | 69.474 | 3.203 | −9.142 | 0.00 | 0.00 |
| ATOM | 1575 | CA | TYR | 200 | 70.564 | 2.308 | −8.735 | 0.00 | 0.00 |
| ATOM | 1576 | C | TYR | 200 | 71.787 | 3.103 | −8.234 | 0.00 | 0.00 |
| ATOM | 1577 | O | TYR | 200 | 72.272 | 2.749 | −7.158 | 0.00 | 0.00 |
| ATOM | 1578 | CB | TYR | 200 | 70.997 | 1.337 | −9.842 | 0.00 | 0.00 |
| ATOM | 1579 | CG | TYR | 200 | 69.870 | 0.408 | −10.215 | 0.00 | 0.00 |
| ATOM | 1580 | CD1 | TYR | 200 | 68.774 | 0.835 | −10.976 | 0.00 | 0.00 |
| ATOM | 1581 | CD2 | TYR | 200 | 69.886 | −0.909 | −9.751 | 0.00 | 0.00 |
| ATOM | 1582 | CE1 | TYR | 200 | 67.733 | −0.060 | −11.296 | 0.00 | 0.00 |
| ATOM | 1583 | CE2 | TYR | 200 | 68.876 | −1.792 | −10.081 | 0.00 | 0.00 |
| ATOM | 1584 | CZ | TYR | 200 | 67.788 | −1.361 | −10.822 | 0.00 | 0.00 |
| ATOM | 1585 | OH | TYR | 200 | 66.837 | −2.309 | −11.092 | 0.00 | 0.00 |
| ATOM | 1586 | N | VAL | 201 | 72.168 | 4.255 | −8.837 | 0.00 | 0.00 |
| ATOM | 1587 | CA | VAL | 201 | 73.396 | 4.995 | −8.449 | 0.00 | 0.00 |
| ATOM | 1588 | C | VAL | 201 | 73.404 | 5.665 | −7.086 | 0.00 | 0.00 |
| ATOM | 1589 | O | VAL | 201 | 74.041 | 5.039 | −6.227 | 0.00 | 0.00 |
| ATOM | 1590 | CB | VAL | 201 | 73.935 | 5.897 | −9.518 | 0.00 | 0.00 |
| ATOM | 1591 | CG1 | VAL | 201 | 75.323 | 6.414 | −9.212 | 0.00 | 0.00 |
| ATOM | 1592 | CG2 | VAL | 201 | 73.890 | 5.318 | −10.913 | 0.00 | 0.00 |
| ATOM | 1593 | N | PRO | 202 | 72.822 | 6.862 | −6.773 | 0.00 | 0.00 |
| ATOM | 1594 | CA | PRO | 202 | 72.894 | 7.226 | −5.411 | 0.00 | 0.00 |
| ATOM | 1595 | C | PRO | 202 | 72.394 | 6.118 | −4.403 | 0.00 | 0.00 |
| ATOM | 1596 | O | PRO | 202 | 72.895 | 6.154 | −3.286 | 0.00 | 0.00 |
| ATOM | 1597 | CB | PRO | 202 | 72.194 | 8.565 | −5.125 | 0.00 | 0.00 |
| ATOM | 1598 | CG | PRO | 202 | 71.388 | 8.800 | −6.412 | 0.00 | 0.00 |
| ATOM | 1599 | CD | PRO | 202 | 71.990 | 7.913 | −7.499 | 0.00 | 0.00 |
| ATOM | 1600 | N | LEU | 203 | 71.350 | 5.315 | −4.702 | 0.00 | 0.00 |
| ATOM | 1601 | CA | LEU | 203 | 70.983 | 4.268 | −3.724 | 0.00 | 0.00 |
| ATOM | 1602 | C | LEU | 203 | 72.237 | 3.507 | −3.178 | 0.00 | 0.00 |
| ATOM | 1603 | O | LEU | 203 | 72.267 | 3.308 | −1.979 | 0.00 | 0.00 |
| ATOM | 1604 | CB | LEU | 203 | 69.921 | 3.300 | −4.251 | 0.00 | 0.00 |
| ATOM | 1605 | CG | LEU | 203 | 68.509 | 3.843 | −4.341 | 0.00 | 0.00 |
| ATOM | 1606 | CD1 | LEU | 203 | 67.589 | 2.759 | −4.922 | 0.00 | 0.00 |
| ATOM | 1607 | CD2 | LEU | 203 | 68.012 | 4.320 | −2.977 | 0.00 | 0.00 |
| ATOM | 1608 | N | VAL | 204 | 73.033 | 2.873 | −4.038 | 0.00 | 0.00 |
| ATOM | 1609 | CA | VAL | 204 | 74.277 | 2.167 | −3.716 | 0.00 | 0.00 |
| ATOM | 1610 | C | VAL | 204 | 75.342 | 3.152 | −3.125 | 0.00 | 0.00 |
| ATOM | 1611 | O | VAL | 204 | 76.155 | 2.654 | −2.355 | 0.00 | 0.00 |
| ATOM | 1612 | CB | VAL | 204 | 74.623 | 1.416 | −4.946 | 0.00 | 0.00 |
| ATOM | 1613 | CG1 | VAL | 204 | 75.915 | 1.545 | −5.539 | 0.00 | 0.00 |
| ATOM | 1614 | CG2 | VAL | 204 | 73.895 | 0.127 | −5.163 | 0.00 | 0.00 |
| ATOM | 1615 | N | ILE | 205 | 75.546 | 4.379 | −3.659 | 0.00 | 0.00 |
| ATOM | 1616 | CA | ILE | 205 | 76.480 | 5.352 | −3.028 | 0.00 | 0.00 |
| ATOM | 1617 | C | ILE | 205 | 76.015 | 5.548 | −1.535 | 0.00 | 0.00 |
| ATOM | 1618 | O | ILE | 205 | 76.881 | 5.466 | −0.669 | 0.00 | 0.00 |
| ATOM | 1619 | CB | ILE | 205 | 76.524 | 6.686 | −3.862 | 0.00 | 0.00 |
| ATOM | 1620 | CG1 | ILE | 205 | 77.243 | 6.488 | −5.232 | 0.00 | 0.00 |
| ATOM | 1621 | CG2 | ILE | 205 | 77.120 | 7.895 | −3.067 | 0.00 | 0.00 |
| ATOM | 1622 | CD1 | ILE | 205 | 77.130 | 7.859 | −6.035 | 0.00 | 0.00 |
| ATOM | 1623 | N | MET | 206 | 74.706 | 5.797 | −1.220 | 0.00 | 0.00 |
| ATOM | 1624 | CA | MET | 206 | 74.139 | 5.898 | 0.118 | 0.00 | 0.00 |
| ATOM | 1625 | C | MET | 206 | 74.392 | 4.610 | 0.971 | 0.00 | 0.00 |
| ATOM | 1626 | O | MET | 206 | 74.759 | 4.786 | 2.142 | 0.00 | 0.00 |
| ATOM | 1627 | CB | MET | 206 | 72.626 | 6.115 | −0.067 | 0.00 | 0.00 |
| ATOM | 1628 | CG | MET | 206 | 72.303 | 7.434 | −0.695 | 0.00 | 0.00 |
| ATOM | 1629 | SD | MET | 206 | 70.583 | 7.581 | −1.269 | 0.00 | 0.00 |
| ATOM | 1630 | CE | MET | 206 | 69.725 | 7.523 | 0.310 | 0.00 | 0.00 |
| ATOM | 1631 | N | VAL | 207 | 73.937 | 3.402 | 0.535 | 0.00 | 0.00 |
| ATOM | 1632 | CA | VAL | 207 | 74.154 | 2.102 | 1.196 | 0.00 | 0.00 |
| ATOM | 1633 | C | VAL | 207 | 75.676 | 1.848 | 1.486 | 0.00 | 0.00 |
| ATOM | 1634 | O | VAL | 207 | 75.919 | 1.207 | 2.504 | 0.00 | 0.00 |
| ATOM | 1635 | CB | VAL | 207 | 73.489 | 1.019 | 0.345 | 0.00 | 0.00 |
| ATOM | 1636 | CG1 | VAL | 207 | 73.640 | −0.377 | 0.865 | 0.00 | 0.00 |
| ATOM | 1637 | CG2 | VAL | 207 | 72.003 | 1.234 | 0.262 | 0.00 | 0.00 |
| ATOM | 1638 | N | PHE | 208 | 76.617 | 2.067 | 0.547 | 0.00 | 0.00 |
| ATOM | 1639 | CA | PHE | 208 | 78.047 | 1.795 | 0.661 | 0.00 | 0.00 |
| ATOM | 1640 | C | PHE | 208 | 78.642 | 2.727 | 1.803 | 0.00 | 0.00 |
| ATOM | 1641 | O | PHE | 208 | 79.508 | 2.200 | 2.525 | 0.00 | 0.00 |
| ATOM | 1642 | CB | PHE | 208 | 78.644 | 1.998 | −0.753 | 0.00 | 0.00 |
| ATOM | 1643 | CG | PHE | 208 | 80.103 | 1.526 | −0.967 | 0.00 | 0.00 |
| ATOM | 1644 | CD1 | PHE | 208 | 80.473 | 0.279 | −0.430 | 0.00 | 0.00 |
| ATOM | 1645 | CD2 | PHE | 208 | 80.968 | 2.246 | −1.729 | 0.00 | 0.00 |
| ATOM | 1646 | CE1 | PHE | 208 | 81.792 | −0.169 | −0.604 | 0.00 | 0.00 |
| ATOM | 1647 | CE2 | PHE | 208 | 82.287 | 1.798 | −1.904 | 0.00 | 0.00 |
| ATOM | 1648 | CZ | PHE | 208 | 82.740 | 0.631 | −1.364 | 0.00 | 0.00 |
| ATOM | 1649 | N | VAL | 209 | 78.523 | 4.078 | 1.753 | 0.00 | 0.00 |
| ATOM | 1650 | CA | VAL | 209 | 78.948 | 4.989 | 2.806 | 0.00 | 0.00 |
| ATOM | 1651 | C | VAL | 209 | 78.434 | 4.450 | 4.186 | 0.00 | 0.00 |
| ATOM | 1652 | O | VAL | 209 | 79.261 | 4.410 | 5.095 | 0.00 | 0.00 |
| ATOM | 1653 | CB | VAL | 209 | 78.462 | 6.451 | 2.533 | 0.00 | 0.00 |
| ATOM | 1654 | CG1 | VAL | 209 | 78.838 | 7.390 | 3.658 | 0.00 | 0.00 |
| ATOM | 1655 | CG2 | VAL | 209 | 78.953 | 6.972 | 1.175 | 0.00 | 0.00 |
| ATOM | 1656 | N | TYR | 210 | 77.102 | 4.258 | 4.384 | 0.00 | 0.00 |
| ATOM | 1657 | CA | TYR | 210 | 76.500 | 3.667 | 5.588 | 0.00 | 0.00 |
| ATOM | 1658 | C | TYR | 210 | 77.178 | 2.303 | 5.953 | 0.00 | 0.00 |
| ATOM | 1659 | O | TYR | 210 | 77.310 | 2.083 | 7.156 | 0.00 | 0.00 |
| ATOM | 1660 | CB | TYR | 210 | 74.966 | 3.491 | 5.424 | 0.00 | 0.00 |
| ATOM | 1661 | CG | TYR | 210 | 74.411 | 2.584 | 6.544 | 0.00 | 0.00 |
| ATOM | 1662 | CD1 | TYR | 210 | 74.170 | 3.127 | 7.813 | 0.00 | 0.00 |
| ATOM | 1663 | CD2 | TYR | 210 | 74.161 | 1.227 | 6.301 | 0.00 | 0.00 |
| ATOM | 1664 | CE1 | TYR | 210 | 73.651 | 2.333 | 8.842 | 0.00 | 0.00 |
| ATOM | 1665 | CE2 | TYR | 210 | 73.683 | 0.414 | 7.332 | 0.00 | 0.00 |
| ATOM | 1666 | CZ | TYR | 210 | 73.432 | 0.971 | 8.591 | 0.00 | 0.00 |
| ATOM | 1667 | OH | TYR | 210 | 72.969 | 0.164 | 9.591 | 0.00 | 0.00 |
| ATOM | 1668 | N | SER | 211 | 77.429 | 1.363 | 5.013 | 0.00 | 0.00 |
| ATOM | 1669 | CA | SER | 211 | 78.160 | 0.127 | 5.335 | 0.00 | 0.00 |
| ATOM | 1670 | C | SER | 211 | 79.514 | 0.467 | 6.068 | 0.00 | 0.00 |
| ATOM | 1671 | O | SER | 211 | 79.869 | −0.299 | 6.964 | 0.00 | 0.00 |
| ATOM | 1672 | CB | SER | 211 | 78.381 | −0.661 | 4.034 | 0.00 | 0.00 |
| ATOM | 1673 | OG | SER | 211 | 77.277 | −1.409 | 3.530 | 0.00 | 0.00 |
| ATOM | 1674 | N | ARG | 212 | 80.358 | 1.425 | 5.572 | 0.00 | 0.00 |
| ATOM | 1675 | CA | ARG | 212 | 81.583 | 1.873 | 6.236 | 0.00 | 0.00 |
| ATOM | 1676 | C | ARG | 212 | 81.300 | 2.628 | 7.582 | 0.00 | 0.00 |
| ATOM | 1677 | O | ARG | 212 | 82.124 | 2.472 | 8.489 | 0.00 | 0.00 |
| ATOM | 1678 | CB | ARG | 212 | 82.602 | 2.513 | 5.317 | 0.00 | 0.00 |
| ATOM | 1679 | CG | ARG | 212 | 83.051 | 1.759 | 4.118 | 0.00 | 0.00 |
| ATOM | 1680 | CD | ARG | 212 | 84.006 | 2.667 | 3.259 | 0.00 | 0.00 |
| ATOM | 1681 | NE | ARG | 212 | 83.337 | 3.826 | 2.683 | 0.00 | 0.00 |
| ATOM | 1682 | CZ | ARG | 212 | 82.532 | 3.749 | 1.619 | 0.00 | 0.00 |
| ATOM | 1683 | NH1 | ARG | 212 | 82.313 | 2.600 | 0.972 | 0.00 | 0.00 |
| ATOM | 1684 | NH2 | ARG | 212 | 81.786 | 4.813 | 1.299 | 0.00 | 0.00 |
| ATOM | 1685 | N | VAL | 213 | 80.337 | 3.586 | 7.670 | 0.00 | 0.00 |
| ATOM | 1686 | CA | VAL | 213 | 79.950 | 4.258 | 8.926 | 0.00 | 0.00 |
| ATOM | 1687 | C | VAL | 213 | 79.649 | 3.222 | 10.076 | 0.00 | 0.00 |
| ATOM | 1688 | O | VAL | 213 | 79.668 | 3.654 | 11.232 | 0.00 | 0.00 |
| ATOM | 1689 | CB | VAL | 213 | 78.688 | 5.111 | 8.631 | 0.00 | 0.00 |
| ATOM | 1690 | CG1 | VAL | 213 | 77.985 | 5.787 | 9.777 | 0.00 | 0.00 |
| ATOM | 1691 | CG2 | VAL | 213 | 79.006 | 6.128 | 7.519 | 0.00 | 0.00 |
| ATOM | 1692 | N | PHE | 214 | 79.191 | 1.980 | 9.796 | 0.00 | 0.00 |
| ATOM | 1693 | CA | PHE | 214 | 78.971 | 0.905 | 10.743 | 0.00 | 0.00 |
| ATOM | 1694 | C | PHE | 214 | 80.115 | 0.842 | 11.802 | 0.00 | 0.00 |
| ATOM | 1695 | O | PHE | 214 | 79.779 | 0.484 | 12.931 | 0.00 | 0.00 |
| ATOM | 1696 | CB | PHE | 214 | 78.910 | −0.426 | 9.974 | 0.00 | 0.00 |
| ATOM | 1697 | CG | PHE | 214 | 78.678 | −1.631 | 10.873 | 0.00 | 0.00 |
| ATOM | 1698 | CD1 | PHE | 214 | 77.428 | −1.794 | 11.490 | 0.00 | 0.00 |
| ATOM | 1699 | CD2 | PHE | 214 | 79.690 | −2.558 | 11.093 | 0.00 | 0.00 |
| ATOM | 1700 | CE1 | PHE | 214 | 77.189 | −2.897 | 12.315 | 0.00 | 0.00 |
| ATOM | 1701 | CE2 | PHE | 214 | 79.454 | −3.682 | 11.899 | 0.00 | 0.00 |
| ATOM | 1702 | CZ | PHE | 214 | 78.199 | −3.841 | 12.502 | 0.00 | 0.00 |
| ATOM | 1703 | N | GLN | 215 | 81.425 | 1.030 | 11.451 | 0.00 | 0.00 |
| ATOM | 1704 | CA | GLN | 215 | 82.468 | 1.067 | 12.491 | 0.00 | 0.00 |
| ATOM | 1705 | C | GLN | 215 | 82.074 | 2.093 | 13.589 | 0.00 | 0.00 |
| ATOM | 1706 | O | GLN | 215 | 82.067 | 1.679 | 14.761 | 0.00 | 0.00 |
| ATOM | 1707 | CB | GLN | 215 | 83.900 | 1.452 | 11.968 | 0.00 | 0.00 |
| ATOM | 1708 | CG | GLN | 215 | 84.911 | 1.980 | 13.051 | 0.00 | 0.00 |
| ATOM | 1709 | CD | GLN | 215 | 85.353 | 0.838 | 13.980 | 0.00 | 0.00 |
| ATOM | 1710 | OE1 | GLN | 215 | 86.020 | −0.152 | 13.358 | 0.00 | 0.00 |
| ATOM | 1711 | NE2 | GLN | 215 | 85.070 | 0.910 | 15.279 | 0.00 | 0.00 |
| ATOM | 1712 | N | GLU | 216 | 81.969 | 3.362 | 13.300 | 0.00 | 0.00 |
| ATOM | 1713 | CA | GLU | 216 | 81.655 | 4.429 | 14.283 | 0.00 | 0.00 |
| ATOM | 1714 | C | GLU | 216 | 80.268 | 4.227 | 14.995 | 0.00 | 0.00 |
| ATOM | 1715 | O | GLU | 216 | 80.300 | 3.933 | 16.191 | 0.00 | 0.00 |
| ATOM | 1716 | CB | GLU | 216 | 81.855 | 5.817 | 13.692 | 0.00 | 0.00 |
| ATOM | 1717 | CG | GLU | 216 | 81.404 | 7.059 | 14.462 | 0.00 | 0.00 |
| ATOM | 1718 | CD | GLU | 216 | 82.539 | 7.480 | 15.416 | 0.00 | 0.00 |
| ATOM | 1719 | OE1 | GLU | 216 | 83.129 | 6.648 | 16.104 | 0.00 | 0.00 |
| ATOM | 1720 | OE2 | GLU | 216 | 82.767 | 8.704 | 15.326 | 0.00 | 0.00 |
| ATOM | 1721 | N | ALA | 217 | 79.095 | 4.319 | 14.295 | 0.00 | 0.00 |
| ATOM | 1722 | CA | ALA | 217 | 77.771 | 4.051 | 14.891 | 0.00 | 0.00 |
| ATOM | 1723 | C | ALA | 217 | 77.010 | 2.946 | 14.061 | 0.00 | 0.00 |
| ATOM | 1724 | O | ALA | 217 | 76.568 | 3.180 | 12.939 | 0.00 | 0.00 |
| ATOM | 1725 | CB | ALA | 217 | 76.979 | 5.371 | 14.958 | 0.00 | 0.00 |
| ATOM | 1726 | N | LYS | 218 | 77.117 | 1.712 | 14.561 | 0.00 | 0.00 |
| ATOM | 1727 | CA | LYS | 218 | 76.512 | 0.568 | 13.887 | 0.00 | 0.00 |
| ATOM | 1728 | C | LYS | 218 | 74.946 | 0.719 | 13.717 | 0.00 | 0.00 |
| ATOM | 1729 | O | LYS | 218 | 74.543 | 0.981 | 12.573 | 0.00 | 0.00 |
| ATOM | 1730 | CB | LYS | 218 | 76.798 | −0.721 | 14.702 | 0.00 | 0.00 |
| ATOM | 1731 | CG | LYS | 218 | 78.235 | −0.952 | 15.075 | 0.00 | 0.00 |
| ATOM | 1732 | CD | LYS | 218 | 78.323 | −1.936 | 16.240 | 0.00 | 0.00 |
| ATOM | 1733 | CE | LYS | 218 | 79.713 | −1.947 | 16.864 | 0.00 | 0.00 |
| ATOM | 1734 | NZ | LYS | 218 | 79.773 | −2.788 | 18.090 | 0.00 | 0.00 |
| ATOM | 1735 | N | ARG | 219 | 74.170 | 0.717 | 14.812 | 0.00 | 0.00 |
| ATOM | 1736 | CA | ARG | 219 | 72.728 | 0.926 | 14.717 | 0.00 | 0.00 |
| ATOM | 1737 | C | ARG | 219 | 72.107 | 1.677 | 15.892 | 0.00 | 0.00 |
| ATOM | 1738 | O | ARG | 219 | 70.871 | 1.796 | 15.860 | 0.00 | 0.00 |
| ATOM | 1739 | CB | ARG | 219 | 72.118 | −0.485 | 14.624 | 0.00 | 0.00 |
| ATOM | 1740 | CG | ARG | 219 | 72.018 | −1.272 | 15.936 | 0.00 | 0.00 |
| ATOM | 1741 | CD | ARG | 219 | 71.848 | −2.724 | 15.524 | 0.00 | 0.00 |
| ATOM | 1742 | NE | ARG | 219 | 70.681 | −3.343 | 16.145 | 0.00 | 0.00 |
| ATOM | 1743 | CZ | ARG | 219 | 70.759 | −4.639 | 16.592 | 0.00 | 0.00 |
| ATOM | 1744 | NH1 | ARG | 219 | 71.928 | −5.378 | 16.432 | 0.00 | 0.00 |
| ATOM | 1745 | NH2 | ARG | 219 | 69.657 | −5.179 | 17.190 | 0.00 | 0.00 |
| ATOM | 1746 | N | GLN | 220 | 72.873 | 2.426 | 16.721 | 0.00 | 0.00 |
| ATOM | 1747 | CA | GLN | 220 | 72.243 | 3.014 | 17.864 | 0.00 | 0.00 |
| ATOM | 1748 | C | GLN | 220 | 72.785 | 4.370 | 18.259 | 0.00 | 0.00 |
| ATOM | 1749 | O | GLN | 220 | 74.001 | 4.612 | 18.253 | 0.00 | 0.00 |
| ATOM | 1750 | CB | GLN | 220 | 72.464 | 2.087 | 19.065 | 0.00 | 0.00 |
| ATOM | 1751 | CG | GLN | 220 | 71.968 | 0.673 | 18.971 | 0.00 | 0.00 |
| ATOM | 1752 | CD | GLN | 220 | 71.879 | −0.216 | 20.219 | 0.00 | 0.00 |
| ATOM | 1753 | OE1 | GLN | 220 | 71.791 | 0.357 | 21.311 | 0.00 | 0.00 |
| ATOM | 1754 | NE2 | GLN | 220 | 71.834 | −1.566 | 20.089 | 0.00 | 0.00 |
| ATOM | 1755 | N | LEU | 221 | 71.847 | 5.278 | 18.273 | 0.00 | 0.00 |
| ATOM | 1756 | CA | LEU | 221 | 71.978 | 6.632 | 18.682 | 0.00 | 0.00 |
| ATOM | 1757 | C | LEU | 221 | 71.074 | 6.698 | 19.974 | 0.00 | 0.00 |
| ATOM | 1758 | O | LEU | 221 | 69.901 | 7.073 | 19.829 | 0.00 | 0.00 |
| ATOM | 1759 | CB | LEU | 221 | 71.369 | 7.321 | 17.486 | 0.00 | 0.00 |
| ATOM | 1760 | CG | LEU | 221 | 72.215 | 7.670 | 16.271 | 0.00 | 0.00 |
| ATOM | 1761 | CD1 | LEU | 221 | 73.702 | 7.858 | 16.486 | 0.00 | 0.00 |
| ATOM | 1762 | CD2 | LEU | 221 | 71.820 | 6.499 | 15.385 | 0.00 | 0.00 |
| ATOM | 1763 | N | GLN | 222 | 71.590 | 6.541 | 21.195 | 0.00 | 0.00 |
| ATOM | 1764 | CA | GLN | 222 | 70.667 | 6.502 | 22.376 | 0.00 | 0.00 |
| ATOM | 1765 | C | GLN | 222 | 70.481 | 7.859 | 23.094 | 0.00 | 0.00 |
| ATOM | 1766 | O | GLN | 222 | 69.370 | 8.050 | 23.609 | 0.00 | 0.00 |
| ATOM | 1767 | CB | GLN | 222 | 71.203 | 5.416 | 23.333 | 0.00 | 0.00 |
| ATOM | 1768 | CG | GLN | 222 | 71.127 | 4.002 | 22.762 | 0.00 | 0.00 |
| ATOM | 1769 | CD | GLN | 222 | 69.739 | 3.338 | 22.638 | 0.00 | 0.00 |
| ATOM | 1770 | OE1 | GLN | 222 | 69.660 | 2.166 | 22.199 | 0.00 | 0.00 |
| ATOM | 1771 | NE2 | GLN | 222 | 68.665 | 3.984 | 22.990 | 0.00 | 0.00 |
| ATOM | 1772 | N | LYS | 223 | 71.512 | 8.708 | 23.252 | 0.00 | 0.00 |
| ATOM | 1773 | CA | LYS | 223 | 71.287 | 9.956 | 23.961 | 0.00 | 0.00 |
| ATOM | 1774 | C | LYS | 223 | 70.445 | 10.868 | 23.043 | 0.00 | 0.00 |
| ATOM | 1775 | O | LYS | 223 | 71.019 | 11.634 | 22.284 | 0.00 | 0.00 |
| ATOM | 1776 | CB | LYS | 223 | 72.621 | 10.547 | 24.424 | 0.00 | 0.00 |
| ATOM | 1777 | CG | LYS | 223 | 72.424 | 11.808 | 25.243 | 0.00 | 0.00 |
| ATOM | 1778 | CD | LYS | 223 | 73.743 | 12.229 | 25.842 | 0.00 | 0.00 |
| ATOM | 1779 | CE | LYS | 223 | 73.517 | 12.587 | 27.304 | 0.00 | 0.00 |
| ATOM | 1780 | NZ | LYS | 223 | 73.190 | 11.412 | 28.104 | 0.00 | 0.00 |
| ATOM | 1781 | N | ILE | 224 | 69.244 | 11.178 | 23.517 | 0.00 | 0.00 |
| ATOM | 1782 | CA | ILE | 224 | 68.296 | 11.956 | 22.722 | 0.00 | 0.00 |
| ATOM | 1783 | C | ILE | 224 | 68.725 | 13.453 | 22.496 | 0.00 | 0.00 |
| ATOM | 1784 | O | ILE | 224 | 68.221 | 14.006 | 21.517 | 0.00 | 0.00 |
| ATOM | 1785 | CB | ILE | 224 | 66.907 | 11.792 | 23.357 | 0.00 | 0.00 |
| ATOM | 1786 | CG1 | ILE | 224 | 65.811 | 12.285 | 22.414 | 0.00 | 0.00 |
| ATOM | 1787 | CG2 | ILE | 224 | 66.871 | 12.596 | 24.680 | 0.00 | 0.00 |
| ATOM | 1788 | CD1 | ILE | 224 | 64.378 | 11.908 | 22.825 | 0.00 | 0.00 |
| ATOM | 1789 | N | ASP | 225 | 69.274 | 14.198 | 23.498 | 0.00 | 0.00 |
| ATOM | 1790 | CA | ASP | 225 | 69.560 | 15.635 | 23.200 | 0.00 | 0.00 |
| ATOM | 1791 | C | ASP | 225 | 70.484 | 15.778 | 21.942 | 0.00 | 0.00 |
| ATOM | 1792 | O | ASP | 225 | 69.968 | 16.252 | 20.940 | 0.00 | 0.00 |
| ATOM | 1793 | CB | ASP | 225 | 70.186 | 16.372 | 24.381 | 0.00 | 0.00 |
| ATOM | 1794 | CG | ASP | 225 | 69.318 | 16.402 | 25.578 | 0.00 | 0.00 |
| ATOM | 1795 | OD1 | ASP | 225 | 68.135 | 16.851 | 25.307 | 0.00 | 0.00 |
| ATOM | 1796 | OD2 | ASP | 225 | 69.630 | 16.047 | 26.726 | 0.00 | 0.00 |
| ATOM | 1797 | N | LYS | 226 | 71.842 | 15.724 | 22.076 | 0.00 | 0.00 |
| ATOM | 1798 | CA | LYS | 226 | 72.787 | 15.746 | 20.947 | 0.00 | 0.00 |
| ATOM | 1799 | C | LYS | 226 | 72.520 | 16.935 | 19.952 | 0.00 | 0.00 |
| ATOM | 1800 | O | LYS | 226 | 72.165 | 16.685 | 18.794 | 0.00 | 0.00 |
| ATOM | 1801 | CB | LYS | 226 | 72.554 | 14.394 | 20.384 | 0.00 | 0.00 |
| ATOM | 1802 | CG | LYS | 226 | 73.076 | 13.223 | 21.127 | 0.00 | 0.00 |
| ATOM | 1803 | CD | LYS | 226 | 73.295 | 12.026 | 20.221 | 0.00 | 0.00 |
| ATOM | 1804 | CE | LYS | 226 | 71.994 | 11.483 | 19.686 | 0.00 | 0.00 |
| ATOM | 1805 | NZ | LYS | 226 | 72.138 | 10.297 | 18.846 | 0.00 | 0.00 |
| ATOM | 1806 | N | SER | 227 | 72.325 | 18.177 | 20.471 | 0.00 | 0.00 |
| ATOM | 1807 | CA | SER | 227 | 72.140 | 19.391 | 19.681 | 0.00 | 0.00 |
| ATOM | 1808 | C | SER | 227 | 73.322 | 20.294 | 19.904 | 0.00 | 0.00 |
| ATOM | 1809 | O | SER | 227 | 73.232 | 21.248 | 20.724 | 0.00 | 0.00 |
| ATOM | 1810 | CB | SER | 227 | 70.855 | 20.078 | 20.015 | 0.00 | 0.00 |
| ATOM | 1811 | OG | SER | 227 | 69.647 | 19.476 | 19.624 | 0.00 | 0.00 |
| ATOM | 1812 | N | GLU | 228 | 74.203 | 20.256 | 18.956 | 0.00 | 0.00 |
| ATOM | 1813 | CA | GLU | 228 | 75.463 | 21.033 | 18.908 | 0.00 | 0.00 |
| ATOM | 1814 | C | GLU | 228 | 76.154 | 21.062 | 20.319 | 0.00 | 0.00 |
| ATOM | 1815 | O | GLU | 228 | 77.168 | 21.770 | 20.410 | 0.00 | 0.00 |
| ATOM | 1816 | CB | GLU | 228 | 75.101 | 22.480 | 18.526 | 0.00 | 0.00 |
| ATOM | 1817 | CG | GLU | 228 | 75.104 | 22.696 | 17.054 | 0.00 | 0.00 |
| ATOM | 1818 | CD | GLU | 228 | 76.197 | 22.309 | 16.184 | 0.00 | 0.00 |
| ATOM | 1819 | OE1 | GLU | 228 | 77.253 | 23.022 | 16.105 | 0.00 | 0.00 |
| ATOM | 1820 | OE2 | GLU | 228 | 76.044 | 21.186 | 15.472 | 0.00 | 0.00 |
| ATOM | 1821 | N | GLY | 229 | 75.957 | 20.074 | 21.221 | 0.00 | 0.00 |
| ATOM | 1822 | CA | GLY | 229 | 76.550 | 20.073 | 22.572 | 0.00 | 0.00 |
| ATOM | 1823 | C | GLY | 229 | 77.855 | 19.218 | 22.726 | 0.00 | 0.00 |
| ATOM | 1824 | O | GLY | 229 | 78.746 | 19.729 | 23.401 | 0.00 | 0.00 |
| ATOM | 1825 | N | PHE | 230 | 78.075 | 18.133 | 21.940 | 0.00 | 0.00 |
| ATOM | 1826 | CA | PHE | 230 | 79.253 | 17.277 | 22.092 | 0.00 | 0.00 |
| ATOM | 1827 | C | PHE | 230 | 79.369 | 16.271 | 20.897 | 0.00 | 0.00 |
| ATOM | 1828 | O | PHE | 230 | 78.778 | 16.546 | 19.859 | 0.00 | 0.00 |
| ATOM | 1829 | CB | PHE | 230 | 79.190 | 16.576 | 23.499 | 0.00 | 0.00 |
| ATOM | 1830 | CG | PHE | 230 | 80.326 | 15.542 | 23.641 | 0.00 | 0.00 |
| ATOM | 1831 | CD1 | PHE | 230 | 80.212 | 14.224 | 23.202 | 0.00 | 0.00 |
| ATOM | 1832 | CD2 | PHE | 230 | 81.520 | 15.937 | 24.223 | 0.00 | 0.00 |
| ATOM | 1833 | CE1 | PHE | 230 | 81.266 | 13.325 | 23.338 | 0.00 | 0.00 |
| ATOM | 1834 | CE2 | PHE | 230 | 82.570 | 15.039 | 24.361 | 0.00 | 0.00 |
| ATOM | 1835 | CZ | PHE | 230 | 82.439 | 13.731 | 23.915 | 0.00 | 0.00 |
| ATOM | 1836 | N | CYS | 231 | 80.544 | 15.636 | 20.828 | 0.00 | 0.00 |
| ATOM | 1837 | CA | CYS | 231 | 80.698 | 14.598 | 19.787 | 0.00 | 0.00 |
| ATOM | 1838 | C | CYS | 231 | 79.396 | 13.722 | 19.636 | 0.00 | 0.00 |
| ATOM | 1839 | O | CYS | 231 | 79.150 | 13.270 | 18.514 | 0.00 | 0.00 |
| ATOM | 1840 | CB | CYS | 231 | 81.908 | 13.691 | 20.025 | 0.00 | 0.00 |
| ATOM | 1841 | SG | CYS | 231 | 83.387 | 14.505 | 20.626 | 0.00 | 0.00 |
| ATOM | 1842 | N | LEU | 232 | 78.651 | 13.376 | 20.710 | 0.00 | 0.00 |
| ATOM | 1843 | CA | LEU | 232 | 77.372 | 12.649 | 20.637 | 0.00 | 0.00 |
| ATOM | 1844 | C | LEU | 232 | 76.293 | 13.328 | 19.719 | 0.00 | 0.00 |
| ATOM | 1845 | O | LEU | 232 | 75.622 | 12.590 | 19.054 | 0.00 | 0.00 |
| ATOM | 1846 | CB | LEU | 232 | 76.895 | 12.539 | 22.078 | 0.00 | 0.00 |
| ATOM | 1847 | CG | LEU | 232 | 77.182 | 11.299 | 22.871 | 0.00 | 0.00 |
| ATOM | 1848 | CD1 | LEU | 232 | 78.637 | 10.921 | 22.981 | 0.00 | 0.00 |
| ATOM | 1849 | CD2 | LEU | 232 | 76.608 | 11.362 | 24.329 | 0.00 | 0.00 |
| ATOM | 1850 | N | LYS | 233 | 76.158 | 14.671 | 19.665 | 0.00 | 0.00 |
| ATOM | 1851 | CA | LYS | 233 | 75.220 | 15.345 | 18.754 | 0.00 | 0.00 |
| ATOM | 1852 | C | LYS | 233 | 75.276 | 14.759 | 17.282 | 0.00 | 0.00 |
| ATOM | 1853 | O | LYS | 233 | 74.204 | 14.756 | 16.670 | 0.00 | 0.00 |
| ATOM | 1854 | CB | LYS | 233 | 75.486 | 16.863 | 18.709 | 0.00 | 0.00 |
| ATOM | 1855 | CG | LYS | 233 | 76.815 | 17.208 | 18.100 | 0.00 | 0.00 |
| ATOM | 1856 | CD | LYS | 233 | 77.120 | 18.669 | 17.932 | 0.00 | 0.00 |
| ATOM | 1857 | CE | LYS | 233 | 78.502 | 18.528 | 17.289 | 0.00 | 0.00 |
| ATOM | 1858 | NZ | LYS | 233 | 79.249 | 19.768 | 17.279 | 0.00 | 0.00 |
| ATOM | 1859 | N | GLU | 234 | 76.460 | 14.621 | 16.616 | 0.00 | 0.00 |
| ATOM | 1860 | CA | GLU | 234 | 76.566 | 13.976 | 15.277 | 0.00 | 0.00 |
| ATOM | 1861 | C | GLU | 234 | 75.746 | 12.633 | 15.161 | 0.00 | 0.00 |
| ATOM | 1862 | O | GLU | 234 | 75.680 | 12.119 | 14.051 | 0.00 | 0.00 |
| ATOM | 1863 | CB | GLU | 234 | 78.062 | 13.786 | 14.983 | 0.00 | 0.00 |
| ATOM | 1864 | CG | GLU | 234 | 78.884 | 15.041 | 14.735 | 0.00 | 0.00 |
| ATOM | 1865 | CD | GLU | 234 | 78.351 | 16.011 | 13.723 | 0.00 | 0.00 |
| ATOM | 1866 | OE1 | GLU | 234 | 78.282 | 17.229 | 13.831 | 0.00 | 0.00 |
| ATOM | 1867 | OE2 | GLU | 234 | 77.886 | 15.473 | 12.617 | 0.00 | 0.00 |
| ATOM | 1868 | N | HIS | 235 | 75.730 | 11.887 | 16.270 | 0.00 | 0.00 |
| ATOM | 1869 | CA | HIS | 235 | 75.002 | 10.647 | 16.476 | 0.00 | 0.00 |
| ATOM | 1870 | C | HIS | 235 | 73.483 | 10.871 | 16.164 | 0.00 | 0.00 |
| ATOM | 1871 | O | HIS | 235 | 73.065 | 10.338 | 15.138 | 0.00 | 0.00 |
| ATOM | 1872 | CB | HIS | 235 | 75.340 | 10.228 | 17.952 | 0.00 | 0.00 |
| ATOM | 1873 | CG | HIS | 235 | 75.482 | 8.723 | 18.295 | 0.00 | 0.00 |
| ATOM | 1874 | ND1 | HIS | 235 | 74.898 | 8.154 | 19.433 | 0.00 | 0.00 |
| ATOM | 1875 | CD2 | HIS | 235 | 76.174 | 7.703 | 17.703 | 0.00 | 0.00 |
| ATOM | 1876 | CE1 | HIS | 235 | 75.242 | 6.878 | 19.493 | 0.00 | 0.00 |
| ATOM | 1877 | NE2 | HIS | 235 | 76.002 | 6.594 | 18.471 | 0.00 | 0.00 |
| ATOM | 1878 | N | LYS | 236 | 72.846 | 11.965 | 16.668 | 0.00 | 0.00 |
| ATOM | 1879 | CA | LYS | 236 | 71.452 | 12.276 | 16.418 | 0.00 | 0.00 |
| ATOM | 1880 | C | LYS | 236 | 71.284 | 12.725 | 14.933 | 0.00 | 0.00 |
| ATOM | 1881 | O | LYS | 236 | 70.254 | 12.373 | 14.360 | 0.00 | 0.00 |
| ATOM | 1882 | CB | LYS | 236 | 71.030 | 13.259 | 17.447 | 0.00 | 0.00 |
| ATOM | 1883 | CG | LYS | 236 | 69.625 | 13.716 | 17.422 | 0.00 | 0.00 |
| ATOM | 1884 | CD | LYS | 236 | 69.408 | 14.442 | 18.733 | 0.00 | 0.00 |
| ATOM | 1885 | CE | LYS | 236 | 67.951 | 14.774 | 18.890 | 0.00 | 0.00 |
| ATOM | 1886 | NZ | LYS | 236 | 67.202 | 13.545 | 18.715 | 0.00 | 0.00 |
| ATOM | 1887 | N | ALA | 237 | 72.147 | 13.607 | 14.402 | 0.00 | 0.00 |
| ATOM | 1888 | CA | ALA | 237 | 72.160 | 14.020 | 13.024 | 0.00 | 0.00 |
| ATOM | 1889 | C | ALA | 237 | 72.320 | 12.753 | 12.108 | 0.00 | 0.00 |
| ATOM | 1890 | O | ALA | 237 | 71.665 | 12.739 | 11.080 | 0.00 | 0.00 |
| ATOM | 1891 | CB | ALA | 237 | 73.279 | 15.046 | 12.831 | 0.00 | 0.00 |
| ATOM | 1892 | N | LEU | 238 | 73.303 | 11.837 | 12.341 | 0.00 | 0.00 |
| ATOM | 1893 | CA | LEU | 238 | 73.480 | 10.580 | 11.595 | 0.00 | 0.00 |
| ATOM | 1894 | C | LEU | 238 | 72.217 | 9.686 | 11.651 | 0.00 | 0.00 |
| ATOM | 1895 | O | LEU | 238 | 71.878 | 9.172 | 10.589 | 0.00 | 0.00 |
| ATOM | 1896 | CB | LEU | 238 | 74.717 | 9.788 | 12.115 | 0.00 | 0.00 |
| ATOM | 1897 | CG | LEU | 238 | 75.012 | 8.424 | 11.447 | 0.00 | 0.00 |
| ATOM | 1898 | CD1 | LEU | 238 | 75.346 | 8.568 | 9.963 | 0.00 | 0.00 |
| ATOM | 1899 | CD2 | LEU | 238 | 76.123 | 7.662 | 12.177 | 0.00 | 0.00 |
| ATOM | 1900 | N | LYS | 239 | 71.727 | 9.216 | 12.832 | 0.00 | 0.00 |
| ATOM | 1901 | CA | LYS | 239 | 70.470 | 8.451 | 12.918 | 0.00 | 0.00 |
| ATOM | 1902 | C | LYS | 239 | 69.354 | 9.133 | 12.089 | 0.00 | 0.00 |
| ATOM | 1903 | O | LYS | 239 | 68.581 | 8.383 | 11.499 | 0.00 | 0.00 |
| ATOM | 1904 | CB | LYS | 239 | 69.949 | 8.370 | 14.349 | 0.00 | 0.00 |
| ATOM | 1905 | CG | LYS | 239 | 68.826 | 7.425 | 14.652 | 0.00 | 0.00 |
| ATOM | 1906 | CD | LYS | 239 | 68.584 | 7.194 | 16.136 | 0.00 | 0.00 |
| ATOM | 1907 | CE | LYS | 239 | 67.935 | 5.846 | 16.428 | 0.00 | 0.00 |
| ATOM | 1908 | NZ | LYS | 239 | 68.024 | 5.406 | 17.844 | 0.00 | 0.00 |
| ATOM | 1909 | N | THR | 240 | 69.017 | 10.422 | 12.336 | 0.00 | 0.00 |
| ATOM | 1910 | CA | THR | 240 | 68.054 | 11.244 | 11.577 | 0.00 | 0.00 |
| ATOM | 1911 | C | THR | 240 | 68.254 | 11.093 | 10.042 | 0.00 | 0.00 |
| ATOM | 1912 | O | THR | 240 | 67.252 | 10.807 | 9.403 | 0.00 | 0.00 |
| ATOM | 1913 | CB | THR | 240 | 67.934 | 12.712 | 12.119 | 0.00 | 0.00 |
| ATOM | 1914 | OG1 | THR | 240 | 67.546 | 12.744 | 13.533 | 0.00 | 0.00 |
| ATOM | 1915 | CG2 | THR | 240 | 66.905 | 13.528 | 11.291 | 0.00 | 0.00 |
| ATOM | 1916 | N | LEU | 241 | 69.426 | 11.385 | 9.461 | 0.00 | 0.00 |
| ATOM | 1917 | CA | LEU | 241 | 69.717 | 11.198 | 8.022 | 0.00 | 0.00 |
| ATOM | 1918 | C | LEU | 241 | 69.502 | 9.703 | 7.573 | 0.00 | 0.00 |
| ATOM | 1919 | O | LEU | 241 | 69.173 | 9.534 | 6.400 | 0.00 | 0.00 |
| ATOM | 1920 | CB | LEU | 241 | 71.161 | 11.677 | 7.834 | 0.00 | 0.00 |
| ATOM | 1921 | CG | LEU | 241 | 71.289 | 13.196 | 8.072 | 0.00 | 0.00 |
| ATOM | 1922 | CD1 | LEU | 241 | 72.747 | 13.625 | 7.860 | 0.00 | 0.00 |
| ATOM | 1923 | CD2 | LEU | 241 | 70.450 | 13.899 | 7.116 | 0.00 | 0.00 |
| ATOM | 1924 | N | GLY | 242 | 69.875 | 8.688 | 8.382 | 0.00 | 0.00 |
| ATOM | 1925 | CA | GLY | 242 | 69.632 | 7.271 | 8.117 | 0.00 | 0.00 |
| ATOM | 1926 | C | GLY | 242 | 68.102 | 7.081 | 7.972 | 0.00 | 0.00 |
| ATOM | 1927 | O | GLY | 242 | 67.714 | 6.279 | 7.113 | 0.00 | 0.00 |
| ATOM | 1928 | N | ILE | 243 | 67.325 | 7.439 | 9.017 | 0.00 | 0.00 |
| ATOM | 1929 | CA | ILE | 243 | 65.884 | 7.418 | 8.994 | 0.00 | 0.00 |
| ATOM | 1930 | C | ILE | 243 | 65.387 | 8.209 | 7.722 | 0.00 | 0.00 |
| ATOM | 1931 | O | ILE | 243 | 64.332 | 7.830 | 7.218 | 0.00 | 0.00 |
| ATOM | 1932 | CB | ILE | 243 | 65.254 | 7.933 | 10.319 | 0.00 | 0.00 |
| ATOM | 1933 | CG1 | ILE | 243 | 65.394 | 6.822 | 11.394 | 0.00 | 0.00 |
| ATOM | 1934 | CG2 | ILE | 243 | 63.732 | 8.264 | 10.111 | 0.00 | 0.00 |
| ATOM | 1935 | CD1 | ILE | 243 | 64.436 | 5.632 | 11.111 | 0.00 | 0.00 |
| ATOM | 1936 | N | ILE | 244 | 65.957 | 9.397 | 7.354 | 0.00 | 0.00 |
| ATOM | 1937 | CA | ILE | 244 | 65.533 | 10.064 | 6.081 | 0.00 | 0.00 |
| ATOM | 1938 | C | ILE | 244 | 65.472 | 9.022 | 4.911 | 0.00 | 0.00 |
| ATOM | 1939 | O | ILE | 244 | 64.460 | 9.009 | 4.213 | 0.00 | 0.00 |
| ATOM | 1940 | CB | ILE | 244 | 66.443 | 11.250 | 5.744 | 0.00 | 0.00 |
| ATOM | 1941 | CG1 | ILE | 244 | 66.457 | 12.250 | 6.869 | 0.00 | 0.00 |
| ATOM | 1942 | CG2 | ILE | 244 | 66.055 | 11.857 | 4.401 | 0.00 | 0.00 |
| ATOM | 1943 | CD1 | ILE | 244 | 67.431 | 13.459 | 6.665 | 0.00 | 0.00 |
| ATOM | 1944 | N | MET | 245 | 66.554 | 8.258 | 4.646 | 0.00 | 0.00 |
| ATOM | 1945 | CA | MET | 245 | 66.611 | 7.177 | 3.656 | 0.00 | 0.00 |
| ATOM | 1946 | C | MET | 245 | 65.463 | 6.125 | 3.788 | 0.00 | 0.00 |
| ATOM | 1947 | O | MET | 245 | 65.105 | 5.585 | 2.734 | 0.00 | 0.00 |
| ATOM | 1948 | CB | MET | 245 | 67.909 | 6.422 | 3.900 | 0.00 | 0.00 |
| ATOM | 1949 | CG | MET | 245 | 69.188 | 7.090 | 3.600 | 0.00 | 0.00 |
| ATOM | 1950 | SD | MET | 245 | 70.635 | 5.990 | 3.860 | 0.00 | 0.00 |
| ATOM | 1951 | CE | MET | 245 | 70.024 | 4.891 | 5.129 | 0.00 | 0.00 |
| ATOM | 1952 | N | GLY | 246 | 65.224 | 5.553 | 5.000 | 0.00 | 0.00 |
| ATOM | 1953 | CA | GLY | 246 | 64.176 | 4.614 | 5.296 | 0.00 | 0.00 |
| ATOM | 1954 | C | GLY | 246 | 62.810 | 5.182 | 4.849 | 0.00 | 0.00 |
| ATOM | 1955 | O | GLY | 246 | 62.079 | 4.397 | 4.265 | 0.00 | 0.00 |
| ATOM | 1956 | N | THR | 247 | 62.391 | 6.396 | 5.249 | 0.00 | 0.00 |
| ATOM | 1957 | CA | THR | 247 | 61.152 | 6.999 | 4.760 | 0.00 | 0.00 |
| ATOM | 1958 | C | THR | 247 | 61.114 | 7.133 | 3.184 | 0.00 | 0.00 |
| ATOM | 1959 | O | THR | 247 | 59.995 | 7.101 | 2.676 | 0.00 | 0.00 |
| ATOM | 1960 | CB | THR | 247 | 60.821 | 8.295 | 5.558 | 0.00 | 0.00 |
| ATOM | 1961 | OG1 | THR | 247 | 60.599 | 8.072 | 6.963 | 0.00 | 0.00 |
| ATOM | 1962 | CG2 | THR | 247 | 59.561 | 9.047 | 5.009 | 0.00 | 0.00 |
| ATOM | 1963 | N | PHE | 248 | 62.181 | 7.619 | 2.483 | 0.00 | 0.00 |
| ATOM | 1964 | CA | PHE | 248 | 62.283 | 7.689 | 1.019 | 0.00 | 0.00 |
| ATOM | 1965 | C | PHE | 248 | 62.141 | 6.270 | 0.390 | 0.00 | 0.00 |
| ATOM | 1966 | O | PHE | 248 | 61.611 | 6.231 | −0.718 | 0.00 | 0.00 |
| ATOM | 1967 | CB | PHE | 248 | 63.578 | 8.394 | 0.547 | 0.00 | 0.00 |
| ATOM | 1968 | CG | PHE | 248 | 63.643 | 8.542 | −0.993 | 0.00 | 0.00 |
| ATOM | 1969 | CD1 | PHE | 248 | 63.342 | 9.754 | −1.525 | 0.00 | 0.00 |
| ATOM | 1970 | CD2 | PHE | 248 | 63.976 | 7.501 | −1.851 | 0.00 | 0.00 |
| ATOM | 1971 | CE1 | PHE | 248 | 63.364 | 9.932 | −2.899 | 0.00 | 0.00 |
| ATOM | 1972 | CE2 | PHE | 248 | 63.994 | 7.670 | −3.209 | 0.00 | 0.00 |
| ATOM | 1973 | CZ | PHE | 248 | 63.685 | 8.893 | −3.746 | 0.00 | 0.00 |
| ATOM | 1974 | N | THR | 249 | 62.881 | 5.225 | 0.837 | 0.00 | 0.00 |
| ATOM | 1975 | CA | THR | 249 | 62.639 | 3.889 | 0.294 | 0.00 | 0.00 |
| ATOM | 1976 | C | THR | 249 | 61.140 | 3.474 | 0.608 | 0.00 | 0.00 |
| ATOM | 1977 | O | THR | 249 | 60.476 | 3.164 | −0.333 | 0.00 | 0.00 |
| ATOM | 1978 | CB | THR | 249 | 63.542 | 2.807 | 0.942 | 0.00 | 0.00 |
| ATOM | 1979 | OG1 | THR | 249 | 63.556 | 2.703 | 2.337 | 0.00 | 0.00 |
| ATOM | 1980 | CG2 | THR | 249 | 64.967 | 2.852 | 0.359 | 0.00 | 0.00 |
| ATOM | 1981 | N | LEU | 250 | 60.535 | 3.833 | 1.766 | 0.00 | 0.00 |
| ATOM | 1982 | CA | LEU | 250 | 59.089 | 3.514 | 1.999 | 0.00 | 0.00 |
| ATOM | 1983 | C | LEU | 250 | 58.184 | 4.200 | 0.938 | 0.00 | 0.00 |
| ATOM | 1984 | O | LEU | 250 | 57.451 | 3.508 | 0.232 | 0.00 | 0.00 |
| ATOM | 1985 | CB | LEU | 250 | 58.617 | 3.868 | 3.412 | 0.00 | 0.00 |
| ATOM | 1986 | CG | LEU | 250 | 59.394 | 3.135 | 4.496 | 0.00 | 0.00 |
| ATOM | 1987 | CD1 | LEU | 250 | 59.159 | 3.911 | 5.774 | 0.00 | 0.00 |
| ATOM | 1988 | CD2 | LEU | 250 | 58.856 | 1.718 | 4.637 | 0.00 | 0.00 |
| ATOM | 1989 | N | CYS | 251 | 58.416 | 5.496 | 0.732 | 0.00 | 0.00 |
| ATOM | 1990 | CA | CYS | 251 | 57.702 | 6.381 | −0.195 | 0.00 | 0.00 |
| ATOM | 1991 | C | CYS | 251 | 57.973 | 6.022 | −1.683 | 0.00 | 0.00 |
| ATOM | 1992 | O | CYS | 251 | 56.957 | 5.925 | −2.429 | 0.00 | 0.00 |
| ATOM | 1993 | CB | CYS | 251 | 58.210 | 7.804 | 0.147 | 0.00 | 0.00 |
| ATOM | 1994 | SG | CYS | 251 | 56.967 | 9.048 | −0.365 | 0.00 | 0.00 |
| ATOM | 1995 | N | TRP | 252 | 59.219 | 5.627 | −2.004 | 0.00 | 0.00 |
| ATOM | 1996 | CA | TRP | 252 | 59.540 | 5.384 | −3.419 | 0.00 | 0.00 |
| ATOM | 1997 | C | TRP | 252 | 60.035 | 3.907 | −3.734 | 0.00 | 0.00 |
| ATOM | 1998 | O | TRP | 252 | 60.434 | 3.640 | −4.906 | 0.00 | 0.00 |
| ATOM | 1999 | CB | TRP | 252 | 60.418 | 6.430 | −4.031 | 0.00 | 0.00 |
| ATOM | 2000 | CG | TRP | 252 | 59.918 | 7.793 | −3.916 | 0.00 | 0.00 |
| ATOM | 2001 | CD1 | TRP | 252 | 60.406 | 8.823 | −3.138 | 0.00 | 0.00 |
| ATOM | 2002 | CD2 | TRP | 252 | 58.804 | 8.318 | −4.641 | 0.00 | 0.00 |
| ATOM | 2003 | NE1 | TRP | 252 | 59.674 | 9.954 | −3.384 | 0.00 | 0.00 |
| ATOM | 2004 | CE2 | TRP | 252 | 58.704 | 9.696 | −4.293 | 0.00 | 0.00 |
| ATOM | 2005 | CE3 | TRP | 252 | 57.895 | 7.783 | −5.542 | 0.00 | 0.00 |
| ATOM | 2006 | CZ2 | TRP | 252 | 57.703 | 10.499 | −4.798 | 0.00 | 0.00 |
| ATOM | 2007 | CZ3 | TRP | 252 | 56.919 | 8.610 | −6.081 | 0.00 | 0.00 |
| ATOM | 2008 | CH2 | TRP | 252 | 56.792 | 9.950 | −5.689 | 0.00 | 0.00 |
| ATOM | 2009 | N | LEU | 253 | 59.663 | 2.949 | −2.829 | 0.00 | 0.00 |
| ATOM | 2010 | CA | LEU | 253 | 59.877 | 1.552 | −2.972 | 0.00 | 0.00 |
| ATOM | 2011 | C | LEU | 253 | 58.747 | 1.036 | −3.962 | 0.00 | 0.00 |
| ATOM | 2012 | O | LEU | 253 | 59.069 | 1.000 | −5.173 | 0.00 | 0.00 |
| ATOM | 2013 | CB | LEU | 253 | 60.088 | 0.734 | −1.724 | 0.00 | 0.00 |
| ATOM | 2014 | CG | LEU | 253 | 61.482 | 0.397 | −1.296 | 0.00 | 0.00 |
| ATOM | 2015 | CD1 | LEU | 253 | 62.515 | 1.477 | −1.534 | 0.00 | 0.00 |
| ATOM | 2016 | CD2 | LEU | 253 | 61.469 | 0.209 | 0.195 | 0.00 | 0.00 |
| ATOM | 2017 | N | PRO | 254 | 57.474 | 0.690 | −3.614 | 0.00 | 0.00 |
| ATOM | 2018 | CA | PRO | 254 | 56.600 | 0.329 | −4.653 | 0.00 | 0.00 |
| ATOM | 2019 | C | PRO | 254 | 56.466 | 1.439 | −5.672 | 0.00 | 0.00 |
| ATOM | 2020 | O | PRO | 254 | 56.260 | 1.057 | −6.803 | 0.00 | 0.00 |
| ATOM | 2021 | CB | PRO | 254 | 55.326 | −0.071 | −3.974 | 0.00 | 0.00 |
| ATOM | 2022 | CG | PRO | 254 | 55.371 | 0.541 | −2.581 | 0.00 | 0.00 |
| ATOM | 2023 | CD | PRO | 254 | 56.810 | 0.501 | −2.259 | 0.00 | 0.00 |
| ATOM | 2024 | N | PHE | 255 | 56.677 | 2.738 | −5.398 | 0.00 | 0.00 |
| ATOM | 2025 | CA | PHE | 255 | 56.605 | 3.648 | −6.580 | 0.00 | 0.00 |
| ATOM | 2026 | C | PHE | 255 | 57.504 | 3.134 | −7.753 | 0.00 | 0.00 |
| ATOM | 2027 | O | PHE | 255 | 57.048 | 3.178 | −8.905 | 0.00 | 0.00 |
| ATOM | 2028 | CB | PHE | 255 | 57.198 | 5.002 | −6.210 | 0.00 | 0.00 |
| ATOM | 2029 | CG | PHE | 255 | 57.293 | 5.981 | −7.333 | 0.00 | 0.00 |
| ATOM | 2030 | CD1 | PHE | 255 | 56.136 | 6.558 | −7.872 | 0.00 | 0.00 |
| ATOM | 2031 | CD2 | PHE | 255 | 58.493 | 6.174 | −7.994 | 0.00 | 0.00 |
| ATOM | 2032 | CE1 | PHE | 255 | 56.232 | 7.387 | −8.977 | 0.00 | 0.00 |
| ATOM | 2033 | CE2 | PHE | 255 | 58.617 | 6.958 | −9.139 | 0.00 | 0.00 |
| ATOM | 2034 | CZ | PHE | 255 | 57.435 | 7.577 | −9.640 | 0.00 | 0.00 |
| ATOM | 2035 | N | PHE | 256 | 58.830 | 2.954 | −7.528 | 0.00 | 0.00 |
| ATOM | 2036 | CA | PHE | 256 | 59.804 | 2.430 | −8.467 | 0.00 | 0.00 |
| ATOM | 2037 | C | PHE | 256 | 59.561 | 0.962 | −8.906 | 0.00 | 0.00 |
| ATOM | 2038 | O | PHE | 256 | 60.112 | 0.620 | −9.939 | 0.00 | 0.00 |
| ATOM | 2039 | CB | PHE | 256 | 61.214 | 2.630 | −7.922 | 0.00 | 0.00 |
| ATOM | 2040 | CG | PHE | 256 | 61.768 | 3.984 | −8.074 | 0.00 | 0.00 |
| ATOM | 2041 | CD1 | PHE | 256 | 61.787 | 4.593 | −9.319 | 0.00 | 0.00 |
| ATOM | 2042 | CD2 | PHE | 256 | 62.272 | 4.643 | −6.943 | 0.00 | 0.00 |
| ATOM | 2043 | CE1 | PHE | 256 | 62.310 | 5.879 | −9.470 | 0.00 | 0.00 |
| ATOM | 2044 | CE2 | PHE | 256 | 62.781 | 5.933 | −7.093 | 0.00 | 0.00 |
| ATOM | 2045 | CZ | PHE | 256 | 62.789 | 6.552 | −8.336 | 0.00 | 0.00 |
| ATOM | 2046 | N | ILE | 257 | 59.384 | 0.018 | −7.931 | 0.00 | 0.00 |
| ATOM | 2047 | CA | ILE | 257 | 59.046 | −1.381 | −8.133 | 0.00 | 0.00 |
| ATOM | 2048 | C | ILE | 257 | 57.835 | −1.541 | −9.090 | 0.00 | 0.00 |
| ATOM | 2049 | O | ILE | 257 | 57.949 | −2.411 | −9.955 | 0.00 | 0.00 |
| ATOM | 2050 | CB | ILE | 257 | 58.807 | −2.085 | −6.762 | 0.00 | 0.00 |
| ATOM | 2051 | CG1 | ILE | 257 | 59.915 | −1.949 | −5.745 | 0.00 | 0.00 |
| ATOM | 2052 | CG2 | ILE | 257 | 58.470 | −3.590 | −6.977 | 0.00 | 0.00 |
| ATOM | 2053 | CD1 | ILE | 257 | 61.331 | −2.125 | −6.288 | 0.00 | 0.00 |
| ATOM | 2054 | N | VAL | 258 | 56.639 | −0.952 | −8.828 | 0.00 | 0.00 |
| ATOM | 2055 | CA | VAL | 258 | 55.514 | −1.027 | −9.805 | 0.00 | 0.00 |
| ATOM | 2056 | C | VAL | 258 | 56.003 | −0.361 | −11.144 | 0.00 | 0.00 |
| ATOM | 2057 | O | VAL | 258 | 55.726 | −0.917 | −12.209 | 0.00 | 0.00 |
| ATOM | 2058 | CB | VAL | 258 | 54.271 | −0.286 | −9.388 | 0.00 | 0.00 |
| ATOM | 2059 | CG1 | VAL | 258 | 54.532 | 1.103 | −8.878 | 0.00 | 0.00 |
| ATOM | 2060 | CG2 | VAL | 258 | 53.183 | −0.322 | −10.405 | 0.00 | 0.00 |
| ATOM | 2061 | N | ASN | 259 | 56.702 | 0.816 | −11.101 | 0.00 | 0.00 |
| ATOM | 2062 | CA | ASN | 259 | 57.291 | 1.494 | −12.204 | 0.00 | 0.00 |
| ATOM | 2063 | C | ASN | 259 | 58.186 | 0.546 | −13.083 | 0.00 | 0.00 |
| ATOM | 2064 | O | ASN | 259 | 57.886 | 0.442 | −14.279 | 0.00 | 0.00 |
| ATOM | 2065 | CB | ASN | 259 | 58.203 | 2.499 | −11.597 | 0.00 | 0.00 |
| ATOM | 2066 | CG | ASN | 259 | 58.225 | 3.852 | −12.139 | 0.00 | 0.00 |
| ATOM | 2067 | OD1 | ASN | 259 | 57.496 | 4.781 | −11.768 | 0.00 | 0.00 |
| ATOM | 2068 | ND2 | ASN | 259 | 59.179 | 3.827 | −13.078 | 0.00 | 0.00 |
| ATOM | 2069 | N | ILE | 260 | 59.248 | −0.082 | −12.525 | 0.00 | 0.00 |
| ATOM | 2070 | CA | ILE | 260 | 60.084 | −1.045 | −13.199 | 0.00 | 0.00 |
| ATOM | 2071 | C | ILE | 260 | 59.271 | −2.286 | −13.726 | 0.00 | 0.00 |
| ATOM | 2072 | O | ILE | 260 | 59.590 | −2.724 | −14.833 | 0.00 | 0.00 |
| ATOM | 2073 | CB | ILE | 260 | 61.278 | −1.376 | −12.289 | 0.00 | 0.00 |
| ATOM | 2074 | CG1 | ILE | 260 | 62.308 | −2.224 | −13.020 | 0.00 | 0.00 |
| ATOM | 2075 | CG2 | ILE | 260 | 60.834 | −2.101 | −11.011 | 0.00 | 0.00 |
| ATOM | 2076 | CD1 | ILE | 260 | 63.592 | −2.510 | −12.232 | 0.00 | 0.00 |
| ATOM | 2077 | N | VAL | 261 | 58.281 | −2.862 | −12.977 | 0.00 | 0.00 |
| ATOM | 2078 | CA | VAL | 261 | 57.429 | −3.972 | −13.439 | 0.00 | 0.00 |
| ATOM | 2079 | C | VAL | 261 | 56.699 | −3.568 | −14.761 | 0.00 | 0.00 |
| ATOM | 2080 | O | VAL | 261 | 56.798 | −4.369 | −15.698 | 0.00 | 0.00 |
| ATOM | 2081 | CB | VAL | 261 | 56.453 | −4.456 | −12.329 | 0.00 | 0.00 |
| ATOM | 2082 | CG1 | VAL | 261 | 55.244 | −5.148 | −13.031 | 0.00 | 0.00 |
| ATOM | 2083 | CG2 | VAL | 261 | 57.031 | −5.617 | −11.498 | 0.00 | 0.00 |
| ATOM | 2084 | N | HIS | 262 | 55.883 | −2.490 | −14.813 | 0.00 | 0.00 |
| ATOM | 2085 | CA | HIS | 262 | 55.264 | −2.017 | −16.064 | 0.00 | 0.00 |
| ATOM | 2086 | C | HIS | 262 | 56.318 | −1.981 | −17.240 | 0.00 | 0.00 |
| ATOM | 2087 | O | HIS | 262 | 55.930 | −2.430 | −18.313 | 0.00 | 0.00 |
| ATOM | 2088 | CB | HIS | 262 | 54.586 | −0.653 | −15.922 | 0.00 | 0.00 |
| ATOM | 2089 | CG | HIS | 262 | 53.465 | −0.653 | −14.942 | 0.00 | 0.00 |
| ATOM | 2090 | ND1 | HIS | 262 | 52.252 | −1.253 | −15.135 | 0.00 | 0.00 |
| ATOM | 2091 | CD2 | HIS | 262 | 53.411 | −0.044 | −13.715 | 0.00 | 0.00 |
| ATOM | 2092 | CE1 | HIS | 262 | 51.513 | −1.059 | −14.063 | 0.00 | 0.00 |
| ATOM | 2093 | NE2 | HIS | 262 | 52.174 | −0.311 | −13.192 | 0.00 | 0.00 |
| ATOM | 2094 | N | VAL | 263 | 57.475 | −1.291 | −17.132 | 0.00 | 0.00 |
| ATOM | 2095 | CA | VAL | 263 | 58.504 | −1.174 | −18.171 | 0.00 | 0.00 |
| ATOM | 2096 | C | VAL | 263 | 58.952 | −2.564 | −18.769 | 0.00 | 0.00 |
| ATOM | 2097 | O | VAL | 263 | 59.024 | −2.617 | −20.006 | 0.00 | 0.00 |
| ATOM | 2098 | CB | VAL | 263 | 59.703 | −0.370 | −17.619 | 0.00 | 0.00 |
| ATOM | 2099 | CG1 | VAL | 263 | 60.929 | −0.613 | −18.479 | 0.00 | 0.00 |
| ATOM | 2100 | CG2 | VAL | 263 | 59.359 | 1.091 | −17.482 | 0.00 | 0.00 |
| ATOM | 2101 | N | ILE | 264 | 59.446 | −3.546 | −17.983 | 0.00 | 0.00 |
| ATOM | 2102 | CA | ILE | 264 | 59.781 | −4.824 | −18.639 | 0.00 | 0.00 |
| ATOM | 2103 | C | ILE | 264 | 58.498 | −5.462 | −19.314 | 0.00 | 0.00 |
| ATOM | 2104 | O | ILE | 264 | 58.587 | −5.772 | −20.504 | 0.00 | 0.00 |
| ATOM | 2105 | CB | ILE | 264 | 60.426 | −5.898 | −17.684 | 0.00 | 0.00 |
| ATOM | 2106 | CG1 | ILE | 264 | 59.896 | −5.787 | −16.273 | 0.00 | 0.00 |
| ATOM | 2107 | CG2 | ILE | 264 | 61.941 | −6.266 | −17.727 | 0.00 | 0.00 |
| ATOM | 2108 | CD1 | ILE | 264 | 59.531 | −7.266 | −15.829 | 0.00 | 0.00 |
| ATOM | 2109 | N | GLN | 265 | 57.406 | −5.786 | −18.583 | 0.00 | 0.00 |
| ATOM | 2110 | CA | GLN | 265 | 56.161 | −6.286 | −19.136 | 0.00 | 0.00 |
| ATOM | 2111 | C | GLN | 265 | 55.269 | −5.107 | −19.555 | 0.00 | 0.00 |
| ATOM | 2112 | O | GLN | 265 | 54.469 | −4.595 | −18.754 | 0.00 | 0.00 |
| ATOM | 2113 | CB | GLN | 265 | 55.466 | −7.159 | −18.088 | 0.00 | 0.00 |
| ATOM | 2114 | CG | GLN | 265 | 56.191 | −8.295 | −17.424 | 0.00 | 0.00 |
| ATOM | 2115 | CD | GLN | 265 | 55.386 | −8.854 | −16.258 | 0.00 | 0.00 |
| ATOM | 2116 | OE1 | GLN | 265 | 56.010 | −9.102 | −15.217 | 0.00 | 0.00 |
| ATOM | 2117 | NE2 | GLN | 265 | 54.096 | −9.079 | −16.523 | 0.00 | 0.00 |
| ATOM | 2118 | N | ASP | 266 | 55.279 | −4.882 | −20.866 | 0.00 | 0.00 |
| ATOM | 2119 | CA | ASP | 266 | 54.460 | −3.901 | −21.513 | 0.00 | 0.00 |
| ATOM | 2120 | C | ASP | 266 | 53.108 | −4.546 | −21.958 | 0.00 | 0.00 |
| ATOM | 2121 | O | ASP | 266 | 53.111 | −5.571 | −22.639 | 0.00 | 0.00 |
| ATOM | 2122 | CB | ASP | 266 | 55.217 | −3.311 | −22.695 | 0.00 | 0.00 |
| ATOM | 2123 | CG | ASP | 266 | 56.391 | −2.495 | −22.240 | 0.00 | 0.00 |
| ATOM | 2124 | OD1 | ASP | 266 | 56.395 | −2.006 | −21.116 | 0.00 | 0.00 |
| ATOM | 2125 | OD2 | ASP | 266 | 57.321 | −2.301 | −23.013 | 0.00 | 0.00 |
| ATOM | 2126 | N | ASN | 267 | 52.084 | −3.679 | −21.996 | 0.00 | 0.00 |
| ATOM | 2127 | CA | ASN | 267 | 50.686 | −4.000 | −22.423 | 0.00 | 0.00 |
| ATOM | 2128 | C | ASN | 267 | 49.938 | −5.072 | −21.522 | 0.00 | 0.00 |
| ATOM | 2129 | O | ASN | 267 | 49.060 | −5.779 | −22.040 | 0.00 | 0.00 |
| ATOM | 2130 | CB | ASN | 267 | 50.656 | −4.439 | −23.912 | 0.00 | 0.00 |
| ATOM | 2131 | CG | ASN | 267 | 49.227 | −4.372 | −24.498 | 0.00 | 0.00 |
| ATOM | 2132 | OD1 | ASN | 267 | 48.538 | −3.417 | −24.205 | 0.00 | 0.00 |
| ATOM | 2133 | ND2 | ASN | 267 | 48.764 | −5.361 | −25.281 | 0.00 | 0.00 |
| ATOM | 2134 | N | LEU | 268 | 50.117 | −5.110 | −20.172 | 0.00 | 0.00 |
| ATOM | 2135 | CA | LEU | 268 | 49.394 | −5.994 | −19.281 | 0.00 | 0.00 |
| ATOM | 2136 | C | LEU | 268 | 49.129 | −5.163 | −17.967 | 0.00 | 0.00 |
| ATOM | 2137 | O | LEU | 268 | 50.049 | −5.165 | −17.138 | 0.00 | 0.00 |
| ATOM | 2138 | CB | LEU | 268 | 50.281 | −7.216 | −19.078 | 0.00 | 0.00 |
| ATOM | 2139 | CG | LEU | 268 | 49.764 | −8.542 | −18.611 | 0.00 | 0.00 |
| ATOM | 2140 | CD1 | LEU | 268 | 50.880 | −9.519 | −18.873 | 0.00 | 0.00 |
| ATOM | 2141 | CD2 | LEU | 268 | 49.393 | −8.558 | −17.128 | 0.00 | 0.00 |
| ATOM | 2142 | N | ILE | 269 | 47.864 | −4.788 | −17.614 | 0.00 | 0.00 |
| ATOM | 2143 | CA | ILE | 269 | 47.620 | −4.054 | −16.330 | 0.00 | 0.00 |
| ATOM | 2144 | C | ILE | 269 | 46.155 | −3.503 | −16.056 | 0.00 | 0.00 |
| ATOM | 2145 | O | ILE | 269 | 45.328 | −3.444 | −16.975 | 0.00 | 0.00 |
| ATOM | 2146 | CB | ILE | 269 | 48.672 | −2.891 | −16.204 | 0.00 | 0.00 |
| ATOM | 2147 | CG1 | ILE | 269 | 48.741 | −2.397 | −14.710 | 0.00 | 0.00 |
| ATOM | 2148 | CG2 | ILE | 269 | 48.235 | −1.706 | −17.018 | 0.00 | 0.00 |
| ATOM | 2149 | CD1 | ILE | 269 | 49.210 | −3.445 | −13.712 | 0.00 | 0.00 |
| ATOM | 2150 | N | ARG | 270 | 45.859 | −2.948 | −14.842 | 0.00 | 0.00 |
| ATOM | 2151 | CA | ARG | 270 | 44.603 | −2.293 | −14.405 | 0.00 | 0.00 |
| ATOM | 2152 | C | ARG | 270 | 44.962 | −0.869 | −13.817 | 0.00 | 0.00 |
| ATOM | 2153 | O | ARG | 270 | 45.980 | −0.762 | −13.111 | 0.00 | 0.00 |
| ATOM | 2154 | CB | ARG | 270 | 43.936 | −3.164 | −13.328 | 0.00 | 0.00 |
| ATOM | 2155 | CG | ARG | 270 | 43.393 | −4.473 | −13.856 | 0.00 | 0.00 |
| ATOM | 2156 | CD | ARG | 270 | 42.279 | −4.306 | −14.815 | 0.00 | 0.00 |
| ATOM | 2157 | NE | ARG | 270 | 41.726 | −5.580 | −15.169 | 0.00 | 0.00 |
| ATOM | 2158 | CZ | ARG | 270 | 42.262 | −6.381 | −16.089 | 0.00 | 0.00 |
| ATOM | 2159 | NH1 | ARG | 270 | 43.403 | −6.110 | −16.693 | 0.00 | 0.00 |
| ATOM | 2160 | NH2 | ARG | 270 | 41.657 | −7.523 | −16.395 | 0.00 | 0.00 |
| ATOM | 2161 | N | LYS | 271 | 44.125 | 0.185 | −13.973 | 0.00 | 0.00 |
| ATOM | 2162 | CA | LYS | 271 | 44.416 | 1.561 | −13.510 | 0.00 | 0.00 |
| ATOM | 2163 | C | LYS | 271 | 44.773 | 1.678 | −12.002 | 0.00 | 0.00 |
| ATOM | 2164 | O | LYS | 271 | 45.869 | 2.179 | −11.752 | 0.00 | 0.00 |
| ATOM | 2165 | CB | LYS | 271 | 43.461 | 2.619 | −14.049 | 0.00 | 0.00 |
| ATOM | 2166 | CG | LYS | 271 | 43.163 | 2.502 | −15.516 | 0.00 | 0.00 |
| ATOM | 2167 | CD | LYS | 271 | 44.546 | 2.468 | −16.218 | 0.00 | 0.00 |
| ATOM | 2168 | CE | LYS | 271 | 44.433 | 2.347 | −17.748 | 0.00 | 0.00 |
| ATOM | 2169 | NZ | LYS | 271 | 45.606 | 1.513 | −18.235 | 0.00 | 0.00 |
| ATOM | 2170 | N | GLU | 272 | 43.947 | 1.255 | −11.060 | 0.00 | 0.00 |
| ATOM | 2171 | CA | GLU | 272 | 44.236 | 1.442 | −9.627 | 0.00 | 0.00 |
| ATOM | 2172 | C | GLU | 272 | 45.627 | 0.880 | −9.156 | 0.00 | 0.00 |
| ATOM | 2173 | O | GLU | 272 | 46.103 | 1.418 | −8.159 | 0.00 | 0.00 |
| ATOM | 2174 | CB | GLU | 272 | 43.110 | 0.758 | −8.824 | 0.00 | 0.00 |
| ATOM | 2175 | CG | GLU | 272 | 41.677 | 1.022 | −9.237 | 0.00 | 0.00 |
| ATOM | 2176 | CD | GLU | 272 | 40.660 | 1.121 | −8.126 | 0.00 | 0.00 |
| ATOM | 2177 | OE1 | GLU | 272 | 41.090 | 1.356 | −6.943 | 0.00 | 0.00 |
| ATOM | 2178 | OE2 | GLU | 272 | 39.477 | 0.964 | −8.364 | 0.00 | 0.00 |
| ATOM | 2179 | N | VAL | 273 | 46.131 | −0.275 | −9.652 | 0.00 | 0.00 |
| ATOM | 2180 | CA | VAL | 273 | 47.490 | −0.780 | −9.305 | 0.00 | 0.00 |
| ATOM | 2181 | C | VAL | 273 | 48.607 | 0.346 | −9.340 | 0.00 | 0.00 |
| ATOM | 2182 | O | VAL | 273 | 49.265 | 0.508 | −8.312 | 0.00 | 0.00 |
| ATOM | 2183 | CB | VAL | 273 | 47.811 | −1.941 | −10.258 | 0.00 | 0.00 |
| ATOM | 2184 | CG1 | VAL | 273 | 49.118 | −2.681 | −9.908 | 0.00 | 0.00 |
| ATOM | 2185 | CG2 | VAL | 273 | 46.675 | −2.962 | −10.214 | 0.00 | 0.00 |
| ATOM | 2186 | N | TYR | 274 | 48.750 | 1.172 | −10.416 | 0.00 | 0.00 |
| ATOM | 2187 | CA | TYR | 274 | 49.801 | 2.204 | −10.573 | 0.00 | 0.00 |
| ATOM | 2188 | C | TYR | 274 | 49.445 | 3.491 | −9.714 | 0.00 | 0.00 |
| ATOM | 2189 | O | TYR | 274 | 50.315 | 3.880 | −8.946 | 0.00 | 0.00 |
| ATOM | 2190 | CB | TYR | 274 | 50.052 | 2.444 | −12.087 | 0.00 | 0.00 |
| ATOM | 2191 | CG | TYR | 274 | 51.061 | 3.556 | −12.314 | 0.00 | 0.00 |
| ATOM | 2192 | CD1 | TYR | 274 | 50.661 | 4.809 | −12.783 | 0.00 | 0.00 |
| ATOM | 2193 | CD2 | TYR | 274 | 52.405 | 3.283 | −12.085 | 0.00 | 0.00 |
| ATOM | 2194 | CE1 | TYR | 274 | 51.634 | 5.778 | −12.981 | 0.00 | 0.00 |
| ATOM | 2195 | CE2 | TYR | 274 | 53.372 | 4.263 | −12.290 | 0.00 | 0.00 |
| ATOM | 2196 | CZ | TYR | 274 | 52.966 | 5.509 | −12.743 | 0.00 | 0.00 |
| ATOM | 2197 | OH | TYR | 274 | 53.985 | 6.424 | −12.940 | 0.00 | 0.00 |
| ATOM | 2198 | N | ILE | 275 | 48.134 | 3.768 | −9.441 | 0.00 | 0.00 |
| ATOM | 2199 | CA | ILE | 275 | 47.616 | 4.863 | −8.586 | 0.00 | 0.00 |
| ATOM | 2200 | C | ILE | 275 | 48.221 | 4.817 | −7.166 | 0.00 | 0.00 |
| ATOM | 2201 | O | ILE | 275 | 48.543 | 5.876 | −6.636 | 0.00 | 0.00 |
| ATOM | 2202 | CB | ILE | 275 | 46.068 | 4.638 | −8.425 | 0.00 | 0.00 |
| ATOM | 2203 | CG1 | ILE | 275 | 45.354 | 4.632 | −9.818 | 0.00 | 0.00 |
| ATOM | 2204 | CG2 | ILE | 275 | 45.490 | 5.833 | −7.559 | 0.00 | 0.00 |
| ATOM | 2205 | CD1 | ILE | 275 | 43.787 | 4.754 | −9.902 | 0.00 | 0.00 |
| ATOM | 2206 | N | LEU | 276 | 47.982 | 3.678 | −6.487 | 0.00 | 0.00 |
| ATOM | 2207 | CA | LEU | 276 | 48.335 | 3.434 | −5.062 | 0.00 | 0.00 |
| ATOM | 2208 | C | LEU | 276 | 49.703 | 3.956 | −4.655 | 0.00 | 0.00 |
| ATOM | 2209 | O | LEU | 276 | 49.706 | 5.034 | −4.073 | 0.00 | 0.00 |
| ATOM | 2210 | CB | LEU | 276 | 48.215 | 1.934 | −4.779 | 0.00 | 0.00 |
| ATOM | 2211 | CG | LEU | 276 | 47.026 | 1.100 | −5.128 | 0.00 | 0.00 |
| ATOM | 2212 | CD1 | LEU | 276 | 47.317 | −0.335 | −4.697 | 0.00 | 0.00 |
| ATOM | 2213 | CD2 | LEU | 276 | 45.786 | 1.594 | −4.442 | 0.00 | 0.00 |
| ATOM | 2214 | N | LEU | 277 | 50.769 | 3.602 | −5.284 | 0.00 | 0.00 |
| ATOM | 2215 | CA | LEU | 277 | 52.137 | 3.906 | −4.909 | 0.00 | 0.00 |
| ATOM | 2216 | C | LEU | 277 | 52.586 | 5.292 | −5.470 | 0.00 | 0.00 |
| ATOM | 2217 | O | LEU | 277 | 53.652 | 5.771 | −5.064 | 0.00 | 0.00 |
| ATOM | 2218 | CB | LEU | 277 | 52.954 | 2.835 | −5.540 | 0.00 | 0.00 |
| ATOM | 2219 | CG | LEU | 277 | 52.675 | 1.390 | −5.424 | 0.00 | 0.00 |
| ATOM | 2220 | CD1 | LEU | 277 | 53.594 | 0.494 | −6.231 | 0.00 | 0.00 |
| ATOM | 2221 | CD2 | LEU | 277 | 52.724 | 0.948 | −3.973 | 0.00 | 0.00 |
| ATOM | 2222 | N | ASN | 278 | 51.996 | 5.703 | −6.625 | 0.00 | 0.00 |
| ATOM | 2223 | CA | ASN | 278 | 52.233 | 7.003 | −7.206 | 0.00 | 0.00 |
| ATOM | 2224 | C | ASN | 278 | 51.671 | 8.090 | −6.259 | 0.00 | 0.00 |
| ATOM | 2225 | O | ASN | 278 | 52.444 | 9.000 | −5.928 | 0.00 | 0.00 |
| ATOM | 2226 | CB | ASN | 278 | 51.558 | 6.984 | −8.564 | 0.00 | 0.00 |
| ATOM | 2227 | CG | ASN | 278 | 52.413 | 6.323 | −9.663 | 0.00 | 0.00 |
| ATOM | 2228 | OD1 | ASN | 278 | 51.945 | 5.392 | −10.335 | 0.00 | 0.00 |
| ATOM | 2229 | ND2 | ASN | 278 | 53.668 | 6.733 | −9.843 | 0.00 | 0.00 |
| ATOM | 2230 | N | TRP | 279 | 50.348 | 8.099 | −5.942 | 0.00 | 0.00 |
| ATOM | 2231 | CA | TRP | 279 | 49.706 | 8.997 | −4.951 | 0.00 | 0.00 |
| ATOM | 2232 | C | TRP | 279 | 50.449 | 8.788 | −3.592 | 0.00 | 0.00 |
| ATOM | 2233 | O | TRP | 279 | 50.661 | 9.755 | −2.908 | 0.00 | 0.00 |
| ATOM | 2234 | CB | TRP | 279 | 48.212 | 8.785 | −4.804 | 0.00 | 0.00 |
| ATOM | 2235 | CG | TRP | 279 | 47.576 | 9.902 | −3.997 | 0.00 | 0.00 |
| ATOM | 2236 | CD1 | TRP | 279 | 47.004 | 11.047 | −4.433 | 0.00 | 0.00 |
| ATOM | 2237 | CD2 | TRP | 279 | 47.541 | 9.963 | −2.558 | 0.00 | 0.00 |
| ATOM | 2238 | NE1 | TRP | 279 | 46.622 | 11.838 | −3.378 | 0.00 | 0.00 |
| ATOM | 2239 | CE2 | TRP | 279 | 46.989 | 11.203 | −2.221 | 0.00 | 0.00 |
| ATOM | 2240 | CE3 | TRP | 279 | 47.932 | 9.051 | −1.556 | 0.00 | 0.00 |
| ATOM | 2241 | CZ2 | TRP | 279 | 46.795 | 11.570 | −0.882 | 0.00 | 0.00 |
| ATOM | 2242 | CZ3 | TRP | 279 | 47.802 | 9.446 | −0.218 | 0.00 | 0.00 |
| ATOM | 2243 | CH2 | TRP | 279 | 47.192 | 10.677 | 0.103 | 0.00 | 0.00 |
| ATOM | 2244 | N | ILE | 280 | 50.515 | 7.549 | −3.012 | 0.00 | 0.00 |
| ATOM | 2245 | CA | ILE | 280 | 51.164 | 7.399 | −1.670 | 0.00 | 0.00 |
| ATOM | 2246 | C | ILE | 280 | 52.581 | 8.079 | −1.668 | 0.00 | 0.00 |
| ATOM | 2247 | O | ILE | 280 | 52.952 | 8.603 | −0.619 | 0.00 | 0.00 |
| ATOM | 2248 | CB | ILE | 280 | 51.175 | 5.963 | −1.124 | 0.00 | 0.00 |
| ATOM | 2249 | CG1 | ILE | 280 | 52.028 | 5.758 | 0.112 | 0.00 | 0.00 |
| ATOM | 2250 | CG2 | ILE | 280 | 51.781 | 5.015 | −2.185 | 0.00 | 0.00 |
| ATOM | 2251 | CD1 | ILE | 280 | 51.440 | 6.342 | 1.392 | 0.00 | 0.00 |
| ATOM | 2252 | N | GLY | 281 | 53.409 | 7.985 | −2.729 | 0.00 | 0.00 |
| ATOM | 2253 | CA | GLY | 281 | 54.721 | 8.646 | −2.810 | 0.00 | 0.00 |
| ATOM | 2254 | C | GLY | 281 | 54.671 | 10.215 | −2.731 | 0.00 | 0.00 |
| ATOM | 2255 | O | GLY | 281 | 55.716 | 10.838 | −2.924 | 0.00 | 0.00 |
| ATOM | 2256 | N | TYR | 282 | 53.489 | 10.801 | −2.785 | 0.00 | 0.00 |
| ATOM | 2257 | CA | TYR | 282 | 53.226 | 12.249 | −2.859 | 0.00 | 0.00 |
| ATOM | 2258 | C | TYR | 282 | 53.560 | 12.824 | −1.417 | 0.00 | 0.00 |
| ATOM | 2259 | O | TYR | 282 | 53.796 | 14.044 | −1.345 | 0.00 | 0.00 |
| ATOM | 2260 | CB | TYR | 282 | 51.673 | 12.226 | −3.085 | 0.00 | 0.00 |
| ATOM | 2261 | CG | TYR | 282 | 51.023 | 12.220 | −4.482 | 0.00 | 0.00 |
| ATOM | 2262 | CD1 | TYR | 282 | 51.693 | 11.956 | −5.685 | 0.00 | 0.00 |
| ATOM | 2263 | CD2 | TYR | 282 | 49.639 | 12.471 | −4.586 | 0.00 | 0.00 |
| ATOM | 2264 | CE1 | TYR | 282 | 51.046 | 11.916 | −6.915 | 0.00 | 0.00 |
| ATOM | 2265 | CE2 | TYR | 282 | 48.933 | 12.432 | −5.806 | 0.00 | 0.00 |
| ATOM | 2266 | CZ | TYR | 282 | 49.666 | 12.144 | −6.968 | 0.00 | 0.00 |
| ATOM | 2267 | OH | TYR | 282 | 49.040 | 12.089 | −8.170 | 0.00 | 0.00 |
| ATOM | 2268 | N | VAL | 283 | 53.349 | 12.058 | −0.311 | 0.00 | 0.00 |
| ATOM | 2269 | CA | VAL | 283 | 53.713 | 12.452 | 1.002 | 0.00 | 0.00 |
| ATOM | 2270 | C | VAL | 283 | 55.246 | 12.774 | 1.024 | 0.00 | 0.00 |
| ATOM | 2271 | O | VAL | 283 | 55.699 | 13.121 | 2.117 | 0.00 | 0.00 |
| ATOM | 2272 | CB | VAL | 283 | 53.302 | 11.357 | 1.993 | 0.00 | 0.00 |
| ATOM | 2273 | CG1 | VAL | 283 | 51.790 | 11.130 | 1.989 | 0.00 | 0.00 |
| ATOM | 2274 | CG2 | VAL | 283 | 54.021 | 10.039 | 1.731 | 0.00 | 0.00 |
| ATOM | 2275 | N | ASN | 284 | 56.079 | 12.321 | 0.036 | 0.00 | 0.00 |
| ATOM | 2276 | CA | ASN | 284 | 57.465 | 12.684 | −0.024 | 0.00 | 0.00 |
| ATOM | 2277 | C | ASN | 284 | 57.656 | 14.230 | 0.261 | 0.00 | 0.00 |
| ATOM | 2278 | O | ASN | 284 | 58.733 | 14.585 | 0.742 | 0.00 | 0.00 |
| ATOM | 2279 | CB | ASN | 284 | 58.096 | 12.408 | −1.409 | 0.00 | 0.00 |
| ATOM | 2280 | CG | ASN | 284 | 57.358 | 13.050 | −2.551 | 0.00 | 0.00 |
| ATOM | 2281 | OD1 | ASN | 284 | 56.326 | 13.699 | −2.484 | 0.00 | 0.00 |
| ATOM | 2282 | ND2 | ASN | 284 | 57.976 | 12.963 | −3.703 | 0.00 | 0.00 |
| ATOM | 2283 | N | SER | 285 | 56.875 | 15.122 | −0.396 | 0.00 | 0.00 |
| ATOM | 2284 | CA | SER | 285 | 56.903 | 16.549 | −0.264 | 0.00 | 0.00 |
| ATOM | 2285 | C | SER | 285 | 56.839 | 16.908 | 1.241 | 0.00 | 0.00 |
| ATOM | 2286 | O | SER | 285 | 57.919 | 17.014 | 1.809 | 0.00 | 0.00 |
| ATOM | 2287 | CB | SER | 285 | 55.716 | 17.282 | −0.966 | 0.00 | 0.00 |
| ATOM | 2288 | OG | SER | 285 | 55.092 | 18.389 | −0.295 | 0.00 | 0.00 |
| ATOM | 2289 | N | GLY | 286 | 55.795 | 16.460 | 1.927 | 0.00 | 0.00 |
| ATOM | 2290 | CA | GLY | 286 | 55.564 | 16.802 | 3.281 | 0.00 | 0.00 |
| ATOM | 2291 | C | GLY | 286 | 56.404 | 16.070 | 4.366 | 0.00 | 0.00 |
| ATOM | 2292 | O | GLY | 286 | 56.732 | 16.750 | 5.343 | 0.00 | 0.00 |
| ATOM | 2293 | N | PHE | 287 | 56.912 | 14.841 | 4.154 | 0.00 | 0.00 |
| ATOM | 2294 | CA | PHE | 287 | 57.622 | 14.062 | 5.182 | 0.00 | 0.00 |
| ATOM | 2295 | C | PHE | 287 | 58.989 | 14.647 | 5.571 | 0.00 | 0.00 |
| ATOM | 2296 | O | PHE | 287 | 59.435 | 14.349 | 6.678 | 0.00 | 0.00 |
| ATOM | 2297 | CB | PHE | 287 | 57.874 | 12.617 | 4.806 | 0.00 | 0.00 |
| ATOM | 2298 | CG | PHE | 287 | 58.932 | 12.240 | 3.839 | 0.00 | 0.00 |
| ATOM | 2299 | CD1 | PHE | 287 | 58.734 | 12.463 | 2.457 | 0.00 | 0.00 |
| ATOM | 2300 | CD2 | PHE | 287 | 60.143 | 11.724 | 4.305 | 0.00 | 0.00 |
| ATOM | 2301 | CE1 | PHE | 287 | 59.755 | 12.127 | 1.560 | 0.00 | 0.00 |
| ATOM | 2302 | CE2 | PHE | 287 | 61.164 | 11.389 | 3.398 | 0.00 | 0.00 |
| ATOM | 2303 | CZ | PHE | 287 | 60.962 | 11.597 | 2.016 | 0.00 | 0.00 |
| ATOM | 2304 | N | ASN | 288 | 59.687 | 15.347 | 4.653 | 0.00 | 0.00 |
| ATOM | 2305 | CA | ASN | 288 | 60.984 | 15.838 | 4.903 | 0.00 | 0.00 |
| ATOM | 2306 | C | ASN | 288 | 61.089 | 16.686 | 6.212 | 0.00 | 0.00 |
| ATOM | 2307 | O | ASN | 288 | 61.870 | 16.258 | 7.081 | 0.00 | 0.00 |
| ATOM | 2308 | CB | ASN | 288 | 61.354 | 16.617 | 3.680 | 0.00 | 0.00 |
| ATOM | 2309 | CG | ASN | 288 | 61.940 | 15.785 | 2.589 | 0.00 | 0.00 |
| ATOM | 2310 | OD1 | ASN | 288 | 63.134 | 15.559 | 2.557 | 0.00 | 0.00 |
| ATOM | 2311 | ND2 | ASN | 288 | 61.272 | 15.263 | 1.571 | 0.00 | 0.00 |
| ATOM | 2312 | N | PRO | 289 | 60.503 | 17.913 | 6.415 | 0.00 | 0.00 |
| ATOM | 2313 | CA | PRO | 289 | 60.673 | 18.524 | 7.718 | 0.00 | 0.00 |
| ATOM | 2314 | C | PRO | 289 | 59.952 | 17.695 | 8.855 | 0.00 | 0.00 |
| ATOM | 2315 | O | PRO | 289 | 60.210 | 18.045 | 10.012 | 0.00 | 0.00 |
| ATOM | 2316 | CB | PRO | 289 | 60.041 | 19.901 | 7.648 | 0.00 | 0.00 |
| ATOM | 2317 | CG | PRO | 289 | 60.239 | 20.300 | 6.145 | 0.00 | 0.00 |
| ATOM | 2318 | CD | PRO | 289 | 60.154 | 19.011 | 5.382 | 0.00 | 0.00 |
| ATOM | 2319 | N | LEU | 290 | 58.827 | 16.979 | 8.599 | 0.00 | 0.00 |
| ATOM | 2320 | CA | LEU | 290 | 58.241 | 16.136 | 9.655 | 0.00 | 0.00 |
| ATOM | 2321 | C | LEU | 290 | 59.349 | 15.322 | 10.400 | 0.00 | 0.00 |
| ATOM | 2322 | O | LEU | 290 | 59.169 | 15.107 | 11.613 | 0.00 | 0.00 |
| ATOM | 2323 | CB | LEU | 290 | 57.148 | 15.233 | 9.108 | 0.00 | 0.00 |
| ATOM | 2324 | CG | LEU | 290 | 56.015 | 15.864 | 8.342 | 0.00 | 0.00 |
| ATOM | 2325 | CD1 | LEU | 290 | 55.058 | 14.757 | 7.868 | 0.00 | 0.00 |
| ATOM | 2326 | CD2 | LEU | 290 | 55.223 | 16.827 | 9.223 | 0.00 | 0.00 |
| ATOM | 2327 | N | ILE | 291 | 60.291 | 14.685 | 9.651 | 0.00 | 0.00 |
| ATOM | 2328 | CA | ILE | 291 | 61.409 | 13.990 | 10.251 | 0.00 | 0.00 |
| ATOM | 2329 | C | ILE | 291 | 62.342 | 14.914 | 11.131 | 0.00 | 0.00 |
| ATOM | 2330 | O | ILE | 291 | 63.009 | 14.329 | 11.996 | 0.00 | 0.00 |
| ATOM | 2331 | CB | ILE | 291 | 62.214 | 13.293 | 9.106 | 0.00 | 0.00 |
| ATOM | 2332 | CG1 | ILE | 291 | 61.299 | 12.232 | 8.421 | 0.00 | 0.00 |
| ATOM | 2333 | CG2 | ILE | 291 | 63.538 | 12.630 | 9.634 | 0.00 | 0.00 |
| ATOM | 2334 | CD1 | ILE | 291 | 62.003 | 11.632 | 7.161 | 0.00 | 0.00 |
| ATOM | 2335 | N | TYR | 292 | 62.176 | 16.257 | 11.159 | 0.00 | 0.00 |
| ATOM | 2336 | CA | TYR | 292 | 63.060 | 17.077 | 12.003 | 0.00 | 0.00 |
| ATOM | 2337 | C | TYR | 292 | 62.685 | 17.023 | 13.546 | 0.00 | 0.00 |
| ATOM | 2338 | O | TYR | 292 | 63.146 | 17.817 | 14.349 | 0.00 | 0.00 |
| ATOM | 2339 | CB | TYR | 292 | 63.100 | 18.487 | 11.322 | 0.00 | 0.00 |
| ATOM | 2340 | CG | TYR | 292 | 64.441 | 19.164 | 11.676 | 0.00 | 0.00 |
| ATOM | 2341 | CD1 | TYR | 292 | 64.670 | 19.539 | 12.977 | 0.00 | 0.00 |
| ATOM | 2342 | CD2 | TYR | 292 | 65.459 | 19.314 | 10.731 | 0.00 | 0.00 |
| ATOM | 2343 | CE1 | TYR | 292 | 65.865 | 20.064 | 13.372 | 0.00 | 0.00 |
| ATOM | 2344 | CE2 | TYR | 292 | 66.677 | 19.849 | 11.120 | 0.00 | 0.00 |
| ATOM | 2345 | CZ | TYR | 292 | 66.860 | 20.211 | 12.459 | 0.00 | 0.00 |
| ATOM | 2346 | OH | TYR | 292 | 68.024 | 20.730 | 12.993 | 0.00 | 0.00 |
| ATOM | 2347 | N | CYS | 293 | 61.609 | 16.254 | 13.885 | 0.00 | 0.00 |
| ATOM | 2348 | CA | CYS | 293 | 61.156 | 15.906 | 15.268 | 0.00 | 0.00 |
| ATOM | 2349 | C | CYS | 293 | 62.382 | 15.353 | 16.066 | 0.00 | 0.00 |
| ATOM | 2350 | O | CYS | 293 | 62.369 | 15.384 | 17.293 | 0.00 | 0.00 |
| ATOM | 2351 | CB | CYS | 293 | 59.860 | 15.010 | 15.326 | 0.00 | 0.00 |
| ATOM | 2352 | SG | CYS | 293 | 58.480 | 16.041 | 16.011 | 0.00 | 0.00 |
| ATOM | 2353 | N | ARG | 294 | 63.359 | 14.741 | 15.346 | 0.00 | 0.00 |
| ATOM | 2354 | CA | ARG | 294 | 64.587 | 14.227 | 15.842 | 0.00 | 0.00 |
| ATOM | 2355 | C | ARG | 294 | 65.719 | 15.315 | 16.077 | 0.00 | 0.00 |
| ATOM | 2356 | O | ARG | 294 | 66.871 | 14.859 | 16.297 | 0.00 | 0.00 |
| ATOM | 2357 | CB | ARG | 294 | 65.022 | 13.178 | 14.810 | 0.00 | 0.00 |
| ATOM | 2358 | CG | ARG | 294 | 64.114 | 12.025 | 14.548 | 0.00 | 0.00 |
| ATOM | 2359 | CD | ARG | 294 | 63.528 | 11.319 | 15.771 | 0.00 | 0.00 |
| ATOM | 2360 | NE | ARG | 294 | 64.516 | 10.399 | 16.275 | 0.00 | 0.00 |
| ATOM | 2361 | CZ | ARG | 294 | 64.983 | 10.419 | 17.492 | 0.00 | 0.00 |
| ATOM | 2362 | NH1 | ARG | 294 | 64.572 | 11.257 | 18.401 | 0.00 | 0.00 |
| ATOM | 2363 | NH2 | ARG | 294 | 65.903 | 9.557 | 17.797 | 0.00 | 0.00 |
| ATOM | 2364 | N | SER | 295 | 65.600 | 16.611 | 15.807 | 0.00 | 0.00 |
| ATOM | 2365 | CA | SER | 295 | 66.760 | 17.457 | 16.187 | 0.00 | 0.00 |
| ATOM | 2366 | C | SER | 295 | 66.261 | 18.523 | 17.141 | 0.00 | 0.00 |
| ATOM | 2367 | O | SER | 295 | 65.771 | 19.526 | 16.619 | 0.00 | 0.00 |
| ATOM | 2368 | CB | SER | 295 | 67.307 | 17.985 | 14.905 | 0.00 | 0.00 |
| ATOM | 2369 | OG | SER | 295 | 68.527 | 18.521 | 14.611 | 0.00 | 0.00 |
| ATOM | 2370 | N | PRO | 296 | 66.431 | 18.481 | 18.494 | 0.00 | 0.00 |
| ATOM | 2371 | CA | PRO | 296 | 65.960 | 19.644 | 19.221 | 0.00 | 0.00 |
| ATOM | 2372 | C | PRO | 296 | 66.698 | 20.907 | 18.800 | 0.00 | 0.00 |
| ATOM | 2373 | O | PRO | 296 | 66.236 | 21.960 | 19.234 | 0.00 | 0.00 |
| ATOM | 2374 | CB | PRO | 296 | 66.166 | 19.516 | 20.762 | 0.00 | 0.00 |
| ATOM | 2375 | CG | PRO | 296 | 67.030 | 18.319 | 20.791 | 0.00 | 0.00 |
| ATOM | 2376 | CD | PRO | 296 | 67.054 | 17.519 | 19.458 | 0.00 | 0.00 |
| ATOM | 2377 | N | ASP | 297 | 67.808 | 20.910 | 18.009 | 0.00 | 0.00 |
| ATOM | 2378 | CA | ASP | 297 | 68.351 | 22.227 | 17.581 | 0.00 | 0.00 |
| ATOM | 2379 | C | ASP | 297 | 67.282 | 23.057 | 16.788 | 0.00 | 0.00 |
| ATOM | 2380 | O | ASP | 297 | 67.032 | 24.199 | 17.174 | 0.00 | 0.00 |
| ATOM | 2381 | CB | ASP | 297 | 69.638 | 21.952 | 16.784 | 0.00 | 0.00 |
| ATOM | 2382 | CG | ASP | 297 | 70.446 | 23.184 | 16.410 | 0.00 | 0.00 |
| ATOM | 2383 | OD1 | ASP | 297 | 69.919 | 24.229 | 16.061 | 0.00 | 0.00 |
| ATOM | 2384 | OD2 | ASP | 297 | 71.660 | 23.077 | 16.488 | 0.00 | 0.00 |
| ATOM | 2385 | N | PHE | 298 | 66.727 | 22.582 | 15.630 | 0.00 | 0.00 |
| ATOM | 2386 | CA | PHE | 298 | 65.651 | 23.239 | 14.838 | 0.00 | 0.00 |
| ATOM | 2387 | C | PHE | 298 | 64.318 | 23.282 | 15.583 | 0.00 | 0.00 |
| ATOM | 2388 | O | PHE | 298 | 63.574 | 24.189 | 15.267 | 0.00 | 0.00 |
| ATOM | 2389 | CB | PHE | 298 | 65.485 | 22.706 | 13.441 | 0.00 | 0.00 |
| ATOM | 2390 | CG | PHE | 298 | 64.597 | 23.488 | 12.539 | 0.00 | 0.00 |
| ATOM | 2391 | CD1 | PHE | 298 | 63.355 | 22.956 | 12.173 | 0.00 | 0.00 |
| ATOM | 2392 | CD2 | PHE | 298 | 64.948 | 24.766 | 12.091 | 0.00 | 0.00 |
| ATOM | 2393 | CE1 | PHE | 298 | 62.485 | 23.667 | 11.345 | 0.00 | 0.00 |
| ATOM | 2394 | CE2 | PHE | 298 | 64.097 | 25.506 | 11.251 | 0.00 | 0.00 |
| ATOM | 2395 | CZ | PHE | 298 | 62.852 | 24.936 | 10.884 | 0.00 | 0.00 |
| ATOM | 2396 | N | ARG | 299 | 63.763 | 22.110 | 15.990 | 0.00 | 0.00 |
| ATOM | 2397 | CA | ARG | 299 | 62.545 | 22.183 | 16.797 | 0.00 | 0.00 |
| ATOM | 2398 | C | ARG | 299 | 62.565 | 23.296 | 17.864 | 0.00 | 0.00 |
| ATOM | 2399 | O | ARG | 299 | 61.515 | 23.934 | 17.985 | 0.00 | 0.00 |
| ATOM | 2400 | CB | ARG | 299 | 62.093 | 20.863 | 17.378 | 0.00 | 0.00 |
| ATOM | 2401 | CG | ARG | 299 | 60.677 | 20.767 | 17.804 | 0.00 | 0.00 |
| ATOM | 2402 | CD | ARG | 299 | 60.299 | 19.327 | 18.181 | 0.00 | 0.00 |
| ATOM | 2403 | NE | ARG | 299 | 58.932 | 19.361 | 18.627 | 0.00 | 0.00 |
| ATOM | 2404 | CZ | ARG | 299 | 57.915 | 19.224 | 17.783 | 0.00 | 0.00 |
| ATOM | 2405 | NH1 | ARG | 299 | 58.067 | 19.018 | 16.487 | 0.00 | 0.00 |
| ATOM | 2406 | NH2 | ARG | 299 | 56.690 | 19.488 | 18.221 | 0.00 | 0.00 |
| ATOM | 2407 | N | ILE | 300 | 63.569 | 23.380 | 18.791 | 0.00 | 0.00 |
| ATOM | 2408 | CA | ILE | 300 | 63.611 | 24.528 | 19.734 | 0.00 | 0.00 |
| ATOM | 2409 | C | ILE | 300 | 63.586 | 25.893 | 18.950 | 0.00 | 0.00 |
| ATOM | 2410 | O | ILE | 300 | 63.016 | 26.840 | 19.511 | 0.00 | 0.00 |
| ATOM | 2411 | CB | ILE | 300 | 64.762 | 24.377 | 20.773 | 0.00 | 0.00 |
| ATOM | 2412 | CG1 | ILE | 300 | 64.493 | 25.285 | 22.012 | 0.00 | 0.00 |
| ATOM | 2413 | CG2 | ILE | 300 | 66.152 | 24.667 | 20.162 | 0.00 | 0.00 |
| ATOM | 2414 | CD1 | ILE | 300 | 65.538 | 25.020 | 23.115 | 0.00 | 0.00 |
| ATOM | 2415 | N | ALA | 301 | 64.238 | 26.045 | 17.779 | 0.00 | 0.00 |
| ATOM | 2416 | CA | ALA | 301 | 64.211 | 27.191 | 16.930 | 0.00 | 0.00 |
| ATOM | 2417 | C | ALA | 301 | 62.782 | 27.423 | 16.286 | 0.00 | 0.00 |
| ATOM | 2418 | O | ALA | 301 | 62.426 | 28.618 | 16.175 | 0.00 | 0.00 |
| ATOM | 2419 | CB | ALA | 301 | 65.210 | 26.859 | 15.785 | 0.00 | 0.00 |
| ATOM | 2420 | N | PHE | 302 | 62.065 | 26.409 | 15.708 | 0.00 | 0.00 |
| ATOM | 2421 | CA | PHE | 302 | 60.714 | 26.833 | 15.240 | 0.00 | 0.00 |
| ATOM | 2422 | C | PHE | 302 | 59.801 | 27.261 | 16.410 | 0.00 | 0.00 |
| ATOM | 2423 | O | PHE | 302 | 58.830 | 27.948 | 16.099 | 0.00 | 0.00 |
| ATOM | 2424 | CB | PHE | 302 | 60.075 | 25.893 | 14.194 | 0.00 | 0.00 |
| ATOM | 2425 | CG | PHE | 302 | 59.848 | 24.468 | 14.770 | 0.00 | 0.00 |
| ATOM | 2426 | CD1 | PHE | 302 | 60.754 | 23.474 | 14.424 | 0.00 | 0.00 |
| ATOM | 2427 | CD2 | PHE | 302 | 58.746 | 24.177 | 15.569 | 0.00 | 0.00 |
| ATOM | 2428 | CE1 | PHE | 302 | 60.542 | 22.187 | 14.889 | 0.00 | 0.00 |
| ATOM | 2429 | CE2 | PHE | 302 | 58.560 | 22.889 | 16.020 | 0.00 | 0.00 |
| ATOM | 2430 | CZ | PHE | 302 | 59.455 | 21.893 | 15.683 | 0.00 | 0.00 |
| ATOM | 2431 | N | GLN | 303 | 59.904 | 26.698 | 17.634 | 0.00 | 0.00 |
| ATOM | 2432 | CA | GLN | 303 | 59.123 | 27.146 | 18.813 | 0.00 | 0.00 |
| ATOM | 2433 | C | GLN | 303 | 59.517 | 28.620 | 19.138 | 0.00 | 0.00 |
| ATOM | 2434 | O | GLN | 303 | 58.661 | 29.365 | 19.632 | 0.00 | 0.00 |
| ATOM | 2435 | CB | GLN | 303 | 59.365 | 26.232 | 20.010 | 0.00 | 0.00 |
| ATOM | 2436 | CG | GLN | 303 | 58.873 | 24.828 | 19.752 | 0.00 | 0.00 |
| ATOM | 2437 | CD | GLN | 303 | 59.077 | 23.945 | 20.967 | 0.00 | 0.00 |
| ATOM | 2438 | OE1 | GLN | 303 | 58.269 | 23.987 | 21.885 | 0.00 | 0.00 |
| ATOM | 2439 | NE2 | GLN | 303 | 60.152 | 23.186 | 20.988 | 0.00 | 0.00 |
| ATOM | 2440 | N | GLU | 304 | 60.794 | 29.003 | 19.007 | 0.00 | 0.00 |
| ATOM | 2441 | CA | GLU | 304 | 61.303 | 30.354 | 19.170 | 0.00 | 0.00 |
| ATOM | 2442 | C | GLU | 304 | 60.581 | 31.341 | 18.184 | 0.00 | 0.00 |
| ATOM | 2443 | O | GLU | 304 | 60.492 | 32.513 | 18.544 | 0.00 | 0.00 |
| ATOM | 2444 | CB | GLU | 304 | 62.814 | 30.381 | 19.090 | 0.00 | 0.00 |
| ATOM | 2445 | CG | GLU | 304 | 63.478 | 31.639 | 19.595 | 0.00 | 0.00 |
| ATOM | 2446 | CD | GLU | 304 | 63.473 | 31.839 | 21.066 | 0.00 | 0.00 |
| ATOM | 2447 | OE1 | GLU | 304 | 63.507 | 32.913 | 21.649 | 0.00 | 0.00 |
| ATOM | 2448 | OE2 | GLU | 304 | 63.513 | 30.768 | 21.725 | 0.00 | 0.00 |
| ATOM | 2449 | N | LEU | 305 | 60.516 | 31.021 | 16.860 | 0.00 | 0.00 |
| ATOM | 2450 | CA | LEU | 305 | 59.767 | 31.857 | 15.900 | 0.00 | 0.00 |
| ATOM | 2451 | C | LEU | 305 | 58.256 | 31.822 | 16.170 | 0.00 | 0.00 |
| ATOM | 2452 | O | LEU | 305 | 57.667 | 32.907 | 16.090 | 0.00 | 0.00 |
| ATOM | 2453 | CB | LEU | 305 | 60.008 | 31.530 | 14.376 | 0.00 | 0.00 |
| ATOM | 2454 | CG | LEU | 305 | 59.184 | 32.344 | 13.310 | 0.00 | 0.00 |
| ATOM | 2455 | CD1 | LEU | 305 | 59.606 | 33.796 | 13.228 | 0.00 | 0.00 |
| ATOM | 2456 | CD2 | LEU | 305 | 59.407 | 31.680 | 11.935 | 0.00 | 0.00 |
| ATOM | 2457 | N | LEU | 306 | 57.599 | 30.636 | 16.264 | 0.00 | 0.00 |
| ATOM | 2458 | CA | LEU | 306 | 56.146 | 30.615 | 16.525 | 0.00 | 0.00 |
| ATOM | 2459 | C | LEU | 306 | 55.799 | 31.622 | 17.698 | 0.00 | 0.00 |
| ATOM | 2460 | O | LEU | 306 | 54.863 | 32.405 | 17.497 | 0.00 | 0.00 |
| ATOM | 2461 | CB | LEU | 306 | 55.637 | 29.179 | 16.733 | 0.00 | 0.00 |
| ATOM | 2462 | CG | LEU | 306 | 55.784 | 28.300 | 15.503 | 0.00 | 0.00 |
| ATOM | 2463 | CD1 | LEU | 306 | 55.370 | 26.899 | 15.867 | 0.00 | 0.00 |
| ATOM | 2464 | CD2 | LEU | 306 | 54.919 | 28.815 | 14.366 | 0.00 | 0.00 |
| ATOM | 2465 | N | CYS | 307 | 56.347 | 31.433 | 18.898 | 0.00 | 0.00 |
| ATOM | 2466 | CA | CYS | 307 | 56.116 | 32.388 | 19.902 | 0.00 | 0.00 |
| ATOM | 2467 | C | CYS | 307 | 57.186 | 33.428 | 19.641 | 0.00 | 0.00 |
| ATOM | 2468 | O | CYS | 307 | 58.354 | 33.106 | 19.927 | 0.00 | 0.00 |
| ATOM | 2469 | CB | CYS | 307 | 56.230 | 31.815 | 21.309 | 0.00 | 0.00 |
| ATOM | 2470 | SG | CYS | 307 | 54.827 | 30.888 | 21.901 | 0.00 | 0.00 |
| ATOM | 2471 | N | LEU | 308 | 56.863 | 34.660 | 19.313 | 0.00 | 0.00 |
| ATOM | 2472 | CA | LEU | 308 | 57.935 | 35.567 | 18.975 | 0.00 | 0.00 |
| ATOM | 2473 | C | LEU | 308 | 58.904 | 35.717 | 20.173 | 0.00 | 0.00 |
| ATOM | 2474 | O | LEU | 308 | 58.567 | 36.270 | 21.235 | 0.00 | 0.00 |
| ATOM | 2475 | CB | LEU | 308 | 57.351 | 36.943 | 18.658 | 0.00 | 0.00 |
| ATOM | 2476 | CG | LEU | 308 | 56.351 | 36.951 | 17.547 | 0.00 | 0.00 |
| ATOM | 2477 | CD1 | LEU | 308 | 55.967 | 38.398 | 17.216 | 0.00 | 0.00 |
| ATOM | 2478 | CD2 | LEU | 308 | 56.936 | 36.272 | 16.295 | 0.00 | 0.00 |
| ATOM | 2479 | N | ARG | 309 | 60.121 | 35.353 | 19.863 | 0.00 | 0.00 |
| ATOM | 2480 | CA | ARG | 309 | 61.301 | 35.437 | 20.713 | 0.00 | 0.00 |
| ATOM | 2481 | C | ARG | 309 | 61.201 | 34.837 | 22.150 | 0.00 | 0.00 |
| ATOM | 2482 | O | ARG | 309 | 62.158 | 35.080 | 22.905 | 0.00 | 0.00 |
| ATOM | 2483 | CB | ARG | 309 | 61.427 | 36.995 | 20.897 | 0.00 | 0.00 |
| ATOM | 2484 | CG | ARG | 309 | 61.330 | 37.803 | 19.646 | 0.00 | 0.00 |
| ATOM | 2485 | CD | ARG | 309 | 61.623 | 39.285 | 19.834 | 0.00 | 0.00 |
| ATOM | 2486 | NE | ARG | 309 | 62.987 | 39.543 | 20.307 | 0.00 | 0.00 |
| ATOM | 2487 | CZ | ARG | 309 | 64.045 | 39.626 | 19.496 | 0.00 | 0.00 |
| ATOM | 2488 | NH1 | ARG | 309 | 63.906 | 39.463 | 18.172 | 0.00 | 0.00 |
| ATOM | 2489 | NH2 | ARG | 309 | 65.293 | 39.875 | 19.918 | 0.00 | 0.00 |
| ATOM | 2490 | N | ARG | 310 | 60.416 | 33.778 | 22.419 | 0.00 | 0.00 |
| ATOM | 2491 | CA | ARG | 310 | 60.238 | 33.280 | 23.776 | 0.00 | 0.00 |
| ATOM | 2492 | C | ARG | 310 | 60.046 | 31.748 | 23.834 | 0.00 | 0.00 |
| ATOM | 2493 | O | ARG | 310 | 59.157 | 31.206 | 23.159 | 0.00 | 0.00 |
| ATOM | 2494 | CB | ARG | 310 | 58.993 | 33.952 | 24.356 | 0.00 | 0.00 |
| ATOM | 2495 | CG | ARG | 310 | 59.138 | 35.402 | 24.730 | 0.00 | 0.00 |
| ATOM | 2496 | CD | ARG | 310 | 57.772 | 36.041 | 25.028 | 0.00 | 0.00 |
| ATOM | 2497 | NE | ARG | 310 | 57.075 | 35.512 | 26.208 | 0.00 | 0.00 |
| ATOM | 2498 | CZ | ARG | 310 | 57.353 | 35.859 | 27.475 | 0.00 | 0.00 |
| ATOM | 2499 | NH1 | ARG | 310 | 56.596 | 35.415 | 28.479 | 0.00 | 0.00 |
| ATOM | 2500 | NH2 | ARG | 310 | 58.379 | 36.646 | 27.770 | 0.00 | 0.00 |
| ATOM | 2501 | N | SER | 311 | 60.830 | 31.111 | 24.681 | 0.00 | 0.00 |
| ATOM | 2502 | CA | SER | 311 | 60.739 | 29.678 | 24.960 | 0.00 | 0.00 |
| ATOM | 2503 | C | SER | 311 | 60.478 | 29.493 | 26.481 | 0.00 | 0.00 |
| ATOM | 2504 | O | SER | 311 | 61.083 | 30.235 | 27.281 | 0.00 | 0.00 |
| ATOM | 2505 | CB | SER | 311 | 62.046 | 28.975 | 24.529 | 0.00 | 0.00 |
| ATOM | 2506 | OG | SER | 311 | 62.082 | 27.568 | 24.804 | 0.00 | 0.00 |
| ATOM | 2507 | N | SER | 312 | 59.574 | 28.548 | 26.855 | 0.00 | 0.00 |
| ATOM | 2508 | CA | SER | 312 | 59.250 | 28.452 | 28.274 | 0.00 | 0.00 |
| ATOM | 2509 | C | SER | 312 | 60.290 | 27.586 | 29.083 | 0.00 | 0.00 |
| ATOM | 2510 | O | SER | 312 | 59.838 | 26.876 | 30.006 | 0.00 | 0.00 |
| ATOM | 2511 | CB | SER | 312 | 57.822 | 27.866 | 28.324 | 0.00 | 0.00 |
| ATOM | 2512 | OG | SER | 312 | 56.866 | 28.432 | 27.441 | 0.00 | 0.00 |
| ATOM | 2513 | N | LEU | 313 | 61.479 | 28.196 | 29.207 | 0.00 | 0.00 |
| ATOM | 2514 | CA | LEU | 313 | 62.666 | 27.715 | 29.860 | 0.00 | 0.00 |
| ATOM | 2515 | C | LEU | 313 | 63.651 | 28.918 | 29.867 | 0.00 | 0.00 |
| ATOM | 2516 | O | LEU | 313 | 63.540 | 29.839 | 29.011 | 0.00 | 0.00 |
| ATOM | 2517 | CB | LEU | 313 | 63.248 | 26.479 | 29.131 | 0.00 | 0.00 |
| ATOM | 2518 | CG | LEU | 313 | 62.667 | 25.083 | 29.286 | 0.00 | 0.00 |
| ATOM | 2519 | CD1 | LEU | 313 | 61.711 | 24.789 | 30.426 | 0.00 | 0.00 |
| ATOM | 2520 | CD2 | LEU | 313 | 62.075 | 24.639 | 27.976 | 0.00 | 0.00 |
| ATOM | 2521 | N | LYS | 314 | 64.725 | 28.877 | 30.661 | 0.00 | 0.00 |
| ATOM | 2522 | CA | LYS | 314 | 65.674 | 29.977 | 30.551 | 0.00 | 0.00 |
| ATOM | 2523 | C | LYS | 314 | 66.544 | 29.936 | 29.259 | 0.00 | 0.00 |
| ATOM | 2524 | O | LYS | 314 | 67.164 | 30.972 | 28.998 | 0.00 | 0.00 |
| ATOM | 2525 | CB | LYS | 314 | 66.557 | 29.897 | 31.815 | 0.00 | 0.00 |
| ATOM | 2526 | CG | LYS | 314 | 65.847 | 30.215 | 33.122 | 0.00 | 0.00 |
| ATOM | 2527 | CD | LYS | 314 | 65.146 | 31.579 | 33.225 | 0.00 | 0.00 |
| ATOM | 2528 | CE | LYS | 314 | 64.150 | 31.544 | 34.398 | 0.00 | 0.00 |
| ATOM | 2529 | NZ | LYS | 314 | 63.338 | 32.757 | 34.503 | 0.00 | 0.00 |
| ATOM | 2530 | N | ALA | 315 | 66.407 | 28.939 | 28.348 | 0.00 | 0.00 |
| ATOM | 2531 | CA | ALA | 315 | 67.281 | 28.832 | 27.199 | 0.00 | 0.00 |
| ATOM | 2532 | C | ALA | 315 | 68.770 | 28.840 | 27.725 | 0.00 | 0.00 |
| ATOM | 2533 | O | ALA | 315 | 69.664 | 29.342 | 27.033 | 0.00 | 0.00 |
| ATOM | 2534 | CB | ALA | 315 | 66.978 | 29.922 | 26.178 | 0.00 | 0.00 |
| ATOM | 2535 | N | TYR | 316 | 69.035 | 28.128 | 28.852 | 0.00 | 0.00 |
| ATOM | 2536 | CA | TYR | 316 | 70.279 | 28.007 | 29.503 | 0.00 | 0.00 |
| ATOM | 2537 | C | TYR | 316 | 71.174 | 27.242 | 28.525 | 0.00 | 0.00 |
| ATOM | 2538 | O | TYR | 316 | 70.711 | 26.194 | 28.026 | 0.00 | 0.00 |
| ATOM | 2539 | CB | TYR | 316 | 70.033 | 27.327 | 30.847 | 0.00 | 0.00 |
| ATOM | 2540 | CG | TYR | 316 | 71.171 | 27.314 | 31.819 | 0.00 | 0.00 |
| ATOM | 2541 | CD1 | TYR | 316 | 72.071 | 26.266 | 31.818 | 0.00 | 0.00 |
| ATOM | 2542 | CD2 | TYR | 316 | 71.237 | 28.298 | 32.783 | 0.00 | 0.00 |
| ATOM | 2543 | CE1 | TYR | 316 | 73.049 | 26.174 | 32.798 | 0.00 | 0.00 |
| ATOM | 2544 | CE2 | TYR | 316 | 72.216 | 28.211 | 33.772 | 0.00 | 0.00 |
| ATOM | 2545 | CZ | TYR | 316 | 73.102 | 27.153 | 33.760 | 0.00 | 0.00 |
| ATOM | 2546 | OH | TYR | 316 | 74.061 | 27.036 | 34.726 | 0.00 | 0.00 |
| ATOM | 2547 | N | GLY | 317 | 72.482 | 27.401 | 28.684 | 0.00 | 0.00 |
| ATOM | 2548 | CA | GLY | 317 | 73.328 | 26.788 | 27.720 | 0.00 | 0.00 |
| ATOM | 2549 | C | GLY | 317 | 73.384 | 25.252 | 27.813 | 0.00 | 0.00 |
| ATOM | 2550 | O | GLY | 317 | 74.343 | 24.732 | 28.392 | 0.00 | 0.00 |
| ATOM | 2551 | N | ASN | 318 | 72.837 | 24.746 | 26.765 | 0.00 | 0.00 |
| ATOM | 2552 | CA | ASN | 318 | 72.720 | 23.385 | 26.324 | 0.00 | 0.00 |
| ATOM | 2553 | C | ASN | 318 | 72.034 | 23.469 | 24.919 | 0.00 | 0.00 |
| ATOM | 2554 | O | ASN | 318 | 70.982 | 24.167 | 24.862 | 0.00 | 0.00 |
| ATOM | 2555 | CB | ASN | 318 | 71.880 | 22.515 | 27.271 | 0.00 | 0.00 |
| ATOM | 2556 | CG | ASN | 318 | 72.541 | 22.233 | 28.577 | 0.00 | 0.00 |
| ATOM | 2557 | OD1 | ASN | 318 | 71.839 | 22.169 | 29.579 | 0.00 | 0.00 |
| ATOM | 2558 | ND2 | ASN | 318 | 73.857 | 22.123 | 28.649 | 0.00 | 0.00 |
| ATOM | 2559 | N | GLY | 319 | 72.190 | 22.504 | 24.017 | 0.00 | 0.00 |
| ATOM | 2560 | CA | GLY | 319 | 71.492 | 22.665 | 22.753 | 0.00 | 0.00 |
| ATOM | 2561 | C | GLY | 319 | 71.934 | 24.051 | 22.171 | 0.00 | 0.00 |
| ATOM | 2562 | O | GLY | 319 | 71.027 | 24.887 | 22.035 | 0.00 | 0.00 |
| ATOM | 2563 | N | TYR | 320 | 73.239 | 24.353 | 21.979 | 0.00 | 0.00 |
| ATOM | 2564 | CA | TYR | 320 | 73.773 | 25.621 | 21.548 | 0.00 | 0.00 |
| ATOM | 2565 | C | TYR | 320 | 72.730 | 26.352 | 20.695 | 0.00 | 0.00 |
| ATOM | 2566 | O | TYR | 320 | 72.457 | 25.975 | 19.542 | 0.00 | 0.00 |
| ATOM | 2567 | CB | TYR | 320 | 75.123 | 25.390 | 20.841 | 0.00 | 0.00 |
| ATOM | 2568 | CG | TYR | 320 | 76.327 | 25.649 | 21.751 | 0.00 | 0.00 |
| ATOM | 2569 | CD1 | TYR | 320 | 77.566 | 25.900 | 21.202 | 0.00 | 0.00 |
| ATOM | 2570 | CD2 | TYR | 320 | 76.182 | 25.565 | 23.118 | 0.00 | 0.00 |
| ATOM | 2571 | CE1 | TYR | 320 | 78.676 | 26.054 | 22.021 | 0.00 | 0.00 |
| ATOM | 2572 | CE2 | TYR | 320 | 77.285 | 25.721 | 23.947 | 0.00 | 0.00 |
| ATOM | 2573 | CZ | TYR | 320 | 78.532 | 25.961 | 23.393 | 0.00 | 0.00 |
| ATOM | 2574 | OH | TYR | 320 | 79.679 | 26.091 | 24.244 | 0.00 | 0.00 |
| ATOM | 2575 | N | SER | 321 | 72.426 | 27.546 | 21.202 | 0.00 | 0.00 |
| ATOM | 2576 | CA | SER | 321 | 71.390 | 28.398 | 20.647 | 0.00 | 0.00 |
| ATOM | 2577 | C | SER | 321 | 71.956 | 29.150 | 19.439 | 0.00 | 0.00 |
| ATOM | 2578 | O | SER | 321 | 72.508 | 30.242 | 19.539 | 0.00 | 0.00 |
| ATOM | 2579 | CB | SER | 321 | 70.831 | 29.297 | 21.761 | 0.00 | 0.00 |
| ATOM | 2580 | OG | SER | 321 | 69.425 | 29.191 | 22.005 | 0.00 | 0.00 |
| ATOM | 2581 | N | SER | 322 | 71.553 | 28.628 | 18.308 | 0.00 | 0.00 |
| ATOM | 2582 | CA | SER | 322 | 71.906 | 29.091 | 16.982 | 0.00 | 0.00 |
| ATOM | 2583 | C | SER | 322 | 70.747 | 29.807 | 16.199 | 0.00 | 0.00 |
| ATOM | 2584 | O | SER | 322 | 70.860 | 29.872 | 14.974 | 0.00 | 0.00 |
| ATOM | 2585 | CB | SER | 322 | 72.510 | 27.862 | 16.286 | 0.00 | 0.00 |
| ATOM | 2586 | OG | SER | 322 | 71.613 | 26.757 | 16.125 | 0.00 | 0.00 |
| ATOM | 2587 | N | ASN | 323 | 69.613 | 30.058 | 16.761 | 0.00 | 0.00 |
| ATOM | 2588 | CA | ASN | 323 | 68.567 | 30.779 | 16.162 | 0.00 | 0.00 |
| ATOM | 2589 | C | ASN | 323 | 69.027 | 32.252 | 16.015 | 0.00 | 0.00 |
| ATOM | 2590 | O | ASN | 323 | 69.981 | 32.711 | 16.699 | 0.00 | 0.00 |
| ATOM | 2591 | CB | ASN | 323 | 67.461 | 30.781 | 17.191 | 0.00 | 0.00 |
| ATOM | 2592 | CG | ASN | 323 | 67.177 | 29.878 | 18.342 | 0.00 | 0.00 |
| ATOM | 2593 | OD1 | ASN | 323 | 67.757 | 28.790 | 18.677 | 0.00 | 0.00 |
| ATOM | 2594 | ND2 | ASN | 323 | 66.351 | 30.336 | 19.312 | 0.00 | 0.00 |
| ATOM | 2595 | N | GLY | 324 | 68.393 | 32.982 | 15.113 | 0.00 | 0.00 |
| ATOM | 2596 | CA | GLY | 324 | 68.742 | 34.412 | 15.039 | 0.00 | 0.00 |
| ATOM | 2597 | C | GLY | 324 | 68.606 | 35.080 | 16.445 | 0.00 | 0.00 |
| ATOM | 2598 | O | GLY | 324 | 69.173 | 36.167 | 16.597 | 0.00 | 0.00 |
| ATOM | 2599 | N | ASN | 325 | 67.569 | 34.696 | 17.211 | 0.00 | 0.00 |
| ATOM | 2600 | CA | ASN | 325 | 67.299 | 35.108 | 18.553 | 0.00 | 0.00 |
| ATOM | 2601 | C | ASN | 325 | 68.175 | 34.289 | 19.555 | 0.00 | 0.00 |
| ATOM | 2602 | O | ASN | 325 | 68.598 | 33.160 | 19.247 | 0.00 | 0.00 |
| ATOM | 2603 | CB | ASN | 325 | 65.835 | 34.723 | 18.792 | 0.00 | 0.00 |
| ATOM | 2604 | CG | ASN | 325 | 64.857 | 35.706 | 18.178 | 0.00 | 0.00 |
| ATOM | 2605 | OD1 | ASN | 325 | 63.634 | 35.526 | 18.329 | 0.00 | 0.00 |
| ATOM | 2606 | ND2 | ASN | 325 | 65.214 | 36.745 | 17.401 | 0.00 | 0.00 |
| ATOM | 2607 | N | THR | 326 | 68.206 | 34.714 | 20.817 | 0.00 | 0.00 |
| ATOM | 2608 | CA | THR | 326 | 68.895 | 34.008 | 21.922 | 0.00 | 0.00 |
| ATOM | 2609 | C | THR | 326 | 70.413 | 33.767 | 21.675 | 0.00 | 0.00 |
| ATOM | 2610 | O | THR | 326 | 70.793 | 32.678 | 21.201 | 0.00 | 0.00 |
| ATOM | 2611 | CB | THR | 326 | 68.151 | 32.674 | 22.275 | 0.00 | 0.00 |
| ATOM | 2612 | OG1 | THR | 326 | 66.717 | 32.785 | 22.294 | 0.00 | 0.00 |
| ATOM | 2613 | CG2 | THR | 326 | 68.626 | 32.016 | 23.614 | 0.00 | 0.00 |
| ATOM | 2614 | N | GLY | 327 | 71.182 | 34.874 | 21.579 | 0.00 | 0.00 |
| ATOM | 2615 | CA | GLY | 327 | 72.611 | 34.764 | 21.424 | 0.00 | 0.00 |
| ATOM | 2616 | C | GLY | 327 | 73.096 | 34.584 | 19.965 | 0.00 | 0.00 |
| ATOM | 2617 | O | GLY | 327 | 73.803 | 33.587 | 19.755 | 0.00 | 0.00 |
| ATOM | 2618 | N | GLU | 328 | 72.479 | 35.240 | 18.940 | 0.00 | 0.00 |
| ATOM | 2619 | CA | GLU | 328 | 72.913 | 35.171 | 17.564 | 0.00 | 0.00 |
| ATOM | 2620 | C | GLU | 328 | 74.480 | 35.212 | 17.396 | 0.00 | 0.00 |
| ATOM | 2621 | O | GLU | 328 | 74.920 | 34.645 | 16.395 | 0.00 | 0.00 |
| ATOM | 2622 | CB | GLU | 328 | 72.166 | 36.287 | 16.824 | 0.00 | 0.00 |
| ATOM | 2623 | CG | GLU | 328 | 72.969 | 37.630 | 16.948 | 0.00 | 0.00 |
| ATOM | 2624 | CD | GLU | 328 | 72.145 | 38.850 | 16.655 | 0.00 | 0.00 |
| ATOM | 2625 | OE1 | GLU | 328 | 72.117 | 39.178 | 15.440 | 0.00 | 0.00 |
| ATOM | 2626 | OE2 | GLU | 328 | 71.563 | 39.479 | 17.538 | 0.00 | 0.00 |
| ATOM | 2627 | N | GLN | 329 | 75.254 | 36.040 | 18.150 | 0.00 | 0.00 |
| ATOM | 2628 | CA | GLN | 329 | 76.717 | 36.092 | 18.148 | 0.00 | 0.00 |
| ATOM | 2629 | C | GLN | 329 | 77.347 | 34.799 | 17.577 | 0.00 | 0.00 |
| ATOM | 2630 | O | GLN | 329 | 77.228 | 33.699 | 18.114 | 0.00 | 0.00 |
| ATOM | 2631 | CB | GLN | 329 | 77.241 | 36.291 | 19.567 | 0.00 | 0.00 |
| ATOM | 2632 | CG | GLN | 329 | 76.507 | 37.286 | 20.443 | 0.00 | 0.00 |
| ATOM | 2633 | CD | GLN | 329 | 75.835 | 38.541 | 19.964 | 0.00 | 0.00 |
| ATOM | 2634 | OE1 | GLN | 329 | 76.479 | 39.546 | 19.598 | 0.00 | 0.00 |
| ATOM | 2635 | NE2 | GLN | 329 | 74.458 | 38.613 | 20.013 | 0.00 | 0.00 |
| ATOM | 2636 | OXT | GLN | 329 | 77.961 | 34.937 | 16.519 | 0.00 | 0.00 |
| TER | 2637 | GLN | | 329 | | | | | |
| END | | | | | | | | | |

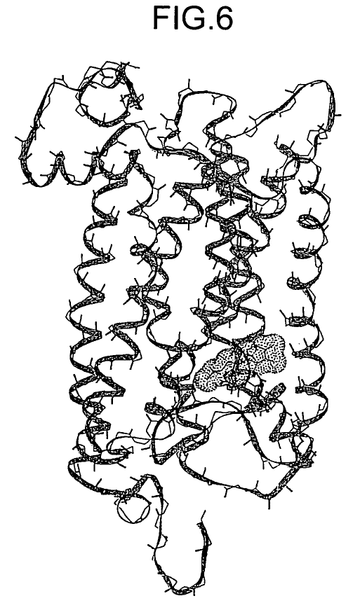

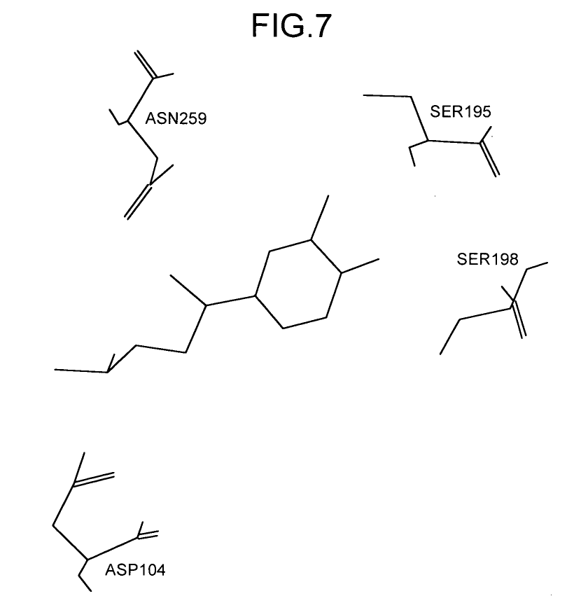

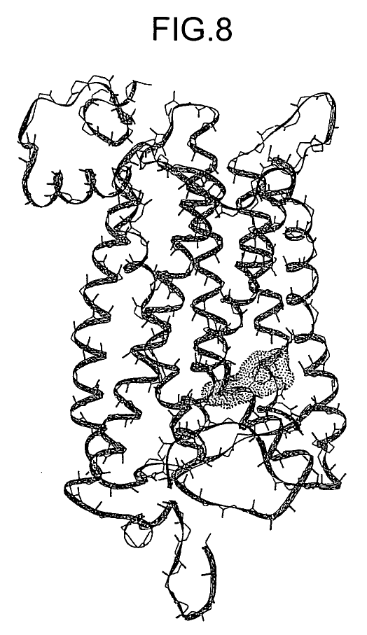

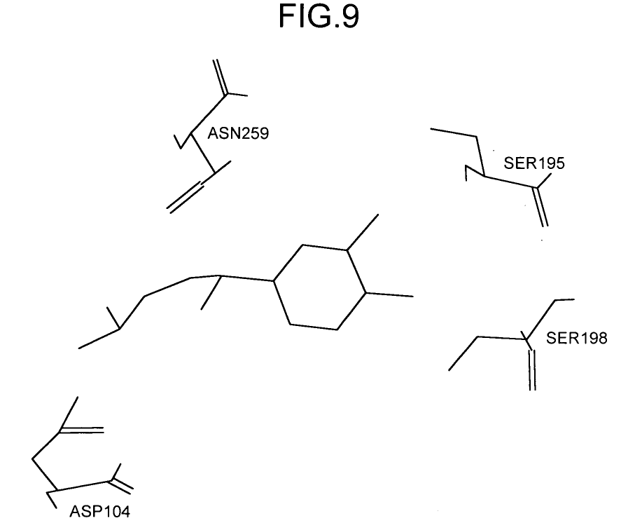

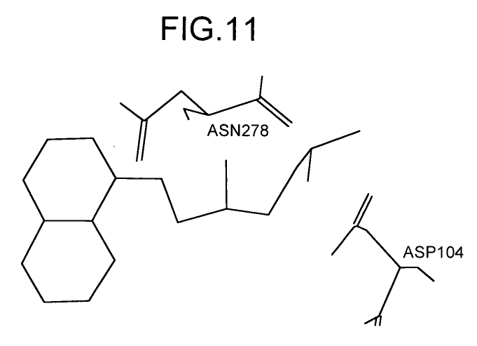

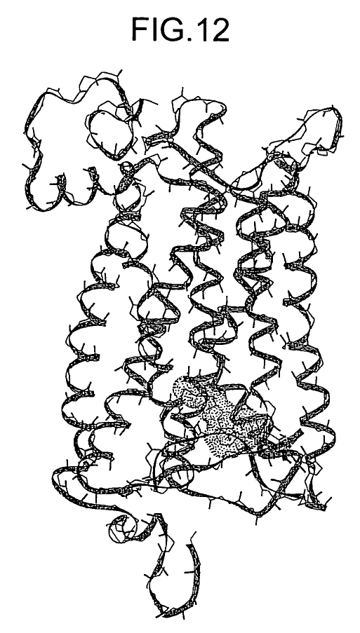

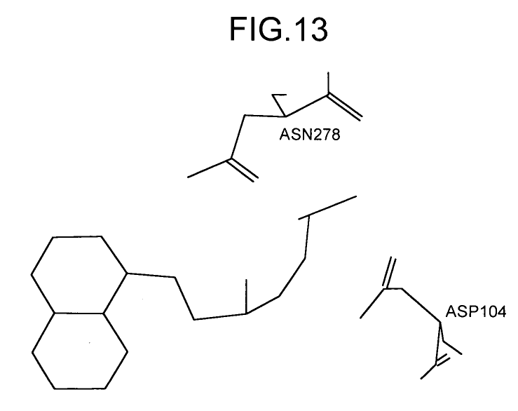

Based on the resulting atomic coordinates, the steric structure model of QRHUB2 (β2 adrenaline receptor) was produced using BIOCES (version 3.10) manufactured by NEC Corporation, and a position into which the agonist (isoproterenol) is placed and a position into which the antagonist (propranolol) is placed were compared. The results are shown in FIGS. 6 to 14.

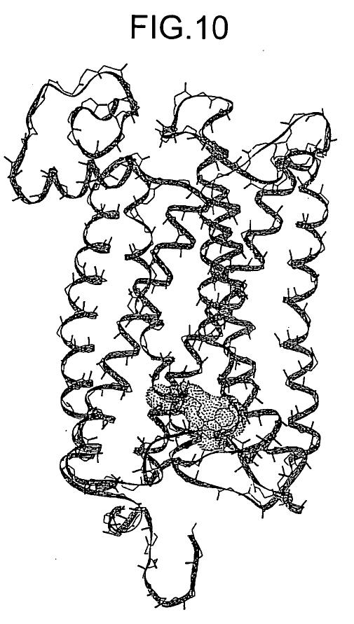

From FIGS. 6 to 9, it is seen that this antagonist (isoproterenol) has optical isomers (R isomer and S isomer), and the R isomer binds with the receptor better. From FIGS. 10 to 13, it is seen that a position of antagonist (propranolol) binding is clearly different from a position of agonist (isopretenol) binding.

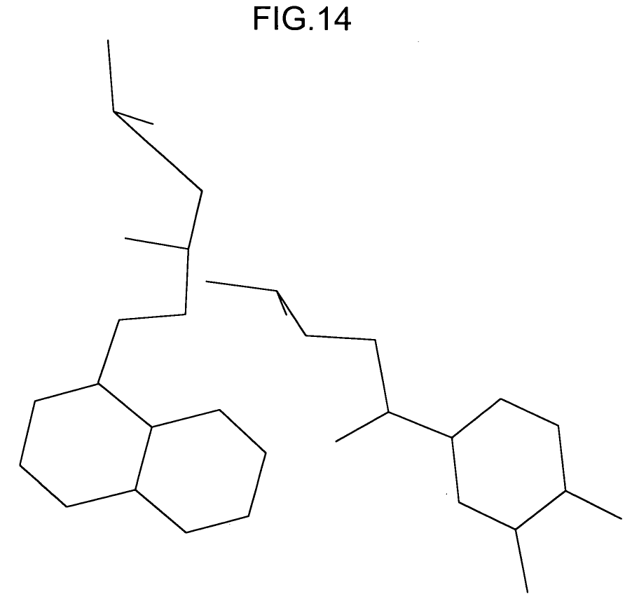

FIG. 14 shows relative arrangement in the receptor of the antagonist (propranolol) (left) and the agonist (isoproterenol) (right) in the β2 adrenaline receptor obtained from the above-mentioned model. From FIG. 14, it is seen that the agonist and the antagonist are introduced into pockets at different positions, respectively, as predicted from an experiment of site-specific mutation.

### Example 2

### Construction of Steric Structures of Amino Acid Sequences Registered in GCRDb, GPCRDB, ExPASy and ORDB

Regarding amino acid sequences of all GPCRs registered in GCRDb, GPCRDB, ExPASy and ORDB, atomic coordinates defining the steric structure were obtained using 1F88 as a reference protein as in Example 1. Each amino acid sequence was obtained from GCRDb: http://www.gcrdb.uthscsa.edu/, GPCRDB: http://www.gpcr.org/7tm/, ExPASy: http://www.expasv.ch/cqi-bin/sm-qpcr.pl, ORDB: http://ycmi.med.yale.edu/senselab/ordb/. In alignments of these amino acid sequences, the E values calculated by PSI-BLAST were as follows:

TABLE 2

| | 1e−3˜ | 1e−11˜ | 1e−51˜ | 1e−100 | |
|---|---|---|---|---|---|
| | 1e−10 | 1e−50 | 1e−100 | or less | Total |
| GCRDb | 5 | 71 | 709 | 46 | 831 |
| GPCRDB | 0 | 0 | 13 | 570 | 583 |
| ExPASY | 0 | 15 | 44 | 318 | 377 |
| ORDB | 0 | 78 | 232 | 107 | 417 |

There was one E value of 0.001 (1e-3) or more in GPCRDB and ORDB, respectively, and these were removed from the Table 2. Therefore, the number of GPCRs for which the steric structure was produced was as follows: GCRDb:831, GPCRDB:584, ORDB:418, ExPASY:377 (total: 2210).

### Example 3

### Construction of Steric Structures of Amino Acid Sequences Registered in GeneBank

Regarding sequences of human, Drosophila, nematode, yeast, thearecress and the like registered in databases such as GeneBank and the like, atomic coordinates defining the steric structure were obtained as in Example 1. Alignment was performed by employing PSAI-BLAST and using 1F88 as a reference protein as in Example 1 and, when the E value was 0.1 or less, it was judged to be GPCRs. Sequences of Drosophila, nematode, yeast and thearecress were obtained from GeneBank: ftp://ncbi.nlm.nih.gov/qenbank/genomes/, and a sequence of human was obtained from ftp://ftp.expasy.ch/databases/swiss-prot/special selection/human.seq, http://cdna.ims.u-tokyo.ac.jp/.

The numbers of sequences for which construction of the steric structure was performed are as follows:

TABLE 3

| | Registered | Total | Overlapped | |
|---|---|---|---|---|
| | sequence | ORF | ORF | Non-overlapped |
| | number | number | number | ORF number |
| Human | 6032 | 272 | 156 | 116 |
| Drosophila | 14100 | 147 | 5 | 142 |
| Nematode | 15808 | 541 | 19 | 522 |
| Thearecress | 403 | 5 | 0 | 5 |
| Yeast | 6295 | 5 | 1 | 4 |

In Table 3, the “registered sequence number” denotes the number of all sequences registered in databases, the “total ORF number” denotes the number of all ORFs (Open Reading Frame) which were determined to be GPCRs, the “overlapped ORF number” denotes the number of ORFs overlapped with sequences of the above four databases (GCRDb, GPCRDb, ORDB, ExPASy), and the “non-overlapped ORF number” denotes the number of ORFs which were not overlapped with sequences of the above-mentioned four databases and were newly determined to be GPCRs, and for which atomic coordinates defining the steric structure were obtained.

### Example 4

### Construction of Steric Structures of Amino Acid Sequences Registered in PIR, SwissPlot and DAD

Further, sequences were obtained from PIR: http://www-nbrf.georgetown.edu/pir/, Swiss Plot: http://www.expasy.ch/sprot/sprot-top.html, TrEMBL: http://www.expasy.ch/sprot/sprot-top.html, TrEMBLNEW: http://www.expasy.ch/sprot/sprot-top.html, DAD: ftp://ftp.ddbj.nig.ac.jp, and atomic coordinates defining the steric structure were obtained as in Example 1. Alignment was performed employing PSAI-BLAST and using 1F88 as a reference protein as in Example 1 and, when the E value was 0.1 or less, it was determined to be GPCRs.

The number of sequences registered in each database are as follows:

TABLE 4

| | |
|---|---|
| PIR 65 | 182,162 |
| SWISSPROT 39 | 87,252 |
| TrEMBL 14 | 296,917 |
| TrEMBLNEW | 71,201 |
| DAD 12 | 514,763 |

Regarding the foregoing total 1,152,295, the number of sequences from which overlapping is omitted is 481,870 and, among them, 5,335 sequences were determined to be GPCRs. Among them, the number of sequences from which overlapping with the above-mentioned four databases (GCRDb, GPCRDb, ORDB, ExPASy) are omitted was 3,475 and, regarding them, atomic coordinates defining the steric structure were obtained.

The present invention has been explained in detail by referring to specific embodiments, but it would be apparent to a person skilled in the art that various variations and modifications can be made without departing from the spirit and the scope of the present invention.

The present application is based on Japanese Patent Application (Patent Application No.2000-279155) filed on Sep. 14, 2000 and Japanese Patent Application (Patent Application No. 2000-350378) filed on Nov. 17, 2000, the entire content of which is incorporated herein by reference.

### INDUSTRIAL APPLICABILITY

The excellent points of the present invention compared with the previous methods are as follows:

(1) Alignment is performed by using the amino acid sequence database “GCRDb” as a profile, a model of an arbitrary G-protein-coupled receptor is produced, for example, based on the steric structure of rhodopsin (PDB ID:1F88), models of a 7-transmembrane a helix structure which is the common nature of GPCRs structures and loop structures of a variety of lengths connecting them are reproduced, whereby, the function of GCRDb serving as an information transmitting mechanism can be presumed.

(2) In particular, by using a crystal structure of rhodopsin (PDB ID:1F88) as a reference protein, it becomes possible to remarkably improve the steric structure model in a precision, although only structures having different helix arrangements which are not classified into GCRDb of bacteriorhodopsin could be obtained previously.

(3) The amino acid sequence database “GCRDb” contains only amino acid sequences of GPCRs and, by using alignment software, for example, PSI-BLAST as a motif profile, it is possible to produce a model even when homology is low (20% or less) as compared with PDB:1F88 and, at the same time, suitable alignment is provided regarding arbitrary GPCRs, and an excellent method which does not give unnecessary alignment is provided regarding amino acid sequences other than GPCRs. Previously, it has been said that when homology is reduced to 20% or less, there is a possibility that a structure greatly differs and, therefore, this is a limit of homology modeling (Chochia, C. and Lesk, A. M., EMBO J. 5, 823-826 (1986)). However, a structure of seven helices arrangement of GPCRs obtained by the present method is well conserved, showing that this can be the steric structure model which is reliable in generation of drugs.

(4) In particular, the modeling software “FAMS” developed by the present inventors adopts an algorism for calculating structures similar to those of natural proteins, from proteins having high homology to proteins having low homology, and from a main chain to a side chain of an amino acid and, by performing modeling based on 1F88 which is a detailed steric structure coordinate present in PDB experimentally obtained using this, the reproductivity of a side chain of GPCRs has been remarkably improved.

(5) The database of the present invention contains atomic coordinates defining steric structures of GPCRs obtained from almost all sequences which can be currently obtained, of human, Drosophila, nematode, thearecress, yeast and the like, and proteins having presumed function of GPCRs, and is a database covering steric structures of almost all GPCRs.

As described above, the present method is a method which can construct the steric structure of arbitrary GPCRs with good precision, and the database of the present invention containing atomic coordinates defining the thus obtained GPCRs steric structures is extremely useful for designing, in particular, drug molecules such as medicines, agricultural chemicals and the like.

1. A method for constructing the steric structure of a transmembrane protein, comprising:
selecting a protein having the steric structure to be referred to from the known steric structures of 7-transmembrane G-protein-coupled receptors using a database of amino acid sequences of the receptors as a profile;
performing alignment of a desired protein having an unknown steric structure and, based on the alignment information; and
producing the steric structure of the desired protein having an unknown steric structure as a G-protein-coupled receptor.

2. The method according to claim 1, wherein the performing alignment includes selecting a protein having the steric structure to be referred to from a database of known steric structures of G-protein-coupled receptors, and juxtaposing an amino acid sequence of the selected reference protein and an amino acid sequence of the desired protein.

3. The method according to claim 2, wherein the steric structure to be referred to is the steric structure of rhodopsin (PDB ID:1F88).

4. The method according to any one of claims 1 to 3, wherein the database of amino acid sequences to be used as a profile is selected from the group consisting of GCRDb, GPCRDB, ExPASy and ORDB.

5. The method according to any one of claims 1 to 4, wherein the database of amino acid sequences is used as a motif profile of PSI-BLAST.

6. The method according to claim 5, wherein in the alignment performed by PSI-BLAST, the identity between a final site-specific score matrix and the reference protein are calculated as an E value and the production of the steric structure is performed when the E value has a value of 0.1 or less.

7. The method according to any one of claims 1 to 6, further comprising performing re-alignment is so as to the given alignment information, in view of a combination of a disulfide bond and, the producing of the steric structure is performed based on the re-alignment information.

8. The method according to any one of claims 1 to 7, wherein the producing comprises:
obtaining a coordinate with regard to a Cα atom in an amino acid from the steric structure to be referred to based on the resulting alignment information;
optimizing an atomic coordinate of Cα so as to minimize an objective function;
adding other atoms of a main chain to the optimized atomic coordinate of Cα to optimize an atomic coordinate of the main chain so as to minimize an objective function; and
adding other atoms of a side chain to the optimized atomic coordinate of the main chain to optimize so as to minimize an objective function.

9. An atomic coordinate defining the steric structure of a 7-transmembrane G-protein-coupled receptor obtained by the method according to any one of claims 1 to 8.

10. A computer readable recording medium in which the atomic coordinate according to claim 9 is recorded.

11. A database comprising the atomic coordinate according to claim 9.

12. A method for presuming the function of a protein, comprising:
selecting a protein having the steric structure to be referred to from the known steric structures of 7-transmembrane G-protein-coupled receptors using a database of amino acid sequences of the receptors as a profile;
performing alignment of a desired protein having an unknown function; and
judging that the desired protein is a 7-transmembrane G-protein-coupled receptor when the steric structure of the desired protein can be produced.

13. The method according to claim 12, wherein the performing alignment comprises:
selecting the steric structure to be referred to from a database of the known steric structures of G-protein-coupled receptors;
juxtaposing an amino acid sequence of the selected reference protein and an amino acid sequence of the desired protein, the database of amino acid sequences used as a profile is selected from the group consisting of GCRDb, GPCRDB, ExPASy and ORDB; and
judging that the steric structure of the desired protein can be produced when the alignment has reliability of 98% or more.

14. A method for designing a drug molecule, comprising:
identifying, retrieving, assessing or designing a desired drug molecule based on the interaction between the steric structure of a drug candidate molecule and the steric structure of a 7-transmembrane G-protein-coupled receptor produced by using the atomic coordinate according to claim 9, or the atomic coordinate of the recording medium according to claim 10 or the atomic coordinate of the database according to claim 11.

15. A method for screening a medicine or an agricultural chemical, comprising:
examining the drug molecule obtained by the method according to claim 14 for its activity and safety as a medicine or an agricultural chemical by a desired pharmacological or physiological test; and
selecting a drug molecule having the desired nature.

16. A method for manufacturing a medicine or an agricultural chemical, comprising:
formulating by blending the drug molecule obtained by the method according to claim 15 and a pharmaceutically or agriculturally or horticulturally acceptable carrier.

17. A drug molecule obtained by the method according to claim 14.


SEQUENCE LISTING (12 sequences)

SEQ ID NO: 1 - protein, 9 aa, Bos taurus
YFVFGPTGC

SEQ ID NO: 2 - protein, 28 aa, Bos taurus
GWSRYIPEGMQCSCGIDYYTPHEETNNE

SEQ ID NO: 3 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 4 - protein, 348 aa, Bos taurus
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAAQQQESATTQKAEKEVTRMVIIMVIAFLICWLPYAGVAFYIFTHQGSDFGPIFMTIPAFFAKTSAVYNPVIYIMMNKQFRNCMVTTLCCGKNPLGDDEASTTVSKTETSQVAPA

SEQ ID NO: 5 - protein, 10 aa, Artificial Sequence
  Description of Artificial Sequence Synthetic peptide
GWSRYIPEGM

SEQ ID NO: 6 - protein, 10 aa, Artificial Sequence
  Description of Artificial Sequence Synthetic peptide
QXSCGIDYYT

SEQ ID NO: 7 - protein, 8 aa, Artificial Sequence
  Description of Artificial Sequence Synthetic peptide
PHEETNNE

SEQ ID NO: 8 - protein, 329 aa, Artificial Sequence
  Description of Artificial Sequence Synthetic illustrative protein
FLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQ

SEQ ID NO: 9 - protein, 25 aa, Artificial Sequence
  Description of Artificial Sequence Synthetic illustrative peptide
AKGERQSPVDIDTHTAKYDPSLKPL

SEQ ID NO: 10 - protein, 24 aa, Artificial Sequence
  Description of Artificial Sequence Synthetic illustrative peptide
ARGNREASSPNVDTHSARYDLKPL

SEQ ID NO: 11 - protein, 354 aa, Artificial Sequence
  Description of Artificial Sequence Synthetic illustrative protein
FLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQ

SEQ ID NO: 12 - protein, 332 aa, Artificial Sequence
  Description of Artificial Sequence Synthetic illustrative protein
FSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAAQQQESATTQKAEKEVTRMVIIMVIAFLICWLPYAGVAFYIFTHQGSDFGPIFMTIPAFFAKTSAVYNPVIYIMMNKQFRNCMVTTLCCGKNPLGDDEASTTVSKTETSQ